(19) United States
(12) Patent Application Publication
(10) Pub. No.: US 2016/0151337 A1
(43) Pub. Date: Jun. 2, 2016

(54) ANNELATED PYRROLES AND THEIR USE AS CRAC INHIBITORS

(71) Applicant: Grünenthal GmbH, Aachen (DE)

(72) Inventors: Felix VOSS, Dusseldorf (DE); Stefanie RITTER, Koln (DE); Sonja NORDHOFF, Aachen (DE); Sebastian WACHTEN, Aachen (DE); Stefan OBERBÖRSCH, Aachen (DE); Achim KLESS, Aachen (DE)

(21) Appl. No.: 15/019,238

(22) Filed: Feb. 9, 2016

Related U.S. Application Data
(63) Continuation of application No. PCT/EP2014/002218, filed on Aug. 13, 2014.

(30) Foreign Application Priority Data
Aug. 13, 2013 (EP) 13004028.0

(51) Int. Cl. A61K 31/437; A61K 31/444; A61K 31/5377; A61K 31/497; A61K 31/506; A61K 31/501; C07D 471/04; A61K 31/635

(52) U.S. Cl. CPC A61K 31/437; C07D 471/04; A61K 31/444; A61K 31/635; A61K 31/497; A61K 31/506; A61K 31/501; A61K 31/5377

(57) ABSTRACT

The invention relates to substituted bicyclic pyrroloheterocyclyl compounds useful as ICRAC inhibitors, to pharmaceutical compositions containing these compounds and to these compounds for the use in the treatment and/or prophylaxis of diseases and/or disorders, in particular inflammatory diseases and/or inflammatory disorders.

### FIELD OF THE INVENTION

The invention relates to biologically active compounds, namely substituted annelated pyrroles, useful for inhibition of the Calcium Release Activated Calcium channel (CRAC) and hence for inhibition of the Calcium Release Activated Calcium current (ICRAC), to pharmaceutical compositions containing these compounds and also to these compounds for use in immunosuppression and in the treatment and/or prophylaxis of conditions, diseases and/or disorders, in particular immune disorders, inflammatory conditions and allergic diseases.

### BACKGROUND OF THE INVENTION

Calcium-conducting channels in the plasma membrane can appear very diverse (Parekh & Putney, Physiol Rev 85: 757-810, 2005) including voltage-gated ion channels (VOC's), receptor-operated ion channels (ROC's), but also store-operated channels (SOC's; Putney, Cell Calcium 7: 1-12, 1986) that are activated in response to a decrease of the intraluminal Calcium concentration within i.e. the endoplasmic reticulum (ER). The latter have been demonstrated to serve as the main Calcium entry mechanisms in non-excitable cells.

Amongst the distinct SOCs, the CRAC current (ICRAC) is certainly characterized best and displays biophysical features such as high selectivity for Calcium ions, low conductance, and inward rectification (Hoth & Penner, Nature 355: 253-256, 1992; Hoth & Penner, J Physiol 465: 359-386, 1993; Parekh & Penner, Physiol Rev 77: 901-930, 1997; Lepple-Wienhues & Cahalan, Biophys J 71: 787-794, 1996; Kerschbaum & Cahalan, Science 283: 836-839, 1999). There's substantial evidence that the channels conducting CRAC predominantly rely on two proteins, Orai1 and Stim1 (Roos et al., JCB 169: 435-445, 2005; Feske et al., Nature, 441: 179-185, 2006; Peinelt et al., Nature Cell Biology 8: 771-773, 2006). Orai1 constitutes the channel pore within the plasma membrane (Prakriya et al., Nature, 443: 230-233, 2006; Vig et al. Curr Biol. 16: 2073-2079, 2006), whereas Stim1 has been demonstrated to function as the sensor of the luminal Calcium concentration (Liou et al., Curr Biol. 15: 1235-1241, 2005; Zhang et al. PNAS 103: 9357-9362, 2006).

In a physiological setting, ICRAC is activated in response to the engagement of cell-surface receptors that positively couple to phospholipase C (PLC). PLC increases the concentration of the soluble messenger inositol-1,4,5-trisphosphate (IP3), which opens ER membrane-resident IP3-receptors. Thus, IP3 triggers the release of Calcium from internal stores resulting in a drop of the luminal Calcium concentration (Lewis, Adv. in Second Messenger Phosphoprotein Res 33: 279-307, 1999), which is sensed by Stim1. The Stim1 molecule undergoes conformational changes inducing clustering with other Stim1 molecules just underneath the plasma membrane. At these sites, Stim1 can open the Orai1 pore by bridging the ER-PM gap with its C-terminal tail (Zhang et al., Nature, 437: 902-905, 2005; Luik et al., JCB 174: 815-825, 2006; Soboloff et al., J Biol Chem 281: 20661-20665, 2006, Wu et al., JCB 174: 803-813, 2006; Li et al., J Biol Chem 282: 29448-29456, 2007).

The above described process serves in signaling pathways of immune cells such as lymphocytes and mast cells. I.e. the activation of antigen or Fc receptors stimulates the release of Calcium from intracellular stores, and subsequent activation of ICRAC that impacts on downstream processes such as gene expression and cytokine release (Feske, Nature Reviews 7: 690-702, 2007; Gwack et al., J Biol Chem 282: 16232-16243, 2007; Oh-hora & Rao, Curr Opin Immunol. 20: 250-258, 2008).

The major contribution ICRAC provides to these signaling events has been convincingly demonstrated in patients suffering from severe combined immunodeficiency (SCID) due to a defect in T-cell activation. T cells and fibroblasts from these patients exhibited a strong attenuation of store-operated Calcium entry carried by ICRAC (Feske et al., Nature, 441: 179-185, 2006). This suggests CRAC channel modulators to serve as treatment in disease states caused by activated inflammatory cells.

The activation of antigen or Fc receptors stimulates the release of Calcium from intracellular stores and subsequent, sustained activation of ICRAC. Calcium carried by ICRAC activates calcineurin (CaN), which dephosphorylates the transcription factor NFAT. Upon dephosphorylation, NFAT shuttles into the nucleus and regulates gene expression in various ways depending on the nature of the stimulus as well as on the cell/tissue type.

NFAT participates in the transactivation of cytokine genes that regulate T-cell proliferation and other genes that control immune responses. Taking into account that the expression of cytokines such as IL-2, IL-4, IL-5, IL-8, IL-13, tumor necrosis factor alpha (TNFα), granulocyte colony-stimulating factor (G-CSF), and gamma-interferon (INFγ) is prone to be controlled via transcriptional elements for NFAT, the impact of the ICRAC/CaN/NFAT signaling pathway on pro-inflammatory processes becomes apparent. The inhibition of this pathway has been demonstrated to be efficacious in patients by the use of drugs such as CsA and FK506, which act by inhibiting CaN.

A hallmark of ICRAC signaling in immune cells is that downstream processes such as gene expression rely on sustained Calcium entry rather than transient signals. However, Calcium entry is essential for other processes that can be independent of CaN/NFAT. Direct, Calcium-mediated release of substances (degranulation) such as histamine, heparin, and TNFα occur in i.e. mast cells, and are of rather acute nature. On the molecular level, this already points towards a differentiation potential for ICRAC blockers from calcineurin inhibitors.

Recent findings suggest that CRAC channel modulators can serve as treatment in disease states caused by the activation of inflammatory cells without side effects observed under treatments with i.e. steroids. Such diseases may include but are not limited to asthma, chronic obstructive pulmonary disease, rheumatoid arthritis, inflammatory bowel disease, glomerulonephritis, neuroinflammatory diseases such as multiple sclerosis, and disorders of the immune system.

Several compounds have been reported in the art as CRAC channel modulators. For instance, certain substituted biaryl compounds (WO 2007/087441 A2), pyrazole carboxamide derivatives (e.g. WO 2009/089305 A1 and WO 2010/122089 A1), thiophene derivatives (e.g. WO 2009/076454 A2 and WO 2009/035818 A1), indole derivatives (WO 2011/036130 A1), aza-indole derivatives (e.g. WO 2013/092467 A1, WO 2013/092444 A1 and WO 2013/092463 A1), aza-benzoxazines (WO 2013/050341 A1) and benzoxazines (WO 2013/050270 A1) have been disclosed for modulating CRAC channel activity. There is no teaching in the art that specifically substituted bicyclic compounds, bearing a pyrrole annelated with a saturated aza-heterocyclic system, may be suitable to modulate CRAC channel activity.

Annelated pyrroles, such as pyrrolopiperidines, are known as biologically active compounds from WO 2007/136603 A2 or from WO 2003/027114 A1.

### SUMMARY OF THE INVENTION

The present invention describes a new class of small molecule that is useful for the inhibition of the calcium release activated calcium channel current (thereafter ICRAC inhibitors).

It was therefore an object of the invention to provide novel compounds, preferably having advantages over the prior-art compounds. The compounds should be suitable in particular as pharmacological active ingredients in pharmaceutical compositions, preferably in pharmaceutical compositions for the treatment and/or prophylaxis of disorders or diseases which are at least partially mediated by CRAC channels.

This object is achieved by the subject matter described herein.

It has surprisingly been found that the substituted compounds of general formula (I), as given below, display potent inhibitory activity against to CRAC channels and are therefore particularly suitable for the prophylaxis and/or treatment of disorders or diseases which are at least partially mediated by CRAC channels.

A first aspect of the present invention therefore relates to a compound of general formula (I),

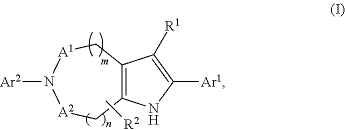

wherein
A¹ and A² represent direct bond or C(═O), with the proviso that 0 or 1 of A¹ and A² represents C(═O);
m and n independently denote 0, 1, 2 or 3, with the proviso that the sum [n+m] is 1, 2, 3 or 4;
R¹ denotes H, F, Cl, Br, I, CN, CF₃, CF₂H, CFH₂, CO₂H, CO₂R¹³, R¹³, OH, O—R¹³, NH₂, N(H)R¹³, N(R¹³)₂,
R² represents 0 to 4 substituents, each independently selected from F, Cl, Br, CN, CF₃, CF₂H, CFH₂, R¹³, OH, O—R¹³, NH₂, N(H)R¹³ and N(R¹³)₂;
Ar¹ represents phenyl or 5- or 6-membered heteroaryl, in each case unsubstituted or substituted with one, two, three or four substituents, independently selected from F, Cl, Br, CN, CF₃, CF₂H, CFH₂, R¹³ and O—R¹³;
   or
   C₃₋₆-cycloalkyl or 3 to 7 membered heterocycloalkyl, in each case unsubstituted or mono- or polysubstituted;
Ar² represents phenyl or 5- or 6-membered heteroaryl, wherein said phenyl or said heteroaryl may be unsubstituted or mono- or polysubstituted and may be condensed with a 4-, 5-, 6- or 7-membered ring, being carbocyclic or heterocyclic, wherein said condensed ring may be saturated, partially unsaturated or aromatic and may be unsubstituted or mono- or polysubstituted;
and
each R¹³ independently of each other denotes
   C₁₋₈-alkyl, unsubstituted or mono- or polysubstituted;
   or
   C₃₋₆-cycloalkyl or 3 to 7 membered heterocycloalkyl, in each case unsubstituted or mono- or polysubstituted;
   or
   C₃₋₆-cycloalkyl or 3 to 7 membered heterocycloalkyl, in each case unsubstituted or mono- or polysubstituted, and in each case connected via a C₁₋₄-aliphatic group, unsubstituted or mono- or polysubstituted;
      optionally in the form of a single stereoisomer or a mixture of stereoisomers, in the form of the free compound or a physiologically acceptable salt thereof or a physiologically acceptable solvate thereof;
      in which “mono- or polysubstituted” with respect to “alkyl”, “aliphatic group”, “cycloalkyl” and “heterocycloalkyl” relates in each case independently of one another, with respect to the corresponding residues or groups, to the substitution of one or more hydrogen atoms each independently of one another by at least one substituent selected from the group consisting of F; Cl; CN; CF₃; CF₂H; CFH₂; CF₂Cl; CFCl₂; C₁₋₈-alkyl; C₃₋₆-cycloalkyl; 3 to 7 membered heterocycloalkyl; aryl; heteroaryl; aryl, heteroaryl, C₃₋₆-cycloalkyl or 3 to 6 membered heterocycloalkyl, each connected via a C₁₋₄-aliphatic group; C(═O)—(C₁₋₈-alkyl); C(═O)—(C₃₋₆-cycloalkyl); C(═O)-(3 to 7 membered heterocycloalkyl); C(═O)-(aryl); C(═O)-(heteroaryl); C(═O)OH; C(═O)—O(C₁₋₈-alkyl); C(═O)—O(C₃₋₆-cycloalkyl); C(═O)—O(3 to 7 membered heterocycloalkyl); C(═O)—O(aryl); C(═O)—O(heteroaryl); C(═O)—NH₂; C(═O)—N(H)(C₁₋₈-alkyl); C(═O)—N(H)(C₃₋₆-cycloalkyl); C(═O)—N(H)(3 to 7 membered heterocycloalkyl); C(═O)—N(H)(aryl); C(═O)—N(H)(heteroaryl); C(═O)—N(C₁₋₈-alkyl)(C₁₋₈-alkyl); C(═O)—N(C₁₋₈-alkyl)(C₃₋₆-cycloalkyl); C(═O)—N(C₁₋₈-alkyl)(3 to 7 membered heterocycloalkyl); C(═O)—N(C₁₋₈-alkyl)(aryl); C(═O)—N(C₁₋₈-alkyl)(heteroaryl); OH; ═O; O—(C₁₋₈-alkyl); O—(C₃₋₆-cycloalkyl); O-(3 to 7 membered heterocycloalkyl); O-(aryl); O-(heteroaryl); OCF₃; OCF₂H; OCFH₂; OCF₂Cl; OCFCl₂; O—(C₂₋₄-alkyl)-OH; O—(C₂₋₄-alkyl)-O(C₁₋₈-alkyl); O—C(═O)—(C₁₋₈-alkyl); O—C(═O)—(C₃₋₆-cycloalkyl); O—C(═O)-(3 to 7 membered heterocycloalkyl); O—C(═O)-(aryl); C(═O)-(heteroaryl); O—C(═O)—NH₂; O—C(═O)—N(H)(C₁₋₈-alkyl); O—C(═O)—N(H)(C₃₋₆-cycloalkyl); O—C(═O)—N(H)(3 to 7 membered heterocycloalkyl); O—C(═O)—N(H)(aryl); O—C(═O)—N(H)(heteroaryl); O—C(═O)—N(C₁₋₈-alkyl)(C₁₋₈-alkyl); O—C(═O)—N(C₁₋₈-alkyl)(C₃₋₆-cycloalkyl); O—C(═O)—N(C₁₋₈-alkyl)(3 to 7 membered heterocycloalkyl); O—C(═O)—N(C₁₋₈-alkyl)(aryl); O—C(═O)—N(C₁₋₈-alkyl)(heteroaryl); NH₂; N(H)(C₁₋₈-alkyl); N(H)(C₃₋₈-cycloalkyl); N(H)(3 to 7 membered heterocycloalkyl); N(H)(aryl); N(H)(heteroaryl); N(C₁₋₈-alkyl)(C₁₋₈-alkyl); N(C₁₋₈-alkyl)(C₃₋₈-cycloalkyl); N(C₁₋₈-alkyl)(3 to 7 membered heterocycloalkyl); N(C₁₋₈-alkyl)(aryl); N(C₁₋₈-alkyl)(heteroaryl); N(H)—C(═O)—(C₁₋₈-alkyl); N(H)—C(═O)—(C₃₋₈-cycloalkyl); N(H)—C(═O)-(3 to 7 membered heterocycloalkyl); N(H)—C(═O)-(aryl); N(H)—C(═O)-(heteroaryl); N(C₁₋₈-alkyl)-C(═O)—(C₁₋₈-alkyl); N(C₁₋₈-alkyl)-C(═O)—(C₃₋₈-cycloalkyl); N(C₁₋₈-alkyl)-C(═O)-(3 to 7 membered heterocycloalkyl); N(C₁₋₈-alkyl)-C(═O)-(aryl); N(C₁₋₈-alkyl)-C(═O)-(heteroaryl); N(H)—S(═O)₂—(C₁₋₈-alkyl); N(H)—S(═O)₂—(C₃₋₈-cycloalkyl); N(H)—S(═O)₂-(3 to 7 membered heterocycloalkyl); N(H)—S(═O)₂-(aryl); N(H)—S(═O)₂-(heteroaryl); N(C₁₋₈-alkyl)-S(═O)₂—(C₁₋₈-alkyl); N(C₁₋₈-alkyl)-S(═O)₂—(C₃₋₈-cycloalkyl); N(C₁₋₈-alkyl)-S(═O)₂-(3 to 7 membered heterocycloalkyl); N(C₁₋₈-alkyl)-S(═O)₂-(aryl); N(C₁₋₈-alkyl)-S(═O)₂-(heteroaryl); N(H)—C(═O)—O(C₁₋₈-alkyl); N(H)—C(═O)—O(C₃₋₈-cycloalkyl); N(H)—C(═O)—O(3 to 7 membered heterocycloalkyl); N(H)—C(═O)—O(aryl); N(H)—C(═O)—O(heteroaryl); N(C₁₋₈-alkyl)-C(═O)—O(C₁₋₈-alkyl); N(C₁₋₈-alkyl)-C(═O)—O(C₃₋₈-cycloalkyl); N(C₁₋₈-alkyl)-C(═O)—O(3 to 7 membered heterocycloalkyl); N(C₁₋₈-alkyl)-C(═O)—O(aryl); N(C₁₋₈-alkyl)-C(═O)—O(heteroaryl); N(H)—C(═O)—NH₂; N(H)—C(═O)—N(H)(C₁₋₈-alkyl); N(H)—C(═O)—N(H)(C₃₋₈-cycloalkyl); N(H)—C(═O)—N(H)(3 to 7 membered heterocycloalkyl); N(H)—C(═O)—N(H)(aryl); N(H)—C(═O)—N(H)(heteroaryl); N(C₁₋₈-alkyl)-C(═O)—NH₂; N(C₁₋₈-alkyl)-C(═O)—N(H)(C₁₋₈-alkyl); N(C₁₋₈-alkyl)-C(═O)—N(H)(C₃₋₈-cycloalkyl); N(C₁₋₈-alkyl)-C(═O)—N(H)(3 to 7 membered heterocycloalkyl); N(C₁₋₈-alkyl)-C(═O)—N(H)(aryl); N(C₁₋₈-alkyl)-C(═O)—N(H)(heteroaryl); N(H)—C(═O)—N(C₁₋₈-alkyl)(C₁₋₈-alkyl); N(H)—C(═O)—N(C₁₋₈-alkyl)(C₃₋₈-cycloalkyl); N(H)—C(═O)—N(C₁₋₈-alkyl)(3 to 7 membered heterocycloalkyl); N(H)—C(═O)—N(C₁₋₈-alkyl)(aryl); N(H)—C(═O)—N(C₁₋₈-alkyl)(heteroaryl); N(C₁₋₈-alkyl)-C(═O)—N(C₁₋₈-alkyl)(C₁₋₈-alkyl); N(C₁₋₈-alkyl)-C(═O)—N(C₁₋₈-alkyl)(C₃₋₈-cycloalkyl); N(C₁₋₈-alkyl)-C(═O)—N(C₁₋₈-alkyl)(3 to 7 membered heterocycloalkyl); N(C₁₋₈-alkyl)-C(═O)—N(C₁₋₈-alkyl)(aryl); N(C₁₋₈-alkyl)-C(═O)—N(C₁₋₈-alkyl)(heteroaryl); S—(C₃₋₈-cycloalkyl); S-(3 to 7 membered heterocycloalkyl); S-(aryl); S-(heteroaryl); SCF₃; S(═O)₂OH; S(═O)—(C₁₋₈-alkyl); S(═O)—(C₃₋₈-cycloalkyl); S(═O)-(3 to 7 membered heterocycloalkyl); S(═O)-(aryl); S(═O)-(heteroaryl); S(═O)₂—(C₁₋₈-alkyl); S(═O)₂—(C₃₋₈-cycloalkyl); S(═O)₂-(3 to 7 membered heterocycloalkyl); S(═O)₂-(aryl); S(═O)₂-(heteroaryl); S(═O)₂—O(C₁₋₈-alkyl); S(═O)₂—O(C₃₋₈-cycloalkyl); S(═O)₂—O(3 to 7 membered heterocycloalkyl); S(═O)₂—O(aryl); S(═O)₂—O(heteroaryl); S(═O)₂—N(H)(C₁₋₈-alkyl); S(═O)₂—N(H)(C₃₋₈-cycloalkyl); S(═O)₂—N(H)(3 to 7 membered heterocycloalkyl); S(═O)₂—N(H)(aryl); S(═O)₂—N(H)(heteroaryl); S(═O)₂—N(C₁₋₈-alkyl)(C₁₋₈-alkyl); S(═O)₂—N(C₁₋₈-alkyl)(C₃₋₈-cycloalkyl); S(═O)₂—N(C₁₋₈-alkyl)(3 to 7 membered heterocycloalkyl); S(═O)₂—N(C₁₋₈-alkyl)(aryl); S(═O)₂—N(C₁₋₈-alkyl)(heteroaryl);
      in which “mono- or polysubstituted” with respect to “aryl” and “heteroaryl” relates, with respect to the corresponding residues, in each case independently of one another, to the substitution of one or more hydrogen atoms each independently of one another by at least one substituent selected from the group consisting of F; Cl; Br; NO₂; CN; CF₃; CF₂H; CFH₂; CF₂Cl; CFCl₂; C₁₋₈-alkyl; C₃₋₈-cycloalkyl; 3 to 7 membered heterocycloalkyl; aryl; heteroaryl; aryl, heteroaryl, C₃₋₈-cycloalkyl or 3 to 6 membered heterocycloaliphatic, each connected via a C₁₋₄-aliphatic group; C(═O)H; C(═O)—(C₁₋₈-alkyl); C(═O)—(C₃₋₈-cycloalkyl); C(═O)-(3 to 7 membered heterocycloalkyl); C(═O)-(aryl); C(═O)-(heteroaryl); C(═O)OH; C(═O)—O(C₁₋₈-alkyl); C(═O)—O(C₃₋₈-cycloalkyl); C(═O)—O(3 to 7 membered heterocycloalkyl); C(═O)—O(aryl); C(═O)—O(heteroaryl); C(═O)—NH₂; C(═O)—N(H)(C₁₋₈-alkyl); C(═O)—N(H)(C₃₋₈-cycloalkyl); C(═O)—N(H)(3 to 7 membered heterocycloalkyl); C(═O)—N(H)(aryl); C(═O)—N(H)(heteroaryl); C(═O)—N(C₁₋₈-alkyl)(C₁₋₈-alkyl); C(═O)—N(C₁₋₈-alkyl)(C₃₋₈-cycloalkyl); C(═O)—N(C₁₋₈-alkyl)(3 to 7 membered heterocycloalkyl); C(═O)—N(C₁₋₈-alkyl)(aryl); C(═O)—N(C₁₋₈-alkyl)(heteroaryl); OH; O—(C₁₋₈-alkyl); O—(C₃₋₈-cycloalkyl); O-(3 to 7 membered heterocycloalkyl); O-(aryl); O-(heteroaryl); OCF₃; OCF₂H; OCFH₂; OCF₂Cl; OCFCl₂; O—(C₂₋₄-alkyl)-OH; O—(C₂₋₄-alkyl)-O(C₁₋₈-alkyl); O—C(═O)—(C₁₋₈-alkyl); O—C(═O)—(C₃₋₈-cycloalkyl); O—C(═O)-(3 to 7 membered heterocycloalkyl); O—C(═O)-(aryl); C(═O)-(heteroaryl); O—C(═O)—NH₂; O—C(═O)—N(H)(C₁₋₈-alkyl); O—C(═O)—N(H)(C₃₋₈-cycloalkyl); O—C(═O)—N(H)(3 to 7 membered heterocycloalkyl); O—C(═O)—N(H)(aryl); O—C(═O)—N(H)(heteroaryl); O—C(═O)—N(C₁₋₈-alkyl)(C₁₋₈-alkyl); O—C(═O)—N(C₁₋₈-alkyl)(C₃₋₈-cycloalkyl); O—C(═O)—N(C₁₋₈-alkyl)(3 to 7 membered heterocycloalkyl); O—C(═O)—N(C₁₋₈-alkyl)(aryl); O—C(═O)—N(C₁₋₈-alkyl)(heteroaryl); NH₂; N(H)(C₁₋₈-alkyl); N(H)(C₃₋₈-cycloalkyl); N(H)(3 to 7 membered heterocycloalkyl); N(H)(aryl); N(H)(heteroaryl); N(C₁₋₈-alkyl)(C₁₋₈-alkyl); N(C₁₋₈-alkyl)(C₃₋₈-cycloalkyl); N(C₁₋₈-alkyl)(3 to 7 membered heterocycloalkyl); N(C₁₋₈-alkyl)(aryl); N(C₁₋₈-alkyl)(heteroaryl); N(H)—C(═O)—(C₁₋₈-alkyl); N(H)—C(═O)—(C₃₋₈-cycloalkyl); N(H)—C(═O)-(3 to 7 membered heterocycloalkyl); N(H)—C(═O)-(aryl); N(H)—C(═O)-(heteroaryl); N(C₁₋₈-alkyl)-C(═O)—(C₁₋₈-alkyl); N(C₁₋₈-alkyl)-C(═O)—(C₃₋₈-cycloalkyl); N(C₁₋₈-alkyl)-C(═O)-(3 to 7 membered heterocycloalkyl); N(C₁₋₈-alkyl)-C(═O)-(aryl); N(C₁₋₈-alkyl)-C(═O)-(heteroaryl); N(H)—S(═O)₂—(C₁₋₈-alkyl); N(H)—S(═O)₂—(C₃₋₈-cycloalkyl); N(H)—S(═O)₂-(3 to 7 membered heterocycloalkyl); N(H)—S(═O)₂-(aryl); N(H)—S(═O)₂-(heteroaryl); N(C₁₋₈-alkyl)-S(═O)₂—(C₁₋₈-alkyl); N(C₁₋₈-alkyl)-S(═O)₂—(C₃₋₈-cycloalkyl); N(C₁₋₈-alkyl)-S(═O)₂-(3 to 7 membered heterocycloalkyl); N(C₁₋₈-alkyl)-S(═O)₂-(aryl); N(C₁₋₈-alkyl)-S(═O)₂-(heteroaryl); N(H)—C(═O)—O(C₁₋₈-alkyl); N(H)—C(═O)—O(C₃₋₈-cycloalkyl); N(H)—C(═O)—O(3 to 7 membered heterocycloalkyl); N(H)—C(═O)—O(aryl); N(H)—C(═O)—O(heteroaryl); N(C₁₋₈-alkyl)-C(═O)—O(C₁₋₈-alkyl); N(C₁₋₈-alkyl)-C(═O)—O(C₃₋₈-cycloalkyl); N(C₁₋₈-alkyl)-C(═O)—O(3 to 7 membered heterocycloalkyl); N(C₁₋₈-alkyl)-C(═O)—O(aryl); N(C₁₋₈-alkyl)-C(═O)—O(heteroaryl); N(H)—C(═O)—NH₂; N(H)—C(═O)—N(H)(C₁₋₈-alkyl); N(H)—C(═O)—N(H)(C₃₋₈-cycloalkyl); N(H)—C(═O)—N(H)(3 to 7 membered heterocycloalkyl); N(H)—C(═O)—N(H)(aryl); N(H)—C(═O)—N(H)(heteroaryl); N(C₁₋₈-alkyl)-C(═O)—NH₂; N(C₁₋₈-alkyl)-C(═O)—N(H)(C₁₋₈-alkyl); N(C₁₋₈-alkyl)-C(═O)—N(H)(C₃₋₈-cycloalkyl); N(C₁₋₈-alkyl)-C(═O)—N(H)(3 to 7 membered heterocycloalkyl); N(C₁₋₈-alkyl)-C(═O)—N(H)(aryl); N(C₁₋₈-alkyl)-C(═O)—N(H)(heteroaryl); N(H)—C(═O)—N(C₁₋₈-alkyl)(C₁₋₈-alkyl); N(H)—C(═O)—N(C₁₋₈-alkyl)(C₃₋₈-cycloalkyl); N(H)—C(═O)—N(C₁₋₈-alkyl)(3 to 7 membered heterocycloalkyl); N(H)—C(═O)—N(C₁₋₈-alkyl)(aryl); N(H)—C(═O)—N(C₁₋₈-alkyl)(heteroaryl); N(C₁₋₈-alkyl)-C(═O)—N(C₁₋₈-alkyl)(C₁₋₈-alkyl); N(C₁₋₈-alkyl)-C(═O)—N(C₁₋₈-alkyl)(C₃₋₈-cycloalkyl); N(C₁₋₈-alkyl)-C(═O)—N(C₁₋₈-alkyl)(3 to 7 membered heterocycloalkyl); N(C₁₋₈-alkyl)-C(═O)—N(C₁₋₈-alkyl)(aryl); N(C₁₋₈-alkyl)-C(═O)—N(C₁₋₈-alkyl) heteroaryl); SH; S—(C₁₋₈-alkyl); S—(C₃₋₈-cycloalkyl); S-(3 to 7 membered heterocycloalkyl); S-(aryl); S-(heteroaryl); SCF₃; S(═O)₂OH; S(═O)—(C₁₋₈-alkyl); S(═O)—(C₃₋₈-cycloalkyl); S(═O)-(3 to 7 membered heterocycloalkyl); S(═O)-(aryl); S(═O)-(heteroaryl); S(═O)₂—(C₁₋₈-alkyl); S(═O)₂—(C₃₋₈-cycloalkyl); S(═O)₂-(3 to 7 membered heterocycloalkyl); S(═O)₂-(aryl); S(═O)₂-(heteroaryl); S(═O)₂—O(C₁₋₈-alkyl); S(═O)₂—O(C₃₋₈-cycloalkyl); S(═O)₂—O(3 to 7 membered heterocycloalkyl); S(═O)₂—O(aryl); S(═O)₂—O(heteroaryl); S(═O)₂—N(H)(C₁₋₈-alkyl); S(═O)₂—N(H)(C₃₋₈-cycloalkyl); S(═O)₂—N(H)(3 to 7 membered heterocycloalkyl); S(═O)₂—N(H)(aryl); S(═O)₂—N(H)(heteroaryl); S(═O)₂—N(C₁₋₈-alkyl)(C₁₋₈-alkyl); S(═O)₂—N(C₁₋₈-alkyl)(C₃₋₈-cycloalkyl); S(═O)₂—N(C₁₋₈-alkyl)(3 to 7 membered heterocycloalkyl); S(═O)₂—N(C₁₋₈-alkyl)(aryl); S(═O)₂—N(C₁₋₈-alkyl) heteroaryl).

### DETAILED DESCRIPTION

The term “single stereoisomer” preferably means in the sense of the present invention an individual enantiomer or diastereomer. The term “mixture of stereoisomers” means in the sense of this invention the racemate and mixtures of enantiomers and/or diastereomers in any mixing ratio.

The term “physiologically acceptable salt” preferably comprises in the sense of this invention a salt of at least one compound according to the present invention and at least one physiologically acceptable acid or base.

A physiologically acceptable salt of at least one compound according to the present invention and at least one physiologically acceptable acid or one physiologically acceptable base preferably refers in the sense of this invention to a salt of at least one compound according to the present invention with at least one inorganic or organic acid or with at least one inorganic or organic base respectively which is physiologically acceptable—in particular when used in human beings and/or other mammals.

The term “physiologically acceptable solvate” preferably comprises in the sense of this invention an adduct of one compound according to the present invention and/or a physiologically acceptable salt of at least one compound according to the present invention with distinct molecular equivalents of one solvent or more solvents.

The terms “C₁₋₈-alkyl”, “C₂₋₄-alkyl” and “C₁₋₄-alkyl” comprise in the sense of this invention acyclic saturated or unsaturated aliphatic hydrocarbon residues, which can be branched or unbranched and also unsubstituted or mono- or polysubstituted, which contain 1 to 8 or 2 to 4 or 1 to 4 carbon atoms respectively, i.e. C₁₋₈-alkanyls, C₂₋₈-alkenyls and C₂₋₈-alkynyls as well as C₁₋₄-alkanyls, C₂₋₄-alkanyls, C₂₋₄-alkenyls and C₂₋₄-alkynyls, respectively. Alkenyls comprise at least one C—C-double bond (a C═C-bond) and alkynyls comprise at least one C—C triple bond (a C≡C-bond). Preferably, alkyls are selected from the group consisting of alkanyl and alkenyl residues, more preferably are alkanyl residues. Hence, preferred “C₁₋₈-alkyl” is “C₁₋₈-alkanyl” and preferred “C₁₋₄-alkyl” is “C₁₋₄-alkanyl”. Preferred C₁₋₈-alkyl residues are selected from the group consisting of methyl, ethyl, n-propyl, 2-propyl, n-butyl, isobutyl, sec.-butyl, tert.-butyl, n-pentyl, isopentyl, neopentyl, n-hexyl, n-heptyl and n-octyl. Preferred C₁₋₄-alkyl residues are selected from the group consisting of methyl, ethyl, n-propyl, 2-propyl, n-butyl, isobutyl, sec.-butyl and tert.-butyl.

The term “C₃₋₆-cycloalkyl” means for the purposes of this invention cyclic aliphatic hydrocarbons containing 3, 4, 5 or 6 carbon atoms, wherein the hydrocarbons in each case can be saturated or unsaturated (but not aromatic), unsubstituted or mono- or polysubstituted. The C₃₋₆-cycloalkyl can be bound to the respective superordinate general structure via any desired and possible ring member of the cycloalkyl. The C₃₋₆-cycloalkyl can also be condensed with further saturated, (partially) unsaturated, (hetero)cyclic, aromatic or heteroaromatic ring systems, i.e. with cycloalkyl, heterocycloalkyl, aryl or heteroaryl residues, which in each case can in turn be unsubstituted or mono- or polysubstituted. A preferred C₃₋₆-cycloalkyl is selected from the group consisting of cyclopropyl, cyclobutyl, cyclopentyl, cyclohexyl, cyclopentenyl and cyclohexenyl, in particular cyclopropyl.

The terms “3 to 7 membered heterocycloalkyl” or “3-7-membered heterocycloalkyl”, mean for the purposes of this invention heterocycloaliphatic saturated or unsaturated (but not aromatic) residues having 3 to 7, i.e. 3, 4, 5, 6 or 7 ring members, in which in each case at least one, if appropriate also two, three or four carbon atoms are replaced by a heteroatom or a heteroatom group each selected independently of one another from the group consisting of O, S, S(═O), S(═O)₂, N, NH and N(C₁₋₆-alkyl) such as N(CH₃), wherein the ring members can be unsubstituted or mono- or polysubstituted. The 3 to 7 membered heterocycloalkyl can also be condensed with further saturated, (partially) unsaturated, (hetero)cyclic, aromatic or heteroaromatic ring systems, i.e. with cycloalkyl, heterocycloalkyl, aryl or heteroaryl residues, which in each case can in turn be unsubstituted or mono- or polysubstituted. The heterocycloalkyl can be bound to the superordinate general structure via any desired and possible ring member of the heterocycloalkyl if not indicated otherwise.

The term “aryl” means for the purpose of this invention aromatic hydrocarbons containing 6 to 14 carbon atoms. Each aryl residue can be unsubstituted or mono- or polysubstituted, wherein the aryl substituents can be the same or different and in any desired and possible position of the aryl. The aryl can be bound to the superordinate general structure via any desired and possible ring member of the aryl residue. The aryl residues can also be condensed with further saturated, (partially) unsaturated, (hetero)cyclic, aromatic or heteroaromatic ring systems, i.e. with a cycloalkyl, heterocycloalkyl, aryl or heteroaryl residue, which can in turn be unsubstituted or mono- or polysubstituted. Examples of condensed aryl residues are benzodioxolanyl and benzodioxanyl. Preferably, aryl is selected from the group consisting of phenyl, 1-naphthyl, 2-naphthyl, fluorenyl and anthracenyl, each of which can be respectively unsubstituted or mono- or polysubstituted. A particularly preferred aryl is phenyl, unsubstituted or mono- or polysubstituted.

The term “heteroaryl” for the purpose of this invention represents a 5- or 6-membered cyclic aromatic residue containing at least 1, if appropriate also 2, 3, 4 or 5 heteroatoms, wherein the heteroatoms are each selected independently of one another from the group S, N and O and the heteroaryl residue can be unsubstituted or mono- or polysubstituted; in the case of substitution on the heteroaryl, the substituents can be the same or different and be in any desired and possible position of the heteroaryl. The binding to the superordinate general structure can be carried out via any desired and possible ring member of the heteroaryl residue if not indicated otherwise. The heteroaryl can also be part of a bi- or polycyclic system having up to 14 ring members, wherein the ring system can be formed with further saturated, (partially) unsaturated, (hetero)cyclic or aromatic or heteroaromatic rings, i.e. with cycloalkyl, heterocycloalkyl, aryl or heteroaryl residues, which can in turn be unsubstituted or mono- or polysubstituted. It is preferable for the heteroaryl residue to be selected from the group consisting of benzofuranyl, benzoimidazolyl, benzo-thienyl, benzothiadiazolyl, benzothiazolyl, benzotriazolyl, benzooxazolyl, benzooxadiazolyl, quinazolinyl, quinoxalinyl, carbazolyl, quinolinyl, dibenzofuranyl, dibenzothienyl, furyl (furanyl), imidazolyl, imidazo-thiazolyl, indazolyl, indolizinyl, indolyl, isoquinolinyl, isoxazoyl, isothiazolyl, indolyl, naphthyridinyl, oxazolyl, oxadiazolyl, phenazinyl, phenothiazinyl, phthalazinyl, pyrazolyl, pyridyl (2-pyridyl, 3-pyridyl, 4-pyridyl), pyrrolyl, pyridazinyl, pyrimidinyl, pyrazinyl, purinyl, phenazinyl, thienyl (thiophenyl), triazolyl, tetrazolyl, thiazolyl, thiadiazolyl and triazinyl.

The term “connected via a C₁₋₄-aliphatic group” with respect to aryl, heteroaryl, heterocycloalkyl and cycloalkyl mean for the purpose of the invention that these residues have the above-defined meanings and that each of these residues is bound to the respective superordinate general structure via a C₁₋₄-aliphatic group. The C₁₋₄-aliphatic group can in all cases be branched or unbranched, unsubstituted or mono- or polysubstituted. The C₁₋₄-aliphatic group can in all cases be furthermore saturated or unsaturated, i.e. can be a C₁₋₄-alkylene group, a C₂₋₄-alkenylene group or a C₂₋₄-alkynylene group. Preferably, the C₁₋₄-aliphatic group is a C₁₋₄-alkylene group or a C₂₋₄-alkenylene group, more preferably a C₁₋₄-alkylene group. Preferred C₁₋₄-alkylene groups are methylene, 1,1-ethylene and 1,2-ethylene.

In relation to the terms “alkyl” and “aliphatic group”, in particular “alkyl” and “alkylene”, as well as “cycloalkyl” and “heterocycloalkyl”, the term “mono- or polysubstituted” refers in the sense of this invention, with respect to the corresponding residues or groups, to the single substitution or multiple substitution, e.g. disubstitution, trisubstitution, tetrasubstitution, or pentasubstitution, of one or more hydrogen atoms each independently of one another by at least one substituent selected from the group consisting of F; Cl; CN; CF₃; CF₂H; CFH₂; CF₂Cl; CFCl₂; C₁₋₈-alkyl; C₃₋₆-cycloalkyl; 3 to 7 membered heterocycloalkyl; aryl; heteroaryl; aryl, heteroaryl, C₃₋₆-cycloalkyl or 3 to 7 membered heterocycloalkyl each connected via a C₁₋₄-aliphatic group; C(═O)—(C₁₋₈-alkyl); C(═O)—(C₃₋₆-cycloalkyl); C(═O)-(3 to 7 membered heterocycloalkyl); C(═O)-(aryl); C(═O)-(heteroaryl); C(═O)OH; C(═O)—O(C₁₋₈-alkyl); C(═O)—O(C₃₋₆-cycloalkyl); C(═O)—O(3 to 7 membered heterocycloalkyl); C(═O)—O(aryl); C(═O)—O(heteroaryl); C(═O)—NH₂; C(═O)—N(H)(C₁₋₈-alkyl); C(═O)—N(H)(C₃₋₆-cycloalkyl); C(═O)—N(H)(3 to 7 membered heterocycloalkyl); C(═O)—N(H)(aryl); C(═O)—N(H)(heteroaryl); C(═O)—N(C₁₋₈-alkyl)(C₁₋₈-alkyl); C(═O)—N(C₁₋₈-alkyl)(C₃₋₆-cycloalkyl); C(═O)—N(C₁₋₈-alkyl) (3 to 7 membered heterocycloalkyl); C(═O)—N(C₁₋₈-alkyl)(aryl); C(═O)—N(C₁₋₈-alkyl)(heteroaryl); OH; ═O; O—(C₁₋₈-alkyl); O—(C₃₋₆-cycloalkyl); O-(3 to 7 membered heterocycloalkyl); O-(aryl); O-(heteroaryl); OCF₃; OCF₂H; OCFH₂; OCF₂Cl; OCFCl₂; O—(C₂₋₄-alkyl)-OH; O—(C₂₋₄-alkyl)-O(C₁₋₈-alkyl); O—C(═O)—(C₁₋₈-alkyl); O—C(═O)—(C₃₋₆-cycloalkyl); O—C(═O)-(3 to 7 membered heterocycloalkyl); O—C(═O)-(aryl); C(═O)-(heteroaryl); O—C(═O)—NH₂; O—C(═O)—N(H)(C₁₋₈-alkyl); O—C(═O)—N(H)(C₃₋₆-cycloalkyl); O—C(═O)—N(H)(3 to 7 membered heterocycloalkyl); O—C(═O)—N(H)(aryl); O—C(═O)—N(H)(heteroaryl); O—C(═O)—N(C₁₋₈-alkyl)(C₁₋₈-alkyl); O—C(═O)—N(C₁₋₈-alkyl)(C₃₋₆-cycloalkyl); O—C(═O)—N(C₁₋₈-alkyl)(3 to 7 membered heterocycloalkyl); O—C(═O)—N(C₁₋₈-alkyl)(aryl); O—C(═O)—N(C₁₋₈-alkyl)(heteroaryl); NH₂; N(H)(C₁₋₈-alkyl); N(H)(C₃₋₆-cycloalkyl); N(H)(3 to 7 membered heterocycloalkyl); N(H)(aryl); N(H)(heteroaryl); N(C₁₋₈-alkyl)(C₁₋₈-alkyl); N(C₁₋₈-alkyl)(C₃₋₆-cycloalkyl); N(C₁₋₈-alkyl)(3 to 7 membered heterocycloalkyl); N(C₁₋₈-alkyl)(aryl); N(C₁₋₈-alkyl)(heteroaryl); N(H)—C(═O)—(C₁₋₈-alkyl); N(H)—O(═O)—(C₃₋₆-cycloalkyl); N(H)—C(═O)-(3 to 7 membered heterocycloalkyl); N(H)—C(═O)-(aryl); N(H)—C(═O)-(heteroaryl); N(C₁₋₈-alkyl)-O(═O)—(C₁₋₈-alkyl); N(C₁₋₈-alkyl)-O(═O)—(C₃₋₆-cycloalkyl); N(C₁₋₈-alkyl)-O(═O)-(3 to 7 membered heterocycloalkyl); N(C₁₋₈-alkyl)-O(═O)-(aryl); N(C₁₋₈-alkyl)-C(═O)-(heteroaryl); N(H)—S(═O)₂—(C₁₋₈-alkyl); N(H)—S(═O)₂—(C₃₋₆-cycloalkyl); N(H)—S(═O)₂-(3 to 7 membered heterocycloalkyl); N(H)—S(═O)₂-(aryl); N(H)—S(═O)₂-(heteroaryl); N(C₁₋₈-alkyl)-S(═O)₂—(C₁₋₈-alkyl); N(C₁₋₈-alkyl)-S(═O)₂—(C₃₋₆-cycloalkyl); N(C₁₋₈-alkyl)-S(═O)₂-(3 to 7 membered heterocycloalkyl); N(C₁₋₈-alkyl)-S(═O)₂-(aryl); N(C₁₋₈-alkyl)-S(═O)₂-(heteroaryl); N(H)—O(═O)—O(C₁₋₈-alkyl); N(H)—O(═O)—O(C₃₋₆-cycloalkyl); N(H)—C(═O)—O(3 to 7 membered heterocycloalkyl); N(H)—C(═O)—O(aryl); N(H)—C(═O)—O(heteroaryl); N(C₁₋₈-alkyl)-O(═O)—O(C₁₋₈-alkyl); N(C₁₋₈-alkyl)-O(═O)—O(C₃₋₆-cycloalkyl); N(C₁₋₈-alkyl)-C(═O)—O(3 to 7 membered heterocycloalkyl); N(C₁₋₈-alkyl)-O(═O)—O(aryl); N(C₁₋₈-alkyl)-O(═O)—O(heteroaryl); N(H)—C(═O)—NH₂; N(H)—O(═O)—N(H)(C₁₋₈-alkyl); N(H)—O(═O)—N(H)(C₃₋₆-cycloalkyl); N(H)—C(═O)—N(H)(3 to 7 membered heterocycloalkyl); N(H)—C(═O)—N(H)(aryl); N(H)—C(═O)—N(H)(heteroaryl); N(C₁₋₈-alkyl)-C(═O)—NH₂; N(C₁₋₈-alkyl)-C(═O)—N(H)(C₁₋₈-alkyl); N(C₁₋₈-alkyl)-C(═O)—N(H)(C₃₋₆-cycloalkyl); N(C₁₋₈-alkyl)-C(═O)—N(H)(3 to 7 membered heterocycloalkyl); N(C₁₋₈-alkyl)-C(═O)—N(H)(aryl); N(C₁₋₈-alkyl)-C(═O)—N(H)(heteroaryl); N(H)—C(═O)—N(C₁₋₈-alkyl)(C₁₋₈-alkyl); N(H)—C(═O)—N(C₁₋₈-alkyl)(C₃₋₆-cycloalkyl); N(H)—C(═O)—N(C₁₋₈-alkyl)(3 to 7 membered heterocycloalkyl); N(H)—C(═O)—N(C₁₋₈-alkyl)(aryl); N(H)—C(═O)—N(C₁₋₈-alkyl)(heteroaryl); N(C₁₋₈-alkyl)-C(═O)—N(C₁₋₈-alkyl)(C₁₋₈-alkyl); N(C₁₋₈-alkyl)-C(═O)—N(C₁₋₈-alkyl)(C₃₋₆-cycloalkyl); N(C₁₋₈-alkyl)-C(═O)—N(C₁₋₈-alkyl)(3 to 7 membered heterocycloalkyl); N(C₁₋₈-alkyl)-C(═O)—N(C₁₋₈-alkyl)(aryl); N(C₁₋₈-alkyl)-C(═O)—N(C₁₋₈-alkyl)(heteroaryl); S—(C₃₋₆-cycloalkyl); S-(3 to 7 membered heterocycloalkyl); S-(aryl); S-(heteroaryl); SCF₃; S(═O)₂OH; S(═O)—(C₁₋₈-alkyl); S(═O)—(C₃₋₆-cycloalkyl); S(═O)-(3 to 7 membered heterocycloalkyl); S(═O)-(aryl); S(═O)-(heteroaryl); S(═O)₂—(C₁₋₈-alkyl); S(═O)₂—(C₃₋₆-cycloalkyl); S(═O)₂-(3 to 7 membered heterocycloalkyl); S(═O)₂-(aryl); S(═O)₂-(heteroaryl); S(═O)₂—O(C₁₋₈-alkyl); S(═O)₂—O(C₃₋₆-cycloalkyl); S(═O)₂—O(3 to 7 membered heterocycloalkyl); S(═O)₂—O(aryl); S(═O)₂—O(heteroaryl); S(═O)₂—N(H)(C₁₋₈-alkyl); S(═O)₂—N(H)(C₃₋₆-cycloalkyl); S(═O)₂—N(H)(3 to 7 membered heterocycloalkyl); S(═O)₂—N(H)(aryl); S(═O)₂—N(H)(heteroaryl); S(═O)₂—N(C₁₋₈-alkyl)(C₁₋₈-alkyl); S(═O)₂—N(C₁₋₈-alkyl)(C₃₋₆-cycloalkyl); S(═O)₂—N(C₁₋₈-alkyl)(3 to 7 membered heterocycloalkyl); S(═O)₂—N(C₁₋₈-alkyl)(aryl); S(═O)₂—N(C₁₋₈-alkyl)(heteroaryl).

The term “polysubstituted” with respect to polysubstituted residues and groups includes the polysub-stitution of these residues and groups either on different or on the same atoms, for example trisubstituted on the same carbon atom, as in the case of CF₃, CH₂CF₃ or 1,1-difluorocyclohexyl, or at various points, as in the case of CH(OH)—CH═CH—CHCl₂ or 1-chloro-3-fluorocyclohexyl. A substituent can if appropriate for its part in turn be mono- or polysubstituted. The multiple substitution can be carried out using the same or using different substituents.

Preferred substituents of “alkyl” and “aliphatic group”, in particular “alkyl” and “alkylene”, as well as of “aliphatic group”, “cycloalkyl” and “heterocycloalkyl” are selected from the group consisting of F; Cl; CF₃; CN; ═O; C₁₋₄-alkyl; C₃₋₆-cycloalkyl; 3 to 7 membered heterocycloalkyl; C(═O)—C₁₋₄-alkyl; C(═O)—OH; C(═O)—O—C₁₋₄-alkyl; C(═O)—NH₂; C(═O)—N(H)(C₁₋₄-alkyl); C(═O)—N(C₁₋₄-alkyl)₂; OH; O—C₁₋₄-alkyl; O—C(═O)—C₁₋₄-alkyl; O—(C₂₋₄-alkyl)-OH; O—(C₂₋₄-alkyl)-O—C₁₋₄-alkyl; OCF₂H; OCFH₂; OCF₃; NH₂; N(H)(C₁₋₄-alkyl); N(C₁₋₄-alkyl)₂; N(H)—C(═O)—C₁₋₄-alkyl; N(C₁₋₄-alkyl)-C(═O)—C₁₋₄-alkyl; N(H)—S(═O)₂—C₁₋₄-alkyl; N(C₁₋₄-alkyl)-S(═O)₂—C₁₋₄-alkyl; N(H)—C(═O)—NH₂; N(H)—C(═O)—N(H)(C₁₋₄-alkyl); N(H)—C(═O)—N(C₁₋₄-alkyl)₂; S—C₁₋₄-alkyl; S(═O)—C₁₋₄-alkyl; S(═O)₂—C₁₋₄-alkyl; S(═O)₂OH; S(═O)₂O—C₁₋₄-alkyl and S(═O)₂—NH₂; S(═O)₂—N(H)(C₁₋₄-alkyl); and S(═O)₂—N(C₁₋₄-alkyl)₂.

More preferred substituents of “alkyl” and “aliphatic group”, in particular “alkyl” and “alkylene”, as well as of “aliphatic group”, “cycloalkyl” and “heterocycloalkyl” are selected from the group consisting of F; Cl; CF₃; CN; ═O; C₁₋₄-alkyl; C(═O)—C₁₋₄-alkyl; C(═O)—OH; C(═O)—O—C₁₋₄-alkyl; C(═O)—NH₂; C(═O)—N(H)(C₁₋₄-alkyl); C(═O)—N(C₁₋₄-alkyl)₂; OH; O—C₁₋₄-alkyl; O—C(═O)—C₁₋₄-alkyl; O—(C₂₋₄-alkyl)-OH; O—(C₂₋₄-alkyl)-O—C₁₋₄-alkyl; 0CF₃; NH₂; N(H)(C₁₋₄-alkyl); N(C₁₋₄-alkyl)₂; N(H)—C(═O)—C₁₋₄-alkyl N(C₁₋₄-alkyl)-C(═O)—C₁₋₄-alkyl; N(H)—S(═O)₂—C₁₋₄-alkyl; N(C₁₋₄-alkyl)-S(═O)₂—C₁₋₄-alkyl; N(H)—C(═O)—NH₂; N(H)—C(═O)—N(H)(C₁₋₄-alkyl); N(H)—C(═O)—N(C₁₋₄-alkyl)₂; S(═O)—C₁₋₄-alkyl; S(═O)₂—C₁₋₄-alkyl; S(═O)₂OH; S(═O)₂O—C₁₋₄-alkyl; S(═O)₂—NH₂; S(═O)₂—N(H)(C₁₋₄-alkyl) and S(═O)₂—N(C₁₋₄-alkyl)₂.

Most preferred substituents of “alkyl” and “aliphatic group”, in particular “alkyl” and “alkylene” are selected from the group consisting of F; Cl; CF₃; C(═O)—OH; C(═O)—NH₂; C(═O)—N(H)(C₁₋₄-alkyl); C(═O)—N(C₁₋₄-alkyl)₂; OH; O—C₁₋₄-alkyl; O—(C₂₋₄-alkyl)-OH; O—(C₂₋₄-alkyl)-O—C₁₋₄-alkyl; NH₂; N(H)(C₁₋₄-alkyl); N(C₁₋₄-alkyl)₂; N(H)—O(═O)—C₁₋₄-alkyl; N(C₁₋₄-alkyl)-O(═O)—C₁₋₄-alkyl; N(H)—S(═O)₂—C₁₋₄-alkyl; N(C₁₋₄-alkyl)-S(═O)₂—C₁₋₄-alkyl; S(═O)—C₁₋₄-alkyl; S(═O)₂—C₁₋₄-alkyl, S(═O)₂—NH₂, S(═O)₂—N(C₁₋₄-alkyl)₂ and S(═O)₂—N(H)(C₁₋₄-alkyl).

Particularly preferred substituents of “cycloalkyl” and “heterocycloalkyl” are selected from the group consisting of F; Cl; CF₃; CN; ═O); C₁₋₄-alkyl; CO₂H; C(═O)O—C₁₋₄-alkyl; CONH₂; C(═O)N(H)(C₁₋₄-alkyl); C(═O)N(C₁₋₄-alkyl)₂; OH; O—C₁₋₄-alkyl; OCF₃; O—(C₂₋₄-alkyl)-OH; O—(C₂₋₄-alkyl)-O—C₁₋₄-alkyl; O—C(═O)—C₁₋₄-alkyl; NH₂; NH—C₁₋₄-alkyl; N(C₁₋₄-alkyl)₂; NH—C(═O)—C₁₋₄-alkyl; N(C₁₋₄-alkyl)-O(═O)—C₁₋₄-alkyl; S(═O)—C₁₋₄-alkyl; S(═O)₂—C₁₋₄-alkyl; S(═O)₂—NH₂, S(═O)₂—N(C₁₋₄-alkyl)₂ and S(═O)₂—N(H)—C₁₋₄-alkyl.

In relation to the terms “aryl” and “heteroaryl”, the term “mono- or polysubstituted” refers in the sense of this invention, with respect to the corresponding residues or groups, to the single substitution or multiple substitution, e.g. disubstitution, trisubstitution, tetrasubstitution, or pentasubstitution, of one or more hydrogen atoms each independently of one another by at least one substituent selected from the group consisting of F; Cl; Br; NO₂; CN; CF₃; CF₂H; CFH₂; CF₂Cl; CFCl₂; C₁₋₈-alkyl; C₃₋₆-cycloalkyl; 3 to 7 membered heterocycloalkyl; aryl; heteroaryl; aryl, heteroaryl, C₃₋₆-cycloalkyl or 3 to 7 membered heterocycloalkyl, each connected via a C₁₋₄-aliphatic group; C(═O)H; C(═O)—(C₁₋₈-alkyl); C(═O)—(C₃₋₆-cycloalkyl); C(═O)-(3 to 7 membered heterocycloalkyl); C(═O)-(aryl); C(═O)-(heteroaryl); C(═O)OH; C(═O)—O(C₁₋₈-alkyl); C(═O)—O(C₃₋₆-cycloalkyl); C(═O)—O(3 to 7 membered heterocycloalkyl); C(═O)—O(aryl); C(═O)—O(heteroaryl); C(═O)—NH₂; C(═O)—N(H)(C₁₋₈-alkyl); C(═O)—N(H)(C₃₋₆-cycloalkyl); C(═O)—N(H)(3 to 7 membered heterocycloalkyl); C(═O)—N(H)(aryl); C(═O)—N(H)(heteroaryl); C(═O)—N(C₁₋₈-alkyl)(C₁₋₈-alkyl); C(═O)—N(C₁₋₈-alkyl)(C₃₋₆-cycloalkyl); C(═O)—N(C₁₋₈-alkyl)(3 to 7 membered heterocycloalkyl); C(═O)—N(C₁₋₈-alkyl)(aryl); C(═O)—N(C₁₋₈-alkyl)(heteroaryl); OH; ═O; O—(C₁₋₈-alkyl); O—(C₃₋₆-cycloalkyl); O-(3 to 7 membered heterocycloalkyl); O-(aryl); O-(heteroaryl); OCF₃; OCF₂H; OCFH₂; OCF₂Cl; OCFCl₂; O—(C₂₋₄-alkyl)-OH; O—(C₂₋₄-alkyl)-O(C₁₋₈-alkyl); O—C(═O)—(C₁₋₈-alkyl); O—C(═O)—(C₃₋₆-cycloalkyl); O—C(═O)-(3 to 7 membered heterocycloalkyl); O—C(═O)-(aryl); C(═O)-(heteroaryl); O—C(═O)—NH₂; O—C(═O)—N(H)(C₁₋₈-alkyl); O—C(═O)—N(H)(C₃₋₆-cycloalkyl); O—C(═O)—N(H)(3 to 7 membered heterocycloalkyl); O—C(═O)—N(H)(aryl); O—C(═O)—N(H)(heteroaryl); O—C(═O)—N(C₁₋₈-alkyl)(C₁₋₈-alkyl); O—C(═O)—N(C₁₋₈-alkyl)(C₃₋₆-cycloalkyl); O—C(═O)—N(C₁₋₈-alkyl)(3 to 7 membered heterocycloalkyl); O—C(═O)—N(C₁₋₈-alkyl)(aryl); O—C(═O)—N(C₁₋₈-alkyl)(heteroaryl); NH₂; N(H)(C₁₋₈-alkyl); N(H)(C₃₋₆-cycloalkyl); N(H)(3 to 7 membered heterocycloalkyl); N(H)(aryl); N(H)(heteroaryl); N(C₁₋₈-alkyl)(C₁₋₈-alkyl); N(C₁₋₈-alkyl)(C₃₋₆-cycloalkyl); N(C₁₋₈-alkyl)(3 to 7 membered heterocycloalkyl); N(C₁₋₈-alkyl)(aryl); N(C₁₋₈-alkyl)(heteroaryl); N(H)—C(═O)—(C₁₋₈-alkyl); N(H)—O(═O)—(C₃₋₆-cycloalkyl); N(H)—O(═O)-(3 to 7 membered heterocycloalkyl); N(H)—O(═O)-(aryl); N(H)—O(═O)-(heteroaryl); N(C₁₋₈-alkyl)-C(═O)—(C₁₋₈-alkyl); N(C₁₋₈-alkyl)-O(═O)—(C₃₋₆-cycloalkyl); N(C₁₋₈-alkyl)-O(═O)-(3 to 7 membered heterocycloalkyl); N(C₁₋₈-alkyl)-O(═O)-(aryl); N(C₁₋₈-alkyl)-O(═O)-(heteroaryl); N(H)—S(═O)₂—(C₁₋₈-alkyl); N(H)—S(═O)₂—(C₃₋₆-cycloalkyl); N(H)—S(═O)₂-(3 to 7 membered heterocycloalkyl); N(H)—S(═O)₂-(aryl); N(H)—S(═O)₂-(heteroaryl); N(C₁₋₈-alkyl)-S(═O)₂—(C₁₋₈-alkyl); N(C₁₋₈-alkyl)-S(═O)₂—(C₃₋₆-cycloalkyl); N(C₁₋₈-alkyl)-S(═O)₂-(3 to 7 membered heterocycloalkyl); N(C₁₋₈-alkyl)-S(═O)₂-(aryl); N(C₁₋₈-alkyl)-S(═O)₂-(heteroaryl); N(H)—C(═O)—O(C₁₋₈-alkyl); N(H)—O(═O)—O(C₃₋₆-cycloalkyl); N(H)—O(═O)—O(3 to 7 membered heterocycloalkyl); N(H)—O(═O)—O(aryl); N(H)—O(═O)—O(heteroaryl); N(C₁₋₈-alkyl)-O(═O)—O(C₁₋₈-alkyl); N(C₁₋₈-alkyl)-O(═O)—O(C₃₋₆-cycloalkyl); N(C₁₋₈-alkyl)-O(═O)—O(3 to 7 membered heterocycloalkyl); N(C₁₋₈-alkyl)-O(═O)—O(aryl); N(C₁₋₈-alkyl)-O(═O)—O(heteroaryl); N(H)—C(═O)—NH₂; N(H)—O(═O)—N(H)(C₁₋₈-alkyl); N(H)—O(═O)—N(H)(C₃₋₆-cycloalkyl); N(H)—O(═O)—N(H)(3 to 7 membered heterocycloalkyl); N(H)—C(═O)—N(H)(aryl); N(H)—C(═O)—N(H)(heteroaryl); N(C₁₋₈-alkyl)-C(═O)—NH₂; N(C₁₋₈-alkyl)-C(═O)—N(H)(C₁₋₈-alkyl); N(C₁₋₈-alkyl)-C(═O)—N(H)(C₃₋₆-cycloalkyl); N(C₁₋₈-alkyl)-C(═O)—N(H)(3 to 7 membered heterocycloalkyl); N(C₁₋₈-alkyl)-C(═O)—N(H)(aryl); N(C₁₋₈-alkyl)-C(═O)—N(H)(heteroaryl); N(H)—C(═O)—N(C₁₋₈-alkyl)(C₁₋₈-alkyl); N(H)—C(═O)—N(C₁₋₈-alkyl)(C₃₋₆-cycloalkyl); N(H)—C(═O)—N(C₁₋₈-alkyl)(3 to 7 membered heterocycloalkyl); N(H)—C(═O)—N(C₁₋₈-alkyl)(aryl); N(H)—C(═O)—N(C₁₋₈-alkyl)(heteroaryl); N(C₁₋₈-alkyl)-C(═O)—N(C₁₋₈-alkyl)(C₁₋₈-alkyl); N(C₁₋₈-alkyl)-C(═O)—N(C₁₋₈-alkyl)(C₃₋₆-cycloalkyl); N(C₁₋₈-alkyl)-C(═O)—N(C₁₋₈-alkyl)(3 to 7 membered heterocycloalkyl); N(C₁₋₈-alkyl)-C(═O)—N(C₁₋₈-alkyl)(aryl); N(C₁₋₈-alkyl)-C(═O)—N(C₁₋₈-alkyl) heteroaryl); SH; S—(C₁₋₈-alkyl); S—(C₃₋₆-cycloalkyl); S-(3 to 7 membered heterocycloalkyl); S-(aryl); S-(heteroaryl); SCF₃; S(═O)₂OH; S(═O)—(C₁₋₈-alkyl); S(═O)—(C₃₋₆-cycloalkyl); S(═O)-(3 to 7 membered heterocycloalkyl); S(═O)-(aryl); S(═O)-(heteroaryl); S(═O)₂—(C₁₋₈-alkyl); S(═O)₂—(C₃₋₆-cycloalkyl); S(═O)₂-(3 to 7 membered heterocycloalkyl); S(═O)₂-(aryl); S(═O)₂-(heteroaryl); S(═O)₂—O(C₁₋₈-alkyl); S(═O)₂—O(C₃₋₆-cycloalkyl); S(═O)₂—O(3 to 7 membered heterocycloalkyl); S(═O)₂—O(aryl); S(═O)₂—O(heteroaryl); S(═O)₂—N(H)(C₁₋₈-alkyl); S(═O)₂—N(H)(C₃₋₆-cycloalkyl); S(═O)₂—N(H)(3 to 7 membered heterocycloalkyl); S(═O)₂—N(H)(aryl); S(═O)₂—N(H)(heteroaryl); S(═O)₂—N(C₁₋₈-alkyl)(C₁₋₈-alkyl); S(═O)₂—N(C₁₋₈-alkyl)(C₃₋₆-cycloalkyl); S(═O)₂—N(C₁₋₈-alkyl)(3 to 7 membered heterocycloalkyl); S(═O)₂—N(C₁₋₈-alkyl)(aryl); S(═O)₂—N(C₁₋₈-alkyl)(heteroaryl).

Preferred substituents of “aryl” and “heteroaryl” are selected from the group consisting of F; Cl; Br; NO₂; CN; CF₃; CF₂H; CFH₂; CF₂Cl; CFCl₂; C₁₋₄-alkyl; aryl; heteroaryl; C₃₋₆-cycloalkyl; 3 to 7 membered heterocycloalkyl; aryl, heteroaryl, C₃₋₆-cycloalkyl or 3 to 7 membered heterocycloalkyl, each connected via a C₁₋₄-aliphatic group; C(═O)—C₁₋₄-alkyl; C(═O)aryl; C(═O)heteroaryl; C(═O)—OH; C(═O)—O—C₁₋₄-alkyl; C(═O)O-aryl; C(═O)O-heteroaryl; CO—NH₂; C(═O)—N(H)C₁₋₄-alkyl; C(═O)—N(C₁₋₄-alkyl)₂; C(═O)NH-aryl; C(═O)N(aryl)₂; C(═O)NH-heteroaryl; C(═O)N(heteroaryl)₂; C(═O)N(C₁₋₄-alkyl)(aryl); C(═O)N(C₁₋₄-alkyl) (heteroaryl); C(═O)N(heteroaryl)(aryl); OH; OCF₃; OCF₂H; OCFH₂; OCF₂Cl; OCFCl₂; O—C₁₋₄-alkyl; O—(C₂₋₄-alkyl)-OH; O—(C₂₋₄-alkyl)O—C₁₋₄-alkyl; O—(C₃₋₆-cycloalkyl); O-(3 to 7 membered heterocycloalkyl); O-aryl; O-heteroaryl; O—C(═O)—C₁₋₄-alkyl; O—C(═O)aryl; O—C(═O)heteroaryl; O—(C═O)—N(H)C₁₋₄-alkyl; O—C(═O)—N(C₁₋₄-alkyl)₂; NH₂; N(H)C₁₋₄-alkyl; N(C₁₋₄-alkyl)₂; N(H)—C(═O)—C₁₋₄-alkyl; N(H)—C(═O)-aryl; N(H)—C(═O)-heteroaryl; N(H)—C(═O)—O—C₁₋₄-alkyl; N(H)—C(═O)—NH₂; N(H)—C(═O)—N(H)C₁₋₄-alkyl; N(H)—C(═O)—N(C₁₋₄-alkyl)₂; N(C₁₋₄-alkyl)-C(═O)C₁₋₄-alkyl; N(C₁₋₄-alkyl)-C(═O)—O—C₁₋₄-alkyl; N(C₁₋₄-alkyl)-C(═O)—NH₂; N(C₁₋₄-alkyl)-C(═O)—N(H)C₁₋₄-alkyl; N(C₁₋₄-alkyl)-C(═O)—N(C₁₋₄-alkyl)₂; SCF₃; SCF₂H; SCFH₂; SCF₂Cl; SCFCl₂; S—C₁₋₄-alkyl; S-aryl; S-heteroaryl; S(═O)—C₁₋₄-alkyl; S(═O)₂—C₁₋₄-alkyl; S(═O)₂-aryl; S(═O)₂-heteroaryl; S(═O)₂—OH; S(═O)₂—OC₁₋₄-alkyl; S(═O)₂O-aryl; S(═O)₂O-heteroaryl; S(═O)₂—NH₂; S(═O)₂—N(H)C₁₋₄-alkyl, S(═O)₂—N(H)-aryl; S(═O)₂—N(H)-heteroaryl and S(═O)₂—N(C₁₋₄-alkyl)₂.

More preferred substituents of “aryl” and “heteroaryl” are selected from the group consisting of F; Cl; CF₂H; CFH₂; CF₃; CN; C₁₋₄-alkyl; C(═O)—OH; C(═O)—O—C₁₋₄-alkyl; CO—NH₂; C(═O)—N(H)C₁₋₄-alkyl; C(═O)—N(C₁₋₄-alkyl)₂; OH; O—C₁₋₄-alkyl; O—(C₃₋₆-cycloalkyl); O-(3 to 7 membered heterocycloalkyl); O—C(═O)—C₁₋₄-alkyl; O—(C₂₋₄-alkyl)-OH; O—(C₂₋₄-alkyl)O—C₁₋₄-alkyl; OCF₃; OCHF₂; OCH₂F; NH₂; N(H)C₁₋₄-alkyl; N(C₁₋₄-alkyl)₂; N(H)—C(═O)—C₁₋₄-alkyl; N(C₁₋₄-alkyl)-C(═O)C₁₋₄-alkyl; N(H)—S(═O)₂—C₁₋₄-alkyl; N(C₁₋₄-alkyl)-S(═O)₂(C₁₋₄-alkyl); N(H)—C(═O)—NH₂; N(H)—C(═O)—N(H)C₁₋₄-alkyl; N(H)—C(═O)—N(C₁₋₄-alkyl)₂; N(C₁₋₄-alkyl)-C(═O)—NH₂; N(C₁₋₄-alkyl)-C(═O)—N(H)C₁₋₄-alkyl; N(C₁₋₄-alkyl)-C(═O)—N(C₁₋₄-alkyl)₂; S(═O)—C₁₋₄-alkyl; S(═O)₂C₁₋₄-alkyl; S(═O)₂—NH₂; S(═O)₂—N(H)C₁₋₄-alkyl and S(═O)₂—N(C₁₋₄-alkyl)₂.

The compounds according to the invention are defined by substituents, for example by R^{A}, R^{B} and R^{c} (1^{st} generation substituents) which are for their part if appropriate themselves substituted (2^{nd} generation substituents). Depending on the definition, these substituents of the substituents can for their part be resubstituted (3^{rd} generation substituents). If, for example, R^{A}=C₁₋₄-alkyl (1^{st} generation substituent), then the C₁₋₄-alkyl can for its part be substituted, for example with a N(H)C₁₋₄-alkyl (2^{nd} generation substituent). This produces the functional group R^{A}=(C₁₋₄-alkyl-N(H)—C₁₋₄-alkyl). The N(H)—C₁₋₄-alkyl can then for its part be resubstituted, for example with CI (3^{rd} generation substituent). Overall, this produces the functional group R^{A}=C₁₋₄-alkyl-N(H)—C₁₋₄-alkyl-CI, wherein the C₁₋₄-alkyl of the N(H)C₁₋₄-alkyl is substituted by Cl.

However, in a preferred embodiment, the 3^{rd} generation substituents may not be resubstituted, i.e. there are then no 4^{th} generation substituents.

In another preferred embodiment, the 2^{nd} generation substituents may not be resubstituted, i.e. there are then not even any 3^{rd} generation substituents. In other words, in this embodiment, in the case of general formula (I), for example, the functional groups for R¹ to R¹³ can each if appropriate be substituted; however, the respective substituents may then for their part not be resubstituted.

In some cases, the compounds according to the invention are defined by substituents which are or carry a cycloalkyl or a heterocycloalkyl, respectively, in each case unsubstituted or mono- or polysubstituted, or which form together with the carbon atom(s) or heteroatom(s) connecting them, as the ring member or as the ring members, a ring, for example a cycloaliphatic or a heterocycloaliphatic ring system. Both these cycloaliphatic or heterocycloaliphatic ring systems and the (hetero)cycloaliphatic ring systems formed in this manner can if appropriate be condensed with a cycloalkyl, preferably a C₃₋₆-cycloalkyl, or with a heterocycloalkyl, preferably a 3 to 7 membered heterocycloalkyl, e.g. with a cycloalkyl such as cyclohexyl, or a heterocycloalkyl such as morpholinyl, wherein the cycloaliphatic or heterocycloalkyls condensed in this way can for their part be respectively unsubstituted or mono- or polysubstituted.

Within the scope of the present invention, the symbol

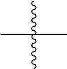

used in the formulae denotes a link of a corresponding residue to the respective superordinate general structure.

In one embodiment of the present invention, A¹ and A² each represent direct bond.

In another embodiment of the present invention, A¹ represents direct bond and A² represents C(═O). In another embodiment of the present invention, A¹ and A² each represent direct bond. In yet another embodiment of the present invention, A² represents direct bond and A¹ represents C(═O).

In one embodiment of the present invention, m and n independently denote 0, 1, 2 or 3, with the proviso that the sum [n+m] is 1, 2, 3 or 4. Preferably, the sum [n+m] is 2 or 3. Even more preferably, A¹ and A² each represent direct bond and the sum [n+m] is 2 or 3.

In one embodiment of the present invention, the compound according to formula (I) is selected from one of the compounds according to formula (I-1) to (I-18),

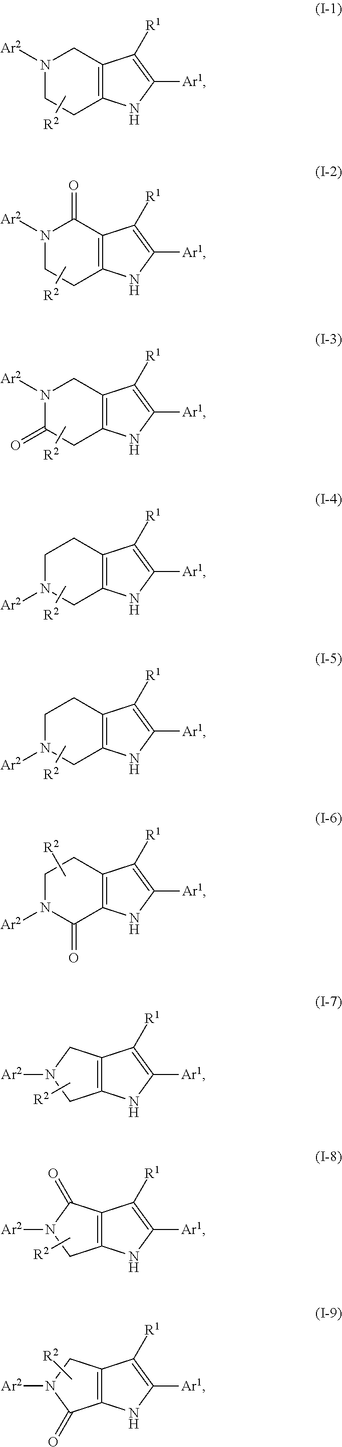

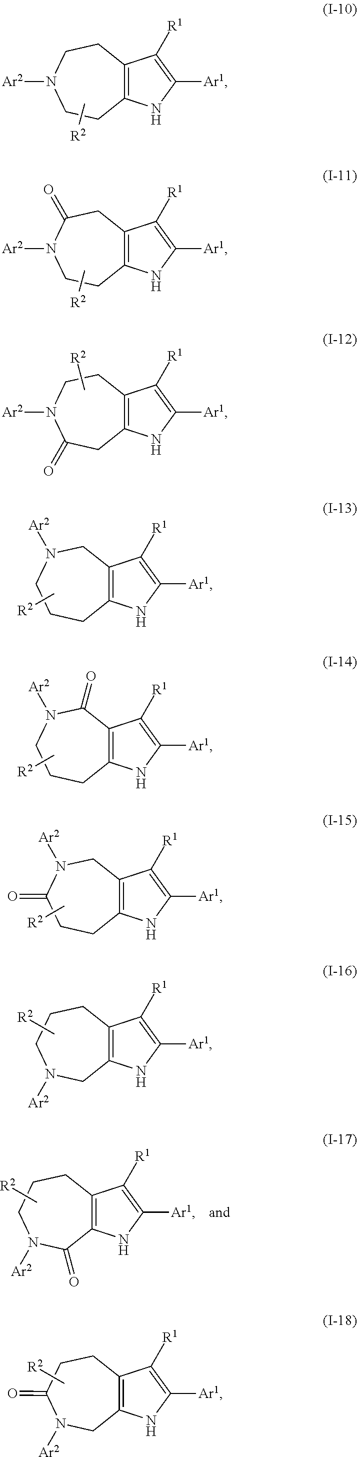

wherein R¹, R², Ar¹ and Ar² are defined as before.

Particularly preferred are the compounds according to formula (I-1), (I-7), (I-9) and (I-13), more particularly preferred is a compound according to formula (I-1).

In one preferred embodiment of the present invention, the compound according to formula (I) is selected from one of the compounds according to formula (I-1) to (I-18), wherein R² represents 0 to 2 substituents, each independently selected from F, Cl, Br, CN, CF₃, CF₂H, CFH₂, R¹³, OH, O—R¹³, NH₂, N(H)R¹³ and N(R¹³)₂.

In one embodiment of the present invention, the compound according to formula (I) is characterized in that R¹ is selected from the group consisting of H, F, Cl, Br, I, CN, CF₃, CF₂H, CFH₂, CO₂H, CO₂R¹³, R¹³, OH, O—R¹³, NH₂, N(H)R¹³ and N(R¹³)₂.

In one preferred embodiment of the present invention, R¹ is selected from H, F, Cl, CN, CH₃, cyclopropyl, CF₃, CF₂H, CFH₂, CO₂H, CO₂CH₃, OH, OCH₃, O-cyclopropyl, OCF₂H, OCFH₂ and OCF₃, preferably R¹ represents H. More preferably, the compound according to formula (I) is selected from one of the compounds according to formula (I-1) to (I-18), wherein R¹ is selected from H, F, Cl, CN, CH₃, cyclopropyl, CF₃, CF₂H, CFH₂, CO₂H, CO₂CH₃, OH, OCH₃, O-cyclopropyl, OCF₂H, OCFH₂ and OCF₃, more preferably, wherein R¹ represents H, F or CI, even more preferably R¹ represents H or F.

In one particularly preferred embodiment of the present invention, R¹ represents H. In another particularly preferred embodiment of the present invention, R¹ represents F.

In one embodiment of the present invention, the compound according to formula (I) is characterized in that R² represents 0 to 4 substituents, preferably 0 to 2 substituents, each independently selected from F, Cl, Br, CN, CF₃, CF₂H, CFH₂, R¹³, OH, O—R¹³, NH₂, N(H)R¹³ or N(R¹³)₂.

In one preferred embodiment of the present invention, R² represents 0 to 4 substituents, preferably 0 to 2 substituents, each independently selected from F, Cl, CN, CH₃, cyclopropyl, CF₃, OH or OCH₃, preferably R² represents 0 substituents. More preferably, the compound according to formula (I) is selected from one of the compounds according to formula (I-1) to (I-18), wherein R² represents 0 to 2 substituents, each independently selected from F, Cl, CN, CH₃, cyclopropyl, CF₃, OH or OCH₃, more preferably, wherein R² represents 0 substituents.

In one particularly preferred embodiment of the present invention, the compound according to formula (I) is selected from one compound (I-a) to (I-d),

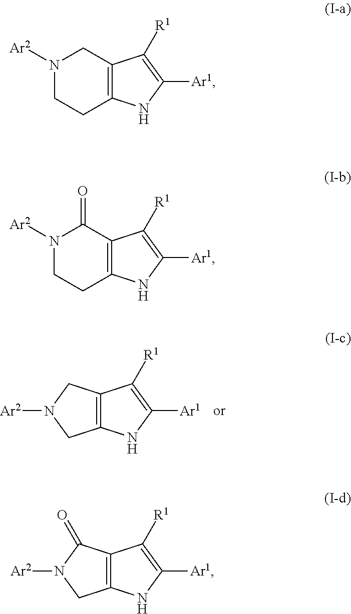

wherein R¹, Ar¹ and Ar² are defined as before,
particularly preferred, the compound according to formula (I) is selected from compound (I-a) or (I-b),
wherein R¹, Ar¹ and Ar² are defined as before,
more particularly preferred, the compound according to formula (I) is compound (I-a),
wherein R¹, Ar¹ and Ar² are defined as before.

In one embodiment of the present invention, the compound according to formula (I) is characterized in that Ar¹ represents phenyl or 5- or 6-membered heteroaryl, in each case unsubstituted or substituted with one, two, three or four substituents, independently selected from F, Cl, Br, CN, CF₃, CF₂H, CFH₂, R¹³ and O—R¹³; or

Ar¹ represents C₃₋₆-cycloalkyl or 3 to 7 membered heterocycloalkyl, in each case unsubstituted or mono- or polysubstituted.

In another embodiment of the present invention, Ar¹ represents phenyl or 5- or 6-membered heteroaryl, in each case unsubstituted or substituted with one, two, three or four substituents, independently selected from F, Cl, Br, CN, CF₃, CF₂H, CFH₂, R¹³ and O—R¹³. Particularly preferred, Ar¹ represents phenyl, pyridinyl, pyrazinyl, pyrimidinyl, pyridazinyl, thienyl, furanyl, pyrrolyl, isoxazolyl, oxazolyl, isothiazolyl, thiazolyl, pyrazolyl, imidazolyl, oxadiazolyl, thiadiazolyl, triazolyl or tetrazolyl, each case unsubstituted or substituted with one, two, three or four substituents, independently selected from F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃, preferably independently selected from F, CI or CH₃.

In one preferred embodiment of the present invention, Ar¹ represents substructure (II),

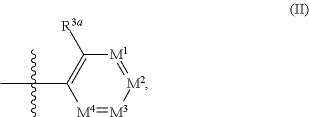

wherein
R^{1a} denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃, preferably F, CI or CH₃,
and M¹, M², M³ and M⁴ independently represent N, CH or CR^{3b},
wherein R^{3b} denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃,
with the proviso, that 0 or 1 of the substituents M¹, M², M³ and M⁴ represent N.

In yet another embodiment of the present invention, Ar¹ represents C₃₋₆-cycloalkyl or 3 to 7 membered heterocycloalkyl, in each case unsubstituted or mono- or polysubstituted. Particularly preferred, Ar¹ represents cyclopropyl, cyclopentyl, cyclopentenyl, cyclohexyl or cyclohexenyl, unsubstituted or substituted with one or more substituents, independently selected from F, Cl, CN, CH₃, cyclopropyl, CF₃, CF₂H, CFH₂, OH, OCH₃, O-cyclopropyl, OCF₂H, OCFH₂ and OCF₃.

Also particularly preferred, Ar¹ represents 2- or 3-tetrahydrofuranyl, 2- or 3- or 4-tetrahydropyranyl, 2- or 3- or 4-piperidinyl, unsubstituted or substituted with one or more substituents, independently selected from F, Cl, CN, CH₃, cyclopropyl, CF₃, CF₂H, CFH₂, C(═O)CH₃, OCH₃, O-cyclopropyl, OCF₂H, OCFH₂ and OCF₃.

Particularly preferred, Ar¹ is selected from 2,6-difluorophenyl, 2,6-difluoro-4-methoxyphenyl, 2-chlorophenyl, 2-chloro-4-fluorophenyl, 2-chloro-6-fluorophenyl, 5-fluoro-4-methyl-pyridin-3-yl, 4-methyl-[1,2,3]-thiadiazol-5-yl, 1,3-dimethyl-pyrazol-4-yl, 2,4-difluorophenyl, 2,4-dimethoxyphenyl, 3-fluoro-pyridin-4-yl, 3,5-difluoro-pyridin-4-yl and 2-fluoro-pyridin-3-yl.

In one embodiment of the present invention, the compound according to formula (I) is characterized in that the substituent Ar² bears an ortho-substituent.

One embodiment of the present invention is therefore a compound according to formula (I), characterized in that Ar² represents substructure (III),

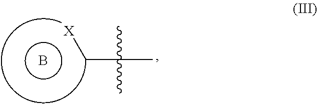

wherein
X represents CR⁴ or NR⁵,
wherein R⁴ denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃,
and
R⁵ denotes CF₃, CF₂H, CFH₂, CH₃, CH₂CH₃ or cyclopropyl,
   and B is phenyl or 5- or 6-membered heteroaryl, including the structural element “C—X”,
   wherein B may be unsubstituted or mono- or polysubstituted and wherein B may be condensed with a 4-, 5-, 6- or 7-membered ring, being carbocyclic or heterocyclic, wherein said condensed ring may be saturated, partially unsaturated or aromatic and may be unsubstituted or mono- or polysubstituted,
wherein said substituents are independently selected from the group consisting of F; Cl; Br; CN; CF₃; CF₂H; CFH₂; CF₂Cl; CFCl₂; R¹³; R¹⁴; C(═O)OH; C(═O)—R¹³; C(═O)R¹⁴; C(═O)—OR¹³; C(═O)—OR¹⁴; C(═O)NH₂; C(═O)—N(H)R¹³; C(═O)—N(R¹³)₂; C(═O)—N(H)R¹⁴; C(═O)—N(R¹⁴)₂; C(═O)—N(R¹³)(R¹⁴); C(═O)—N(R^{a})(R^{b}); OH; OR¹³; OCF₃; OCF₂H; OCFH₂; OCF₂Cl; OCFCl₂; OR¹⁴; O—C(═O)R¹³; O—C(═O)R¹⁴; O—C(═O)—N(H)R¹³; O—C(═O)—N(H)R¹⁴; O—C(═O)—N(R¹³)₂; O—C(═O)—N(R¹⁴)₂; O—C(═O)—N(R¹³)(R¹⁴); O—C(═O)—N(R^{a})(R^{b}); NH₂; N(H)R¹³; N(R¹³)₂; N(H)R¹⁴; N(R¹⁴)₂; N(R¹³)(R¹⁴); N(R^{a})(R^{b}); NH—C(═O)—R¹⁴; NH—C(═O)—R¹³; N(R¹³)—C(═O)—R¹³; N(R¹³)—C(═O)—R¹⁴; NH—S(═O)₂—R¹³; N(R¹³)—S(═O)₂—R¹³; NH—S(═O)₂—R¹⁴; N(R¹³)—S(═O)₂—R¹⁴; N(H)—C(═O)—OR¹³; N(H)—C(═O)—OR¹⁴; N(R¹³)—C(═O)—OR¹³; N(R¹³)—C(═O)—OR¹⁴; N(H)—C(═O)—NH₂; N(H)—C(═O)—N(H)R¹³; N(H)—C(═O)—N(H)R¹⁴; N(H)—C(═O)—N(R¹³)₂; N(H)—C(═O)—N(R¹⁴)₂; N(H)—C(═O)—N(R¹³)(R¹⁴); N(H)—C(═O)—N(R^{a})(R^{b}); N(R¹³)—C(═O)—NH₂; N(R¹³)—C(═O)—N(H)R¹³; N(R¹³)—C(═O)—N(H)R¹⁴; N(R¹³)—C(═O)—N(R¹³)₂; N(R¹³)—C(═O)—N(R¹⁴)₂; N(R¹³)—C(═O)—N(R¹³)(R¹⁴); N(R¹³)—C(═O)—N(R^{a})(R^{b}); SH; S—R¹³; SCF₃; S—R¹⁴; S(═O)₂OH; S(═O)₂—R¹³; S(═O)₂—R¹⁴; S(═O)—R¹³; S(═O)—R¹⁴; S(═O)₂—OR¹³; S(═O)₂—OR¹⁴; S(═O)₂—N(H)(R¹³); S(═O)₂—N(R¹³)₂; S(═O)₂—N(H)(R¹⁴); S(═O)₂—N(R¹³)(R¹⁴); S(═O)₂—N(R^{a})(R^{b});
   wherein
   each R¹³ independently of each other denotes
      C₁₋₈-alkyl, unsubstituted or mono- or polysubstituted; or
      C₃₋₆-cycloalkyl or 3 to 7 membered heterocycloalkyl, in each case unsubstituted or mono- or polysubstituted; or
      C₃₋₆-cycloalkyl or 3 to 7 membered heterocycloalkyl, in each case unsubstituted or mono- or polysubstituted, and in each case connected via a C₁₋₄-aliphatic group, unsubstituted or mono- or polysubstituted;
   each R¹⁴ independently of each other denotes
      aryl and heteroaryl, in each case independently of one another unsubstituted or mono- or polysubstituted, or
      aryl and heteroaryl, in each case independently of one another unsubstituted or mono- or polysubstituted and in each case connected via a C₁₋₄-aliphatic group, unsubstituted or mono- or polysubstituted;
   and R^{a} and R^{b} together with the N-atom connecting them form a 3 to 7 membered heterocycloalkyl, unsubstituted or mono- or polysubstituted.

Preferably, Ar² is selected from the group consisting of

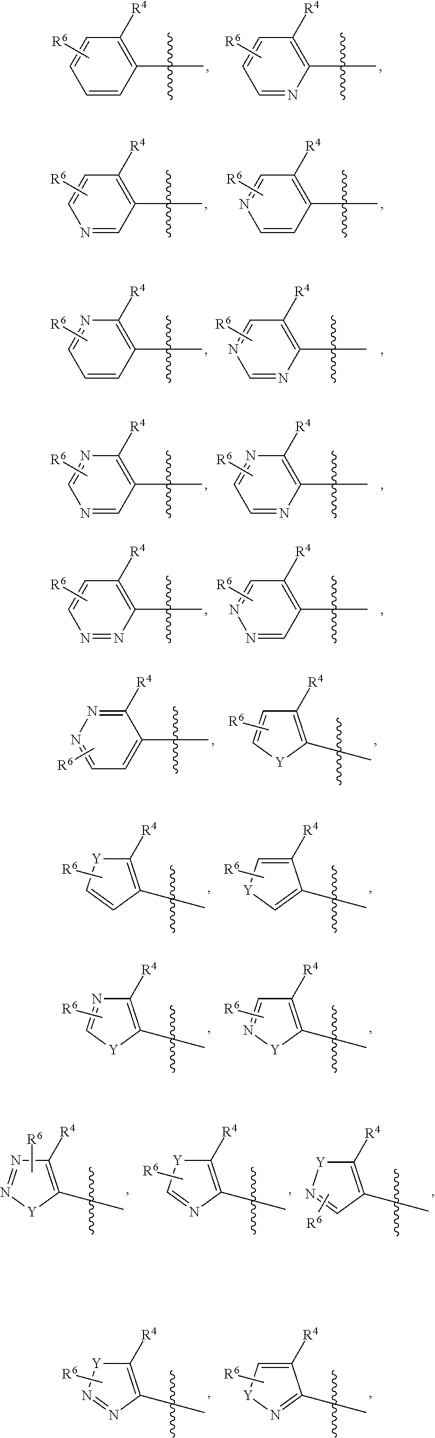

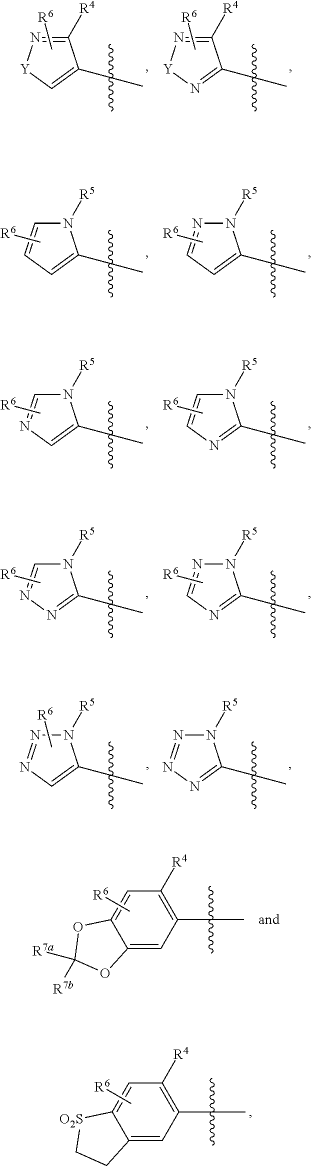

wherein
Y represents O, S or NR⁸;
R⁴ denotes F, Cl, CN, CF₃, CF₂H, CFH₂, CH₃, CH₂CH₃, cyclopropyl, OCF₃, OCF₂H, OCH₃ or OCH₂CH₃;
R⁵ denotes CF₃, CF₂H, CFH₂, cyclopropyl, CH₃ or CH₂CH₃;
R^{7a} and R^{7b} each independently represent H, F or C₁₋₄-alkyl;
R⁸ denotes H, C₁₋₄-alkyl or C(═O)C₁₋₄-alkyl, wherein C₁₋₄-alkyl may be unsubstituted or substituted by one or more substituents selected from F, Cl, OH, NH₂, N(H)C₁₋₄-alkyl, N(C₁₋₄-alkyl)₂, N(H)C(═O)C₁₋₄-alkyl, N(C₁₋₄-alkyl)C(═O)C₁₋₄-alkyl, OH, OCH₃ and OCH₂CH₃;
and
R⁶ denotes 0, 1, 2 or 4 substituents, independently selected from the group consisting of F; Cl; Br; CN; CF₃; CF₂H; CFH₂; CF₂Cl; CFCl₂; R¹³; R¹⁴; C(═O)OH; C(═O)—R¹³; C(═O)R¹⁴; C(═O)—OR¹³; C(═O)—OR¹⁴; C(═O)NH₂; C(═O)—N(H)R¹³; C(═O)—N(R¹³)₂; C(═O)—N(H)R¹⁴; C(═O)—N(R¹⁴)₂; C(═O)—N(R¹³)(R¹⁴); C(═O)—N(R^{a})(R^{b}); OH; OR¹³; OCF₃; OCF₂H; OCFH₂; OCF₂Cl; OCFCl₂; OR¹⁴; O—C(═O)R¹³; O—C(═O)R¹⁴; O—C(═O)—N(H)R¹³; O—C(═O)—N(H)R¹⁴; O—C(═O)—N(R¹³)₂; O—C(═O)—N(R¹⁴)₂; O—C(═O)—N(R¹³)(R¹⁴); O—C(═O)—N(R^{a})(R^{b}); NH₂; N(H)R¹³; N(R¹³)₂; N(H)R¹⁴; N(R¹⁴)₂; N(R¹³)(R¹⁴); N(R^{a})(R^{b}); NH—C(═O)—R¹⁴; NH—C(═O)—R¹³; N(R¹³)—C(═O)—R¹³; N(R¹³)—C(═O)—R¹⁴; NH—S(═O)₂—R¹³; N(R¹³)—S(═O)₂—R¹³; NH—S(═O)₂—R¹⁴; N(R¹³)—S(═O)₂—R¹⁴; N(H)—C(═O)—OR¹³; N(H)—C(═O)—OR¹⁴; N(R¹³)—C(═O)—OR¹³; N(R¹³)—C(═O)—OR¹⁴; N(H)—C(═O)—NH₂; N(H)—C(═O)—N(H)R¹³; N(H)—C(═O)—N(H)R¹⁴; N(H)—C(═O)—N(R¹³)₂; N(H)—C(═O)—N(R¹⁴)₂; N(H)—C(═O)—N(R¹³)(R¹⁴); N(H)—C(═O)—N(R^{a})(R^{b}); N(R¹³)—C(═O)—NH₂; N(R¹³)—C(═O)—N(H)R¹³; N(R¹³)—C(═O)—N(H)R¹⁴; N(R¹³)—C(═O)—N(R¹³)₂; N(R¹³)—C(═O)—N(R¹⁴)₂; N(R¹³)—C(═O)—N(R¹³)(R¹⁴); N(R¹³)—C(═O)—N(R^{a})(R^{b}); SH; S—R¹³; SCF₃; S—R¹⁴; S(═O)₂OH; S(═O)₂—R¹³; S(═O)₂—R¹⁴; S(═O)—R¹³; S(═O)—R¹⁴; S(═O)₂—OR¹³; S(═O)₂—OR¹⁴; S(═O)₂—N(H)(R¹³); S(═O)₂—N(R¹³)₂; S(═O)₂—N(H)(R¹⁴); S(═O)₂—N(R¹³)(R¹⁴) and S(═O)₂—N(R^{a})(R^{b}); wherein R¹³, R¹⁴ and R^{a} and R^{b} are defined as before.

In one preferred embodiment of the present invention, R⁶ is selected from the group consisting of
F, Cl, Br, CN, C(═O)O—C₁₋₄-alkyl, CF₃, CF₂H, CFH₂, C(═O)NH₂, C(═O)—N(H)C₁₋₄-alkyl, C(═O)—N(C₁₋₄-alkyl)₂, C(═O)—N(H)(C₁₋₄-alkylene-OH), OCF₃, OCF₂H, OCFH₂, C₁₋₄-alkyl, OH, O—C₁₋₄-alkyl, S(═O)C₁₋₄-alkyl, SO₂—C₁₋₄-alkyl, SO₂—OF₃, SO₂—N(H)C₁₋₄-alkyl, SO₂—N(C₁₋₄-alkyl)₂, CH₂S(═O)C₁₋₄-alkyl, CH₂SO₂—C₁₋₄-alkyl, CH₂N(H)SO₂—C₁₋₄-alkyl, CH₂SO₂—N(H)C₁₋₄-alkyl, CH₂SO₂—N(C₁₋₄-alkyl)₂,
C₃₋₆-cycloalkyl, preferably selected from the group consisting of cyclopropyl, cyclobutyl, cyclopentyl or cyclohexyl,
wherein the C₃₋₆-cycloalkyl is unsubstituted or mono- or di-substituted with at least one substituent selected from the group consisting of F, Cl, CN, OH, OCH₃, CF₃, CH₃ and CH₂CH₃,
3 to 7 membered heterocycloalkyl, preferably selected from the group consisting of oxetanyl, pyrrolidinyl, pyrrolinyl, pyrazolinyl, isoxazolinyl, oxazolinyl, isoxazolinyl, oxadiazolinyl, tetrahydropyranyl, dihydropyrazinyl, piperidinyl and morpholinyl,
wherein the 3 to 7 membered heterocycloalkyl is unsubstituted or mono- or di-substituted with at least one substituent selected from the group consisting of F, Cl, CN, OH, OCH₃, =0, CF₃, CH₃ and CH₂CH₃,
phenyl and
heteroaryl, preferably selected from the group consisting of thiazolyl, thiadiazolyl, oxazolyl, isoxazolyl, thienyl, pyrrolyl, pyrazolyl, oxadiazolyl, tetrazolyl, triazolyl, pyridyl, pyrazinyl and pyrimidinyl, wherein said phenyl or said heteroaryl is unsubstituted or mono- or di-substituted with at least one substituent selected from the group consisting of F, Cl, Br, CN, CF₃, OCF₃, OH, NH₂, CH₃, OCH₃, CH₂CH₃ and OCH₂CH₃.

In another embodiment of the present invention, Ar² is selected from the group consisting of

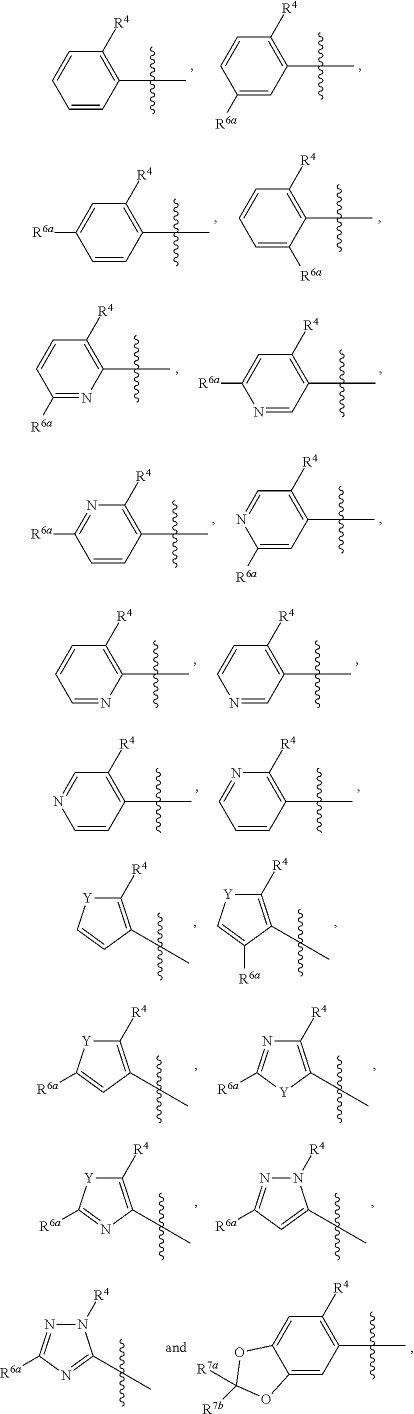

wherein Y represents S, O or NR⁵, preferably Y represents S;
R⁴ denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃;
R⁵ denotes CF₃, CF₂H, CFH₂, cyclopropyl, CH₃ or CH₂CH₃;
R^{7a} and R^{7b} each independently represent H, F or C₁₋₄-alkyl;
   and R^{6a} is selected from the group consisting of
F, Cl, Br, CN, C(═O)O—C₁₋₄-alkyl, CF₃, CF₂H, CFH₂, C(═O)NH₂, C(═O)—N(H)C₁₋₄-alkyl, C(═O)—N(C₁₋₄-alkyl)₂, C(═O)—N(H)(C₁₋₄-alkylene-OH), OCF₃, OCF₂H, OCFH₂, C₁₋₄-alkyl, OH, O—C₁₋₄-alkyl, S(═O)C₁₋₄-alkyl, SO₂—C₁₋₄-alkyl, SO₂—CF₃, SO₂—N(H)C₁₋₄-alkyl, SO₂—N(C₁₋₄-alkyl)₂, CH₂S(═O)C₁₋₄-alkyl, CH₂SO₂—C₁₋₄-alkyl, CH₂N(H)SO₂—C₁₋₄-alkyl, CH₂SO₂—N(H)C₁₋₄-alkyl, CH₂SO₂—N(C₁₋₄-alkyl)₂,
cyclopropyl, cyclobuytl, cyclopentyl, cyclohexyl,
oxetanyl, pyrrolidinyl, piperidinyl and morpholinyl,
phenyl and
oxazolyl, isoxazolyl, thiazolyl, thienyl, pyrrolyl, pyrazolyl, oxadiazolyl, tetrazolyl, pyridinyl, pyrazinyl and pyrimidinyl,
   wherein said cyclopropyl, cyclobuytl, cyclopentyl, cyclohexyl, pyrrolidinyl, piperidinyl, morpholinyl, phenyl, oxazolyl, isoxazolyl, thiazolyl, thienyl, pyrrolyl, pyrazolyl, oxadiazolyl, tetrazolyl, pyridinyl, pyrazinyl and pyrimidinyl may be unsubstituted or substituted by one or two substituents, independently selected from F, Cl, CN, CF₃, CH₃, CH₂CH₃, OH, OCH₃ or OCF₃.

Another embodiment of the present invention relates to a compound according to formula (I), characterized in that the compound is selected from one orf the compounds (I-a), (I-b) or (I-c), in particular from compound (I-a),

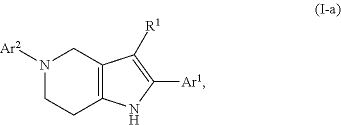

wherein R¹ represents H or F;
Ar¹ represents substructure (II),

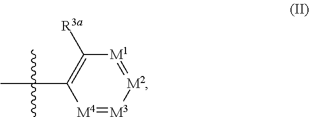

wherein
R^{3a} denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃, preferably F, CI or CH₃,
M¹, M² and M³ independently represent N or CH,
and M⁴ represents N, CH or CR^{3b},
   wherein R^{3b} denotes F, Cl, CN, CF₃, CF₂H, CFH₂, CH₃, CH₂CH₃, OCH₃ or OCH₂CH₃,
   with the proviso, that 0 or 1 of the substituents M¹, M², M³ and M⁴ represent N,
      and Ar² is selected from the group consisting of

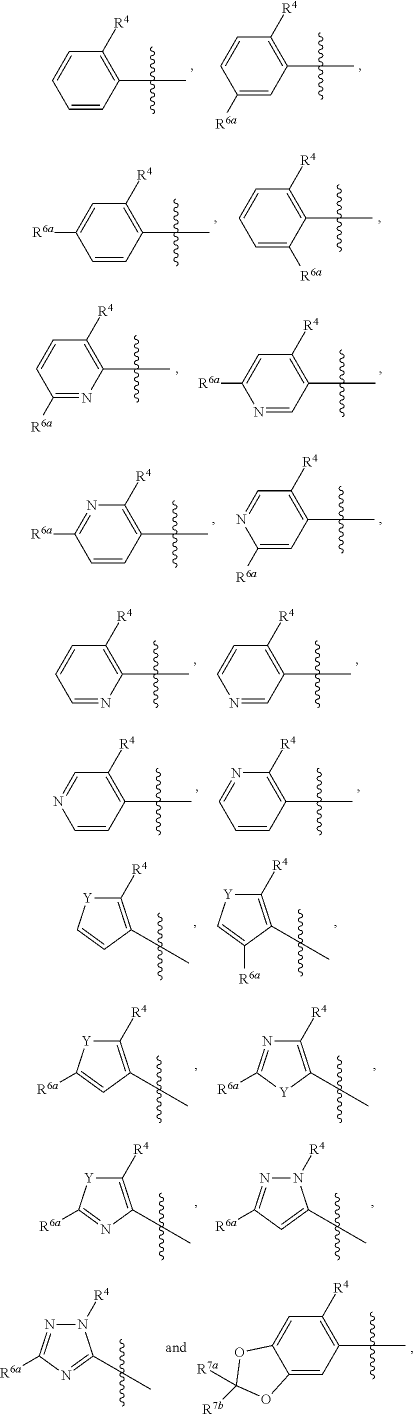

wherein Y represents S, O or NR⁵, preferably Y represents S;
R⁴ denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃;
R⁵ denotes CF₃, CF₂H, CFH₂, cyclopropyl, CH₃ or CH₂CH₃;
R^{7a} and R^{7b} each independently represent H, F or C₁₋₄-alkyl;
and R^{6a} is selected from the group consisting of
   F, Cl, Br, CN, C(═O)O—C₁₋₄-alkyl, C(═O)—N(H)C₁₋₄-alkyl, C(═O)—N(H)(C₁₋₄-alkylene-OH), CF₃, CF₂H, CFH₂, OCF₃, C₁₋₄-alkyl, OH, O—C₁₋₄-alkyl, S(═O)C₁₋₄-alkyl, SO₂—C₁₋₄-alkyl, SO₂—CF₃, SO₂—N(H)C₁₋₄-alkyl, SO₂—N(C₁₋₄-alkyl)₂, CH₂S(═O)C₁₋₄-alkyl, CH₂SO₂—C₁₋₄-alkyl, CH₂N(H)SO₂—C₁₋₄-alkyl, CH₂SO₂—N(H)C₁₋₄-alkyl, CH₂SO₂—N(C₁₋₄-alkyl)₂,
   cyclopropyl, cyclobuytl, cyclopentyl, cyclohexyl,
   pyrrolidinyl, piperidinyl and morpholinyl,
   phenyl and
   oxazolyl, isoxazolyl, thiazolyl, thienyl, pyrrolyl, pyrazolyl, oxadiazolyl, tetrazolyl, pyridinyl, pyrazinyl and pyrimidinyl,
      wherein said cyclopropyl, cyclobuytl, cyclopentyl, cyclohexyl, pyrrolidinyl, piperidinyl, morpholinyl, phenyl, oxazolyl, isoxazolyl, thiazolyl, thienyl, pyrrolyl, pyrazolyl, oxadiazolyl, tetrazolyl, pyridinyl, pyrazinyl and pyrimidinyl may be unsubstituted or substituted by one or two substituents, independently selected from F, Cl, CN, CF₃, CH₃, CH₂CH₃, OH, OCH₃ or OCF₃,
in the form of a single stereoisomer or a mixture of stereoisomers, in the form of the free compound or a physiologically acceptable salt and/or a physiologically acceptable solvate thereof.

Particularly preferred compounds according to the invention are selected from the group consisting of
1 2-(2,6-Difluoro-phenyl)-5-(5-methyl-2-pyridin-3-yl-thiazol-4-yl)-1,5,6,7-tetrahydro-pyrrolo[3,2-c]-pyridin-4-one
2 4-[2-(2,6-Difluoro-phenyl)-4,5,6,7-tetrahydro-1H-pyrrolo[3,2-c]pyridin-5-yl]-5-methyl-2-pyridin-3-yl-thiazole
3 5-(6-Chloro-4-methyl-pyridin-3-yl)-2-(2,6-difluoro-phenyl)-1,5,6,7-tetrahydro-pyrrolo[3,2-c]pyridin-4-one
4 5-(6-Chloro-4-methyl-pyridin-3-yl)-2-(2,6-difluoro-phenyl)-4,5,6,7-tetrahydro-1H-pyrrolo[3,2-c]-pyridine
5 2-(2,6-Difluoro-phenyl)-5-(5-methyl-2-pyridin-2-yl-thiazol-4-yl)-1,5,6,7-tetrahydro-pyrrolo[3,2-c]-pyridin-4-one
6 4-[2-(2,6-Difluoro-phenyl)-4,5,6,7-tetrahydro-1H-pyrrolo[3,2-c]pyridin-5-yl]-5-methyl-2-pyridin-2-yl-thiazole
7 2-(2,6-Difluoro-phenyl)-5-[3-(trifluoromethyl)phenyl]-4,5,6,7-tetrahydro-1H-pyrrolo[3,2-c]pyridine
8 2-[3-[2-(2,6-Difluoro-phenyl)-4,5,6,7-tetrahydro-1H-pyrrolo[3,2-c]pyridin-5-yl]-4-methyl-phenyl]-thiazole
9 5-(6-Chloro-2,2-difluoro-1,3-benzodioxol-5-yl)-2-(2,6-difluoro-phenyl)-4,5,6,7-tetrahydro-1H-pyrrolo[3,2-c]pyridine
10 2-(2,6-Difluoro-phenyl)-5-(4-methyl-pyridin-3-yl)-4,5,6,7-tetrahydro-1H-pyrrolo[3,2-c]pyridine
11 5-(4-Chloro-2-methyl-phenyl)-2-(2,6-difluoro-phenyl)-4,5,6,7-tetrahydro-1H-pyrrolo[3,2-c]pyridine
12 4-[2-(2,6-Difluoro-phenyl)-4,5,6,7-tetrahydro-1H-pyrrolo[3,2-c]pyridin-5-yl]-3-methyl-benzonitrile
13 4-[2-(2,6-Difluoro-phenyl)-4,5,6,7-tetrahydro-1H-pyrrolo[3,2-c]pyridin-5-yl]-3-methyl-benzoic acid methyl ester
14 4-[2-(2,6-Difluoro-phenyl)-4,5,6,7-tetrahydro-1H-pyrrolo[3,2-c]pyridin-5-yl]-N,N,3-trimethyl-benzenesulfonic acid amide
15 5-(6-Chloro-2-methyl-pyridin-3-yl)-2-(2,6-difluoro-phenyl)-4,5,6,7-tetrahydro-1H-pyrrolo[3,2-c]-pyridine
16 2-(2,6-Difluoro-phenyl)-5-(4-methyl-6-methylsulfonyl-pyridin-3-yl)-4,5,6,7-tetrahydro-1H-pyrrolo[3,2-c]pyridine
17 4-[2-(2-Chloro-6-fluoro-phenyl)-4,5,6,7-tetrahydro-1H-pyrrolo[3,2-c]pyridin-5-yl]-5-methyl-2-pyridin-3-yl-thiazole
18 5-[2-(2,6-Difluoro-phenyl)-4,5,6,7-tetrahydro-1H-pyrrolo[3,2-c]pyridin-5-yl]-4-methyl-2-pyridin-3-yl-thiazole
19 2-(2,6-Difluoro-phenyl)-5-[2-methyl-5-(trifluoromethyl)-2H-pyrazol-3-yl]-4,5,6,7-tetrahydro-1H-pyrrolo[3,2-c]pyridine
20 5-(2,2-Difluoro-6-methyl-1,3-benzodioxol-5-yl)-2-(2,6-difluoro-phenyl)-4,5,6,7-tetrahydro-1H-pyrrolo[3,2-c]pyridine
21 2-(2,6-Difluoro-phenyl)-5-(2,5-dimethoxphenyl)-4,5,6,7-tetrahydro-1H-pyrrolo[3,2-c]pyridine
22 2-(2,6-Difluoro-phenyl)-5-o-tolyl-4,5,6,7-tetrahydro-1H-pyrrolo[3,2-c]pyridine
23 4-[2-(2,6-Difluoro-phenyl)-4,5,6,7-tetrahydro-1H-pyrrolo[3,2-c]pyridin-5-yl]-5-methyl-2-phenyl-thiazole
24 4-[2-(2,6-Difluoro-phenyl)-4,5,6,7-tetrahydro-1H-pyrrolo[3,2-c]pyridin-5-yl]-5-methyl-2-pyridin-4-yl-thiazole
25 5-(5-Bromo-6-methyl-pyridin-2-yl)-2-(2,6-difluoro-phenyl)-4,5,6,7-tetrahydro-1H-pyrrolo[3,2-c]-pyridine
26 4-[2-(2,6-Difluoro-phenyl)-4,5,6,7-tetrahydro-1H-pyrrolo[3,2-c]pyridin-5-yl]-5-methyl-2-pyrazin-2-yl-thiazole
27 4-[2-(2,6-Difluoro-phenyl)-4,5,6,7-tetrahydro-1H-pyrrolo[2,3-c]pyridin-6-yl]-5-methyl-2-pyridin-3-yl-thiazole
28 2-(2,6-Difluoro-phenyl)-5-(2-methoxy-4-methylsulfonyl-phenyl)-4,5,6,7-tetrahydro-1H-pyrrolo[3,2-c]pyridine
29 3-[2-(2,6-Difluoro-phenyl)-4,5,6,7-tetrahydro-1H-pyrrolo[3,2-c]pyridin-5-yl]-4-methyl-benzonitrile
30 4-[2-(2,6-Difluoro-phenyl)-4,5,6,7-tetrahydro-1H-pyrrolo[3,2-c]pyridin-5-yl]-5-methyl-2-pyrimidin-5-yl-thiazole
31 4-[2-(2,6-Difluoro-phenyl)-4,5,6,7-tetrahydro-1H-pyrrolo[3,2-c]pyridin-5-yl]-5-ethyl-2-pyridin-3-yl-thiazole
32 5-(6-Chloro-5-methyl-pyridazin-3-yl)-2-(2,6-difluoro-phenyl)-4,5,6,7-tetrahydro-1H-pyrrolo[3,2-c]-pyridine
33 5-[2-(2,6-Difluoro-phenyl)-4,5,6,7-tetrahydro-1H-pyrrolo[3,2-c]pyridin-5-yl]-6-methoxy-2,3-dihydro-benzo[b]thiophene 1,1-dioxide
34 5-(4-Methyl-6-methylsulfonyl-pyridin-3-yl)-2-phenyl-4,5,6,7-tetrahydro-1H-pyrrolo[3,2-c]pyridine
35 5-(4-Methyl-6-methylsulfonyl-pyridin-3-yl)-2-o-tolyl-4,5,6,7-tetrahydro-1H-pyrrolo[3,2-c]pyridine
36 2-(2-Chloro-6-fluoro-phenyl)-5-(4-methyl-6-methylsulfonyl-pyridin-3-yl)-4,5,6,7-tetrahydro-1H-pyrrolo[3,2-c]pyridine
37 2-(4-Fluorophenyl)-5-(4-methyl-6-methylsulfonyl-pyridin-3-yl)-4,5,6,7-tetrahydro-1H-pyrrolo[3,2-c]pyridine
38 5-(6-Chloro-4-methyl-pyridin-3-yl)-2-(2,6-difluoro-phenyl)-1,5,6,7-tetrahydro-pyrrolo[3,2-c]pyridin-4-one
39 2-(2,6-Difluoro-phenyl)-5-(6-methoxy-4-methyl-pyridin-3-yl)-1,5,6,7-tetrahydro-pyrrolo[3,2-c]-pyridin-4-one
40 2-(2,4-Dimethoxy-phenyl)-5-(5-methyl-2-pyridin-3-yl-thiazol-4-yl)-1,5,6,7-tetrahydro-pyrrolo[3,2-c]pyridin-4-one
41 4-[2-(2-Chloro-6-fluoro-phenyl)-4-oxo-1,5,6,7-tetrahydro-pyrrolo[3,2-c]pyridin-5-yl]-3-methoxy-benzonitrile
42 2-(2-Chloro-6-fluoro-phenyl)-5-(5-methyl-2-pyridin-3-yl-thiazol-4-yl)-1,5,6,7-tetrahydro-pyrrolo-[3,2-c]pyridin-4-one
43 2-(6-Chloro-pyridin-3-yl)-5-(5-methyl-2-pyridin-3-yl-thiazol-4-yl)-1,5,6,7-tetrahydro-pyrrolo[3,2-c]-pyridin-4-one
44 5-(6-Chloro-4-methyl-pyridin-3-yl)-2-(2,4-difluoro-phenyl)-1,5,6,7-tetrahydro-pyrrolo[3,2-c]pyridin-4-one
45 2-(2-Chloro-6-fluoro-phenyl)-5-(6-chloro-4-methyl-pyridin-3-yl)-1,5,6,7-tetrahydro-pyrrolo[3,2-c]-pyridin-4-one
46 2,5-Bis(2,6-difluoro-phenyl)-1,5,6,7-tetrahydro-pyrrolo[3,2-c]pyridin-4-one
47 2-(2,6-Difluoro-phenyl)-5-(2-methyl-thiophen-3-yl)-1,5,6,7-tetrahydro-pyrrolo[3,2-c]pyridin-4-one
48 5-(2-Cyclopropyl-5-methyl-thiazol-4-yl)-2-(2,6-difluoro-phenyl)-1,5,6,7-tetrahydro-pyrrolo[3,2-c]pyridin-4-one
49 2-(2-Chloro-6-fluoro-phenyl)-5-(2,6-difluoro-phenyl)-1,5,6,7-tetrahydro-pyrrolo[3,2-c]pyridin-4-one
50 2-(2-Chloro-6-fluoro-phenyl)-5-(5-chloro-2-methyl-phenyl)-1,5,6,7-tetrahydro-pyrrolo[3,2-c]-pyridin-4-one
51 2-(2-Chloro-6-fluoro-phenyl)-5-(2-methyl-thiophen-3-yl)-1,5,6,7-tetrahydro-pyrrolo[3,2-c]pyridin-4-one
52 2-(2-Chloro-6-fluoro-phenyl)-5-(2-cyclopropyl-5-methyl-thiazol-4-yl)-1,5,6,7-tetrahydro-pyrrolo-[3,2-c]pyridin-4-one
53 2-(2-Chloro-6-fluoro-phenyl)-5-(5-methyl-2-oxazol-2-yl-thiazol-4-yl)-1,5,6,7-tetrahydro-pyrrolo-[3,2-c]pyridin-4-one
54 2-(2,6-Difluoro-phenyl)-5-(5-methyl-2-oxazol-2-yl-thiazol-4-yl)-1,5,6,7-tetrahydro-pyrrolo[3,2-c]pyridin-4-one
55 2-(2,6-Difluoro-phenyl)-5-(2-methyl-thiophen-3-yl)-4,5,6,7-tetrahydro-1H-pyrrolo[3,2-c]pyridine
56 2-Cyclopropyl-4-[2-(2,6-difluoro-phenyl)-4,5,6,7-tetrahydro-1H-pyrrolo[3,2-c]pyridin-5-yl]-5-methyl-thiazole
57 2-[4-[2-(2,6-Difluoro-phenyl)-4,5,6,7-tetrahydro-1H-pyrrolo[3,2-c]pyridin-5-yl]-5-methyl-thiazol-2-yl]-oxazole
58 2-(2,6-Difluoro-phenyl)-5-(2,5-dimethyl-2H-pyrazol-3-yl)-4,5,6,7-tetrahydro-1H-pyrrolo[3,2-c]-pyridine
59 2-(2-Chloro-6-fluoro-phenyl)-5-(2,6-difluoro-phenyl)-4,5,6,7-tetrahydro-1H-pyrrolo[3,2-c]pyridine
60 2-(2-Chloro-6-fluoro-phenyl)-5-(2-methyl-thiophen-3-yl)-4,5,6,7-tetrahydro-1H-pyrrolo[3,2-c]pyridine
61 4-[2-(2-Chloro-6-fluoro-phenyl)-4,5,6,7-tetrahydro-1H-pyrrolo[3,2-c]pyridin-5-yl]-2-cyclopropyl-5-methyl-thiazole
62 2-[4-[2-(2-Chloro-6-fluoro-phenyl)-4,5,6,7-tetrahydro-1H-pyrrolo[3,2-c]pyridin-5-yl]-5-methyl-thiazol-2-yl]-oxazole
63 2-(2-Chloro-6-fluoro-phenyl)-5-(2,5-dimethyl-2H-pyrazol-3-yl)-4,5,6,7-tetrahydro-1H-pyrrolo[3,2-c]pyridine
64 2-(2-Chloro-6-fluoro-phenyl)-5-(5-chloro-2-methyl-phenyl)-4,5,6,7-tetrahydro-1H-pyrrolo[3,2-c]pyridine
65 2-(3-Fluoro-pyridin-4-yl)-5-(5-methyl-2-pyridin-3-yl-thiazol-4-yl)-1,5,6,7-tetrahydro-pyrrolo[3,2-c]pyridin-4-one
66 4-[2-(2,6-Difluoro-phenyl)-3-iodo-4,5,6,7-tetrahydro-1H-pyrrolo[3,2-c]pyridin-5-yl]-5-methyl-2-pyridin-3-yl-thiazole
67 4-[2-(2,6-Difluoro-phenyl)-4,5,6,7-tetrahydro-1H-pyrrolo[3,2-c]pyridin-5-yl]-2-fluoro-5-methyl-benzonitrile
68 6-[2-(2,6-Difluoro-phenyl)-4,5,6,7-tetrahydro-1H-pyrrolo[3,2-c]pyridin-5-yl]-5-methyl-nicotinonitrile
69 2-(2,6-Difluoro-phenyl)-5-(6-ethoxy-4-methyl-pyridin-3-yl)-4,5,6,7-tetrahydro-1H-pyrrolo[3,2-c]-pyridine
70 2-(2,6-Difluoro-phenyl)-5-[4-methyl-6-(methylsulfinyl)-pyridin-3-yl]-4,5,6,7-tetrahydro-1H-pyrrolo-[3,2-c]pyridine
71 5-(4-Chloro-5-fluoro-2-methyl-phenyl)-2-(2,6-difluoro-phenyl)-4,5,6,7-tetrahydro-1H-pyrrolo[3,2-c]pyridine
72 2-Cyclohexyl-5-(4-methyl-6-methylsulfonyl-pyridin-3-yl)-4,5,6,7-tetrahydro-1H-pyrrolo[3,2-c]-pyridine
73 2-(2,6-Difluoro-phenyl)-5-[4-methyl-6-(trifluoromethyl)-pyridin-3-yl]-4,5,6,7-tetrahydro-1H-pyrrolo[3,2-c]pyridine
74 2-Butyl-5-(4-methyl-6-methylsulfonyl-pyridin-3-yl)-4,5,6,7-tetrahydro-1H-pyrrolo[3,2-c]pyridine
75 5-(6-Cyclopropyl-4-methyl-pyridin-3-yl)-2-(2,6-difluoro-phenyl)-4,5,6,7-tetrahydro-1H-pyrrolo[3,2-c]pyridine
76 5-(4-Methyl-6-methylsulfonyl-pyridin-3-yl)-2-tetrahydro-pyran-4-yl-4,5,6,7-tetrahydro-1H-pyrrolo-[3,2-c]pyridine
77 5-[2-(2,6-Difluoro-phenyl)-4,5,6,7-tetrahydro-1H-pyrrolo[3,2-c]pyridin-5-yl]-6-methyl-pyridine-2-carbonitrile
78 2-(2,4-Difluoro-phenyl)-5-(4-methyl-6-methylsulfonyl-pyridin-3-yl)-4,5,6,7-tetrahydro-1H-pyrrolo-[3,2-c]pyridine
79 2-(2-Fluorophenyl)-5-(4-methyl-6-methylsulfonyl-pyridin-3-yl)-4,5,6,7-tetrahydro-1H-pyrrolo[3,2-c]pyridine
80 4-[2-(2,6-Difluoro-phenyl)-4,5,6,7-tetrahydro-1H-pyrrolo[3,2-c]pyridin-5-yl]-N,3-dimethyl-benzamide
81 4-[2-(2,6-Difluoro-phenyl)-4,5,6,7-tetrahydro-1H-pyrrolo[3,2-c]pyridin-5-yl]-N,N,3-trimethyl-benzamide
82 1-[4-[2-(2,6-Difluoro-phenyl)-4,5,6,7-tetrahydro-1H-pyrrolo[3,2-c]pyridin-5-yl]-3-methyl-phenyl]-ethanone
83 5-(4-Methyl-6-methylsulfonyl-pyridin-3-yl)-2-(2-methyl-pyridin-3-yI)-4,5,6,7-tetrahydro-1H-pyrrolo-[3,2-c]pyridine
84 2-(2,6-Difluoro-phenyl)-5-[5-methoxy-2-(trifluoromethyl)-pyrimidin-4-yl]-4,5,6,7-tetrahydro-1H-pyrrolo[3,2-c]pyridine
[85 2-(2,6-Difluoro-phenyl)-5-(5-methyl-2-pyridin-3-yl-thiazol-4-yl)-4,5,6,7-tetrahydro-1H-pyrrolo[3,2-c]pyridin-3-yl]-methanol
86 2-(2,6-Difluoro-phenyl)-5-[2-methyl-4-(trifluoromethylsulfonyl)-phenyl]-4,5,6,7-tetrahydro-1H-pyrrolo[3,2-c]pyridine
87 2-(2,6-Difluoro-phenyl)-5-[2-methyl-4-(methylsulfinyl)-phenyl]-4,5,6,7-tetrahydro-1H-pyrrolo[3,2-c]pyridine
88 2-(2,6-Difluoro-phenyl)-5-(2-methyl-4-methylsulfonyl-phenyl)-4,5,6,7-tetrahydro-1H-pyrrolo[3,2-c]pyridine
89 4-Methyl-5-[5-(4-methyl-6-methylsulfonyl-pyridin-3-yl)-4,5,6,7-tetrahydro-1H-pyrrolo[3,2-c]-pyridin-2-yl]-[1,2,3]thiadiazole
90 2-(3-Fluoro-pyridin-4-yl)-5-(4-methyl-6-methylsulfonyl-pyridin-3-yl)-4,5,6,7-tetrahydro-1H-pyrrolo[3,2-c]pyridine
91 3-Bromo-2-(2,6-difluoro-phenyl)-5-(4-methyl-6-methylsulfonyl-pyridin-3-yl)-4,5,6,7-tetrahydro-1H-pyrrolo[3,2-c]pyridine
92 2-(4,6-Dimethyl-pyridin-3-yl)-5-(4-methyl-6-methylsulfonyl-pyridin-3-yl)-4,5,6,7-tetrahydro-1H-pyrrolo[3,2-c]pyridine
93 5-[4-Methyl-6-(trifluoromethyl)-pyridin-3-yl]-2-tetrahydro-pyran-4-yl-4,5,6,7-tetrahydro-1H-pyrrolo[3,2-c]pyridine
94 2-(2,6-Difluoro-phenyl)-5-(5-fluoro-4-methyl-pyridin-3-yl)-4,5,6,7-tetrahydro-1H-pyrrolo[3,2-c]-pyridine
[95 5-[2-(2,6-Difluoro-phenyl)-4,5,6,7-tetrahydro-1H-pyrrolo[3,2-c]pyridin-5-yl]-4-methyl-pyridin-2-yl]-amine
96 2-(5-Fluoro-4-methyl-pyridin-3-yl)-5-(4-methyl-6-methylsulfonyl-pyridin-3-yl)-4,5,6,7-tetrahydro-1H-pyrrolo[3,2-c]pyridine
97 2-Cyclohexyl-5-[4-methyl-6-(trifluoromethyl)-pyridin-3-yl]-4,5,6,7-tetrahydro-1H-pyrrolo[3,2-c]-pyridine
98 2-(2-Methoxy-pyridin-3-yl)-5-(4-methyl-6-methylsulfonyl-pyridin-3-yl)-4,5,6,7-tetrahydro-1H-pyrrolo[3,2-c]pyridine
99 4-[2-(2,6-Difluoro-phenyl)-4,5,6,7-tetrahydro-1H-pyrrolo[3,2-c]pyridin-5-yl]-5-methyl-2-(1-methyl-1H-pyrazol-3-yl)-thiazole
100 2-(2,6-Difluoro-phenyl)-5-[6-ethoxy-4-(trifluoromethyl)-pyridin-3-yl]-4,5,6,7-tetrahydro-1H-pyrrolo[3,2-c]pyridine
101 2-(2,6-Difluoro-phenyl)-5-(5-fluoro-2-methyl-4-methylsulfonyl-phenyl)-4,5,6,7-tetrahydro-1H-pyrrolo[3,2-c]pyridine
102 6-(2-Cyclohexyl-4,5,6,7-tetrahydro-1H-pyrrolo[3,2-c]pyridin-5-yl)-5-methyl-nicotinonitrile
103 5-(2-Cyclohexyl-4,5,6,7-tetrahydro-1H-pyrrolo[3,2-c]pyridin-5-yl)-6-methoxy-2,3-dihydro-benzo[b]thiophene 1,1-dioxide
104 2-(2,6-Difluoro-phenyl)-5-[4-methyl-6-(methylsulfonyl-methyl)-pyridin-3-yl]-4,5,6,7-tetrahydro-1H-pyrrolo[3,2-c]pyridine
105 2-(2,6-Difluoro-phenyl)-5-[4-(methoxymethyl)-2-methyl-phenyl]-4,5,6,7-tetrahydro-1H-pyrrolo[3,2-c]pyridine
106 5-(5-Cyclopropyl-2-methyl-2H-pyrazol-3-yl)-2-(2,6-difluoro-phenyl)-4,5,6,7-tetrahydro-1H-pyrrolo[3,2-c]pyridine
107 5-(5-Cyclopropyl-1-methyl-1H-pyrazol-3-yl)-2-(2,6-difluoro-phenyl)-4,5,6,7-tetrahydro-1H-pyrrolo[3,2-c]pyridine
108 2-(3-Chloro-pyridin-4-yl)-5-(4-methyl-6-methylsulfonyl-pyridin-3-yl)-4,5,6,7-tetrahydro-1H-pyrrolo[3,2-c]pyridine
109 5-[4-(Azetidin-1-ylsulfonyl)-2-methyl-phenyl]-2-(2,6-difluoro-phenyl)-4,5,6,7-tetrahydro-1H-pyrrolo[3,2-c]pyridine
110 2-Cyclohexyl-5-(5-fluoro-2-methyl-4-methylsulfonyl-phenyl)-4,5,6,7-tetrahydro-1H-pyrrolo[3,2-c]pyridine
111 2-(2,6-Difluoro-phenyl)-5-[4-methyl-6-(trifluoromethyloxy)-pyridin-3-yl]-4,5,6,7-tetrahydro-1H-pyrrolo[3,2-c]pyridine
112 2-(2,6-Difluoro-phenyl)-5-[4-methoxy-6-(trifluoromethyl)-pyridin-3-yl]-4,5,6,7-tetrahydro-1H-pyrrolo[3,2-c]pyridine
113 5-[4-Cyclopropyl-6-(trifluoromethyl)-pyridin-3-yl]-2-(2,6-difluoro-phenyl)-4,5,6,7-tetrahydro-1H-pyrrolo[3,2-c]pyridine
114 4-[2-(2,6-Difluoro-phenyl)-4,5,6,7-tetrahydro-1H-pyrrolo[3,2-c]pyridin-5-yl]-N,3-dimethyl-benzenesulfonic acid amide
115 5-(4-Methyl-6-methylsulfonyl-pyridin-3-yl)-2-tetrahydro-pyran-3-yl-4,5,6,7-tetrahydro-1H-pyrrolo-[3,2-c]pyridine
116 2-(2,6-Difluoro-phenyl)-5-[4-ethoxy-6-(trifluoromethyl)-pyridin-3-yl]-4,5,6,7-tetrahydro-1H-pyrrolo-[3,2-c]pyridine
117 4-[[4-[2-(2,6-Difluoro-phenyl)-4,5,6,7-tetrahydro-1H-pyrrolo[3,2-c]pyridin-5-yl]-3-methyl-phenyl]-sulfonyl]-morpholine
118 2-Cyclopentyl-5-(4-methyl-6-methylsulfonyl-pyridin-3-yl)-4,5,6,7-tetrahydro-1H-pyrrolo[3,2-c]-pyridine
119 2-(2,6-Difluoro-phenyl)-5-[2-methyl-4-(piperidin-1-ylsulfonyl)-phenyl]-4,5,6,7-tetrahydro-1H-pyrrolo[3,2-c]pyridine
120 N-Cyclopropyl-4-[2-(2,6-difluoro-phenyl)-4,5,6,7-tetrahydro-1H-pyrrolo[3,2-c]pyridin-5-yl]-N,3-dimethyl-benzenesulfonic acid amide
121 2-(2,6-Difluoro-phenyl)-5-[2-methyl-4-(pyrrolidin-1-ylsulfonyl)-phenyl]-4,5,6,7-tetrahydro-1H-pyrrolo[3,2-c]pyridine
122 2-(4,4-Difluoro-cyclohexyl)-5-(4-methyl-6-methylsulfonyl-pyridin-3-yl)-4,5,6,7-tetrahydro-1H-pyrrolo[3,2-c]pyridine
123 2-(2,6-Difluoro-phenyl)-5-[2-methyl-5-[1-(trifluoromethyl)-cyclopropyl]-2H-pyrazol-3-yl]-4,5,6,7-tetrahydro-1H-pyrrolo[3,2-c]pyridine
124 2-(2,6-Difluoro-phenyl)-5-[2-ethyl-5-[1-(trifluoromethyl)-cyclopropyl]-2H-pyrazol-3-yl]-4,5,6,7-tetrahydro-1H-pyrrolo[3,2-c]pyridine
125 2-(2,6-Difluoro-phenyl)-5-[1-ethyl-5-[1-(trifluoromethyl)-cyclopropyl]-1H-pyrazol-3-yl]-4,5,6,7-tetrahydro-1H-pyrrolo[3,2-c]pyridine
126 5-(5-Cyclopropyl-2-ethyl-2H-pyrazol-3-yl)-2-(2,6-difluoro-phenyl)-4,5,6,7-tetrahydro-1H-pyrrolo-[3,2-c]pyridine
127 4-[2-(4,6-Dimethyl-pyridin-3-yl)-4,5,6,7-tetrahydro-1H-pyrrolo[3,2-c]pyridin-5-yl]-N,3-dimethyl-benzenesulfonic acid amide
128 4-[2-(4,4-Difluoro-cyclohexyl)-4,5,6,7-tetrahydro-1H-pyrrolo[3,2-c]pyridin-5-yl]-N,3-dimethyl-benzenesulfonic acid amide
129 4-(2-Cyclohexyl-4,5,6,7-tetrahydro-1H-pyrrolo[3,2-c]pyridin-5-yl)-N-methyl-benzenesulfonic acid amide
130 2-(2,6-Difluoro-phenyl)-5-[2-ethyl-5-(trifluoromethyl)-2H-pyrazol-3-yl]-4,5,6,7-tetrahydro-1H-pyrrolo[3,2-c]pyridine
131 4-[2-(2,6-Difluoro-phenyl)-4,5,6,7-tetrahydro-1H-pyrrolo[3,2-c]pyridin-5-yl]-N-ethyl-3-methyl-benzenesulfonic acid amide
132 N-[4-[2-(2,6-Difluoro-phenyl)-4,5,6,7-tetrahydro-1H-pyrrolo[3,2-c]pyridin-5-yl]-3-methyl-phenyl]-N-methyl-methanesulfonic acid amide
133 4-[2-(2,6-Difluoro-phenyl)-4,5,6,7-tetrahydro-1H-pyrrolo[3,2-c]pyridin-5-yl]-5-methyl-2-methyl-sulfonyl-thiazole
134 5-[4-Methyl-6-(trifluoromethyl)-pyridin-3-yl]-2-tetrahydro-pyran-3-yl-4,5,6,7-tetrahydro-1H-pyrrolo[3,2-c]pyridine
135 2-(4,6-Dimethyl-pyridin-3-yl)-5-[4-methyl-6-(trifluoromethyl)-pyridin-3-yl]-4,5,6,7-tetrahydro-1H-pyrrolo[3,2-c]pyridine
136 4-[2-(2,6-Difluoro-phenyl)-4,5,6,7-tetrahydro-1H-pyrrolo[3,2-c]pyridin-5-yl]-3-methyl-N-(2,2,2-trifluoro-ethyl)-benzenesulfonic acid amide
137 2-(2,6-Difluoro-phenyl)-5-[5-(1-methoxy-cyclopropyl)-2-methyl-2H-pyrazol-3-yl]-4,5,6,7-tetrahydro-1H-pyrrolo[3,2-c]pyridine
138 2-(2,6-Difluoro-phenyl)-4-methyl-5-(4-methyl-6-methylsulfonyl-pyridin-3-yl)-4,5,6,7-tetrahydro-1H-pyrrolo[3,2-c]pyridine
139 2-(2,6-Difluoro-phenyl)-5-[2-ethyl-5-(1-methoxy-cyclopropyl)-2H-pyrazol-3-yl]-4,5,6,7-tetrahydro-1H-pyrrolo[3,2-c]pyridine
140 5-[6-(Difluoro-methoxy)-4-methoxy-pyridin-3-yl]-2-(2,6-difluoro-phenyl)-4,5,6,7-tetrahydro-1H-pyrrolo[3,2-c]pyridine
141 2-(5-Fluoro-4-methyl-pyridin-3-yl)-5-[4-methyl-6-(trifluoromethyl)-pyridin-3-yl]-4,5,6,7-tetrahydro-1H-pyrrolo[3,2-c]pyridine
142 2-(2,6-Difluoro-phenyl)-3-fluoro-5-(4-methyl-6-methylsulfonyl-pyridin-3-yl)-4,5,6,7-tetrahydro-1H-pyrrolo[3,2-c]pyridine
143 2-(2,6-Difluoro-phenyl)-5-(6-ethoxy-4-methoxy-pyridin-3-yl)-4,5,6,7-tetrahydro-1H-pyrrolo[3,2-c]-pyridine
144 2-(2,6-Difluoro-phenyl)-3-fluoro-5-(4-ethoxy-6-trifluormethyl-pyridin-3-yl)-4,5,6,7-tetrahydro-1H-pyrrolo[3,2-c]pyridine
   optionally in the form of a single stereoisomer or a mixture of stereoisomers, in the form of the free compound and/or a physiologically acceptable salt or solvate thereof.

The compounds according to the present invention are useful for calcium release-activated calcium (CRAC) channel regulation, preferably for use in CRAC channel inhibition. The substances according to the invention hence act, for example, on the CRAC channel relevant in connection with various diseases, so that they are suitable as a pharmacologically active compound in pharmaceutical compositions.

In another aspect, the present invention therefore also provides pharmaceutical compositions, containing at least one compound according to the invention and optionally one or more suitable, pharmaceutically compatible auxiliaries and/or, if appropriate, one or more further pharmacologically active compounds.

The pharmaceutical composition according to the invention is suitable for administration to adults and children, including toddlers and babies. The pharmaceutical composition according to the invention may be found as a liquid, semisolid or solid pharmaceutical form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, if appropriate pressed into tablets, decanted in capsules or suspended in a liquid, and also be administered as much.

In addition to at least one compound according to the invention, if appropriate in the form of one of its pure stereoisomers, its racemate or in the form of mixtures of the stereoisomers, in particular the enantiomers or diastereomers, in any desired mixing ratio, or if appropriate in the form of a corresponding salt or respectively in the form of a corresponding solvate, the pharmaceutical composition according to the invention conventionally contains further physiologically compatible pharmaceutical auxiliaries which can for example be selected from the group consisting of excipients, fillers, solvents, diluents, surface-active substances, dyes, preservatives, blasting agents, slip additives, lubricants, aromas and binders. Likewise the compound according to the invention, if appropriate in the form of one of its pure stereoisomers, or if appropriate in the form of a corresponding salt or respectively in the form of a corresponding solvate, may also incorporated into the pharmaceutical composition in the form of a prodrug, which releases the active pharmacological agent through normal metabolic processes.

The selection of the physiologically compatible auxiliaries and also the amounts thereof to be used depend on whether the pharmaceutical composition is to be applied orally, subcutaneously, parenterally, intravenously, intraperitoneally, intradermally, intramuscularly, intranasally, buccally, rectally or locally, for example to infections of the skin, the mucous membranes and of the eyes. Preparations in the form of tablets, dragées, capsules, granules, pellets, drops, juices and syrups are preferably suitable for oral application; solutions, suspensions, easily reconstitutable dry preparations and also sprays are preferably suitable for parenteral, topical and inhalative application. The compounds according to the invention used in the pharmaceutical composition according to the invention in a repository in dissolved form or in a plaster, agents promoting skin penetration being added if appropriate, are suitable percutaneous application preparations. Orally or percutaneously applicable preparation forms can release the respective compound according to the invention also in a delayed manner.

The pharmaceutical compositions according to the invention are prepared with the aid of conventional means, devices, methods and process known in the art, such as are described for example in “Remington's Pharmaceutical Sciences”, A. R. Gennaro (Editor), 17^{th} edition, Mack Publishing Company, Easton, Pa., 1985, in particular in Part 8, Chapters 76 to 93. The corresponding description is introduced herewith by way of reference and forms part of the disclosure. The amount to be administered to the patient of the respective substituted compounds according to the invention of the above-indicated general formula I may vary and is for example dependent on the patient's weight or age and also on the type of application, the indication and the severity of the disorder. Conventionally 0.001 to 100 mg/kg, preferably 0.05 to 75 mg/kg, particularly preferably 0.05 to 50 mg of at least one such compound according to the invention are applied per kg of the patient's body weight.

CRAC channels are believed to be involved in a variety of diseases or disorders in mammals such as humans. These include inflammatory disorders, allergic disorders and disorders of the immune system as well as disorders involving platelet or thrombotic activity.

Examples of allergic disorders include: rhinitis (such as allergic rhinitis), sinusitis, rhinosinusitis, chronic or recurrent otitis media, drug reactions, insect sting reactions, latex allergy, conjunctivitis, urticaria, anaphylaxis and anaphylactoid reactions, atopic dermatitis and food allergies.

Examples of inflammatory disorders include: inflammatory lung disorders (such as asthma, acute respiratory distress syndrome, acute lung injury, chronic obstructive pulmonary disease, bronchiectasis and cystic fibrosis); chronic inflammatory disorders of joints (such as arthritis, rheumatoid arthritis, osteoarthritis and bone diseases associated with increased bone resorption); inflammatory bowel diseases (such as Barrett's oesophagus, ileitis, ulcerative colitis and Crohn's disease); inflammatory disorders of the eye (such as corneal dystrophy, trachoma, uveitis, sympathetic ophthalmitis and endophthalmitis); inflammatory diseases of the kidney (such as glomerulonephritis, nephrosis, nephritic syndrome and IgA nephropathy); inflammatory diseases of the liver; inflammatory disorders of the skin (such as psoriasis and eczema); inflammatory diseases of the central nervous system (such as chronic demyelinating diseases of the nervous system, multiple sclerosis, AIDS-related neurodegeneration and Alzheimers disease, infectious meningitis, enceophalomyelitis, Parkinson's disease, Huntington's disease, amyo-trophic lateral sclerosis and viral or autoimmune encephalitis); inflammatory diseases of the muscle (such as polymyositis and polymyalgia rheumatica); inflammatory diseases of the heart (such as myocarditis and cardiomyopathy, ischemic heart disease, myocardial infarction and atherosclerosis); other diseases with significant inflammatory components, including tuberculosis; leprosy; allogeneic or xenogeneic transplantation (cells, stem cells, tissues or organs) graft rejection, graft-versus-host disease; pre-eclampsia; endometriosis, chronic liver failure; brain and spinal cord trauma and cancer; and conditions where systemic inflammation of the body may also be present (such as septic shock, hemorrhagic or anaphylactic shock or shock induced by cancer chemotherapy).

Examples of disorders of the immune system include: autoimmune diseases of the central and peripheral nervous system (such as multiple sclerosis, myasthenia gravis, Eaton-Lambert Myasthenic syndrome); autoimmune neurophathies (such as Guillain-Barre); autoimmune diseases of the eye (such as autoimmune uveitis); autoimmune diseases of the blood (such as autoimmune haemolytic anemia, pernicious anemia, and autoimmune thrombocytopenia e.g. Idiopathic Thrombocytopaenic Purpura); autoimmune diseases of the vasculature (such as temporal arteritis, anti-phospholipid syndrome, vasculitides e.g. Wegener's granulomatosis and Behcet's disease); autoimmune diseases of the skin (such as alopecia areata, psoriasis, dermatitis herpetiformis, pemphigus vulgaris, bullous pemphigoid and vitiligo); autoimmune disease of the gastrointestinal tract (such as coeliac disease, Crohn's disease, ulcerative colitis, primary biliary cirrhosis and autoimmune hepatitis); autoimmune disorders of the endocrine glands (such as Type1 diabetes mellitus, autoimmune thyroiditis, Grave's disease, Hashimoto's thyroiditis, autoimmune oophoritis and orchitis); autoimmune disorder of the adrenal gland (such as Addisons disease); autoimmune disorders of the exocrine glands (such as Sjogren's syndrome); and multi system autoimmune diseases including connective tissue and musculoskeletal system diseases (such as rheumatoid arthritis, systemic lupus erythematosus, scleroderma, polymyositis, dermatomyositis), spondyloarthropathies (such as ankylosing spondylitis and psoriatic arthritis).

Examples of conditions where anti-platelet or anti-thrombotic activity is useful for treatment and/or prophylaxis include: ischemic heart disease, myocardial infarction, cerebrovascular accident (stroke) and vascular thrombosis (venous, arterial and intra-cardiac).

Further diseases or conditions which may be treated by the compounds of the invention include conditions where mast cells and basophils contribute to pathology, such as mast cell leukaemia, mastocytosis, endometriosis and basophil leukaemia.

The term “disorders and/or diseases which are mediated, at least in part, by CRAC channels”, is intended to include each of or all of the above disease states.

It is believed that the compounds of formula (I), having ICRAC inhibitory activity, may inhibit mast cell degranulation and/or inhibit T cell activation. Compounds having such activity may be particularly suitable for the treatment of a number of diseases and conditions, for example asthma; allergies such as allergic rhinitis; and nasal polyposis.

Another aspect of the present invention therefore relates to a compound according to the first aspect of the present invention for the treatment and/or prophylaxis of a or more disorder and/or disease, selected from the group consisting of glomerulonephritis, uveitis, hepatic diseases or disorders, especially hepatitis, renal diseases or disorders, chronic obstructive pulmonary disease (COPD), rheumatoid arthritis (RA), multiple sclerosis, inflammatory bowel disease (IBD), especially Barrett's oesophagus, ileitis, ulcerative colitis or Crohn's Disease, vasculitis, dermatitis, dermatomyositis, atopic dermatitis, scleroderma, osteoarthritis, inflammatory muscle disease, allergic rhinitis, vaginitis, interstitial cystitis, osteoporosis, eczema, psoriasis, allogeneic or xenogeneic transplantation (cells, stem cells, tissues or organs) graft rejection, graft-versus-host disease, lupus erythematosus, type I diabetes, pulmonary fibrosis, thyroiditis, myasthenia gravis, autoimmune hemolytic anemia, cystic fibrosis, chronic relapsing hepatitis, hepatitis, primary biliary cirrhosis, allergic conjunctivitis, asthma, nasal polyposis; Sjogren's syndrome, cancer and other proliferative diseases, and autoimmune diseases or disorders.

Another embodiment of this aspect of the present invention relates to a compound according to the first aspect of the present invention for the treatment and/or prophylaxis of autoimmune diseases, in particular rheumatoid arthritis and psoriatic arthritis.

Another embodiment of this aspect of the present invention relates to a compound according to the first aspect of the present invention for the treatment and/or prophylaxis of inflammatory disorders of the skin, in particular psoriasis as and/or eczema, most preferably psoriasis.

Another embodiment of this aspect of the present invention relates to a compound according to the first aspect of the present invention for the treatment and/or prophylaxis of chronic inflammatory disorders of the joints, in particular arthritis, rheumatoid arthritis and/or osteoarthritis arthritis, most preferably rheumatoid arthritis (RA).

Yet another embodiment of this aspect of the present invention relates to a compound according to the first aspect of the present invention for the treatment and/or prophylaxis of inflammatory bowel diseases, in particular Barrett's oesophagus, ileitis, ulcerative colitis and Crohn's disease.

Yet another embodiment of this aspect of the present invention relates to a compound according to the first aspect of the present invention for the treatment and/or prophylaxis of allogeneic or xenogeneic transplantation graft rejection, in particular transplantation grafts of cells, stem cells, tissues and/or organs.

Yet another embodiment of this aspect of the present invention relates to a compound according to the first aspect of the present invention for the treatment and/or prophylaxis of autoimmune diseases of the central and peripheral nervous system, in particular multiple sclerosis, myasthenia gravis and/or Eaton-Lambert Myasthenic syndrome, most preferably multiple sclerosis.

Yet another embodiment of this aspect of the present invention relates to a compound according to the first aspect of the present invention for the treatment and/or prophylaxis of inflammatory lung disorders, in particular asthma, acute respiratory distress syndrome, acute lung injury, chronic obstructive pulmonary disease, bronchiectasis and/or cystic fibrosis, most preferably asthma.

Yet another embodiment of this aspect of the present invention relates to a compound according to the first aspect of the present invention for the treatment and/or prophylaxis of allergies, in particular allergic rhinitis.

Another aspect of the present invention provides the use of at least one compound according to the first aspect of the present invention for the preparation of a pharmaceutical composition for the treatment and/or prophylaxis of one or more of the above mentioned diseases and/or disorders.

Another aspect of the invention provides the use of at least one compound according to the first aspect of the present invention for the preparation of a pharmaceutical composition for the treatment and/or prophylaxis of one or more of the diseases and/or disorders, selected from the group consisting of inflammatory disorders and/or autoimmune diseases and/or allergic disorders, preferably selected from the group consisting of psoriasis and/or psoriatic arthritis; rheumatoid arthritis; inflammatory bowel disease; asthma and allergic rhinitis.

Another aspect of the present invention is a method for the treatment and/or prophylaxis, in particular for of one or more of the above mentioned diseases and/or disorders,

in a mammal, in particular in a human, in need of treatment and/or prophylaxis of the respective disease and/or disorder,
which comprises the administration of an effective amount of at least one compound according to the first aspect of the present invention or the administration of a pharmaceutical composition according to the invention to the mammal.

One embodiment of the invention is a method for the treatment and/or prophylaxis of disorders and/or diseases, selected from the group consisting of inflammatory disorders and/or autoimmune diseases and/or allergic disorders, preferably selected from the group consisting of psoriasis and/or psoriatic arthritis; rheumatoid arthritis; inflammatory bowel disease; asthma and allergic rhinitis,

in a mammal, in particular in a human, in need of treatment and/or prophylaxis of the respective disease and/or disorder,
which comprises the administration of an effective amount of at least one compound according to the first aspect of the present invention or the administration of a pharmaceutical composition according to the invention to the mammal.

The term “effective amount” according to the present invention means that administered amount of the compound or the pharmaceutical composition that will result in a therapeutically desired biological or medical response of a tissue, system, mammal or human.

A therapeutically desired biological or medical response is understood to be an improved treatment, healing, prevention, or amelioration of a disease, disorder, or side effect, or a decrease in the rate of advancement of a disease or disorder in a mammal, as compared to a corresponding mammal who has not been administered such amount. The term “therapeutically desired biological or medical response” includes also the enhancement of a normal physiological function.

The term “compounds according to the first aspect of the present invention” in foregoing aspects of the invention encompasses all possible stereoisomers and tautomers as well as the respective corresponding acids, bases, salts and solvates.

The embodiments and in particular the preferred embodiments of any aspect of the present invention apply to all other aspects of the inventions respectively.

### Preparation Schemes

Compounds of the invention may be made by the methods depicted in the reaction schemes below and described for examples of the invention. The following reaction schemes are illustrative only and various modifications of the methods may be made by those skilled in the art in order to obtain compounds of the invention. Compounds described in this invention may be synthesized through starting from building blocks represented through the generic structure IM-5. These Building blocks can be synthesized according to Scheme 1 starting from a substituted or non-substituted beta-amino acid.

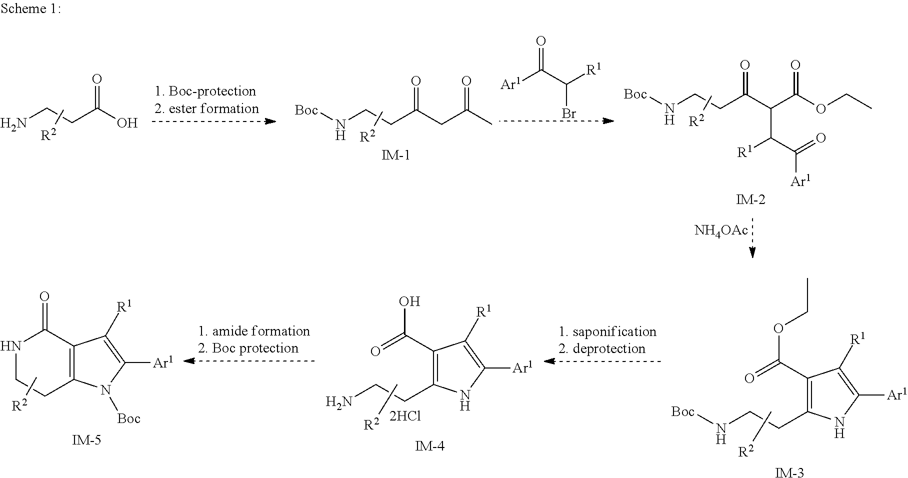

Intermediates IM-5 can be coupled with aryl-halides mediated by a transition metal such as Pd(0) or cooper(I) to yield intermediates IM-6. These can be directly converted through Boc-deprotection to the target molecules of the structure TM-1 or alternatively can be converted in 2 steps to target molecules of type TM-2. In an alternative manner, TM-1 can be converted to TM-2 through reduction with a hydride source such as BH₃ (Scheme 2).

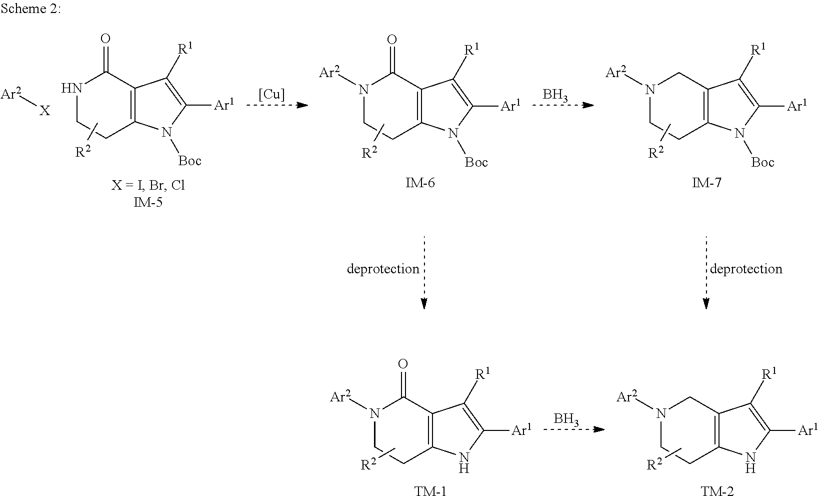

Intermediate building blocks IM-11 can be synthesized starting from protected and substituted or non-substituted pipiridine-3-ones through a Stork enamine formation followed by nucleophilic addition to a alpha-bromo-ketone compound to yield intermediates of type IM-8. These can be condensed with an ammonia synthone, such as NH₄OAc, to yield intermediates IM-10, which after Boc-protection of the pyrrole and debenzylation yield building blocks IM-11 (Scheme 3).

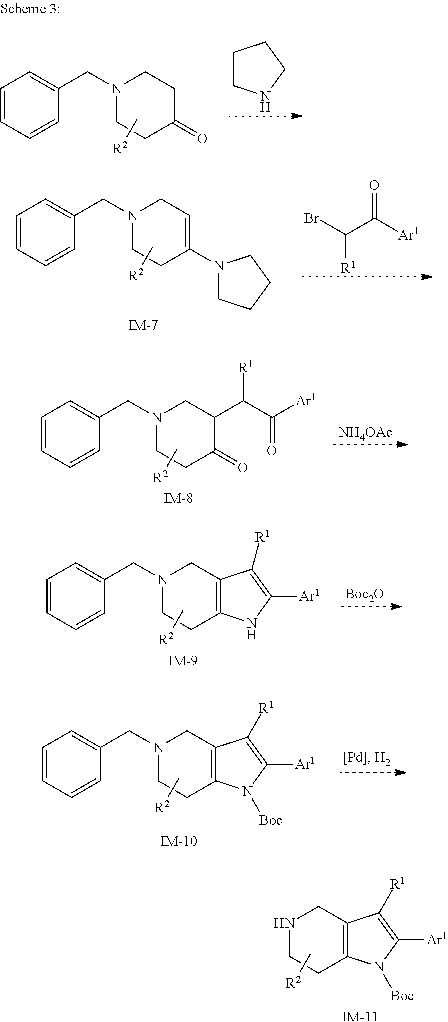

Intermediates IM-9, displayed in Scheme 3 can alternatively be synthesized starting from 3-alkyl-4 amino pyridines through an ortho-lithiation/acylation sequence to yield intermediates IM-12 which can be condensed, under acidic conditions, to the aza-indoles IM-13. These can be reduced in two steps the required intermediates IM-9.

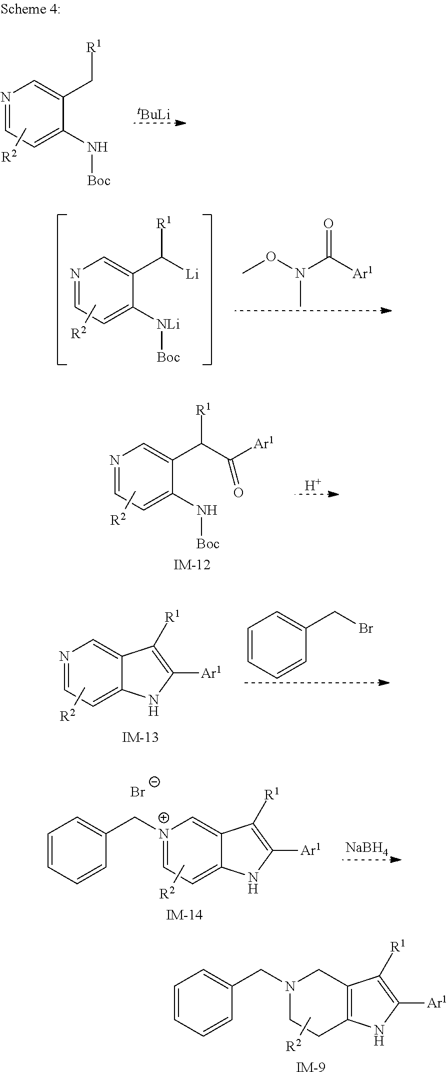

Target molecules TM-2 can be synthesized starting from intermediates IM-11 through a transition metal catalyzed cross coupling with the respective aryl halides or triflates (Scheme 5).

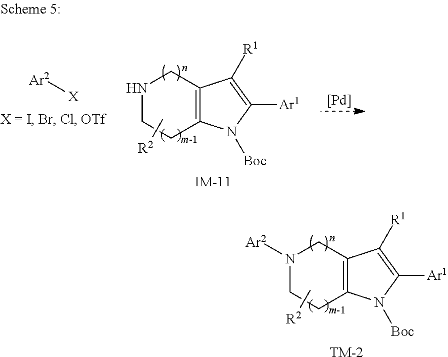

Alternatively target compounds TM-2 may be realized in a similar fashion as it described in Schemes 4 and 5 with shifting the C—N bond forming reaction into the first step of the reaction sequence (Scheme 6).

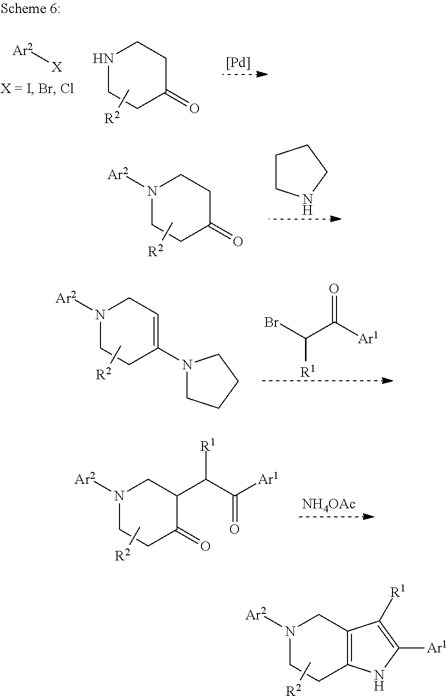

The 7 membered ring intermediates IM-21 and IM-22 may be synthesized according to Scheme 7. Synthesis of the target molecules may be realized according to Scheme 5.

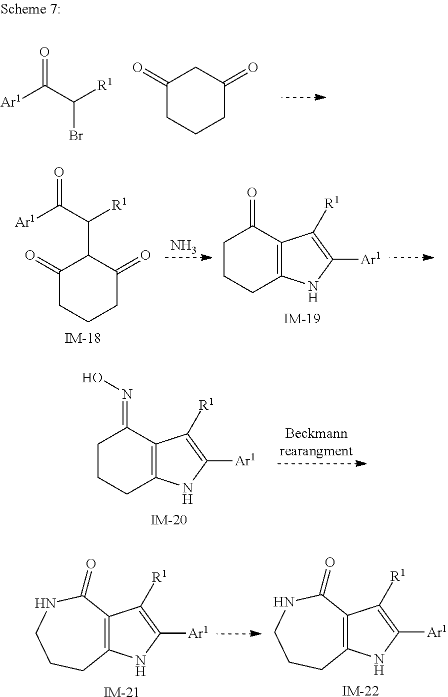

The 5 membered ring intermediates IM-26 and IM-27 may be synthesized according to Scheme 8. Synthesis of the target molecules may be realized according to Scheme 5.

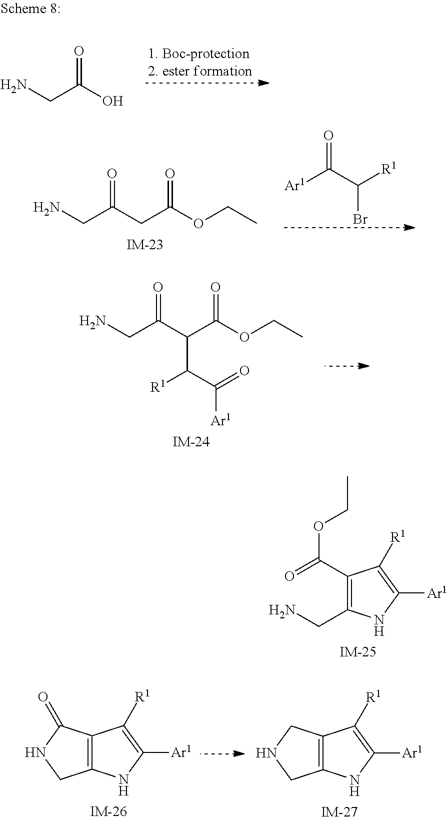

The following examples of the invention were prepared according to the reaction schemes 1 to 8. These examples are, however, not construed to limit the scope of the invention in any manner.

### EXAMPLES

Starting materials and reagents are available from commercial suppliers such as for example Acros, Aldrich, Apollo, Fluka, FluoroChem, Lancaster, Manchester Organics, MatrixScientific, Maybridge, Merck, TCI, Oakwood, etc., or the synthesis has been described as such in the literature or the materials may be prepared by conventional methods known to those skilled in the art.

All the intermediate products and exemplary compounds were analytically characterized by means of ¹H-NMR spectroscopy. In addition, mass spectrometry tests (MS, m/z for [M+H]⁺) were carried out for all the exemplary compounds and selected intermediate products.

### EXAMPLES

Starting materials and reagents are available from commercial suppliers such as for example Acros, Aldrich, Apollo, Fluka, FluoroChem, Lancaster, Manchester Organics, MatrixScientific, Maybridge, Merck, TCI, Oakwood, etc., or the synthesis has been described as such in the literature or the materials may be prepared by conventional methods known to those skilled in the art.

All the intermediate products and exemplary compounds were analytically characterized by means of ¹H-NMR spectroscopy. In addition, mass spectrometry tests (MS, m/z for [M+H]⁺) were carried out for all the exemplary compounds and selected intermediate products.

### ABBREVIATIONS

The indication “equivalents” (“eq.” or “eq” or “equiv.”) means molar equivalents, “RT” or “rt” means room temperature (23±7° C.), “RM” means reaction mixture, “M” are indications of concentration in mol/l, “aq.” means aqueous, “sat.” means saturated, “sol.” means solution, “conc.” means concentrated.

BH₃.DMS: borane-dimethylsulfide complex; Boc: tert-butyloxycarbonyl; (Boc)₂O: i-tert-butyldicarbonate; CC: column chromatography; CDI: ,1′-carbonyldiimidazole; Cy: cyclohexane; DDQ: 2,3-dichloro-5,6-dicyano-1,4-benzoquinone; DMAP: 4-(dimethylamino)-pyridine; DMF: N,N-dimethylformamide; DIPEA: diisopropylethylamine; EDC.HCl: N-(3-dimethylaminopropyl)-N′-ethylcarbodiimide hydrochloride; Et₂O: diethyl ether; EtOH: ethanol; EtOAc: ethyl acetate; h: hour(s); Hex: hexane(s); HOBT: 1-hydroxybenzo-triazole; LDA: lithium diisopropylamide; LiHMDS: lithium hexamethyldisilazide; MeCN: acetonitrile; MeOH: methanol; min: minute(s); NBS: N-bromosuccinimide; NH₄OAc: ammonium acetate; PE: petroleum ether; Pd₂(dba)₃: tris(dibenzylideneacetone)dipalladium(0); rac-BINAP: racemic (2,2′-bis(diphenylphosphino)-1,1′-binaphthyl); TFA: trifluoroacetic acid; THF: tetrahydrofuran; TMEDA: N,N,N′,N′-tetramethyl-ethan-1,2-diamine; X-phos: dicyclohexyl(2′,4′,6′-triisopropylbiphenyl-2-yl)phosphane.

### Analytical and Purification Methods:

The mixing ratios of solvents or eluents for chromatography are specified in v/v.

Liquid Chromatography with Mass Spectrometry Detection: LC-MS

### Method 1:

Agilent LC-MS 1200 Rapid Resolution with detector MSD6140; Detection: MM-ES+APCI+DAD (254 nm); Fragmentation: 50 V [pos/neg]; Column: Agilent SB-C18, 2.1×30 mm, 3.5 micron; Column temperature: 30° C.; Flow rate: 0.8 mL/min; Runtime: 4 min.

Eluent: A: Water; B: EtOH with 1 vol-% formic acid

Gradient: t=0 min: 95/5 (A/B); t=1.00 min: 95/5 (A/B); t=4.00 min: 0/100 (A/B)

### Method 2:

Agilent 1290 Infinity UHPLC-TOF system; Detection: Agilent G4212A DAD (190-400 nm)+Agilent 6224 TOF; Column: Zorbax SB-C18 Rapid Resolution HD, 2.1×50 mm; Column temperature: 80° C. Flow rate: 2.3 mL/min; Runtime: 1.38 min.

Eluent: A: Water with 0.1 vol-% formic acid; B: MeCN with 0.1 vol-% formic acid

Gradient: t=0 min: 98/2 (A/B); t=1.20 min: 0/100 (A/B); t=1.29 min.: 0/100 (A/B); t=1.31 min 98/2 (A/B); t=1.39 min: 98/2 (A/B).

### Method 3:

Applied Biosystem LCMS/MS API 2000; Detection: UV, 220 and 260 nm; Column: Zorbax Extend C18 4.6×50 mm, 5 micron; Column temperature: 30° C.; Flow rate: 1.2 mL/min; Runtime: 5 min.

Eluent: A: Water with 0.05 vol-% formic acid; B: MeCN

Gradient: t=0 min.: 90/10 (A/B); t=1.50 min: 70/30 (A/B); t=3.00 min: 10/90 (A/B); t=4.00 min: 10/90 (A/B); t=5.00 min: 90/10 (A/B).

### Method 4:

Applied Biosystem LCMS/MS API 2000; Detection: UV, 220 and 260 nm; Column: Gemini C-18 (15×4.6); Column temperature: 30° C.; Flow rate: 0.7 mL/min; Runtime: 5.1 min.

Eluent: A: Water with 0.05 vol-% formic acid; B: MeCN

Gradient: t=0 min: 70/30 (A/B); t=3.80 min: 5/95 (A/B); t=5.00 min: 5/95 (A/B); t=5.10 min: 70/30 (A/B).

### Chromatography

Büchi MPLC system (Stationary phase: silica gel, 40-50μ) or PuriFlash 430 (Stationary phase: Interchime-cartridges).

### NMR Spectroscopy

Bruker Advance II 400 MHz and Bruker Advance II 600 MHz spectrometer

### Building Block Synthesis

### Building Block 1: 2-(2,6-Difluorophenyl)-4,5,6,7-tetrahydro-1H-pyrrolo[3,2-c]pyridine Hydrochloride (BB-1)

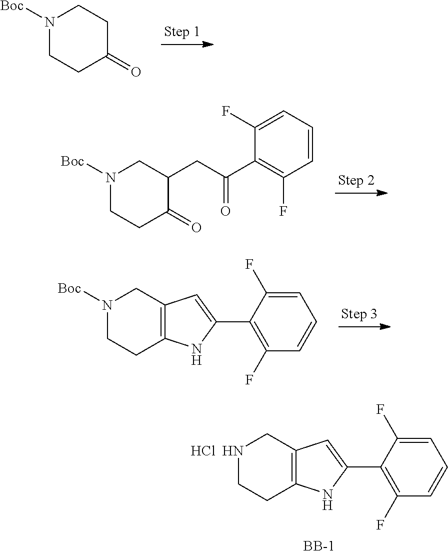

Step 1:

A solution of tert-butyl 4-oxopiperidine-1-carboxylate (7.63 g, 38.3 mmol) and pyrrolidine (6.54 g, 92 mmol) in benzene (70 mL) was heated to reflux under Dean-Stark conditions for 5 h. The volatiles were removed under reduced pressure and the residue was dissolved in THF (30 mL). NEt₃ (5.8 g, 57.5 mmol) and 2-bromo-1-(2,6-difluorophenyl)ethanone (9.0 g, 38.3 mmol) were added and the mixture was heated to 60° C. for 18 h. The volatiles were removed under reduced pressure and the residue was treated with 0.5M HCl (50 mL) and was extracted with Et₂O. The combined organic layers were dried and the volatiles were removed under reduced pressure to yield the desired compound of step 1 (10.0 g, 74%). LC-MS (method 1): m/z: [(M-Boc)+H]⁺=254.2 (MW-Boc calc.=252.08), Rₜ=3.6 min.

Step 2:

The crude compound of step 1 (5 g, 14.2 mmol) and NH₄OAc (5.4 g, 70 mmol) were dissolved in EtOH (50 mL) and the suspension was heated to 80° C. for 1 h. The volatiles were removed under reduced pressure and the residue was dissolved in CH₂Cl₂ (500 mL) and was washed with sat. Na₂CO₃ (50 mL). The organic layer was dried and the volatiles were removed under reduced pressure. The residue was purified by CC (100 g, silica gel, CH₂Cl₂) to yield the desired compound of step 2 (1.17 g, 25%). LC-MS (method 1): m/z: [(M-Boc)+H]⁺=235.2 (MW calc.=233.09); Rₜ=4.0 min.

Step 3:

A solution of the intermediate from step 2 (860 mg, 2.57 mmol) in CH₂Cl₂ (25 mL) was treated at it with TFA (5 mL) and the resulting mixture was stirred for 1 h. The volatiles were removed under reduced pressure, the residue was dissolved in CH₂Cl₂ (50 mL) and was washed with sat. NaHCO₃ (25 mL) and was treated with 1 M HCl (50 mL). The precipitate formed was filtered and the solid was washed with CH₂Cl₂ and was dried under reduced pressure to yield BB-1 (550 mg, 79%).

¹H-NMR (400 MHz, DMSO-d₆): δ (ppm)=2.91 (t, J=6 Hz, 2H); 3.36 (m, 2H); 4.08 (m, 2H); 6.35 (d, J=1.6 Hz, 1H); 7.17 (m, 2H); 7.30 (m, 1H); 9.34 (br s, 2H); 11.08 (br s, 1H).

LC-MS (method 1): m/z: [M+H]⁺=235.2 (MW calc.=234.24); Rₜ=2.3 min.

### Building Block 2: tert-butyl 2-(2,6-difluorophenyl)-4,5,6,7-tetrahydro-1H-pyrrolo[3,2-c]pyridine-1-carboxylate (BB-2)

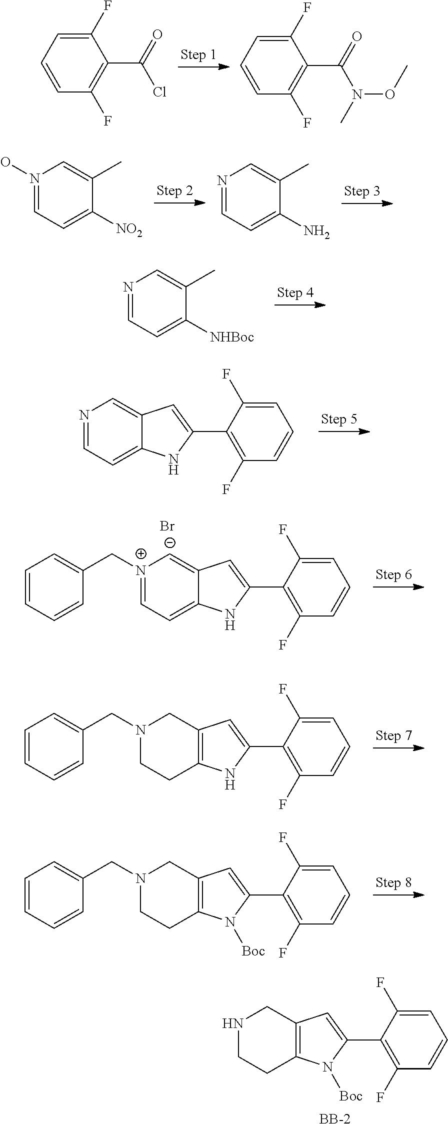

Step 1:

2,6-difluoro benzoyl chloride (75.0 g, 426 mmol) was added dropwise to a solution of N,O-dimethyl-hydroxyl-amine hydrochloride (62.6 g, 639 mmol) and NEt₃ (180 mL, 1.28 mol) in dry CH₂Cl₂ (800 mL) at 0° C. and the resulting solution was stirred at it for 16 h under N₂ atmosphere. The RM was diluted with CH₂Cl₂ (200 mL) and successively washed with water and brine, dried and the volatiles were removed under reduced pressure. The residue was purified by CC (silica gel, 100 g, Cy/EtOAc) to yield the desired compound (74.0 g, 87%).

Step 2:

A solution of 3-methyl-4-nitropyridine N-oxide (30.0 g, 195 mmol) and 10% Pd—C(6.0 g) in EtOH (450 mL) was stirred at it under H₂ (5 bar) for 36 h. The RM was filtered through a pad of Celite™ and the volatiles were removed under reduced pressure to yield the desired compound (20.0 g, 95%).

Step 3:

(Boc)₂O (89.0 mL, 400 mmol) was added to a solution of the intermediate from step 2 (36.0 g, 333 mmol) in dry THF (400 mL) and the mixture was stirred at it for 10 h. The volatiles were removed under reduced pressure and the residue was purified by CC (silica gel, CH₂Cl₂/MeOH) to yield the desired compound (55.0 g, 80%).

Step 4:

t-BuLi (1.5 M in pentane, 576 mL, 865 mmol) was added over 15 min to a solution of the intermediate of step 3 (60.0 g, 289 mmol) and TMEDA (130 mL, 865 mmol) in dry THF (2.0 L) at −70° C. and the mixture was stirred at −50° C. for 1 h. A solution of the intermediate of step 1 (69.5 g, 346 mmol) in dry THF (400 mL) was added at −70° C. and the RM was stirred at −70° C. for 1 h and was then gradually warmed to rt. To the solution HCl (5 M, 1500 mL) was slowly added and the mixture was thereafter heated to 70° C. for 5 h. The mixture was chilled and the pH was adjusted to 7 through the addition of NaHCO₃. The mixture was extracted with EtOAc, the combined organic layers were washed with brine, dried and the volatiles were removed under reduced pressure. The residue was purified by CC (silica gel, CH₂Cl₂/MeOH) to yield the desired compound (16.0 g, 25%).

Step 5:

Benzyl bromide (16.4 mL, 139 mmol) was added to a solution of the intermediate of step 4 (16.0 g, 69.6 mmol) in MeCN (500 mL) and the resulting mixture was heated to reflux for 34 h. The volatiles were removed under reduced pressure and the residue was triturated with Et₂O, filtered and dried under reduced pressure to yield the desired compound (17.0 g, 78%)

Step 6:

NaBH₄ (8.0 g, 212 mmol) was added to a solution of the intermediate from step 5 (17.0 g, 42.3 mmol) in mixture of MeOH (200 mL) and water (200 mL) at 0° C. and the mixture was stirred at it for 1 h and heated to reflux for 5 h. The volatiles were removed under reduced pressure and residue was dissolved in EtOAc and was washed with water, brine. The organic layer was dried and the volatiles were removed under reduced pressure to yield the desired crude compound which was used without further purification.

Step 7:

To a solution of NEt₃ (11.3 mL, 80.2 mmol) and (Boc)₂O (18 mL, 80.24 mmol) a solution of the crude compound from step 6 (13.0 g, 40.1 mmol) and DMAP (4.9 g, 40.123 mmol) in dry THF (300 mL) was added at it and the resulting mixture was heated to reflux for 10 h. The RM was chilled and the volatiles were removed under reduced pressure. The residue was dissolved in EtOAc and was washed with water and brine. The organic layer was dried and the volatiles were removed under reduced pressure. The residue was purified by CC (silica gel; Cy/EtOAc) to yield the desired compound (11.0 g, 64%).

Step 8:

A solution of the intermediate of step 7 (11.0 g, 25.9 mmol) and Pd(OH)₂ (20% Pd, 2.5 g) in MeOH (350 mL) was stirred under H₂ atmosphere (3 bar) for 1 h. The RM was filtered through a pad of Celite™ and the volatiles were removed under reduced pressure to yield the desired compound (7.0 g, 81%).

¹H-NMR (400 MHz, DMSO-d₆): δ (ppm)=2.75-2.77 (m, 2H), 2.94-2.97 (t, J=6 Hz, 2H), 3.63 (s, 2H), 6.17 (s, 1H), 7.13-7.17 (m, 2H), 7.40-7.45 (m, 1H).

### Building Block 3: tert-butyl 2-(2,6-difluorophenyl)-4-oxo-4,5,6,7-tetrahydro-1H-pyrrolo[3,2-c]pyridine-1-carboxylate (BB-3)

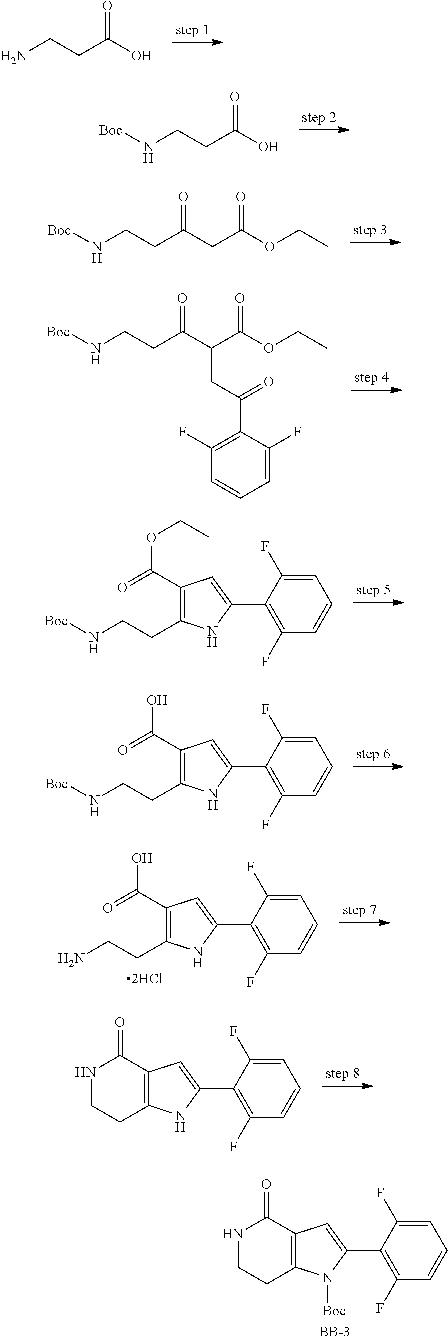

Step 1:

To a solution of 3-amino propanoic acid (50 g, 561.8 mmol) in 1,4-dioxane (250 mL) and 3.5 M NaOH (150 mL) at 0° C. was added (Boc)₂O (184 g, 843 mmol) was added and the RM was stirred at it for 16 h. The volatiles were removed under reduced pressure and the pH of the residue adjusted to 6 with 1N HCl. The obtained precipitate was filtered, washed with water and the solid was dried under reduced pressure. The solid was re-dissolved in CH₂Cl₂ (500 mL) and the solution was filtered over a pad of silica gel. The volatiles were removed under reduced pressure to yield the desired compound (85 g, 80%).

Step 2:

To a solution of the intermediate of step 1 (20.0 g, 106 mmol) in dry THF (200 mL) was added CDI (25.7 g, 159 mmol) at 0° C. and the RM was stirred at it for 3 h. A solution of MgCl₂ (15.1 g, 159 mmol) and potassium ethyl malonate (27.0 g, 159 mmol) in THF (200 mL) was added and the RM was heated to 60° C. for 16 h. The volatiles were removed under reduced pressure and the residue was treated with 5% aqueous KHSO₄ (100 mL) and water (100 mL) and the aqueous layer was extracted with EtOAc. The combined organic layers were dried and the volatiles were removed under reduced pressure. The residue was purified by CC (silica gel, eluent, EtOAc/Hex) to yield the desired compound (23.0 g, 63%).

Step 3:

To a solution of the intermediate of step 2 (5.00 g, 19.3 mmol) in dry acetone (250 mL), K₂CO₃ (3.99 g, 29.0 mmol) and 2,6-difluoro phenacyl bromide (4.52 g, 19.3 mmol) were added at rt. The RM was stirred at 60° C. for 48 h. The volatiles were removed under reduced pressure and the residue was treated with water and was extracted with EtOAc. The combined organic layers were dried and the volatiles were removed under reduced pressure. The residue was purified by CC (silica gel, EtOAc/Hex) to yield the desired compound (6.0 g).

Step 4:

A solution of the intermediate of step 3 (6.0 g, 14.5 mmol) and NH₄OAc (11.2 g, 145 mmol, 10 eq) in EtOH (100 mL) was stirred at it for 16 h. The volatiles were removed under reduced pressure and water (100 mL), and saturated aqueous NaHCO₃ solution (100 mL) was added. The aqueous layer was extracted with EtOAc, the combined organic layers were dried and the volatiles were removed under reduced pressure. The residue was purified by CC (silica gel, EtOAc/Hex) to yield the desired compound (3.3 g).

Step 5:

To a solution of the intermediate of step 4 (3.3 g, 8.37 mmol) in EtOH (100 mL), 0.1 M NaOH (50 mL) was added and the RM was stirred at 90° C. for 16 h. The volatiles were removed under reduced pressure and the residue was diluted with water (100 mL). The pH of the aqueous layer was adjusted to ˜6 through the addition of 1N HCl at 10° C. and the formed precipitate was isolated through filtration and dried under reduced pressure to yield the desired compound (1.7 g).

Step 6:

To A solution of the intermediate of step 5 (2.00 g, 5.46 mmol) in 1,4-dioxane (40 mL) was added 4N HCl in dioxane (40 mL) was added and the RM was stirred for at it for 10 h. The volatiles were removed under reduced pressure and the residue was triturated with Et₂O and pentane. The formed precipitate was isolated through filtration and was dried under reduced pressure to yield the desired compound (1.4 g, 75%).

Step 7:

To a solution of the intermediate from step 6 (1.40 g, 4.13 mmol) in a mixture of CH₂Cl₂ (40 mL) and DMF (40 mL), DIPEA (2.66 g, 20.7 mmol), EDCHCI (720 mg, 4.13 mmol), HOBt (557 mg, 4.13 mmol) were consecutively added and the RM was stirred under N₂ at it for 24 h. The RM was diluted with ice water and was extracted with CH₂Cl₂. The combined organic layers were dried and the volatiles were removed under reduced pressure. The residue was purified by CC (silica gel, CH2Cl2/MeOH) to yield the desired compound (480 mg, 47%).

Step 8:

To a solution of the intermediate of step 7 (100 mg, 0.403 mmol) in CH₂Cl₂ (100 mL) were consecutively added Et₃N (0.084 mL, 0.604 mmol), DMAP (9.8 mg, 0.0806 mmol), (Boc)₂O (131 mg, 0.604 mmol) were added and the RM was stirred at 10° C. for 2 h. The RM was diluted with water and was extracted with CH₂Cl₂. The combined organic layers were dried and the volatiles were removed under reduced pressure. The residue was purified by CC (silica gel, CH₂Cl₂/MeOH) to yield BB-3 (80 mg, 57%). LC-MS (method 4): m/z: [M+H]⁺=349.0 (MW calc.=348.13); Rₜ=3.3 min.

¹H NMR (300 MHz; DMSO-d₆): δ (ppm)=1.28 (s, 9H), 3.09-3.14 (t, J=9 Hz, 2H), 3.44-3.48 (t, J=6 Hz, 2H), 6.55 (s, 1H), 7.18-7.23 (t, J=6 Hz, 2H), 7.37 (s, 1H), 7.47-7.47 (q, J=6 Hz, 1H) ppm.

### Building Block 4: tert-butyl 2-(2-chloro-6-fluorophenyl)-4-oxo-4,5,6,7-tetrahydro-1H-pyrrolo[3,2-c]pyridine-1-carboxylate (BB-4)

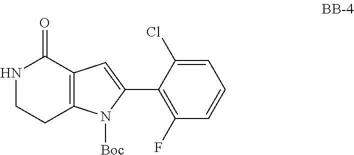 [SMILES: CC(C)(C)OC(=O)n1c(-c2c(F)cccc2Cl)cc2c1CCNC2=O]

BB-4 was synthesized in analogy to the preparation of BB-3 with substituting 2,6-difluoro phenacyl bromide in step 3 with 2-bromo-1-(2-chloro-6-fluorophenyl)ethanone.

LC-MS (method 4): m/z: [M+H]⁺=365.0 (MW calc.=364.1); Rₜ=3.44 min.

¹H NMR (300 MHz; DMSO-d₆): δ (ppm)=1.24 (s, 9H), 3.11-3.15 (t, J=6 Hz, 2H), 3.44-3.48 (t, J=6 Hz, 2H), 6.48 (s, 1H), 7.30-7.52 (m, 4H) ppm.

### Building Block 5: tert-butyl 2-(2, chloro-6-fluorophenyl)-4,5,6,7-tetrahydro-1H-pyrrolo (3,2-C)pyridine-1-carboxylate (BB-5)

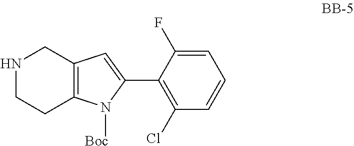 [SMILES: CC(C)(C)OC(=O)n1c(-c2c(F)cccc2Cl)cc2c1CCNC2]

Building block 5 was synthesized in analogy to the synthesis of building block 2 with substituting 2,6-difluoro benzoyl chloride in step 1 with 2-chloro-6-fluoro benzoyl chloride.

¹H-NMR (DMSO-d₆, 400 MHz), δ (ppm)=9.92 (s, 1H), 7.51-7.42 (m, 2H), 7.32 (t, J=8.8 Hz, 1H), 6.30 (s, 1H), 4.10 (s, 2H), 3.43-3.31 (m, 2H), 3.19-3.09 (m, 2H).

### Building Block 6: tert-butyl 2-(2,4-dimethoxphenyl)-4-oxo-4,5,6,7-tetrahydro-1H-pyrrolo[3,2-c]pyridine-1-carboxylate (BB-6)

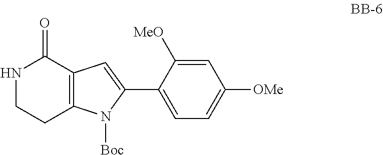 [SMILES: COc1ccc(-c2cc3c(n2C(=O)OC(C)(C)C)CCNC3=O)c(OC)c1]

BB-6 was synthesized in analogy to the preparation of BB-3 with substituting 2,6-difluoro phenacyl bromide in step 3 with 2-bromo-1-(2,4-dimethoxyphenyl)ethanone.

LC-MS (method 4): m/z: [M+H]⁺=373.0 (MW calc.=372.17); Rₜ=3.20 min.

¹H NMR (300 MHz; DMSO-d₆): δ (ppm)=1.28 (s, 9H), 3.00-3.04 (t, J=6 Hz, 2H), 3.42-3.46 (t, J=6 Hz, 2H), 3.68 (s, 3H), 3.79 (s, 3H), 6.18 (s, 1H), 6.53-6.57 (m, 2H), 7.11-7.14 (d, J=9 Hz, 1H), 7.22 (s, 1H) ppm.

### Building Block 7: tert-butyl 2-(2,4-difluorophenyl)-4-oxo-4,5,6,7-tetrahydro-1H-pyrrolo[3,2-c]pyridine-1-carboxylate (BB-7)

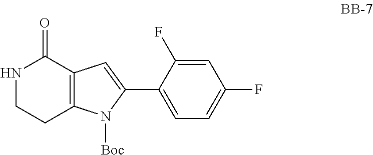 [SMILES: CC(C)(C)OC(=O)n1c(-c2ccc(F)cc2F)cc2c1CCNC2=O]

BB-7 was synthesized in analogy to the preparation of BB-3 with substituting 2,6-difluoro phenacyl bromide in step 3 with 2-bromo-1-(2,4-difluorophenyl)ethanone.

LC-MS (method 4): m/z: [M+H]⁺=349.0 (MW calc.=348.13); Rₜ=3.44 min.

¹H NMR (400 MHz; DMSO-d₆): δ (ppm)=1.31 (s, 9H), 3.06-3.11 (t, J=9 Hz, 2H), 3.42-3.48 (t, J=9 Hz, 2H), 6.47 (s, 1H), 7.13-7.18 (t, J=6 Hz, 1H), 7.34 (m, 2H), 7.43-7.52 (q, J=9 Hz, 1H) ppm.

### Building Block 8: tert-butyl 2-(3-fluoropyridin-4-yl)-4-oxo-4,5,6,7-tetrahydro-1H-pyrrolo[3,2-c]pyridine-1-carboxylate (BB-8)

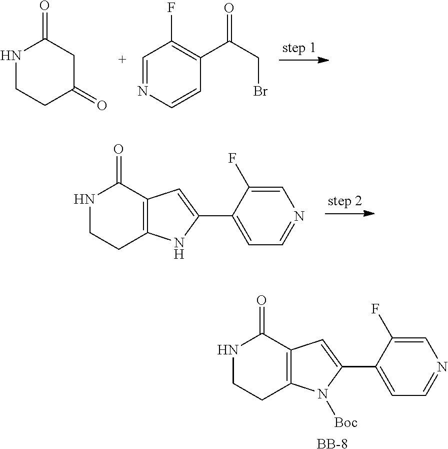

Step 1:

To a solution of piperidine-2,4-dione (3 g, 26.54 mmol) in EtOH (100 mL) in a sealed tube, 2-bromo-1-(3-fluoropyridin-4-yl)ethanone (9.49 g, 31.85 mmol) and NH₄OAc (8.2 g, 106.19 mmol) were added and the RM was stirred at it for 16. The solvent was evaporated under reduced pressure and water (100 mL) was added. The precipitated solid was filtered, dried under vacuum to yield the desired product (2.1 g, 70%).

Step 2:

To a solution of the intermediate from step 1 (4 g, 17.316 mmol) in dry CH₂Cl₂ (300 mL), Et₃N (2.62 g, 25.974 mmol), DMAP (0.422 g, 3.46 mmol) and (Boc)₂O (5.66 g, 25.974 mmol) were added at 0° C. and the RM was stirred at 10° C. for 2 h. Water (100 mL) was added and the mixture was extracted with CH₂Cl₂ (100 mL). The organic layer was dried over Na₂SO₄, filtered and concentrated under reduced pressure. The residue was purified by CC (silica gel, 100-200 mesh; eluent: MeOH—CH₂Cl₂ (3:97)) to afford compound BB-8 (3.3 g, 58%).

LC-MS (method 4): m/z: [M+H]⁺=332.0 (MW calc.=331.13); Rₜ=2.50 min.

¹H NMR (400 MHz; DMSO-d₆): δ (ppm)==1.33 (s, 9H), 3.09-3.14 (t, J=9 Hz, 2H), 3.44-3.48 (t, J=6 Hz, 2H), 6.70 (s, 1H), 7.40 (s, 1H), 7.52-7.55 (t, J=6 Hz, 2H), 8.47-8.49 (d, J=6 Hz, 1H), 8.61 (s, 1H) ppm.

### Building Block 9: tert-butyl 2-(2,6-difluorophenyl)-4,5,6,7-tetrahydro-1H-pyrrolo[2,3-c]pyridine-1-carboxylate (BB-9)

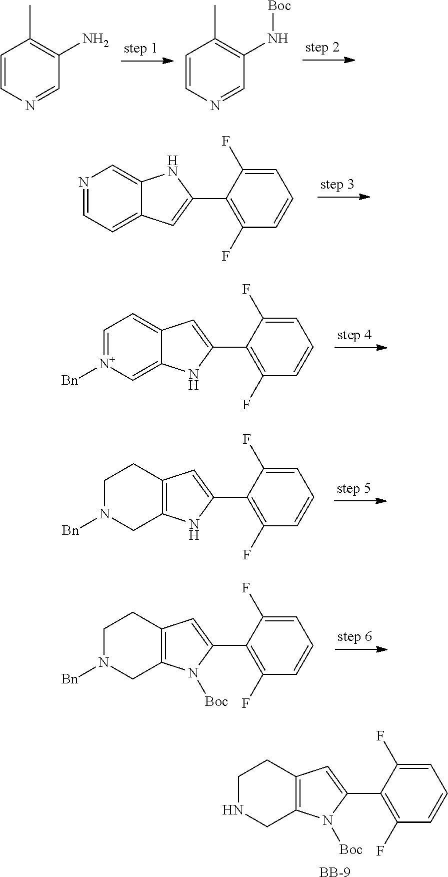

Step 1:

1M LiHMDS in THF (445 mL, 444 mmol) and (Boc)₂O (59.4 mL, 267 mmol) were added a solution of 3-amino 4-methyl pyridine (24.0 g, 222 mmol) in dry THF (700 mL) at 0° C. After stirring for 4 h at rt, the RM was quenched with aqueous saturated NH₄Cl and was extracted with EtOAc. The combined organic layers were washed with water and brine, dried and the volatiles were removed under reduced pressure. The residue was purified by CC (silica gel, CH₂Cl₂/MeOH) to yield the desired compound (21.0 g, 46%).

Step 2:

1.5M t-BuLi in pentane (193 mL, 289 mmol) was added to a solution of the intermediate of step 1 (20.0 g, 96.1 mmol) and TMEDA (43.5 mL, 289 mmol) in dry THF (600 mL) at −70° C. under inert atmosphere and the RM was stirred for 1 h at −50° C. A solution of ethyl 2,6-difluorobenzoate (21.5 g, 115 mmol) in dry THF (200 mL) was added at −70° C. and the RM was stirred for 1 h at −70° C., 1 h at 0° C. and 2 h at rt. The RM was quenched with 5.5M HCl (600 mL) and heated at 70° C. for 5 h. The mixture was cooled to rt, neutralized through the addition of NaHCO₃ and extracted with EtOAc. The combined organic layers were dried and the volatiles were removed under reduced pressure. The residue was purified by CC (silica gel, CH₂Cl₂/MeOH) to yield the desired compound (5.5 g, 25%).

Step 3:

Benzyl bromide (4 mL, 35.8 mmol) was added to a solution of the intermediate of step 2 (5.5 g, 23.9 mmol) in MeCN (120 mL) and heated at reflux for 16 h. The volatiles were removed under reduced pressure and the residue was washed with Et₂O and hexane to yield the desired compound (6.5 g).

Step 4:

NaBH₄ (3.8 g, 102 mmol) was added to a solution of the intermediate of step 3 (6.5 g, 20.3 mmol) in mixture of MeOH (100 mL) and water (100 mL) at 0° C. The mixture was stirred at it for 1 h and then heated at reflux for 5 h. The volatiles were removed under reduced pressure and the residue was dissolved in EtOAc and was washed with water and brine. The organic layer was dried and the volatiles were removed under reduced pressure to yield the desired compound (5.5 g).

Step 5:

NEt₃ (4.8 mL, 35.0 mmol) and (Boc)₂O (59.4 mL, 267 mmol) were added to a solution of the intermediate of step 4 (5.5 g, 17.0 mmol) and DMAP (2.5 g, 20.4 mmol) in dry THF (150 mL) at it and the resulting mixture was heated to reflux for 10 h. The RM was cooled to it and the volatiles were removed under reduced pressure. The residue was dissolved in EtOAc and was washed with water and brine, dried and the volatiles were removed under reduced pressure. The residue was purified by CC (silica gel, Hex/EtOAc) to yield the desired compound (3.8 g, 52%).

Step 6:

20% Pd(OH)₂/C (800 mg) was added to a solution of the intermediate of step 5 (3.8 g, 8.96 mmol) in MeOH (150 mL) at rt. The RM was stirred under H₂ atmosphere (5 bar) at it for 1 h. The RM was filtered over a pad of Celite™ and the volatiles were removed under reduced pressure to yield the desired compound (1.6 g, 56%).

¹H NMR (400 MHz; DMSO-d₆): δ (ppm)=7.48-7.39 (m, 1H), 7.20-7.11 (m, 2H), 6.22 (s, 1), 4.00 (s, 2H), 2.93 (t, J=7.2 Hz, 2H), 2.47 (t, J=7.2 Hz, 2H), 1.26 (s, 9H) ppm.

### Building Block 10: tert-butyl 2-phenyl-4,5,6,7-tetrahydro-1H-pyrrolo[3,2-c]pyridine-1-carboxylate (BB-10)

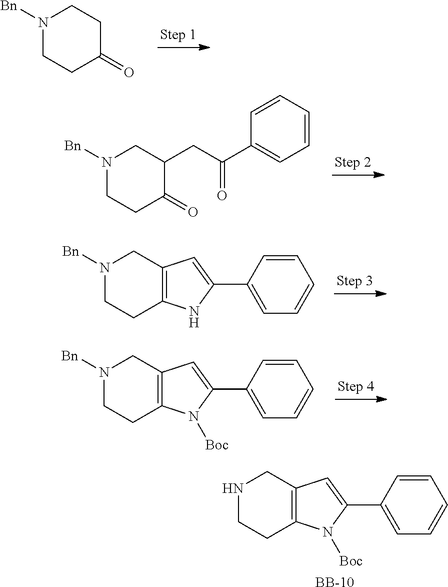

A solution of 1-benzylpiperidin-4-one (2.49 g, 13.2 mmol) and pyrrolidine (2.25 g, 31.7 mmol) in toluene (28 mL) was heated to reflux under Dean-Stark conditions for 5 h. The volatiles were removed under reduced pressure and the residue was dissolved in THF (16 mL). NEt₃ (2.75 mL, 19.8 mmol) and 2-bromo-1-phenylethanone (2.63 g, 13.2 mmol) were added and the mixture was heated in the dark to 60° C. for 18 h. The volatiles were removed under reduced pressure and the residue was treated with 0.5M HCl (50 mL) and was extracted with Et₂O. The combined organic layers were dried and the volatiles were removed under reduced pressure to yield the desired compound of step 1 which was used for the next step without further purification.

Step 2:

The crude compound of step 1 (4.05 g) and NH₄OAc (5.09 g, 66 mmol) were dissolved in EtOH (54 mL) and the suspension was heated to 80° C. for 1 h. The volatiles were removed under reduced pressure and the residue was dissolved in CH₂Cl₂ and was washed with sat. Na₂CO₃ and brine. The organic layer was dried and the volatiles were removed under reduced pressure. The residue was purified by chromatography (Interchim® cartridge 30SiHP/40 g, Cy/EtOAc) to yield the desired compound of step 2 (1.4 g, 37%).

LC-MS (method 2): m/z: [(M-Boc)+H]⁺=289.17 (MW calc.=288.39); Rₜ=0.47 min.

Step 3:

To a solution of the intermediate of step 2 (580 mg, 2.01 mmol) in MeCN (8.5 mL) were consecutively added DMAP (5 mg, 0.04 mmol), NEt₃ and (Boc)₂O (658 mg, 3.01 mmol) and the mixture was stirred at it for 3 d. 0.1 M aqueous NaOH was added and the mixture was extracted with EtOAc. The combined organic layers were dried and the volatiles were removed under reduced pressure. The residue was purified by chromatography (Interchim® cartridge 30SiHP/25 g, Cy/EtOAc) to yield the desired compound (500 mg, 64%).

LC-MS (method 2): m/z: [(M-Boc)+H]⁺=389.3 (MW calc.=388.50); Rₜ=0.79 min.

Step 4:

A solution of the intermediate of step 3 (360 mg, 0.927 mmol) in MeOH (11 mL) was hydrogenated in an H-Cube® continuous-flow hydrogenation reactor (30 mm 10% Pd/C cartridge; flow: 0.3 mL/min; 10 bar H₂) at 60° C. The volatiles were removed under reduced pressure and the residue was purified by chromatography (Interchim® cartridge 30SiHP/12 g, EtOAc/EtOH/NH₃) to yield BB-10 (130 mg, 47%).

LC-MS (method 2): m/z: [(M-Boc)+H]⁺=299.2 (MW calc.=298.38); Rₜ=0.68 min.

¹H NMR (400 MHz; DMSO-d₆): δ (ppm)=7.37-7.31 (m, 2H), 7.29-2.21 (m, 3H), 6.01 (s, 1H), 3.59 (s, 2H), 2.92 (t, J=5.3 Hz, 2H), 2.71 (t, J=5.3 Hz, 2H), 1.25 (s, 9H).

### Building Block 11: tert-butyl 2-o-tolyl-4,5,6,7-tetrahydro-1H-pyrrolo[3,2-c]pyridine-1-carboxylate (BB-11)

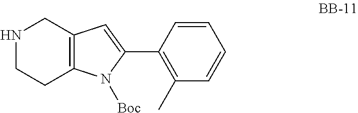 [SMILES: Cc1ccccc1-c1cc2c(n1C(=O)OC(C)(C)C)CCNC2]

BB-11 was synthesized in analogy to the preparation of BB-10 with substituting 2-2-bromo-1-phenylethanone in step 1 with 2-bromo-1-o-tolylethanone.

LC-MS (method 2): m/z: [(M-Boc)+H]⁺=313.3 (MW calc.=312.41); Rₜ=0.75 min.

¹H NMR (400 MHz; DMSO-d₆): δ (ppm)=7.35-7.05 (m, 4H), 5.86 (s, 1H), 3.60 (s, 2H), 2.92 (t, J=5 Hz, 2H), 2.73 (t, J=5 Hz, 2H), 2.08 (s, 3H), 1.14 (s, 9H).

### Building Block 12: 2-(4-fluorophenyl)-4,5,6,7-tetrahydro-1H-pyrrolo[3,2-c]pyridine (BB-12)

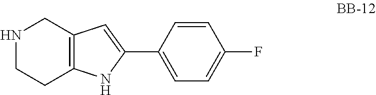 [SMILES: Fc1ccc(-c2cc3c([nH]2)CCNC3)cc1]

BB-12 was synthesized in analogy to the preparation of BB-10 with substituting 2-2-bromo-1-phenylethanone in step 1 with 2-bromo-1-(4-fluorophenyl)ethanone and without doing the Boc-protection in step 3.

LC-MS (method 2): m/z: [(M-Boc)+H]⁺=217.2 (MW calc.=216.25); Rₜ=0.54 min.

¹H NMR (400 MHz; DMSO-d₆): δ (ppm)=10.75 (s, 1H), 7.57-7.51 (m, 2H), 7.15-7.08 (m, 2H), 6.15 (d, J=2.5 Hz, 1H), 3.61 (s, 2H), 2.92 (t, J=5.8 Hz, 2H), 2.52 (t, J=5.8 Hz, 2H).

### Building Block 13: 2-cyclohexyl-4,5,6,7-tetrahydro-1H-pyrrolo[3,2-c]pyridine hydrochloride (BB-13)

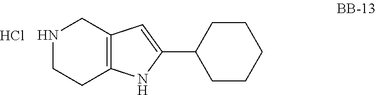 [SMILES: Cl ; c1c(C2CCCCC2)[nH]c2c1CNCC2]

BB-13 was synthesized in analogy to the preparation of BB-1.

### Building Block 14: 2-butyl-4,5,6,7-tetrahydro-1H-pyrrolo[3,2-c]pyridine hydrochloride (BB-14)

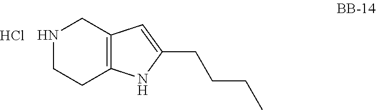

BB-14 was synthesized in analogy to the preparation of BB-1.

¹H NMR (600 MHz, DMSO-d₆) δ (ppm)=3.97 (t, J=4.6 Hz, 1H), 3.12-3.08 (m, 1H), 2.75 (dt, J=20.7, 6.2 Hz, 2H), 2.46 (td, J=7.5, 3.1 Hz, 2H), 1.87-1.79 (m, 1H), 1.57-1.47 (m, 1H), 1.50-1.36 (m, 2H), 1.29 (tq, J=14.0, 7.6 Hz, 2H), 0.96-0.78 (m, 4H).

### Building Block 15: 5-Bromo-2-cyclopropyl-4-methylpyridine (BB-15)

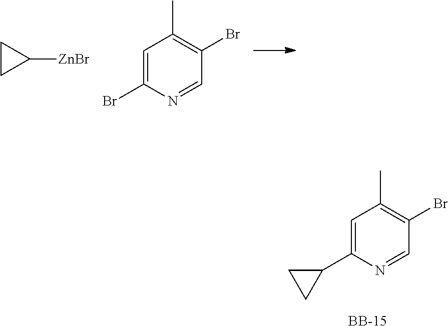

A mixture of 2,5-dibromo-4-methyl-pyridine (500 mg, 1.99 mmol), cyclopropyl zinc bromide (0.5 M in THF, 5.0 mL, 2.49 mmol) and Pd(PPh₃)₄ (40 mg, 20 μmol) in THF (3.2 mL) was heated under an N₂ to 70° C. for 1 h. The RM was cooled to it and was poured into saturated NaHCO₃. The aqueous layer was extracted with Et₂O, the combined organic layers were washed with brine, dried and the volatiles were removed under reduced pressure to yield the desired compound (200 mg, 47%).

LC-MS (Method 2): m/z [M+H]⁺=212.1 (MW calc. 212.09); Rₜ=1.11 min.

### Building Block 16: 2-(tetrahydro-2H-pyran-4-yl)-4,5,6,7-tetrahydro-1H-pyrrolo[3,2-c]pyridine hydrochloride (BB-16)

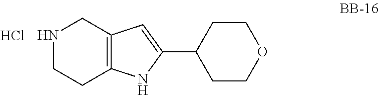 [SMILES: Cl ; c1c(C2CCOCC2)[nH]c2c1CNCC2]

BB-16 was synthesized in analogy to the preparation of BB-1.

### Building Block 17: 2-(2,4-difluorophenyl)-4,5,6,7-tetrahydro-1H-pyrrolo[3,2-c]pyridine hydrochloride (BB-17)

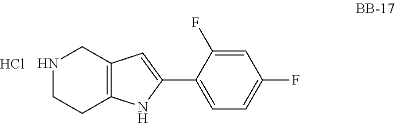 [SMILES: Cl ; Fc1ccc(-c2cc3c([nH]2)CCNC3)c(F)c1]

BB-17 was synthesized in analogy to the preparation of BB-1.

¹H NMR (600 MHz, DMSO-d₆) δ (ppm)=11.31 (s, 1H), 9.20 (s, 2H), 7.71 (td, J=9.0, 6.4 Hz, 1H), 7.28 (dd, J=11.8, 9.3 Hz, 1H), 7.13 (tt, J=8.5, 1.9 Hz, 1H), 6.36 (d, J=2.7 Hz, 1H), 4.08 (d, J=4.3 Hz, 2H), 3.41-3.38 (m, 2H), 2.91 (t, J=6.1 Hz, 3H).

### Building Block 18: 2-(2-fluorophenyl)-4,5,6,7-tetrahydro-1H-pyrrolo[3,2-c]pyridine hydrochloride (BB-18)

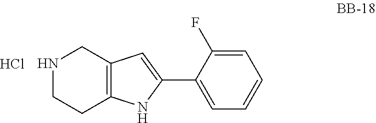 [SMILES: Cl ; Fc1ccccc1-c1cc2c([nH]1)CCNC2]

BB-18 was synthesized in analogy to the preparation of BB-1.

¹H NMR (600 MHz, DMSO-d₆) δ (ppm)=11.32 (s, 1H), 9.37 (s, 2H), 7.75-7.67 (m, 1H), 7.27-7.17 (m, 3H), 6.42-6.39 (m, 1H), 4.06 (s, 2H), 3.39-3.32 (m, 2H), 2.92 (t, J=5.8 Hz, 2H).

### Building Block 19: 2-(2-methylpyridin-3-yl)-4,5,6,7-tetrahydro-1H-pyrrolo[3,2-c]pyridine hydrochloride

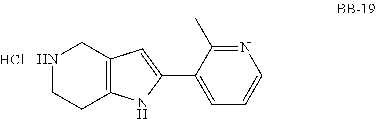 [SMILES: Cl ; Cc1ncccc1-c1cc2c([nH]1)CCNC2]

BB-19 was synthesized in analogy to the preparation of BB-1.

¹H NMR (400 MHz, DMSO-d₆) δ (ppm)=11.75 (s, 1H), 9.32 (s, 2H), 8.59-8.55 (m, 1H), 8.49-8.44 (m, 1H), 7.89-7.83 (m, 1H), 6.48 (d, 1H), 4.11 (s, 2H), 3.39 (m, 2H), 2.97-2.93 (m, 2H), 2.84 (s, 3H).

### Building Block 20: 1-bromo-2-methyl-4-((trifluoromethyl)sulfonyl)benzene (BB-20)

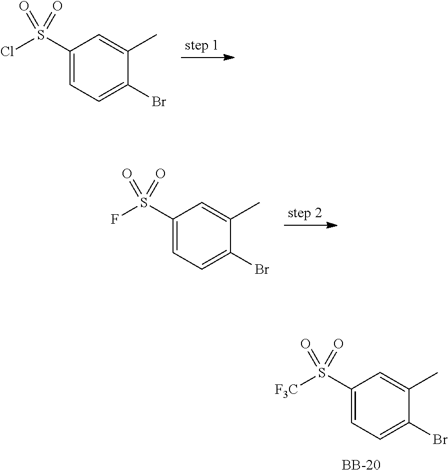

Step 1:

A suspension of 4-bromo-3-methylbenzene-1-sulfonyl chloride (2.25 g, 8.30 mmol), KF (1.94 g, 33.4 mmol), 18-crown-6 (66 mg, 250 μmol) in MeCN (4.5 mL) was stirred at it for 24 h and further KF (0.5 eq.) was added. The mixture was stirred for further 24 h, was diluted with EtOAc and was washed with water, was dried and the volatiles were removed under reduced pressure to yield the desired compound (2.08 g, 98%).

Step 2:

A solution of step 1 intermediate (2.05 g, 8.1 mmol) in pentane (8 mL) was added in 50 min to a solution of trimethyl(trifluoromethyl)silane (“Ruppert's reagent”, 2.55 mL, 17.0 mmol) in pentane (15 mL) at 0° C. and the RM was stirred at it for 1.5 h. The pentane layer decanted, was washed with water, was dried and the volatiles were removed under reduced pressure. The residue was purified through CC (SiO₂, pentane/EtOAc) to yield the desired compound (1.75 g, 71%).

¹H NMR (500 MHz, CDCl₃) δ (ppm)=7.87-7.84 (m, 2H), 7.71-7.69 (m, 1H), 2.54 (s, 3H).

### Building Block 21: 4-methyl-5-(4,5,6,7-tetrahydro-1H-pyrrolo[3,2-c]pyridin-2-yl)-1,2,3-thiadiazole hydrochloride

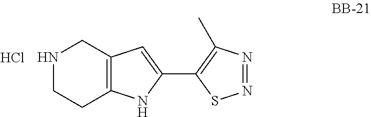

BB-21 was synthesized in analogy to the preparation of BB-1.

¹H NMR (400 MHz, DMSO-d₆) δ (ppm)=11.79 (s, 1H), 9.54 (s, 2H), 6.48 (d, J=2.5 Hz, 1H), 4.08 (s, 2H), 3.39-3.33 (m, 2H), 2.93-2.90 (m, 2H), 2.75 (s, 3H).

### Building Block 22: 2-(3-fluoropyridin-4-yl)-4,5,6,7-tetrahydro-1H-pyrrolo[3,2-c]pyridine hydrochloride

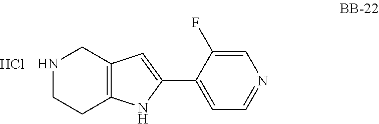 [SMILES: Cl ; Fc1cnccc1-c1cc2c([nH]1)CCNC2]

BB-22 was synthesized in analogy to the preparation of BB-1.

¹H NMR (400 MHz, DMSO-d₆) δ (ppm)=12.10 (s, 1H), 9.33 (s, 2H), 8.72 (d, J=4.5 Hz, 1H), 8.48 (d, J=5.5 Hz, 1H), 8.01-7.95 (m, 1H), 6.86 (s, 1H), 4.12 (s, 2H), 2.98 (t, J=5.5 Hz, 2H), 2.61-2.55 (t, J=5.5 Hz, 2H).

### Building Block 23: 2-(4,6-dimethylpyridin-3-yl)-4,5,6,7-tetrahydro-1H-pyrrolo[3,2-c]pyridine hydrochloride

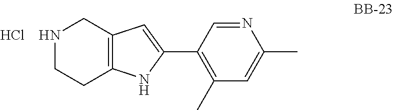 [SMILES: Cl ; Cc1cc(C)c(-c2cc3c([nH]2)CCNC3)cn1]

BB-23 was synthesized in analogy to the preparation of BB-1.

¹H NMR (400 MHz, DMSO-d₆) δ (ppm)=11.94 (s, 1H), 9.57 (s, 2H), 8.78 (s, 1H), 7.81 (s, 1H), 6.49 (d, J=2.5 Building block 24:

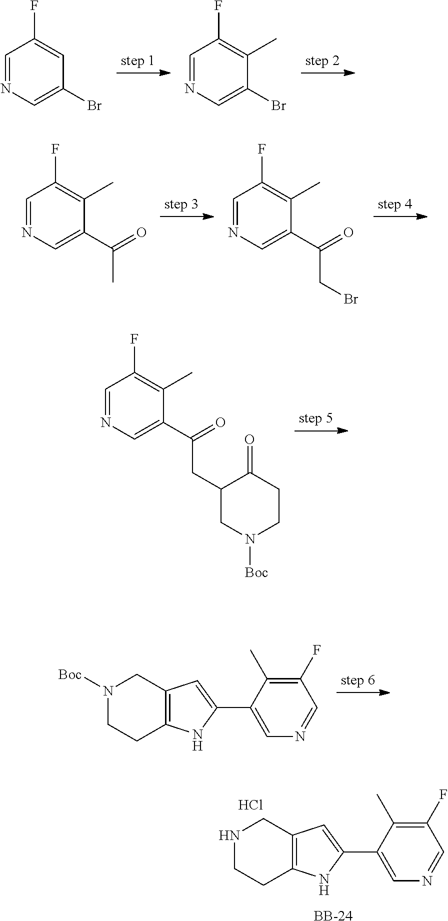

Step 1:

To a solution of LDA (2M solution in Cy/ethylbenzole/THF, 21.3 mL, 42.6 mmol) in dry THF (23 mL) was added dropwise over 10 min a solution of 3-bromo-5-fluoropyridine (5.0 g, 28.4 mmol) in dry THF (23 mL) at −78° C. and stirring continued at −78° C. for 30 min. Subsequently a solution of CH₃I (2.6 mL, 42.6 mmol) in dry THF (23 mL) was added very slowly dropwise and again stirring continued for 30 min at −78° C. The RM was stirred for another 2 h at it before it was quenched with sat. NH₄Cl solution. After further 30 min at it the RM was extracted with EtOAc. The combined organic layers were dried, volatiles removed under reduced pressure and the residue purified by CC (SiO₂, EtOAc/Cy) to yield the desired product (3.6 g, 66%).

Step 2:

A solution of the intermediate from step 1 (3.0 g, 15.8 mmol) and [1,1-Bis(diphenylphosphino)-ferrocene]dichloropalladium(II) (0.7 g, 0.95 mmol) in ethylene glycol (70 mL) was purged with N₂ before 1-(vinyloxy)butane (3.2 g, 31.6 mmol) and NEt₃ (4.4 mL, 31.6 mmol) were added. The RM was stirred at 140° C. for 4 h. Water was added and the mixture extracted with CH₂Cl₂. The combined organic layers were concentrated in vacuo, a solution of HCl (3 M, 30 mL) and THF (10 mL) added and the resulting solution stirred overnight. Water was added and the mixture extracted several times with EtOAc. The combined organic layers were dried, volatiles removed under reduced pressure and the residue purified by CC (SiO₂, Cy/EtAc) to yield the desired compound (0.77 g, 32%).

Step 3:

To a solution of the intermediate from step 2 (0.76 g, 5.0 mmol) in CH₂Cl₂ (8 mL) at 0° C. was added DIPEA (1.3 mL, 7.4 mmol) followed by addition (dropwise) of trimethylsilyltrifluormethanesulfonate (1.4 mL, 7.4 mmol) in CH₂Cl₂ (8 mL). The mixture was stirred at 0° C. for 30 min, then NBS (0.9 g, 5.1 mmol) was added and the RM stirred at it for 30 min. The RM was washed with sat. NaHCO₃ solution, the aqueous layer extracted with CH₂Cl₂ and the combined organic layers dried and volatiles removed under reduced pressure to yield the desired compound. The material was immediately taken to the next step to prevent decomposition of the compound.

Step 4:

A solution of Boc-4-piperidone (1.0 g, 5.0 mmol) and pyrrolidine (1.0 mL, 11.9 mmol) in toluene (16 mL) was refluxed employing a Dean-Stark trap for 4 h. Volatiles were removed under reduced pressure before the residue was dissolved in THF (12 mL). The intermediate from step 3 (1.2 g, 5.0 mmol) and NEt₃ (1.7 mL, 12.4 mmol) were added and the RM stirred in the dark at 60° C. overnight. Volatiles were removed under reduced pressure and the residue taken up in 0.1 m HCl and EtOAc. The layers were separated and the organic layer was washed with 0.1 M HCl, and the aqueous layer extracted again with EtOAc. The combined organic layers were dried and volatiles were removed under reduced pressure to yield the desired compound.

Step 5:

A mixture of the intermediate from step 4 (1.7 g, 5.0 mmol) and NH₄OAc (1.9 g, 24.8 mmol) in EtOH (14 mL) was refluxed for 1 h. Subsequently volatiles were removed under reduced pressure and the residue taken up in EtOAc. The organic layer was washed twice with sat. NaHCO₃ solution and brine before dried. Volatiles were removed under reduced pressure and the residue purified by CC (SiO₂, cyclohexane/EtOAc 3:1) to yield the desired product (0.26 g, 16%).

Step 6:

To a solution of the intermediate from step 5 (0.26 g, 0.79 mmol) in ethanol (2.3 mL) was added at 0° C. acetyl chloride (0.28 mL, 3.9 mmol). The RM was stirred at it overnight. The suspension was diluted with Et₂O and the resulting solid isolated by filtration and washing with diethyl ether to yield the desired compound as HCl salt (0.17 g, 81%).

¹H NMR (400 MHz, DMSO-d₆) δ (ppm)=11.56 (s, 1H), 9.43 (s, 2H), 8.55 (s, 1H), 8.49 (s, 1H), 6.35 (d, J=3.0 Hz, 3H).

### Building Block 24: 2-(2,6-difluorophenyl)-3-fluoro-4,5,6,7-tetrahydro-1H-pyrrolo[3,2-c]pyridine (BB-24)

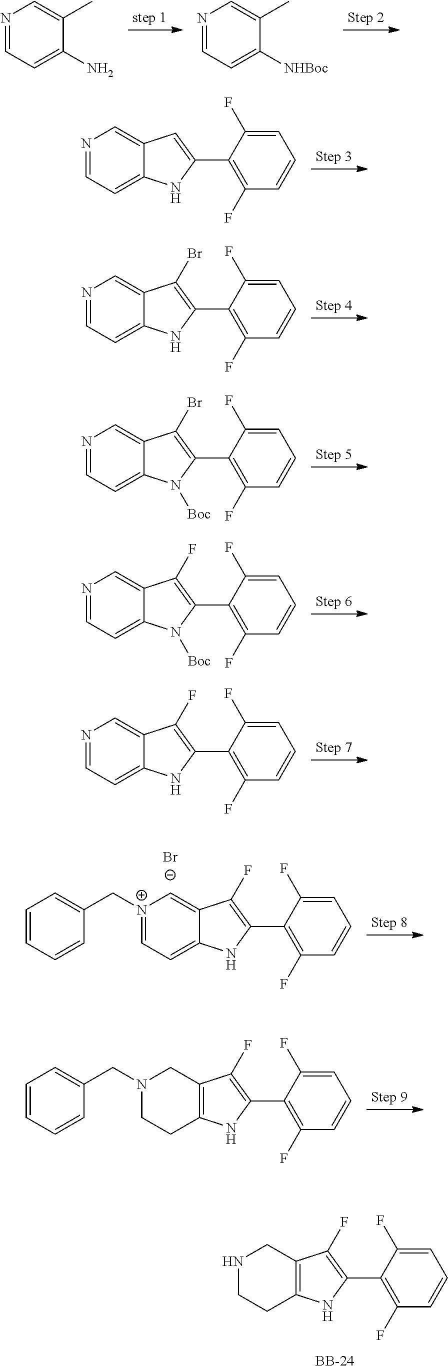

Step 1:

To a solution of 4-amino-3-methylpyridine (25.0 g, 231 mmol) in THF (200 mL) was added Boc₂O (61 ml, 277 mmol) and stirred at RT for 14 h. The RM was concentrated and the residue was purified by CC (SiO₂, MeOH/CH₂Cl₂) to afford (3-methyl-pyridin-4-yl)-carbamic acid tert-butyl ester (35.0 g, 72%). LC-MS (Method 3): m/z [M+H]⁺=209.4 (MW calc.=208.26); Rₜ=2.86 min.

Step 2:

t-BuLi (60 mL, 15% in pentane) was added drop-wise to a solution of (3-methyl-pyridin-4-yl)-carbamic acid tert-butyl ester (10.0 g, 48.1 mmol) and TMEDA (22 mL, 144 mmol) in dry THF (150 mL) at −50° C. After stirring for 1 h at −40° C., a solution of 2,6-difluoro-N-methoxy-N-methyl-benzamide (11.6 g, 144.2 mmol) in THF (30 mL) was added over 10-15 min and stirred at −40° C. for 1 h, at 0° C. for 30 min and then at RT for 3 h. 5.5M HCl (100 mL) was added slowly to the RM and heated at 60° C. for 16 h. The RM was cooled to RT and basified with NaHCO₃ solution to pH-8 and extracted with EtOAc. The combined organic layers were washed with brine, dried and concentrated under reduced pressure. The residue was purified by CC (SiO₂, MeOH/CH₂Cl₂) to afford 2-(2,6-difluoro-phenyl)-1H-pyrrolo[3,2-c]pyridine (2.5 g, 22%).

LC-MS (Method 3): m/z [M+H]⁺=230.9 (MW calc.=230.21); Rₜ=2.75 min.

Step 3:

To a solution of 2-(2,6-difluoro-phenyl)-1H-pyrrolo[3,2-c]pyridine (3.0 g, 13.0 mmol) in CH₂Cl₂ (80 mL) was added NBS (3.5 g, 19.6 mmol) at 0° C. and the RM was stirred at same temperature for 2 h. The RM was diluted with CH₂Cl₂ (100 mL) and washed with sat. NaHCO₃ solution, brine and dried. The solvent was evaporated under reduced pressure to afford 3-bromo-2-(2,6-difluoro-phenyl)-1H-pyrrolo[3,2-c]pyridine (3.5 g, 86%) which was used for next step without further purification.

LC-MS (Method 3): m/z [M+H]⁺=308.8 and 310.8 (MW calc.=309.11); Rₜ=2.99 min.

Step 4:

To a mixture of 3-bromo-2-(2,6-difluoro-phenyl)-1H-pyrrolo[3,2-c]pyridine (3.5 g, 11.3 mmol), NEt₃ (1.5 ml, 11.3 mmol) and DMAP (1.4 g, 11.32 mmol) in THF (40 mL) was added Boc₂O (3.9 mL 17.0 mmol) at 0° C. and the RM was heated at reflux for 16 h. The RM was concentrated under reduced pressure and residue was diluted with EtOAc and subsequently washed with water and brine. The organic layer was concentrated under reduced pressure to give the crude product which was purified by CC (SiO₂, EtOAc/Hex) to yield 3-bromo-2-(2,6-difluoro-phenyl)-pyrrolo[3,2-c]pyridine-1-carboxylic acid tert-butyl ester (3.0 g, 64%).

LC-MS (Method 3): m/z [M+H]⁺=409.2 and 411.1 (MW calc.=409.22); Rₜ=3.86 min.

Step 5:

To a solution of 3-bromo-2-(2,6-difluoro-phenyl)-pyrrolo[3,2-c]pyridine-1-carboxylic acid tert-butyl ester (3.4 g, 8.31 mmol) in THF (30 mL) was added drop-wise n-BuLi (7.0 mL, 1.8 M solution in hexane) at −78° C. and stirred for 30 min at same temperature. A solution of N-fluorodibenzenesulfonimide (3.9 g, 12.5 mmol) in THF (15 mL) was added and the RM was stirred at −78° C. for additional 3 h and subsequently at it for 1 h. The RM was quenched with sat.NH₄Cl solution and extracted with EtOAc. The combined organic layers were washed with water and brine, dried and concentrated under reduced pressure. The residue was purified by CC (SiO₂, EtOAc/Hex) to afford the desired compound (2.0 g, 69%).

LC-MS (Method 3): m/z [M+H]⁺=349.2 (MW calc.=348.32); Rₜ=3.75 min.

Step 6:

To a solution of the intermediate from step 5 (1.0 g, 2.87 mmol) in CH₂Cl₂ (2 mL) was added TFA (8.0 mL) and the mixture was stirred at it for 5 h. The RM was concentrated and the residue was diluted with CH₂Cl₂, washed with NaHCO₃ solution and brine and dried. The solvent was evaporated under reduced pressure to afford 2-(2,6-difluoro-phenyl)-3-fluoro-1H-pyrrolo[3,2-c]pyridine (650 mg, 91%) which was used in next step without further purification.

LC-MS (Method 3): m/z [M+H]⁺=248.8 (MW calc.=248.2); Rₜ=2.90 min.

Step 7:

To a solution of 2-(2,6-difluoro-phenyl)-3-fluoro-1H-pyrrolo[3,2-c]pyridine (500 mg, 2.01 mmol) in acetonitrile (10 mL) was added benzyl bromide (0.24 mL, 2.01 mmol) and the mixture was heated at reflux for 14 h. The RM was concentrated and the residue was triturated with hexane to afford 5-benzyl-2-(2,6-difluoro-phenyl)-3-fluoro-1H-pyrrolo[3,2-c]pyridinium bromide which was directly used for next step.

Step 8:

NaBH₄ (305 mg, 8.04 mmol) was added to a solution of 5-benzyl-2-(2,6-difluoro-phenyl)-3-fluoro-1H-pyrrolo[3,2-c]pyridinium bromide (2.01 mmol) in a mixture of MeOH-water (1:1, 10 mL) at 0° C. and the RM was stirred at it for 1 h and then reflux for 16 h. The RM was concentrated and the residue was taken up in EtOAc, washed with water and dried. The solvent was evaporated under reduced pressure to give the crude product which was purified by CC (SiO₂; 30% EtOAc/Hex) to afford the desired product (300 mg, 43%).

LC-MS (Method 3): m/z [M+H]⁺=343.0 (MW calc.=342.36); Rₜ=3.69 min.

Step 9:

A solution of the intermediate from step 8 (280 mg, 0.81 mmol) in MeOH (5 mL) was degassed with Ar for 15 min followed by the addition of 20% Pd(OH)₂ (140 mg). The RM was stirred under H₂ balloon pressure for 2 h. The mixture was filtered through celite and the filtrate was concentrated under reduced pressure to afford 2-(2,6-difluoro-phenyl)-3-fluoro-4,5,6,7-tetrahydro-1H-pyrrolo[3,2-c]pyridine (180 mg, 87%) which was used in next step without further purification.

LC-MS (Method 3): m/z [M+H]⁺=253.0 (MW calc.=252.24); Rₜ=2.75 min.

### Building Block 25: 2-(4,6-dimethylpyridin-3-yl)-4,5,6,7-tetrahydro-1H-pyrrolo[3,2-c]pyridine hydrochloride

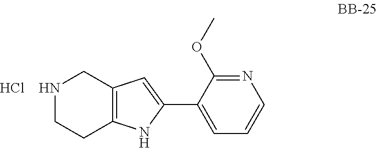 [SMILES: Cl ; COc1ncccc1-c1cc2c([nH]1)CCNC2]

BB-23 was synthesized in analogy to the preparation of BB-1.

¹H NMR (400 MHz, DMSO-d₆) δ (ppm)=11.16 (s, 1H), 9.42 (s, 2H), 7.99-7.95 (m, 2H), 7.02 (dd, J=7.5, 5.0 Hz, 1H), 6.54 (d, J=3.0 Hz, 1H), 4.05 (t, J=4.8 Hz, 2H), 3.97 (s, 3H), 3.40-3.28 (m, 2H), 2.92 (t, J=6.0H.

### Building Block 26: 5-bromo-6-methoxy-2,3-dihydrobenzo[b]thiophene 1,1-dioxide

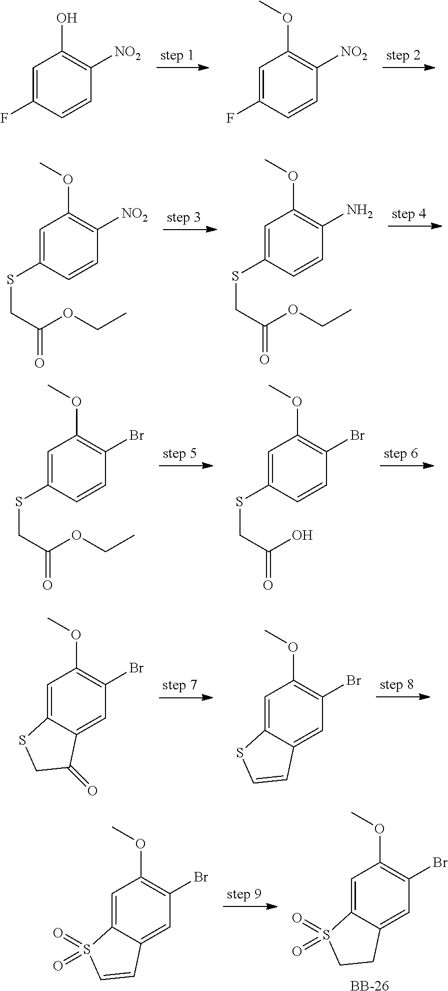

Step 1:

K₂CO₃ (131 g, 955 mmol) and CH₃I (66.7 mL) were added to a solution of 5-fluoro-2-nitrophenol (75.0 g, 478 mmol) in MeCN (750 mL) at it and the resulting mixture was heated to 85° C. for 5 h. The RM was chilled, filtered and washed with MeCN. The volatiles were removed under reduced pressure and the residue was diluted with water and was extracted with EtOAc. The combined organic layers were washed with water, brine, were dried and the volatiles were removed under reduced pressure to give the desired compound (75 g, 94%).

Step 2:

K₂CO₃ (120 g, 876 mmol) and ethyl mercaptoacetate (49 mL) were added to a solution of the intermediate of step 1 (75 g, 438 mmol) in MeCN (750 mL) and the mixture was heated to 80° C. for 18 h. The mixture was filtered and the volatiles were removed under reduced pressure. The residue was diluted with water and was extracted with EtOAc. The combined organic layers were washed with water, brine, were dried and the volatiles were remove under reduced pressure to yield the desired compound (90 g, 76%).

Step 3:

A solution of the intermediate of step 2 (90 g, 330 mmol) in EtOH (400 mL) was added to a suspension of iron powder (55.9 g, 992 mmol) and NH₄Cl (88.3 g, 1.65 mol) in water (800 mL) and MeOH (400 mL) and the RM was heated to 80° C. overnight. The RM was filtered over a pad of celite and the volatiles were removed under reduced pressure. The residue was diluted with water and was extracted with EtOAc. The combined organic layers were washed with water, brine, were dried and the volatiles were removed under reduced pressure to yield the desired compound (80 g, 99%).

Step 4:

A solution of NaNO₂ (24.0 g, 349 mmol) in water (50 mL) was added to a solution of the intermediate of step 3 (80 g, 332 mmol) in aqueous HBr (45%, 80 mL) and water (80 mL) at 0° C. and the RM was stirred for 2 h. The RM was added drop wise in 1 h to a suspension of CuBr (96 g, 671 mmol) in aqueous HBr (45%, 200 mL) at 70° C. and the mixture was heated to 95° C. for 4 h. The mixture was chilled and was diluted with water and was extracted with EtOAc. The combined organic layers were washed with water, brine, dried and the volatiles were removed under reduced pressure. The residue was purified by CC (SiO₂, Cy/EtOAc) to yield the desired compound (65 g, 64%).

Step 5:

LiOH.H₂O (3.3 g, 78.9 mmol) was added to a solution of the intermediate of step 4 (12 g, 39.5 mmol) in a mixture of MeOH (20 mL) and H₂O (20 mL) the mixture was stirred at it for 3 h. The volatiles were removed under reduced pressure and the residue was diluted with water and was washed with EtOAc. The aqueous layer was acidified to pH=2 with diluted HCl and was extracted with EtOAc. The combined organic layers were washed with water, brine, were dried and the volatiles were removed under reduced pressure to yield the desired compound (9 g, 83%).

Step 6:

Oxalyl chloride (4.3 mL, 54.3 mmol) was added to a solution of the intermediate of step 5 in dry CH₂Cl₂ (50 mL) at 0° C. and the mixture was stirred at it for 2 h. The volatiles were removed under reduced pressure and the residue was dissolved in CH₂Cl₂ (50 mL). Anhydrous AlCl₃ (2.65 g, 19.9 mmol) was added portion wise and the mixture was stirred at it for 18 h. Cold water was added and the mixture was extracted with EtOAc. The combined organic layers were washed with water, brine, were dried and the volatiles were removed under reduced pressure to yield the desired compound (2.6 g, 57%).

Step 7:

TFA (3.5 mL, 46.5 mmol) was added to a solution of the intermediate of step 6 (1 g, 46.5 mmol) in CH₂Cl₂ (10 mL) at 0° C. and the mixture was stirred for 10 min. Sodium borohydride (735 mg, 19.4 mmol) was added portion wise and the RM was stirred at it for 16 h. Cold water was added and the mixture was extracted with EtOAc. The combined organic layers were washed with water, brine, were dried and the volatiles were removed under reduced pressure to yield the desired compound (650 mg, 69%).

Step 8:

Oxone (25.4 g, 41.3 mmol) was added to a solution of intermediate of step 7 (2 g, 8.26 mmol) in acetone (30 mL) and water (15 mL) and the mixture was stirred at it overnight. The RM was filtered and the volatiles were removed under reduced. The residue was purified by CC (SiO₂, CyEtOac) to yield the desired compound (1.8 g, 79%).

Step 9:

10% Pd/C (400 mg) was added to a solution of the intermediate of step 8 (4.0 g, 14.5 mmol) in THF (100 mL) and the mixture was stirred under H₂ (40 psi) at it for 6 h. The mixture was filtered over a pad of celite, and the volatiles were removed under pressure to yield BB-5 (2.2 g, 55%).

¹H-NMR (DMSO-d₆): □=7.83 (s, 1H), 7.42 (s, 1H), 3.94 (s, 3H), 3.62 (t, J=7.2 Hz, 2H), 3.27 (t, J=7.2 Hz, 2H).

### Building Block 27: 5-bromo-4-methyl-2-((methylsulfonyl)methyl)pyridine

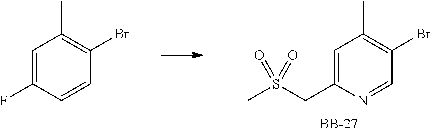

A solution of 5-bromo-2-fluoro-4-methylpyridine (8.00 g, 42.1 mmol) in THF (170 mL) was added to NaHMDS (1 m in THF, 210 mL, 210 mmol) at −30° C. To this mixture dimethyl sulfone (15.9 g, 168 mmol) was added at −30° C. and the RM was stirred at it overnight. Saturated aqueous NH₄Cl was added, the mixture was extracted with EtOAc and the volatiles were removed under reduced pressure. The residue was purified through CC (SiO₂, Hex/EtOAc) to yield the desired compound (1.83 g, 98%).

¹H-NMR (CDCl₃): □=8.62 (s, 1H), 7.37 (s, 1H), 4.34 (s, 2H), 2.92 (s, 3H), 2.43 (s, 3H) ppm.

### Building Block 28: 2-(3-chloropyridin-4-yl)-4,5,6,7-tetrahydro-1H-pyrrolo[3,2-c]pyridine hydrochloride

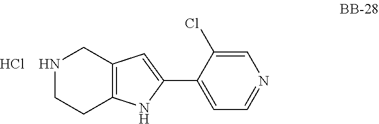 [SMILES: Cl ; Clc1cnccc1-c1cc2c([nH]1)CCNC2]

BB-27 was synthesized in analogy to the preparation of BB-1.

### Building Block 29: 2-(tetrahydro-2H-pyran-3-yl)-4,5,6,7-tetrahydro-1H-pyrrolo[3,2-c]pyridine hydrochloride

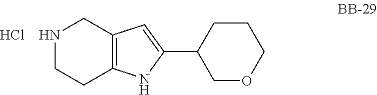 [SMILES: Cl ; c1c(C2CCCOC2)[nH]c2c1CNCC2]

BB-29 was synthesized in analogy to the preparation of BB-1.

### Building Block 30: 2-cyclopentyl-4,5,6,7-tetrahydro-1H-pyrrolo[3,2-c]pyridine hydrochloride

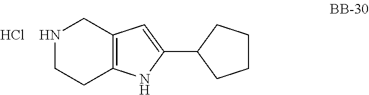 [SMILES: Cl ; c1c(C2CCCC2)[nH]c2c1CNCC2]

BB-30 was synthesized in analogy to the preparation of BB-1.

### Building Block 31: 2-(4,4-difluorocyclohexyl)-4,5,6,7-tetrahydro-1H-pyrrolo[3,2-c]pyridine hydrochloride

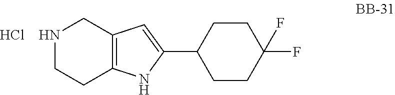 [SMILES: Cl ; FC1(F)CCC(c2cc3c([nH]2)CCNC3)CC1]

BB-31 was synthesized in analogy to the preparation of BB-1.

### SYNTHESIS OF THE REPRESENTATIVE EXAMPLES

### Synthesis Example 1

### 2-(2,6-Difluoro-phenyl)-5-(5-methyl-2-pyridin-3-yl-thiazol-4-yl)-1,5,6,7-tetrahydro-pyrrolo[3,2-c]pyridin-4-one

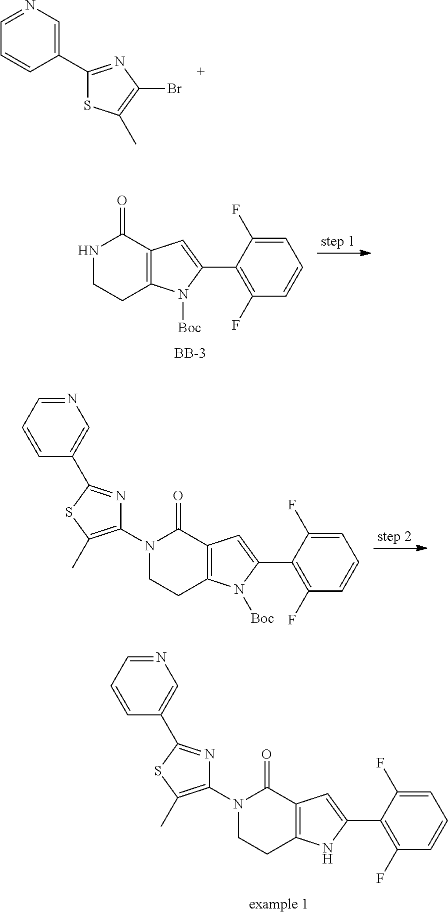

Step 1:

To a solution of BB-4 (100 mg, 0.287 mmol) in toluene (10 mL) in a sealed tube was added K₂CO₃ (119 mg, 0.86 mmol), 4-bromo-5-methyl-2-(pyridin-3-yl)thiazole (87.6 mg, 0.344 mmol), and N,N′-dimethyl ethylene diamine (13 mg, 0.143 mmol) and the mixture was degassed by purging with Ar for 30 min. CuI (27 mg, 0.14 mmol) was added and the RM was heated to 100° C. under Ar for 48 h. The RM was chilled, diluted with toluene and filtered through a plug of Celite™. The volatiles were removed under reduced pressure and the residue was purified by CC (silica gel, CH₂Cl₂/MeOH) to yield the desired compound (100 mg, 60%).

Step 2:

To a solution of the intermediate from step 1 (200 mg, 0.383 mmol) in THF (10 mL), a solution of NaOMe (103 mg, 1.91 mmol) in MeOH (10 mL) was added at it and the RM was stirred for 3 h. All volatiles were removed under reduced pressure, the residue diluted with water and the formed precipitate was isolated through filtration. The obtained solid was purified by flash CC (silica gel, CH₂Cl₂/MeOH) to yield the title compound of example 1 (135 mg, 84%).

LC-MS (method 2): m/z: [M+H]⁺=423.1 (MW calc.=422.10); Rₜ=0.66 min.

¹H NMR (300 MHz; DMSO-d₆): δ (ppm)=2.30 (s, 3H), 3.09-3.13 (t, J=6 Hz, 2H), 3.17 (s, 1H), 3.98 4.02 (t, J=6 Hz, 2H), 6.73 (s, 1H), 7.20-7.26 (t, J=9 Hz, 2H), 7.34-7.56 (q, J=6 Hz, 1H), 7.52-7.56 (dd, J₁=3 Hz, J₂=6 Hz, 1H), 8.23 (d, J=9 Hz, 1H), 9.07 (s, 1H) ppm.

### Synthesis Example 2

### 4-[2-(2,6-Difluoro-phenyl)-4,5,6,7-tetrahydro-1H-pyrrolo[3,2-c]pyridin-5-yl]-5-methyl-2-pyridin-3-yl-thiazole

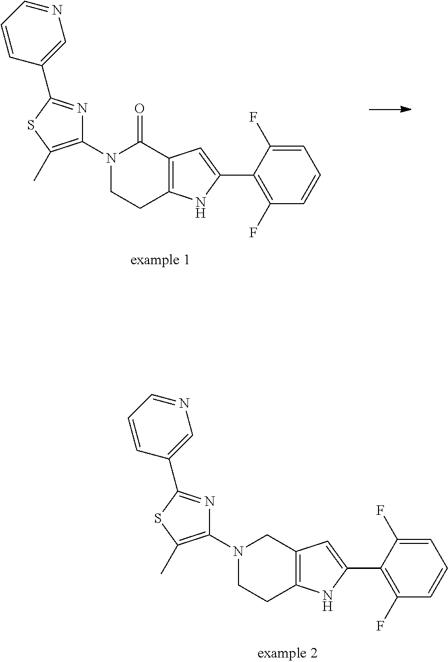 [SMILES: Cc1sc(-c2cccnc2)nc1N1CCc2[nH]c(-c3c(F)cccc3F)cc2C1=O ; Cc1sc(-c2cccnc2)nc1N1CCc2[nH]c(-c3c(F)cccc3F)cc2C1]

To a solution of the title compound of example 1 (200 mg, 0.473 mmol) in dry THF (10 mL), BH₃.DMS (72 mg, 0.95 mmol) was added drop wise and the RM was stirred for 24 h at rt. The RM was cooled to 0° C., MeOH (10 mL) was added and the RM was heated to 80° C. for 3 h. The volatiles were removed under reduced pressure and the residue was purified by CC (silica gel, Hex/EtOAc) yield the title compound of example 2 (65 mg, 35%).

LC-MS (method 2): m/z: [M+H]⁺=409.1 (MW calc.=408.12); Rₜ=0.86 min.

¹H NMR (600 MHz; DMSO-d₆): δ (ppm)=2.40 (s, 3H), 2.85-2.87 (t, J=6 Hz, 2H), 3.42-3.44 (t, J=6 Hz, 2H), 4.19 (s, 2H), 6.33 (s, 1H), 7.13-7.15 (t, J=6 Hz, 2H), 7.22-7.26 (m, 1H), 7.49-7.51 (dd, J₁=9 Hz, J₂=6 Hz, 1H), 8.85-8.90 (d, J=9 Hz, 1H), 8.60-8.61 (t, J=6 Hz, 1H), 9.03 (s, 1H), 10.78 (s, 1H) ppm.

### Synthesis Example 3

### 5-(6-Chloro-4-methyl-pyridin-3-yl)-2-(2,6-difluoro-phenyl)-1,5,6,7-tetrahydro-pyrrolo[3,2-c]pyridin-4-one

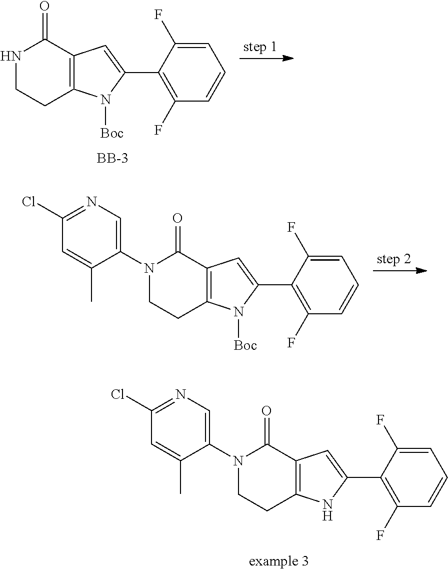

Step 1:

To a solution of BB-3 (100 mg, 0.287 mmol) in toluene (10 mL), K₂CO₃ (119 mg, 0.86 mmol), 5-bromo-2-chloro-4-methylpyridine (77 mg, 0.373 mmol) and N,N′-dimethyl ethylene diamine (13 mg, 0.143 mmol) were added and the mixture was degassed through purging with Ar for 30 min. CuI (27 mg, 0.14 mmol) was added and the RM was heated to 100° C. for 48 h. The RM was chilled, diluted with toluene and filtered over a plug of Celite™. The volatiles were removed under reduced pressure and the residue was purified by flash CC (silica gel, Hex/EtOAc) to yield the desired compound (60 mg, 45%).

Step 2:

To a solution of the intermediate from step 1 (160 mg, 0.338 mmol) in THF (10 mL), a solution of NaOMe (91 mg, 1.69 mmol) in MeOH (10 mL) was added and RM was stirred at it for 3 h. The volatiles were removed under reduced pressure, the residue was diluted with water, and the formed precipitate was isolated through filtration. The residue was purified by CC (silica gel, CH₂Cl₂/MeOH) to yield the title compound of example 3 (80 mg, 63%).

LC-MS (method 2): m/z: [M+H]⁺=374.1 (MW calc.=373.08); Rₜ=0.68 min.

¹H NMR (600 MHz; DMSO-d₆): δ (ppm)=2.22 (s, 3H), 3.05-3.16 (m, 2H), 3.74-3.77 (m, 1H), 4.02-4.07 (m, 1H), 6.69 (s, 1H), 7.20-7.24 (t, J=6 Hz, 2H), 7.35-7.40 (q, J=6 Hz, 1H), 7.50 (s, 1H), 8.30 (s, 1H), 11.62 (s, 1H).

### Synthesis Example 4

### 5-(6-Chloro-4-methyl-pyridin-3-yl)-2-(2,6-difluoro-phenyl)-4,5,6,7-tetrahydro-1H-pyrrolo[3,2-c]pyridine

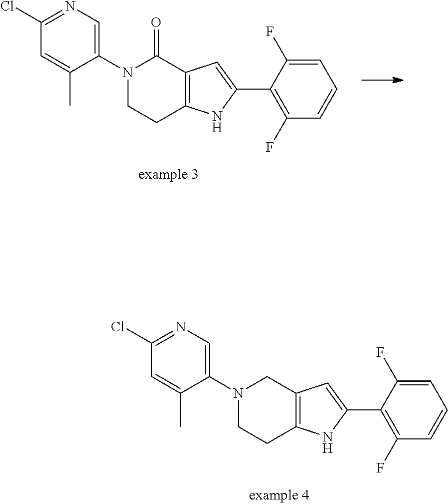 [SMILES: Cc1cc(Cl)ncc1N1CCc2[nH]c(-c3c(F)cccc3F)cc2C1=O ; Cc1cc(Cl)ncc1N1CCc2[nH]c(-c3c(F)cccc3F)cc2C1]

To a solution of the title compound of example 3 (200 mg, 0.473 mmol) in dry THF (10 mL), BH₃.DMS complex (72 mg, 0.947 mmol) was added dropwise and the RM was stirred at it for 16 h. The RM was chilled, MeOH (10 mL) was added and the RM was heated to 80° C. for 3 h. The volatile were removed under reduced pressure and the residue was purified by CC (silica gel, Hex/EtOAc) to yield the title compound of example 4 (65 mg, 35%).

LC-MS (method 2): m/z: [M+H]⁺=360.1 (MW calc.=359.1); Rₜ=0.86 min.

¹H NMR (600 MHz; DMSO-d₆): δ (ppm)=2.31 (s, 3H), 2.77-2.79 (t, J=6 Hz, 2H) 3.23-3.55 (t, J=6 Hz, 2H), 4.03 (s, 2H), 6.33 (s, 1H), 7.13-7.16 (t, J=6 Hz, 2H), 7.22-7.27 (q, J=6 Hz, 1H), 7.33 (s, 1H), 8.09 (s, 1H), 10.82 (s, 1H).

### Synthesis Example 5

### 2-(2,6-Difluoro-phenyl)-5-(5-methyl-2-pyridin-2-yl-thiazol-4-yl)-1,5,6,7-tetrahydro-pyrrolo[3,2-c]pyridin-4-one

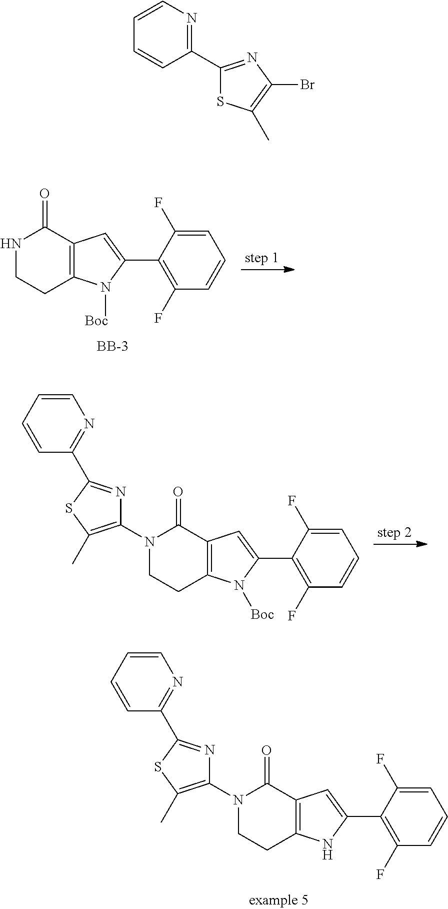

Step 1:

To a solution of BB-3 (500 mg, 1.43 mmol) in toluene (20 mL) was added K₂CO₃ (594 mg, 4.30 mmol), 4-bromo-5-methyl-2-(pyridin-2-yl)thiazole (401 mg, 1.58 mol), N,N′-dimethyl ethylene diamine (63 mg, 0.72 mmol) and the RM was degassed through purging with Ar for 30 min. CuI (136 mg, 0.72 mmol) was added the RM was heated to for 100° C. for 72 h. The RM chilled, diluted with toluene and filtered over a plug of Celite™. The volatiles were removed under reduced pressure and the residue was purified by CC (silica gel, Hex/EtOAc) to yield the desired compound (550 mg, 73%).

Step 2:

To a solution of the intermediate from step 1 (1.1 g, 2.1 mmol) in THF (25 mL), a solution of NaOMe (341 mg, 6.32 mmol) in MeOH (25 mL) was added at and the RM was stirred at it for 3 h. The volatiles were removed under reduced pressure, the residue diluted with water (50 mL) and the formed precipitate was isolated through filtration. The remaining solid was washed with water (25 mL) followed by pentane (25 mL) to yield the title compound of example 5 (700 mg, 79%).

LC-MS (method 2): m/z: [M+H]⁺=323.0 (MW calc.=322.1); Rₜ=0.73 min.

¹H NMR (600 MHz; DMSO-d₆): δ (ppm)=2.30 (s, 3H), 3.10-3.12 (t, J=6 Hz, 2H) 3.99-4.01 (t, J=6 Hz, 2H), 6.73 (s, 1H), 7.21-7.24 (t, J=9 Hz, 2H), 7.35-7.40 (q, J=6 Hz, 1H), 7.46-7.48 (t, J=9 Hz, 1H), 7.93-7.95 (t, J=6 Hz, 1H), 8.03-8.04 (d, J=6 Hz, 1H), 11.63 (br, 1H).

### Synthesis Example 6

### 4-[2-(2,6-Difluoro-phenyl)-4,5,6,7-tetrahydro-1H-pyrrolo[3,2-c]pyridin-5-yl]-5-methyl-2-pyridin-2-yl-thiazole

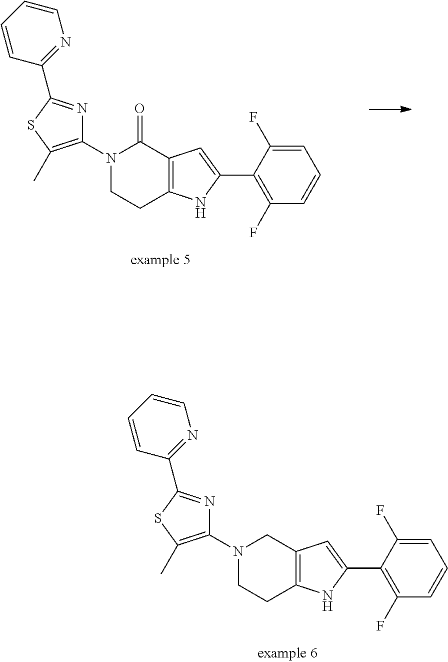 [SMILES: Cc1sc(-c2ccccn2)nc1N1CCc2[nH]c(-c3c(F)cccc3F)cc2C1=O ; Cc1sc(-c2ccccn2)nc1N1CCc2[nH]c(-c3c(F)cccc3F)cc2C1]

To a solution of the title compound of example 5 (100 mg, 0.24 mmol) in dry THF (15 mL) BH₃.DMS complex (36 mg, 0.48 mmol) was added and the mixture was heated to 60° C. for 2 h. The RM was cooled to 0° C., MeOH (10 mL) was added and the RM was heated to 70° C. for 2 h. The volatiles were removed under reduced pressure and the residue was diluted with water and was extracted with EtOAc. The combined organic layers were washed with brine, dried and the volatiles were removed under reduced pressure. The residue was purified by CC (silica gel, Hex/EtOAc) to yield the title compound of example 6 (18 mg, 26%).

LC-MS (method 2): m/z: [M+H]⁺=409.0 (MW calc.=408.12); Rₜ=0.95 min.

¹H NMR (600 MHz; DMSO-d₆): δ (ppm)=2.40 (s, 3H), 2.85-2.87 (t, J=6 Hz, 2H) 3.40-3.42 (t, J=6 Hz, 2H), 4.17 (s, 2H), 6.33 (s, 1H), 7.13-7.15 (t, J=6 Hz, 2H), 7.22-7.26 (q, J=6 Hz, 1H), 7.90-7.92 (t, J=6 Hz, 1H), 8.02-8.03 (d, J=6 Hz, 1H), 8.56-8.57 (d, J=6 Hz, 1H), 10.78 (s, 1H).

### Synthesis Example 7

### 2-(2,6-Difluoro-phenyl)-5-[3-(trifluoromethyl)phenyl]-4,5,6,7-tetrahydro-1H-pyrrolo[3,2-c]pyridine

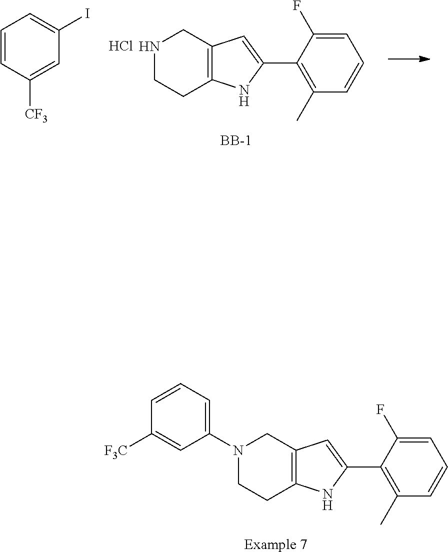 [SMILES: FC(F)(F)c1cccc(I)c1 ; Cl ; Cc1cccc(F)c1-c1cc2c([nH]1)CCNC2 ; "BB-1" ; Cc1cccc(F)c1-c1cc2c([nH]1)CCN(c1cccc(C(F)(F)F)c1)C2 ; "Example 7"]

A suspension of BB-1 (100 g, 0.37 mmol), 1-iodo-3-(trifluoromethyl)benzene (120 mg, 0.44 mmol), CuI (14 mg, 0.074 mmol), K₂PO₄ (236 mg, 1.11 mmol) and ethylene glycol (23 mg, 0.37 mmol) in i-PrOH (0.5 mL) was heated under N₂ for 48 h. The mixture was chilled, water was added and it was extracted with CH₂Cl₂. The combined organic layers were dried and the volatiles were removed under reduced pressure. The reside was purified by CC (silica gel, 18 g, Cy/EtOAc) to yield the title compound of example 7 (45 mg, 32%).

LC-MS (Method 1): m/z: [M+H]⁺=379.2 (MW calc.=378.34); Rₜ=4.3 min.

¹H-NMR (600 MHz, CDCl₃): δ (ppm)=2.89 (t, J=5.6 Hz, 2H), 3.72 (t, J=5.6 Hz, 2H), 4.35 (s, 2H); 6.72 (m, 1H), 6.96 (m, 2H), 7.04 (m, 1H); 7.06 (m, 1H), 7.14 (m, 1H), 7.20 (s, 1H), 7.35 (t, J=8 Hz), 8.78 (br s, 1H) ppm.

### Synthesis Example 8

### 2-[3-[2-(2,6-Difluoro-phenyl)-4,5,6,7-tetrahydro-1H-pyrrolo[3,2-c]pyridin-5-yl]-4-methyl-phenyl]-thiazole

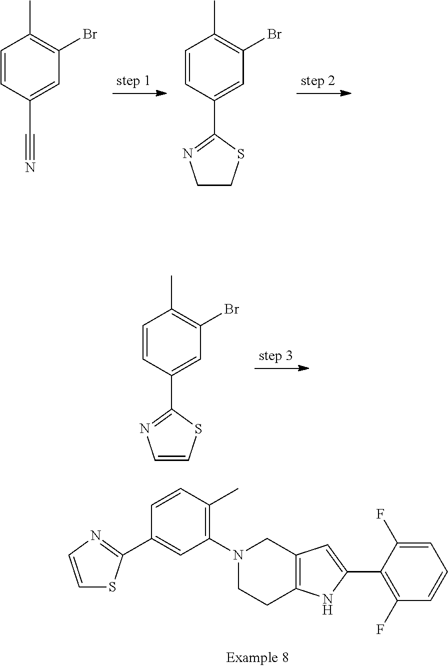

Step 1:

A solution of 3-bromo-4-methylbenzonitrile (1.96 g, 10 mmol) and 2-aminoethanethiol (1.0 g, 13 mmol) in EtOH (30 mL) was heated to 90° C. for 1 h. The mixture was chilled, diluted with Et₂O (250 mL) and was washed with water. The organic layer was dried and the volatiles were removed under reduced pressure to yield the desired compound of step 1.

LC-MS (method 1): m/z: [M+H]⁺=257.1 (MW calc.=256.16), Rₜ=3.8 min.

Step 2:

A solution of the intermediate from step 1 (2.43 g, 9.5 mmol) and DDQ (3.4 g, 15 mmol) in benzene (90 mL) was heated to 90° C. for 2 h. The mixture was chilled and was diluted with Et₂O (200 mL) and was washed with sat. NaHCO₃. The organics layer was dried and the volatiles were removed under reduced pressure. The reside was purified by chromatography (Interchim® cartridge 30SiHP/120 g, Cy/EtOAc) to yield the desired compound of step 2 (2.05 g, 85%).

LC-MS (method 1): m/z: [M+H]⁺=256.2 (MW calc.=254.15); Rₜ=3.9 min.

Step 3:

A degassed suspension of BB-1 (100 mg, 0.37 mmol), the intermediate from step 2 (125 mg, 0.49 mmol), Pd(OAc)₂ (7 mg, 0.031 mmol), rac-BINAP (27 mg, 0.043 mmol) and Cs₂CO₃ (340 mg, 1.04 mmol) in dry toluene (2.4 mL) was heated under N₂ to 120° C. for 48 h. The mixture was chilled and the volatiles were removed under reduced pressure. The reside was purified by chromatography (Interchim® cartridge 30SiHP/40 g, Cy/EtOAc) to yield the title compound of example 8 (60 mg, 40%).

LC-MS (method 1): m/z: [M+H]⁺=408.2 (MW calc=407.48); Rₜ=4.2 min.

¹H-NMR (400 MHz, CDCl₃): δ (ppm)=2.40 (s, 3H), 2.92 (t, J=5.6 Hz, 2H), 3.35 (t, J=5.6 Hz, 2H), 4.11 (s, 2H), 6.70 (m, 1H), 6.95 (m, 2H), 7.06 (m, 1H), 7.27 (d, 1H), 7.29 (d, J=3.6 Hz, 1H); 7.55 (dd, J=8, 2 Hz, 1H)m, 7.79 (d, J=2 Hz, 1H), 7.85 (d, J=3.2 Hz, 1H), 8.83 (br s, 1H) ppm.

### Synthesis of Example 9

### 5-(6-Chloro-2,2-difluoro-1,3-benzodioxol-5-yl)-2-(2,6-difluoro-phenyl)-4,5,6,7-tetrahydro-1H-pyrrolo[3,2-c]pyridine

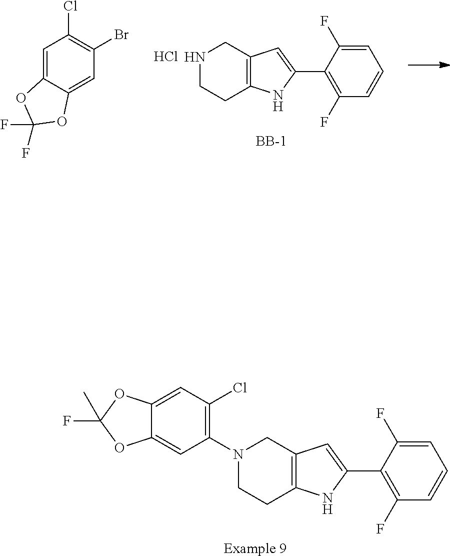 [SMILES: FC1(F)Oc2cc(Cl)c(Br)cc2O1 ; Cl ; Fc1cccc(F)c1-c1cc2c([nH]1)CCNC2 ; "BB-1" ; CC1(F)Oc2cc(Cl)c(N3CCc4[nH]c(-c5c(F)cccc5F)cc4C3)cc2O1 ; "Example 9"]

A degassed suspension of BB-1 (110 mg, 0.41 mmol), 5-bromo-6-chloro-2,2-difluorobenzo[d][1,3]dioxole (110 mg, 0.41 mmol), Pd(OAc)₂ (7 mg, 0.031 mmol), rac-BINAP (27 mg, 0.043 mmol) and Cs₂CO₃ (340 mg, 1.04 mmol) in dry toluene (2.4 mL) was heated under N₂ to 120° C. for 48 h. The mixture was chilled and the volatiles were removed under reduced pressure. The residue was purified by chromate-graphy (Interchim® cartridge 30SiHP/40 g, Cy/EtOAc) to yield the title compound of example 9 (70 mg, 41%).

LC-MS (method 1): m/z: [M+H]⁺=425.1 (MW calc.=424.78); Rₜ=4.4 min.

¹H-NMR (400 MHz, CDCl₃): δ (ppm)=2.85 (t, J=5.6 Hz, 2H), 3.42 (t, J=5.6 Hz, 2H), 4.13 (s, 2H); 6.67 (m, 1H), 6.92-7.00 (m, 3H), 7.07 (m, 1H), 7.13 (s, 1H), 8.80 (br s, 1H).

### Synthesis Example 10

### 2-(2,6-Difluoro-phenyl)-5-(4-methyl-pyridin-3-yl)-4,5,6,7-tetrahydro-1H-pyrrolo[3,2-c]pyridine

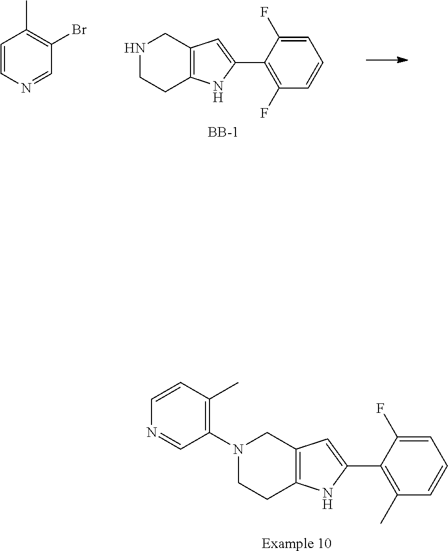

A mixture of BB-1 (200 mg, 0.85 mmol), 3-bromo-4-methyl-pyridine (175 mg, 1.02 mmol) and Cs₂CO₃ (552 mg, 1.7 mmol) in dioxane (4 mL) was degassed with Ar for 30 min. Pd(OAc)₂ (19 mg, 0.085 mmol), rac-BINAP (53 mg, 0.085 mmol) were added to the RM and heated in seal tube at 110° C. for 16 h. The RM was filtered and filtrate was concentrated under reduced pressure to get the crude compound which was purified by CC (silica gel; EtOAc/Hex) to yield the title compound of example 10 (85 mg, 30%). LC-MS (method 3): m/z [M+H]⁺=326.1 (MW calc. 325.36); Rₜ=3.75 min.

¹H NMR (DMSO-d₆, 400 MHz), δ (ppm)=10.82 (s, 1H), 8.30 (s, 1H), 8.12 (d, J=4.72 Hz, 1H), 7.26-7.21 (m, 1H), 7.19-7.12 (m, 3H), 6.32 (d, J=1.44 Hz, 1H), 4.02 (s, 2H), 3.25 (t, J=5.56 Hz, 2H), 2.78 (t, J=5.48 Hz, 2H), 2.30 (s, 3H) ppm.

### Synthesis Example 11

### 5-(4-Chloro-2-methyl-phenyl)-2-(2,6-difluoro-phenyl)-4,5,6,7-tetrahydro-1H-pyrrolo[3,2-c]pyridine

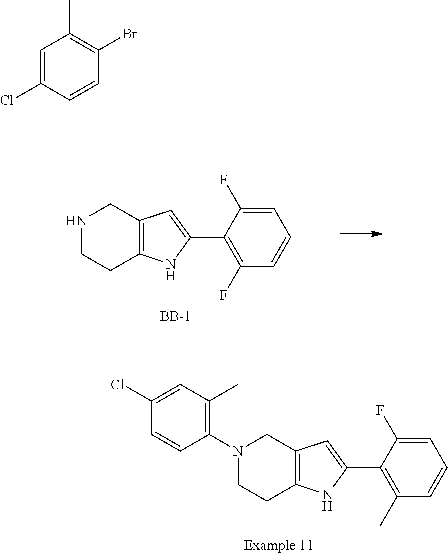

A mixture of BB-1 (300 mg, 1.28 mmol), 1-bromo-4-chloro-2-methyl-benzene (313 mg, 1.53 mmol) and Cs₂CO₃ (832 mg, 2.56 mmol) in dioxane (6 mL) was degassed with Ar for 30 min. Pd(OAc)₂ (29 mg, 0.128 mmol), rac-BINAP (80 mg, 0.128 mmol) were added to the RM and heated in seal tube at 110° C. for 16 h. The RM was filtered and filtrate was concentrated under reduced pressure to get the crude compound which was purified by CC (silica gel; EA/Hex) to yield the title compound of example 11 (110 mg, 24%).

Yield: 24% (110 mg, 0.307 mmol)

LC-MS (method 3): m/z [M+H]⁺=359.0 (MW calc.=358.81); Rₜ=2.48 min.

¹H NMR (DMSO-d₆, 400 MHz), δ (ppm)=10.81 (s, 1H), 7.27-7.21 (m, 2H), 7.20-7.10 (m, 4H), 6.31 (d, J=1.56 Hz, 1H), 3.89 (s, 2H), 3.14 (t, 5.52 Hz, 2H), 2.77 (t, J=5.48 Hz, 2H), 2.27 (s, 3H) ppm.

### Synthesis Example 12

### 4-[2-(2,6-Difluoro-phenyl)-4,5,6,7-tetrahydro-1H-pyrrolo[3,2-c]pyridin-5-yl]-3-methyl-benzonitrile

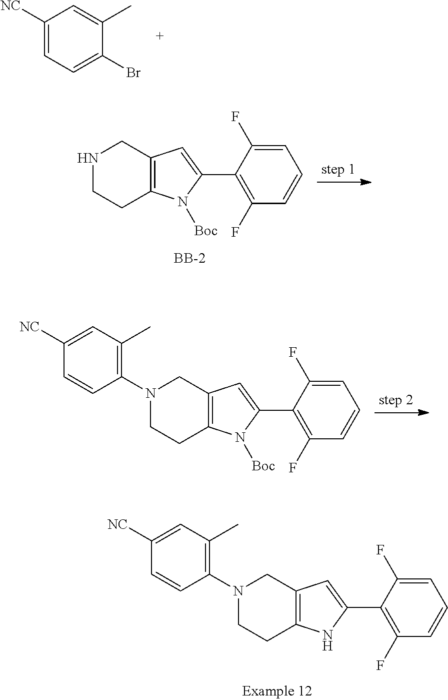

Step 1:

A mixture of BB-2 (150 mg, 0.449 mmol) 4-bromo-3-methylbenzonitrile (132 mg, 0.673 mmol) and Cs₂CO₃ (292 mg, 0.898 mmol) in dioxane (7 mL) was degassed with N₂ for 15 min. Pd₂(dba)₃ (20 mg, 0.022 mmol) and rac-BINAP (27 mg, 0.044 mmol) were added to the RM and heated in seal tube at 110° C. for 14 h. The RM was filtered through a pad of Celite™ and the filtrate was concentrated under reduced pressure to get the crude compound which was purified by CC (silica gel; EtOAc/Hex) to yield the desired compound (120 mg, 60%).

LC-MS (Method 3): m/z [M+H]⁺=450.4 (MW calc. 449.49); Rₜ=4.19 min.

Step 2:

To a solution of the intermediate from step 1 (160 mg, 0.356 mmol) in MeOH (10 mL) was added K₂CO₃ (147.5 mg, 1.06 mmol) and the RM was heated at reflux for 16 h. The RM was concentrated under reduced pressure and diluted with CH₂Cl₂ (100 mL), washed with water (50 mL) and brine (50 mL), dried and the volatiles removed under reduced pressure. The crude compound was purified by CC (silica gel, EtOAc/Hex) to yield the title compound of example 12 (100 mg, 80%).

LC-MS (method 3): m/z [M+H]⁺=350.2 (MW calc. 349.38); Rₜ=3.73 min.

¹H NMR (DMSO-d₆, 400 MHz), δ (ppm)=10.85 (s, 1H), 7.60-7.58 (m, 2H), 7.27-7.21 (m, 1H), 7.18-7.12 (m, 3H), 6.34 (s, 1H), 1.91 (s, 2H), 3.29 (t, J=5.6 Hz, 2H), 2.79 (t, J=5.48 Hz, 2H), 2.32 (s, 3H) ppm.

### Synthesis Example 13

### 4-[2-(2,6-Difluoro-phenyl)-4,5,6,7-tetrahydro-1H-pyrrolo[3,2-c]pyridin-5-yl]-3-methyl-benzoic acid methyl ester

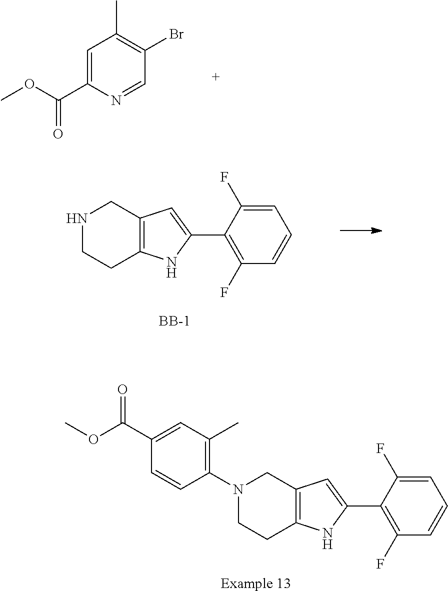 [SMILES: COC(=O)c1cc(C)c(Br)cn1 ; Fc1cccc(F)c1-c1cc2c([nH]1)CCNC2 ; "BB-1" ; COC(=O)c1ccc(N2CCc3[nH]c(-c4c(F)cccc4F)cc3C2)c(C)c1 ; "Example 13"]

A mixture of BB-1 (200 mg, 0.85 mmol) and 4-bromo-3-methyl-benzoic acid methyl ester (293 mg, 1.28 mmol) and Cs₂CO₃ (555 mg, 1.70 mmol) in dioxane (7 mL) was degassed with N₂ for 15 min. Pd(OAc)₂ (9.6 mg, 0.042 mmol) and rac-BINAP (53 mg, 0.085 mmol) were added to the RM and heated at 110° C. in a seal tube for 14 h. The RM was filtered through a pad of Celite™ and the filtrate was concentrated under reduced pressure to get the crude compound which was purified by CC (silica gel; EtOAc/Hex) to yield the title compound of example 13 (130 mg, 40%).

LC-MS (method 3): m/z [M+H]⁺=383.0 (MW calc.=382.40); Rₜ=3.93 min.

¹H NMR (DMSO-d₆, 400 MHz), δ (ppm)=10.84 (s, 1H), 7.77-7.74 (m, 2H), 7.26-7.21 (m, 2H), 7.16 (m, 3H), 4.03 (s, 2H), 3.80 (s, 3H), 3.27 (t, J=5.2 Hz, 2H), 2.80 (t, J=5.18 Hz, 2H), 2.33 (s, 3H) ppm.

### Synthesis Example 14

### 4-[2-(2,6-Difluoro-phenyl)-4,5,6,7-tetrahydro-1H-pyrrolo[3,2-c]pyridin-5-yl]-N,N,3-trimethyl-benzenesulfonic acid amide

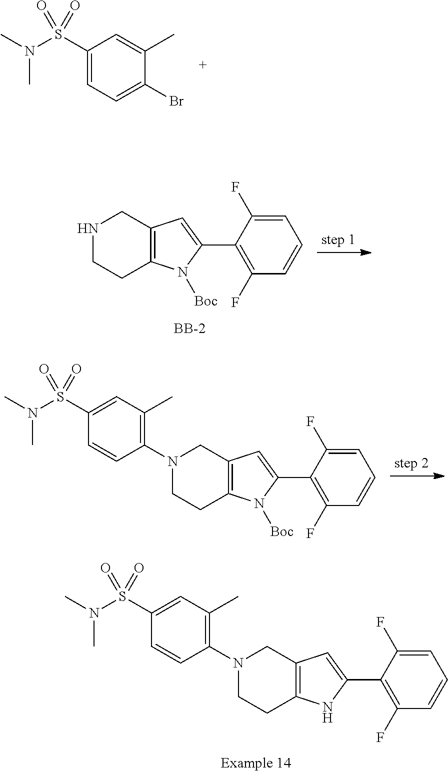

Step 1:

A mixture of BB-2 (250 mg, 0.748 mmol), 4-bromo-3,N—N,trimethylbenzenesulphonamide (312 mg, 1.12 mmol) and Cs₂CO₃ (486 mg, 1.49 mmol) in dioxane (7 mL) was degassed with N₂ for 15 min. Pd₂(dba)₃ (0.0374 mmol) and rac-BINAP (0.0748 mmol) were added to the RM and heated at 110° C. for 14 h in seal tube. The RM was cooled to it and filtered through a pad of Celite™. The filtrate was concentrated under reduced pressure to get the crude compound which was purified by CC (silica gel, EtOAc/Hex) to yield the desired compound (200 mg, 0.376 mmol, 63%).

LC-MS (Method 1): m/z [M+H]⁺=532.0 (MW calc.=531.62); Rt=4.01 min.

Step 2:

To a solution of the intermediate of step 2 (200 mg, 0.376 mmol) in MeOH (10 mL) was added K₂CO₃ (156 mg, 1.12 mmol) and the RM was heated at reflux for 16 h. The RM was concentrated and diluted with CH₂Cl₂ (100 mL), washed with water (50 mL) and brine (50 mL). The organic layer was dried and the volatiles were removed under reduced pressure to get the crude compound which was purified by chromatography (silica gel, EtOAc/Hex) to yield the title compound of example 14 (100 mg, 62%).

LC-MS (method 3): m/z [M+H]⁺=431.8 (MW calc.=431.50); Rₜ=4.01 min.

¹H NMR (DMSO-d₆, 400 MHz), δ (ppm)=10.85 (s, 1H), 7.52-7.50 (m, 2H), 7.26-7.23 (m, 2H), 7.15 (t, J=8.48 Hz, 2H), 6.34 (s, 1H), 4.06 (s, 2H), 3.31 (m, 2H), 2.81 (bs, 2H), 2.58 (s, 6H), 2.37 (s, 3H) ppm.

### Synthesis Example 15

### 5-(6-Chloro-2-methyl-pyridin-3-yl)-2-(2,6-difluoro-phenyl)-4,5,6,7-tetrahydro-1H-pyrrolo[3,2-c]pyridine

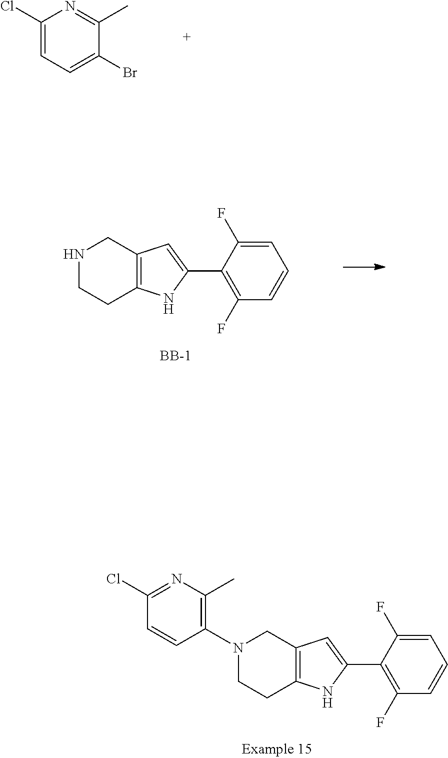

A mixture of BB-1 (200 mg, 0.85 mmol), 3-bromo-6-chloro-2-methyl pyridine (264 mg, 1.28 mmol) and Cs₂CO₃ (555.5 mg, 1.70 mmol) in dioxane (7 mL) was degassed with Ar for 30 min. Pd(OAc)₂ (19 mg, 0.085 mmol), rac-BINAP (53 mg, 0.17 mmol) were added and the RM was heated in a sealed tube to 110° C. for 16 h. The RM was filtered the volatiles were removed under reduced pressure. The residue was purified by CC (silica gel, 20% EtOAc/Hex) to yield the title compound of example 15 (61 mg, 20%) LC-MS (Method 3): m/z [M+H]⁺=360.2 (MW calc.=359.8); Rₜ=3.77 min.

¹H NMR (DMSO-d₆, 400 MHz), δ (ppm)=10.84 (s, 1H), 7.52 (d, J=8.40 Hz, 1H), 7.28-7.21 (m, 2H), 7.18-7.11 (m, 2H), 6.32 (d, J=1.32 Hz, 1H), 3.94 (s, 2H), 3.20 (t, J=5.48 Hz, 2H), 2.78 (t, J=5.24 Hz, 2H), 2.45 (s, 3H) ppm.

### Synthesis Example 16

### 2-(2,6-Difluoro-phenyl)-5-(4-methyl-6-methylsulfonyl-pyridin-3-yl)-4,5,6,7-tetrahydro-1H-pyrrolo[3,2-c]pyridine

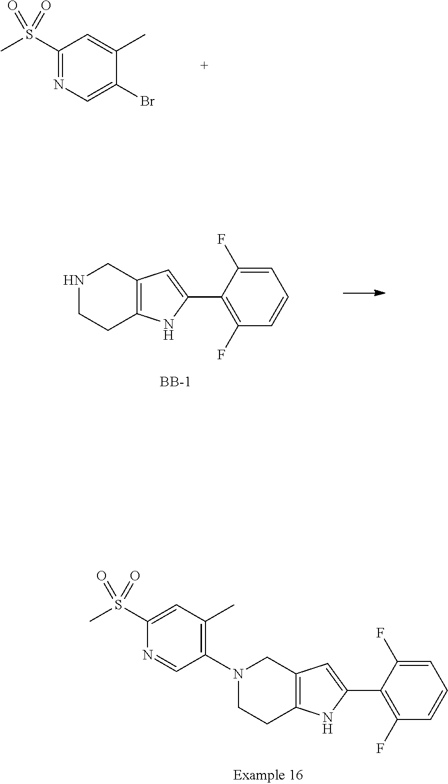 [SMILES: Cc1cc(S(C)(=O)=O)ncc1Br ; Fc1cccc(F)c1-c1cc2c([nH]1)CCNC2 ; "BB-1" ; Cc1cc(S(C)(=O)=O)ncc1N1CCc2[nH]c(-c3c(F)cccc3F)cc2C1]

A mixture of BB-1 (200 mg, 0.85 mmol), 5-bromo-2-methanesulfonyl-4-methyl-pyridine (255 mg, 1.02 mmol) and Cs₂CO₃ (552 mg, 1.7 mmol) in dioxane (5 mL) was degassed with Ar for 30 min. Pd(OAc)₂ (19 mg, 0.085 mmol) and rac-BINAP (53 mg, 0.085 mmol) were added and the RM and was heated in a sealed tube to 110° C. for 16 h. The RM was filtered and the volatiles were removed under reduced pressure. The residue was purified by CC (silica gel EtOAc/Hex) to yield the title compound of example 16 (95 mg, 28%) LC-MS (method 3): m/z [M+H]⁺=404.0 (MW calc. 403.45); Rₜ=3.42 min.

¹H NMR (DMSO-d₆, 400 MHz), δ (ppm)=10.88 (s, 1H), 8.40 (s, 1H), 7.83 (s, 1H), 7.27-7.22 (m, 1H), 7.15 (t, J=8.56 Hz, 2H), 6.35 (s, 1H), 4.20 (s, 2H), 3.38 (t, J=5.36 Hz, 2H), 3.20 (s, 3H), 2.82 (t, J=5.3 Hz, 2H), 2.42 (s, 3H).

### Synthesis Example 17

### 4-[2-(2-Chloro-6-fluoro-phenyl)-4,5,6,7-tetrahydro-1H-pyrrolo[3,2-c]pyridin-5-yl]-5-methyl-2-pyridin-3-yl-thiazole

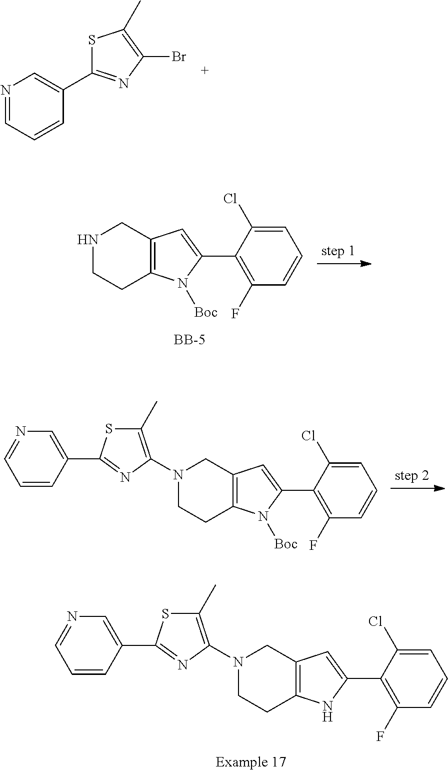

Step 1:

A mixture of BB-5 (200 mg, 0.57 mmol), 4-bromo-5-methyl-2-(pyridin-3-yl)thiazole (290 mg, 1.14 mmol) and Cs₂CO₃ (557 mg, 1.71 mmol) in toluene (5 mL) was degassed with N₂ for 15 min. Pd₂(dba)₃ (15 mg, 0.017 mmol) and rac-BINAP (21 mg, 0.034 mmol) were added to the RM and heated to 120° C. for 14 h in seal tube. The RM was cooled to it and filtered through a pad of Celite™. The filtrate was concentrated under reduced pressure to get the crude compound which was used for the next step without further purification.

Step 2:

To a solution of the intermediate of step 2 in MeOH (5 mL) was added K₂CO₃ (393 mg, 2.85 mmol) and the RM was heated at reflux for 16 h. The RM was concentrated and diluted with CH₂Cl₂ and was washed with water and brine. The organic layer was dried and the volatiles were removed under reduced pressure to get the crude compound which was purified by chromatography (Interchim® cartridge 50SiHP/12 g, Cy/EtOAc) to yield the title compound of example 17 (23 mg, 10%). LC-MS (method 2): m/z [M+H]⁺=424.1 (MW calc.=424.92); Rt=0.86 min.

¹H NMR (DMSO-d₆, 400 MHz), δ (ppm)=10.81 (s, 1H), 9.03 (d, J=1.9 Hz, 1H), 8.61 (dd, J=4.9 Hz, J=1.5 Hz, 1H), 8.19 (dt, J=8 Hz, J=8 Hz, 1H), 7.51-7.49 (m, 1H), 7.39-7.37 (m, 1H), 7.34-7.25 (m, 3H), 6.15 (d, J=2.3 Hz, 1H), 4.19 (s, 2H), 2.44 (t, J=5.6 Hz, 2H), 2.83 (t, J=5.6 Hz, 2H).

### Synthesis Example 18

### 5-[2-(2,6-Difluoro-phenyl)-4,5,6,7-tetrahydro-1H-pyrrolo[3,2-c]pyridin-5-yl]-4-methyl-2-pyridin-3-yl-thiazole

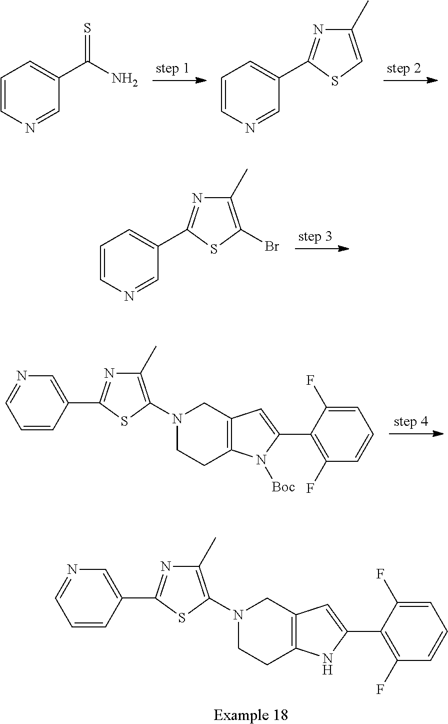

Step 1:

To a mixture of thionicotinamide (3.3 g, 23.8 mmol) in EtOH (100 mL) was added chloroacetone (2.28 mL, 28.6 mmol) and the resulting mixture was heated to reflux for 48 h. The volatiles were removed under reduced pressure and the residue was purified by CC (silica gel; EtOAc/Hex) to yield the desired compound (3.0 g, 71%).

Step 2:

To a suspension of the intermediate of step 1 (500 mg, 2.84 mmol) in CH₂Cl₂ (6 mL) was slowly added bromine (0.43 mL, 8.52 mmol) at 0° C. and the mixture was stirred at it for 1.5 h. The RM was quenched with aqueous NaHCO₃ and extracted with CH₂Cl₂. The combined organic layers were dried and the volatiles were removed under reduced pressure to yield the desired compound (700 mg, 96%).

Step 3:

A mixture BB-2 (200 mg, 0.598 mmol) and the intermediate of step 2 (183 mg, 0.718 mmol) in toluene (5 mL) was degassed through purging with N₂ for 15 min followed by the addition of Cs₂CO₃ (777 mg, 2.39 mmol), rac-BINAP (0.74 mg, 0.119 mmol) and Pd₂(dba)₃ (54 mg, 0.059 mmol). The resulting mixture was heated in sealed tube to 120° C. for 16 h. The RM was filtered over a pad of Celite™ and the volatiles were removed under reduced pressure. The residue was purified by chromatography (silica gel, EtOAc/Hex) to yield the desired compound (100 mg, 32%).

LC-MS (method 3): m/z [M+H]⁺=509.2 (MW calc.=508.58); Rₜ=4.48 min.

Step 4:

To a solution of the intermediate of step 3 (100 mg, 0.196 mmol) in MeOH (10 mL) was added K₂CO₃ (108 mg, 0.787 mmol) and the RM was refluxed for 16 h. The volatiles were removed under reduced pressure and the residue was diluted with CH₂Cl₂, was washed with water and brine, dried and the volatiles were removed under reduced pressure. The residue was purified by CC (silica gel, EtOAc/Hex) to yield the title compound of example 18 (48 mg, 60%).

LC-MS (method 3): m/z [M+H]⁺=409.0 (MW calc.=408.47); Rₜ=3.79 min.

¹H NMR (DMSO-d₆, 400 MHz), δ (ppm)=10.89 (s, 1H), 9.00 (s, 1H), 58 (d, J=4.08 Hz, 1H), 8.17 (d, J=8.24 Hz, 1H), 7.49-7.46 (m, 1H), 7.28-7.13 (m, 1H), 7.15 (t, J=8.56 Hz, 2H), 6.32 (s, 1H), 3.99 (s, 2H), 3.25 (t, J=5.12 Hz, 2H), 2.84 (t, J=5.20 Hz, 2H), 2.36 (s, 3H).

### Synthesis Example 19

### 2-(2,6-Difluoro-phenyl)-5-[2-methyl-5-(trifluoromethyl)-2H-pyrazol-3-yl]-4,5,6,7-tetrahydro-1H-pyrrolo[3,2-c]pyridine

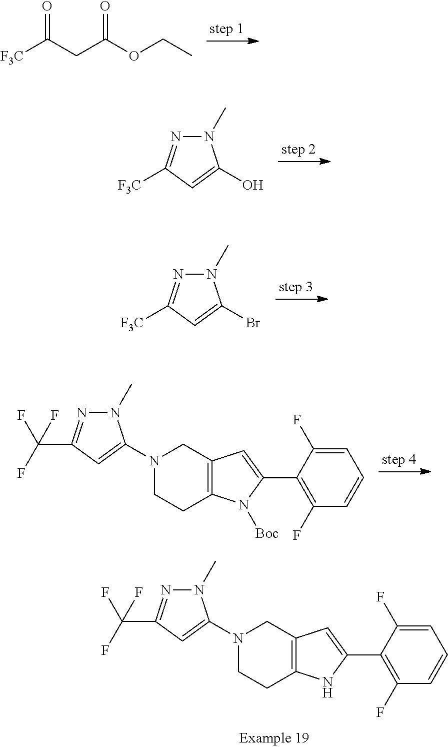 [SMILES: CCOC(=O)CC(=O)C(F)(F)F ; Cn1nc(C(F)(F)F)cc1O ; Cn1nc(C(F)(F)F)cc1Br ; Cn1nc(C(F)(F)F)cc1N1CCc2c(cc(-c3c(F)cccc3F)n2C(=O)OC(C)(C)C)C1 ; Cn1nc(C(F)(F)F)cc1N1CCc2[nH]c(-c3c(F)cccc3F)cc2C1]

Step 1:

To a solution of ethyl 4,4,4-trifluoroacetylacetate (6.0 g, 32.6 mmol) in EtOH (24 mL) was added methylhydrazine (1.78 mL, 32.6 mmol) and HCl (1.2 mL) and the mixture was heated to reflux for 16 h. The volatiles were removed under reduced pressure and the residue was purified by CC (silica gel EtOAc/Hex) to yield the desired compound (4.0 g, 75%)

Step 2:

A mixture of the intermediate of step 1 (3.3 g, 19.9 mmol) and POBr₃ (17.1 g, 59.6 mmol) was heated to 120° C. for 16 h. The RM was cooled to it and ice cold water was added and the RM was basified with 1N NaOH solution to pH-8-9. The mixture was extracted with EtOAc and the combined organic layers were washed with brine, were dried and the volatiles were removed under reduced pressure. The residue was purified by CC (silica gel, EtOAc/Hex) to yield the desired compound (1.59 g, 35%).

Step 3:

A mixture of BB-2 (600 mg, 1.79 mmol) and the intermediate of step 2 (617 mg, 2.69 mmol) in toluene (10 mL) was degassed through purging with N₂ for 15 min followed by addition of Cs₂CO₃ (2.3 g, 7.16 mmol), rac-BINAP (0.358 mmol) and Pd₂(dba)₃ (0.179 mmol) and the resulting mixture was heated in sealed tube to 110° C. for 14 h. The RM was filtered over a pad of Celite™ and the volatiles were removed under reduced pressure. The residue was purified by CC (silica gel, EtOAc/Hex) to yield the desired compound (330 mg, 38%).

LC-MS (Method 1): m/z [M+H]⁺=483.3 (MW calc.=482.45); Rₜ=3.6 min.

Step 4:

To a solution of the intermediate of step 3 (200 mg, 0.414 mmol) in MeOH (10 mL) was added K₂CO₃ (171 mg, 1.24 mmol) and the RM was heated to reflux for 16 h. The volatiles were removed under reduced pressure and the residue was diluted with CH₂Cl₂, was washed with water and brine, dried and the volatiles were removed under reduced pressure. The residue was purified by CC (silica gel, EtOAc/Hex) to yield the title compound of example 19 (80 mg, 51%).

LC-MS (method 3): m/z [M+H]⁺=382.8 (MW calc.=382.33); Rₜ=3.73 min.

¹H NMR (DMSO-d₆, 400 MHz), δ (ppm)=10.87 (s, 1H), 7.27-7.22 (m, 1H), 7.15 (t, J=8.56 Hz, 2H), 6.36 (s, 1H), 6.30 (d, J=1.48 Hz, 1H), 3.98 (s, 2H), 3.76 (s, 3H), 3.23 (t, J=5.64 Hz, 2H), 2.82 (t, J=5.36 Hz, 2H).

### Synthesis Example 20

### 5-(2,2-Difluoro-6-methyl-1,3-benzodioxol-5-yl)-2-(2,6-difluoro-phenyl)-4,5,6,7-tetrahydro-1H-pyrrolo[3,2-c]pyridine

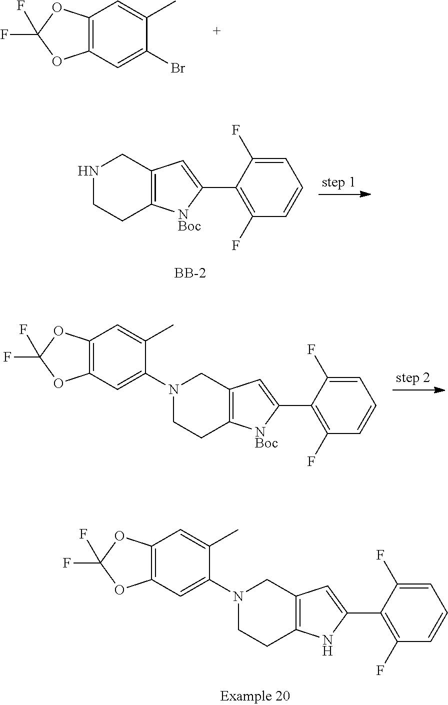 [SMILES: Cc1cc2c(cc1Br)OC(F)(F)O2 ; CC(C)(C)OC(=O)n1c(-c2c(F)cccc2F)cc2c1CCNC2 ; "BB-2" ; Cc1cc2c(cc1N1CCc3c(cc(-c4c(F)cccc4F)n3C(=O)OC(C)(C)C)C1)OC(F)(F)O2 ; Cc1cc2c(cc1N1CCc3[nH]c(-c4c(F)cccc4F)cc3C1)OC(F)(F)O2]

Step 1:

A mixture of BB-2 (200 mg, 0.598 mmol) and 5-bromo-2,2-difluoro-6-methylbenzo[d][1,3]dioxole (298 mg, 1.20 mmol) in toluene (5.1 mL) was degassed through purging with N₂ for 15 min followed by addition of Cs₂CO₃ (578 mg, 1.79 mmol), rac-BINAP (21 mg, 0.036 mmol) and Pd₂(dba)₃ (18 mg, 0.018 mmol) and the resulting mixture was heated in sealed tube to 110° C. for 14 h. The RM was filtered over a pad of Celite™ and the volatiles were removed under reduced pressure. The crude product was used without further purification.

LC-MS (Method 2): m/z [M+H]⁺=505.2 (MW calc.=504.47); Rₜ=1.25 min.

Step 2:

The crude product of step 1 was dissolved in MeOH (5 mL) and K₂CO₃ (2.99 mmol) was added and the mixture was stirred for 2 h at 40° C. and for 48 h at rt. The mixture was diluted with aqueous NH₄Cl and extracted with EtOAc. The combined organic layers were dried and the volatiles were removed under reduced pressure. The residue was purified by chromatography (Interchim® cartridge C18 RP 15 μm/12 g, CH₃CN/H₂O) to yield the title compound of example 20 (18 mg, 7%).

LC-MS (Method 2): m/z [M+H]⁺=505.2 (MW calc.=504.47); Rₜ=1.25 min.

¹H NMR (DMSO-d₆, 400 MHz), δ (ppm)=10.79 (s, 1H), 7.27-7.22 (m, 3H), 7.16-7.13 (m, 2H), 6.30 (d, J=1.5 Hz, 1H), 3.86 (s, 2H), 3.12 (t, J=5.6 Hz, 2H), 2.78 (t, J=5.6 Hz, 2H) 2.29 (s, 3H).

### Synthesis Example 21

### 2-(2,6-Difluoro-phenyl)-5-(2,5-dimethoxyphenyl)-4,5,6,7-tetrahydro-1H-pyrrolo[3,2-c]pyridine

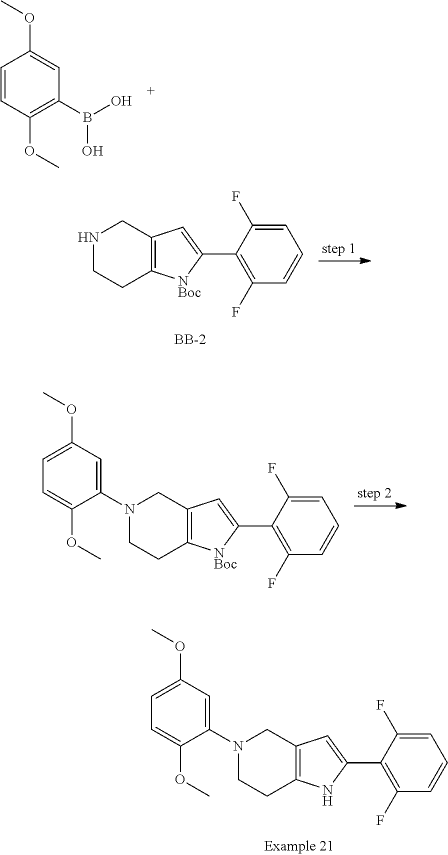 [SMILES: COc1ccc(OC)c(B(O)O)c1 ; CC(C)(C)OC(=O)n1c(-c2c(F)cccc2F)cc2c1CCNC2 ; "BB-2" ; COc1ccc(OC)c(N2CCc3c(cc(-c4c(F)cccc4F)n3C(=O)OC(C)(C)C)C2)c1 ; COc1ccc(OC)c(N2CCc3[nH]c(-c4c(F)cccc4F)cc3C2)c1]

Step 1:

A mixture of BB-2 (299 mg, 0.897 mmol), 2,5-dimethoxyphenylboronic acid (329 mg, 1.79 mmol), copper(II)acetate (325 mg, 1.79 mmol) and NEt₃ (240 μL, 1.79 mmol) and 4 Å molecular sieve in CH₂Cl₂ (5 mL) was stirred at it for 4 d. The RM was filtered and the volatiles were removed under reduced pressure to yield the desired compound which was used for the next step without further purification.

LC-MS (Method 2): m/z [M+H]⁺=471.3 (MW calc.=470.51); Rₜ=1.05 min.

Step 2:

The crude product of step 1 was dissolved in MeOH (3 mL) and K₂CO₃ (619 mg) was added and the resulting suspension was stirred at it for 3 d. Aqueous NH₄Cl was added and the mixture was extracted with EtOAc. The combined organic layers were dried and the volatiles were removed under reduced pressure. The residue was purified by chromatography (Interchim® cartridge 50SiHP/12 g, Cy/EtOAc) to yield the title compound of example 21 (18 mg, 5% over 2 steps).

LC-MS (Method 2): m/z [M+H]⁺=371.1 (MW calc.=370.39); Rₜ=0.71 min.

¹H NMR (DMSO-d₆, 400 MHz), δ (ppm)=10.72 (s, 1H), 7.28-7.18 (m, 1H), 7.17-7.11 (m, 2H), 6.84 (d, J=8.8 Hz, 1H), 6.53-6.51 (m, 1H), 6.49-6.45 (m, 1H), 6.32 (s, 1H), 4.00 (s, 2H), 3.74 (s, 3H), 3.68 (s, 3H), 3.34 (t, J=8.8 Hz, 2H), 2.73 (t, J=8.8 Hz, 2H).

### Synthesis Example 22

### 2-(2,6-Difluoro-phenyl)-5-o-tolyl-4,5,6,7-tetrahydro-1H-pyrrolo[3,2-c]pyridine

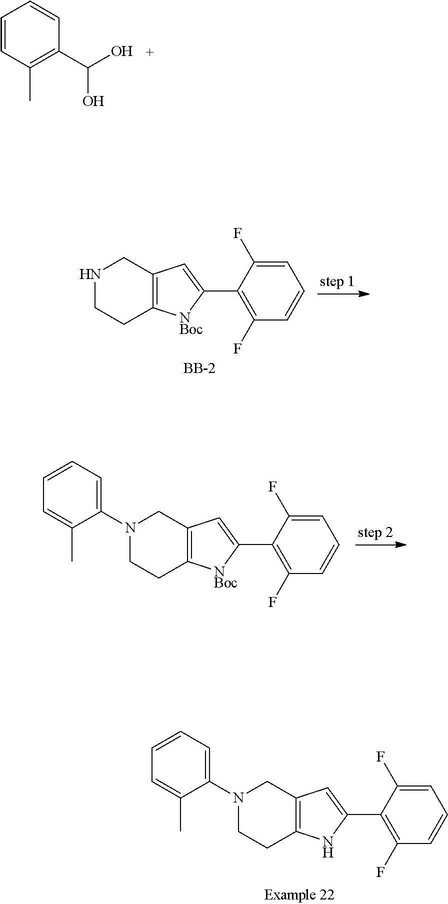

Step 1:

A mixture of BB-2 (174 mg, 0.523 mmol), 2-methylbenzene boronic acid (355 mg, 2.62 mmol), copper(II)acetate (94 mg, 0.523 mmol) and NEt₃ (362 μL, 2.62 mmol) and 4 Å molecular sieve in CH₂Cl₂ (4.6 mL) was stirred at it for 3 d. The RM was filtered and the volatiles were removed under reduced pressure to yield the desired compound which was used for the next step without further purification.

Step 2:

The crude product of step 1 was dissolved in MeOH (2.8 mL) and K₂CO₃ (477 mg) was added and the resulting suspension was stirred at it for 3 d. Aqueous NH₄Cl was added and the mixture was extracted with EtOAc. The combined organic layers were dried and the volatiles were removed under reduced pressure. The residue was purified by chromatography (Interchim® cartridge 50SiHP/12 g, Cy/EtOAc) to yield the title compound of example 22 (33 mg, 20% over 2 steps).

LC-MS (Method 2): m/z [M+H]⁺=325.2 (MW calc.=324.37); Rₜ=0.93 min.

¹H NMR (DMSO-d₆, 400 MHz), δ (ppm)=10.77 (s, 1H), 7.26-7.21 (m, 1H), 7.18-7.11 (m, 2H), 6.95 (dt, J=4.0, 0.4 Hz, 1H) 6.32 (s, 1H), 3.90 (s, 2H), 3.16 (t, J=5.6 Hz, 2H), 2.78 (t, J=5.6 Hz, 2H), 2.28 (s, 3H).

### Synthesis Example 23

### 4-[2-(2,6-Difluoro-phenyl)-4,5,6,7-tetrahydro-1H-pyrrolo[3,2-c]pyridin-5-yl]-5-methyl-2-phenyl-thaizole

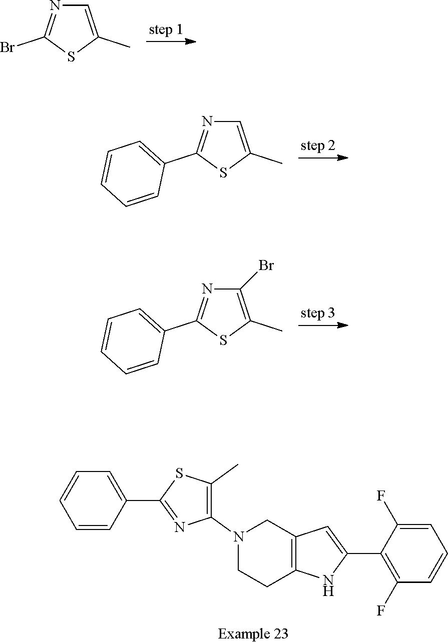

Step 1:

A mixture of 2-bromo-5-methyl-thiazole (2.0 g, 11.2 mmol), phenylboronic acid (1.64 g, 13.5 mmol) and K₂CO₃ (22.5 mmol) in a mixture of dioxane (20 mL) and water (4 mL) was degassed through purging with Ar for 30 min. Pd(PPh₃)₄ (647 mg, 0.56 mmol) was added and the mixture was heated to 110° C. for 5 h. The volatiles were removed under reduced pressure, the residue was diluted with EtOAc, was washed with water and brine, dried and the volatiles were removed under reduced pressure. The residue was purified by CC (silica gel, EtOAc/Hex) to yield the desired compound (1.1 g, 56%).

LC-MS (method 3): m/z [M+H]⁺=175.6 (MW calc.=175.25); Rₜ=2.70 min.

Step 2:

To a solution of the intermediate of step 1 (1.0 g, 5.7 mmol) in CH₂Cl₂ (10 mL) was added bromine (0.88 mL, 17.1 mmol) at 0° C. and the mixture was stirred at it for 2 h. The RM was poured into ice cold water and was extracted with EtOAc. The combined organic layers were washed with water and aqueous NaHCO₃, dried and the volatiles were removed under reduced pressure. The residue was purified by CC (silica gel, EtOAc/Hex) to yield the desired compound (600 mg, 41%).

Step 4:

A mixture of BB-1 (300 mg, 1.18 mmol), the intermediate of step 2 (329 mg, 1.29 mmol) and

Cs₂CO₃ (767 mg, 2.36 mmol) in dioxane (6 mL) was degassed through purging with Ar for 30 min. Pd(OAc)₂ (26 mg, 0.118 mmol), rac-BINAP (73 mg, 0.118 mmol) were added and the mixture was heated in sealed tube to 110° C. for 16 h. The RM was filtered over a pad of Celite™ and the volatiles were removed under reduced pressure. The residue was purified by CC (silica gel, EtOAc/Hex) to yield the title compound of example 23 (43 mg).

LC-MS (method 3): m/z [M+H]⁺=408.1 (MW calc. 407.48); Rₜ=2.73 min.

¹H NMR (DMSO-d₆, 400 MHz), δ (ppm)=10.79 (s, 1H), 7.82 (d, J=6.92 Hz, 2H), 7.48-7.40 (m, 3H), 7.26-7.20 (m, 1H), 7.14 (t, J=8.52 Hz, 2H), 6.32 (s, 1H), 4.16 (s, 2H), 3.40 (t, J=5.60 Hz, 2H), 2.83 (t, J=5.70 Hz, 2H), 2.37 (s, 3H).

### Synthesis Example 24

### 4-[2-(2,6-Difluoro-phenyl)-4,5,6,7-tetrahydro-1H-pyrrolo[3,2-c]pyridin-5-yl]-5-methyl-2-pyridin-4-yl-thiazole

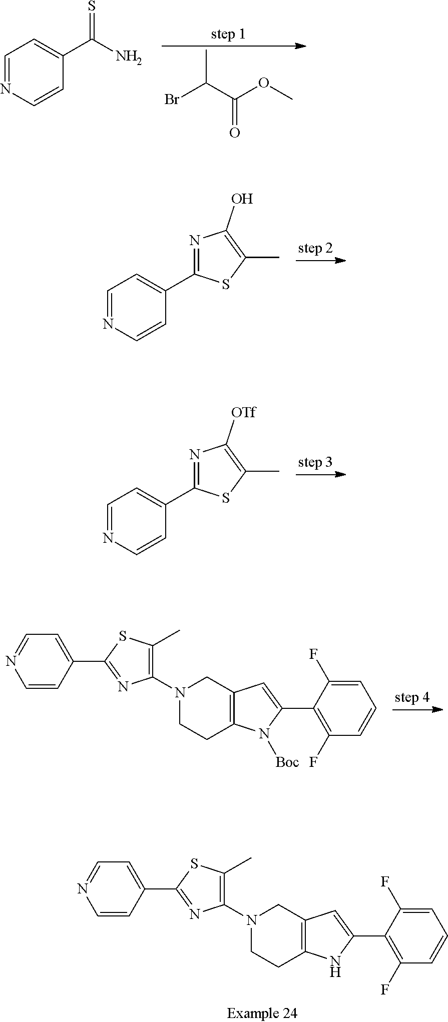

Step 1:

To a solution thioisonicotinamide (6.0 g, 43.5 mmol) in EtOH (90 mL) were added pyridine (5.9 mL, 79.9 mmol) and 2-bromo-propionic acid methyl ester (5.8 mL, 52.17 mmol) and the RM was heated to reflux for 16 h. The volatiles were removed under reduced pressure and the residue was triturated with 5% MeOH/EtOAc (50 mL) to yield the desired compound (3.3 g, 39%).

Step 2:

To a solution of the intermediate of step 1 (1.5 g, 7.81 mmol) in DMF (20 mL) was added NaH (50% in paraffin oil, 975 mg, 20.3 mmol) at 0° C. and the mixture was stirred at it for 20 min. (CF₃SO₂)₂NPh (4.1 g, 11.7 mmol) was added and the RM was stirred at it for 16 h. The RM was quenched with aqueous NH₄Cl and was extracted with EtOAc. The combined organic layers were washed with water and brine, dried and the volatiles were removed under reduced pressure. The residue was purified by CC (silica gel, EtOAc/Hex) to yield the desired compound (1.0 g, 40%).

Step 3:

A mixture of BB-2 (300 mg, 0.898 mmol) and the intermediate of step 2 (291 mg, 0.898 mmol) in toluene (10 mL) was degassed through purging with N₂ for 15 min followed by addition of Cs₂CO₃ (875 mg, 2.69 mmol) X-phos (43 mg, 0.089 mmol, 0.1) and Pd₂(dba)₃ (82 mg, 0.089 mmol). The resulting RM was heated in sealed tube to 110° C. for 14 h. The RM was filtered over a pad of Celite™ and the volatiles were removed under reduced pressure. The residue was purified by CC (neutral alumina, EtOAc/Hex) to afford the desired compound (100 mg, 22%).

LC-MS (method 3): m/z [M+H]⁺=509.2 (MW calc.=508.47); Rₜ=2.86 min.

Step 4:

To a solution of the intermediate of step 3 (100 mg, 0.196 mmol) in MeOH (5 mL) was added K₂CO₃ (108 mg, 0.787 mmol) and the RM was heated to reflux for 16 h. The volatiles were removed under reduced pressure and the residue was diluted with CH₂Cl₂, washed with water and brine and the combined organic layers were dried and the volatiles were removed under reduced pressure. The residue was purified by CC (silica gel, EtOAc/Hex) to yield the title compound of example 24 (45 mg, 56%).

LC-MS (method 3): m/z [M+H]⁺=409.0 (MW calc.=408.47); Rₜ=2.21 min.

¹H NMR (DMSO-d₆, 400 MHz), δ (ppm)=10.80 (s, 1H), 8.63 (d, J=5.96 Hz, 2H), 7.77 (d, J=6.0 Hz, 2H), 7.26 (m, 1H), 7.14 (t, J=8.48 Hz, 2H), 6.32 (s, 2H), 4.19 (s, 2H), 3.43 (t, J=5.56 Hz, 2H), 2.85 (t, J=5.44 Hz, 2H), 2.32 (s, 3H).

### Synthesis Example 25

### 5-(5-Bromo-6-methyl-pyridin-2-yl)-2-(2,6-difluoro-phenyl)-4,5,6,7-tetrahydro-1H-pyrrolo[3,2-c]pyridine

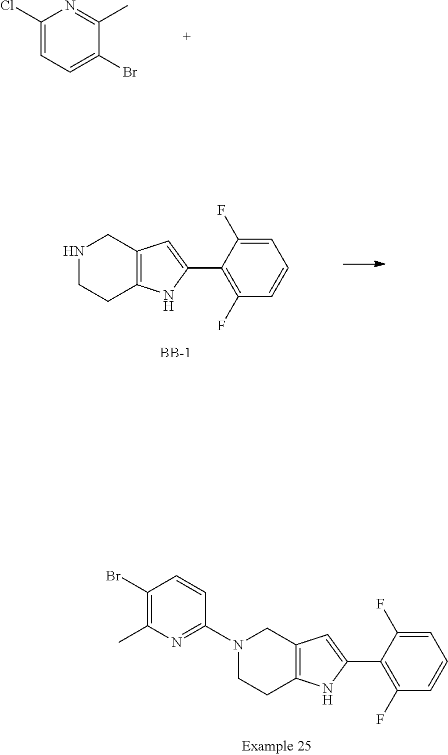

A mixture of BB-1 (200 mg, 0.85 mmol) 3-bromo-6-chloro-2-methyl pyridine (264 mg, 0.128 mmol) and Cs₂CO₃ (556 mg, 1.70 mmol) in dioxane (7 mL) was degassed through purging with Ar for 30 min. Pd(OAc)₂ (19 mg, 0.085 mmol, 0.10 eq.), rac-BINAP (53 mg, 0.17 mmol) were added to the RM and heated in seal tube to 110° C. for 16 h. The RM was filtered and the volatiles were removed under reduced pressure. The residue was purified by CC (silica gel, EtOAc/Hex) to the title compound of example 25 (60 mg, 18%).

LC-MS (Method 1): m/z [M+H]⁺=403.8, 405.8 (MW calc.=404.25); Rₜ=2.42 min.

¹H NMR (DMSO-d₆, 400 MHz), δ (ppm)=10.80 (s, 1H), 7.62 (d, J=8.88 Hz, 1H), 7.28-7.20 (m, 1H), 7.13 (t, J=8.52 Hz, 2H), 6.68 (d, J=8.92 Hz, 1H), 6.34 (s, 1H), 4.48 (s, 2H), 3.89 (t, J=5.44 Hz, 2H), 2.74 (t, J=5.24 Hz, 2H), 2.41 (s, 3H).

### Synthesis Example 26

### 4-[2-(2,6-Difluoro-phenyl)-4,5,6,7-tetrahydro-1H-pyrrolo[3,2-c]pyridin-5-yl]-5-methyl-2-pyrazin-2-yl-thiazole

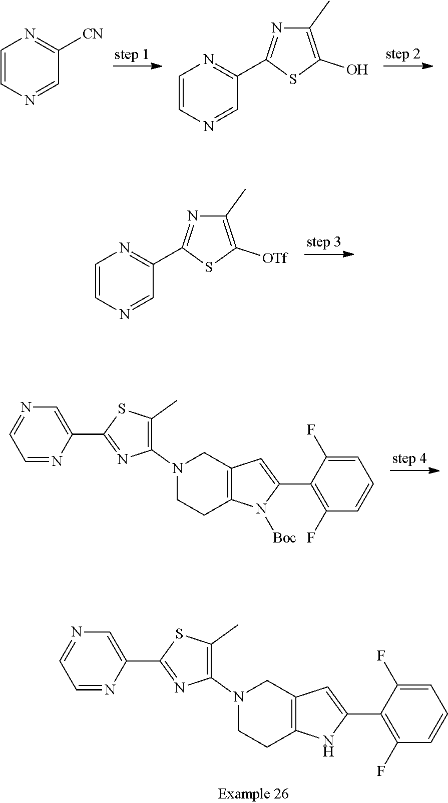 [SMILES: N#Cc1cnccn1 ; Cc1nc(-c2cnccn2)sc1O ; Cc1nc(-c2cnccn2)sc1OS(=O)(=O)C(F)(F)F ; Cc1sc(-c2cnccn2)nc1N1CCc2c(cc(-c3c(F)cccc3F)n2C(=O)OC(C)(C)C)C1 ; Cc1sc(-c2cnccn2)nc1N1CCc2[nH]c(-c3c(F)cccc3F)cc2C1]

Step 1:

To a mixture pyrazine-2-carbonitrile (2.0 g, 19.0 mmol) and 2-mercapto-propionic acid (2.1 g, 19.04 mmol) was added pyridine (3.0 mL, 38.1 mmol) and the mixture was heated to 100° C. for 2 h. The mixture was cooled to it and EtOH (100 mL) was added and the RM was stirred at it for 30 min. The formed precipitate was isolated though filtration and was washed with hex and was dried under reduced pressure to yield the desired compound (3.0 g, 15.5 mmol, 81%).

LC-MS (method 3): m/z [M+H]⁺=194.0 (MW calc.=193.23); Rₜ=2.42 min.

Step 2:

To a solution of the intermediate of step 1 (500 mg, 2.59 mmol) in DMF (10 mL) was added NaH (60% in paraffin oil, 310 mg, 7.77 mmol) at 0° C. and the RM was stirred at it for 20 min, followed by the addition of (CF₃SO₂)₂NPh (1.84 mg, 5.18 mmol) at 0° C. and the mixture was stirred at it for 16 h. The RM was quenched with aqueous NH₄Cl solution and was extracted with EtOAc. The combined organic layers were washed with water and brine, were dried and the volatiles were removed under reduced pressure. The residue was purified by CC (silica gel, EtOAc/Hex) to yield the desired compound (400 mg, 47%).

LC-MS (method 3): m/z [M+H]⁺=325.8 (MW calc.=325.29); Rₜ=3.64 min.

Step 3:

A mixture of BB-2 (200 mg, 0.598 mmol) and the intermediate of step 2 (233 mg, 0.718 mmol) in toluene (10 mL) was degassed through purging with N₂ for 15 min followed by the addition of Cs₂CO₃ (583 mg, 1.79 mmol), X-phos (28 mg, 0.059 mmol) and Pd₂(dba)₃ (54 mg, 0.059 mmol) and the resulting mixture was heated in sealed tube to 110° C. for 14 h. The RM was filtered over a pad of Celite™ and the volatiles were removed under reduced pressure. The residue was purified by CC (silica gel, EtOAc/Hex) to yield the desired compound (80 mg, 26%).

LC-MS (method 3): m/z [M+H]⁺=510.2 (MW calc.=509.57); Rₜ=4.41 min.

Step 4:

To a solution of the intermediate of step 3 (80 mg, 0.157 mmol) in MeOH (5 mL) was added K₂CO₃ (60 mg, 0.471 mmol) and the RM was heated to reflux for 16 h. The volatiles were removed under reduced pressure and the residue was diluted with CH₂Cl₂, was washed with water and brine, dried and the volatiles were removed under reduced pressure. The residue was purified by CC (silica gel, EtOAc/Hex) to yield the title compound of example 26 (40 mg, 0.097 mmol, 62%).

LC-MS (method 3): m/z [M+H]⁺=410.0 (MW calc.=409.46); Rt=3.89 min.

¹H NMR (DMSO-d₆, 400 MHz), δ (ppm)=10.81 (s, 1H), 9.21 (s, 1H), 8.66-8.64 (m, 2H), 7.28-7.21 (m, 1H), 7.14 (t, J=8.48 Hz, 2H), 6.33 (s, 1H), 4.20 (s, 2H), 3.44 (t, J=5.36 Hz, 2H), 2.86 (t, J=5.40 Hz, 2H), 2.42 (s, 3H).

### Synthesis Example 27

### 4-[2-(2,6-Difluoro-phenyl)-4,5,6,7-tetrahydro-1H-pyrrolo[2,3-c]pyridin-6-yl]-5-methyl-2-pyridin-3-yl-thiazole

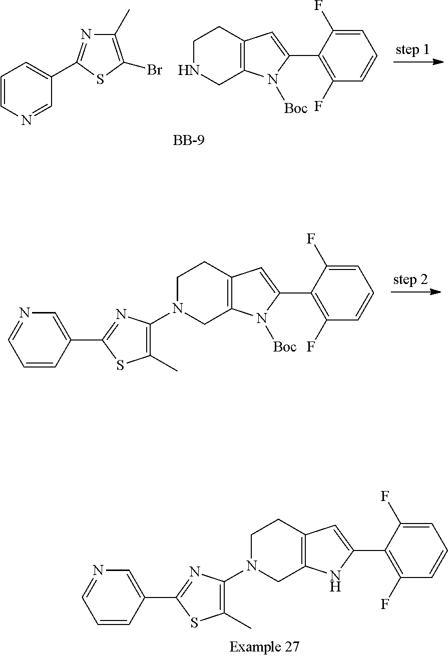

A mixture of BB-9 (200 mg, 0.598 mmol) and 5-bromo-4-methyl-2-(pyridin-3-yl)thiazole (301 mg, 1.20 mmol) in toluene (7.0 mL) was degassed through purging with N₂ for 15 min followed by addition of Cs₂CO₃ (580 mg, 1.79 mmol), rac-BINAP (22 mg, 0.036 mmol) and Pd₂(dba)₃ (18 mg, 0.018 mmol) and the RM was heated in sealed tube to 120° C. for 14 h. The RM was filtered over a pad of Celite™ and the volatiles were removed under reduced pressure. The crude product was purified by chromatography (Interchim® cartridge 50SiHP/12 g, Cy/EtOAc) to yield the desired compound (90 mg, 30%). LC-MS (Method 2): m/z [M+H]⁺=509.3 (MW calc.=508.58); Rₜ=1.14 min.

Step 2:

The intermediate of step 1 (90 mg, 0.177 mmol) was dissolved in MeOH (1.4 mL) and K₂CO₃ (3.66 mg, 2.66 mmol) was added and the mixture was stirred for 10 h at 40° C. The mixture was diluted with aqueous NH₄Cl and extracted with EtOAc. The combined organic layers were dried and the volatiles were removed under reduced pressure. The residue was purified by chromatography (Interchim® cartridge 50SiHP/12 g, Cy/EtOAc) to yield the title compound of example 27 (16 mg, 22%). LC-MS (Method 2): m/z [M+H]⁺=409.2 (MW calc.=408.47); Rₜ=0.90 min.

¹H NMR (DMSO-d₆, 400 MHz), δ (ppm)=10.74 (s, 1H), 9.03 (d, J=1.5 Hz, 1H), 8.61 (dd, J=5.0, 1.5 Hz, 1H), 8.17 (dt, J=8.0, 1.8 Hz, 1H), 7.52-7.49 (m, 1H), 7.26-7.10 (m, 3H), 6.36 (d, J=2.1 Hz, 1H), 4.32 (s, 2H), 3.37 (t, J=5.5 Hz, 2H), 2.71 (t, J=5.5 Hz, 2H) 2.41 (s, 3H).

### Synthesis Example 28

### 2-(2,6-Difluoro-phenyl)-5-(2-methoxy-4-methylsulfonyl-phenyl)-4,5,6,7-tetrahydro-1H-pyrrolo[3,2-c]pyridine

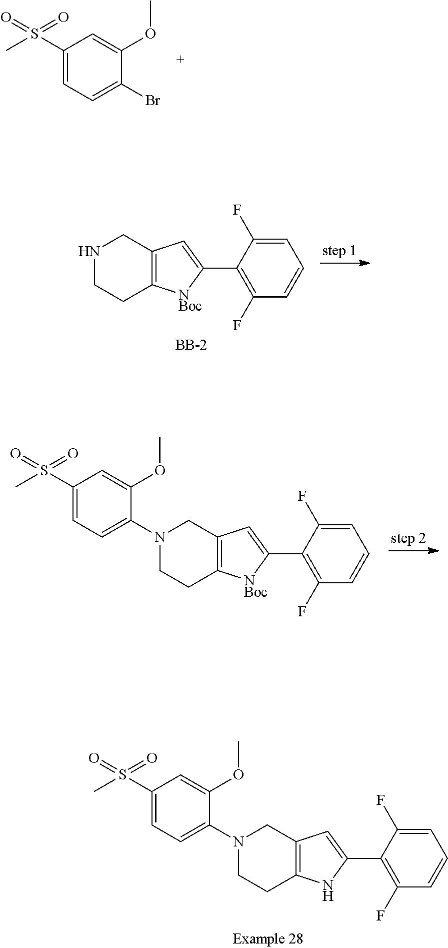 [SMILES: COc1cc(S(C)(=O)=O)ccc1Br ; CC(C)(C)OC(=O)n1c(-c2c(F)cccc2F)cc2c1CCNC2 ; "BB-2" ; COc1cc(S(C)(=O)=O)ccc1N1CCc2c(cc(-c3c(F)cccc3F)n2C(=O)OC(C)(C)C)C1 ; COc1cc(S(C)(=O)=O)ccc1N1CCc2[nH]c(-c3c(F)cccc3F)cc2C1]

Step 1:

A mixture of BB-2 (160 mg, 0.479 mmol) and 1-bromo-2-methoxy-4-(methylsulfonyl)benzene (250 mg, 0.957 mmol) in toluene (5.6 mL) was degassed through purging with N₂ for 15 min followed by addition of Cs₂CO₃ (464 mg, 1.44 mmol), rac-BINAP (17 mg, 0.029 mmol) and Pd₂(dba)₃ (14 mg, 0.014 mmol) and the resulting mixture was heated in sealed tube to 110° C. for 14 h. The RM was filtered over a pad of Celite™ and the volatiles were removed under reduced pressure. The crude product was purified by chromatography (Interchim® cartridge 50SiHP/12 g, Cy/EtOAc) to yield the desired compound (120 mg, 48%).

LC-MS (Method 2): m/z [M+H]⁺=519.3 (MW calc.=518.57); Rₜ=1.07 min.

Step 2:

The intermediate of step 1 (120 mg, 0.231 mmol) was dissolved in MeOH (1.9 mL) and K₂CO₃ (478 mg, 3.47 mmol) was added and the mixture was stirred for 8 h at 40° C. and for 48 h at rt. The mixture was diluted with aqueous NH₄Cl and extracted with EtOAc. The combined organic layers were dried and the volatiles were removed under reduced pressure. The residue was purified by chromatography (Interchim® cartridge 50SiHP/12 g, Cy/EtOAc) to yield the title compound of example 28 (70 mg, 72%).

LC-MS (Method 2): m/z [M+H]⁺=419.1 (MW calc.=418.46); Rₜ=0.78 min.

¹H NMR (DMSO-d₆, 400 MHz), δ (ppm)=10.79 (s, 1H), 7.43-7.41 (m, 1H), 7.37-7.36 (m, 1H), 7.27-7.21 (m, 1H), 7.16-7.09 (m, 3H), 6.34 (s, 1H), 4.16 (s, 2H), 3.91 (s, 3H), 3.49 (t, J=5.3 Hz, 2H), 3.15 (s, 3H), 2.77 (t, J=5.3 Hz, 2H).

### Synthesis Example 29

### 3-[2-(2,6-Difluoro-phenyl)-4,5,6,7-tetrahydro-1H-pyrrolo[3,2-c]pyridin-5-yl]-4-methyl-benzonitrile

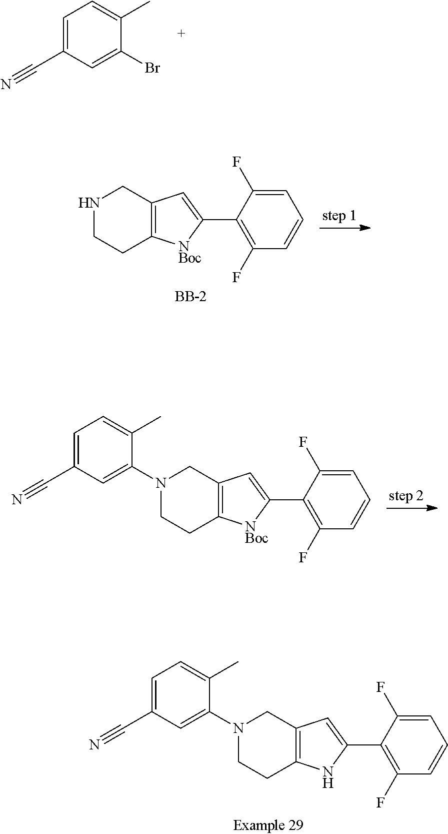

Step 1:

A mixture of BB-2 (200 mg, 0.598 mmol) and 3-bromo-4-methylbenzonitrile (232 mg, 1.20 mmol) in toluene (5.0 mL) was degassed through purging with N₂ for 15 min followed by addition of Cs₂CO₃ (583 mg, 1.79 mmol), rac-BINAP (20 mg, 0.020 mmol) and Pd₂(dba)₃ (18 mg, 0.018 mmol) and the resulting mixture was heated in sealed tube to 110° C. for 14 h. The RM was filtered over a pad of Celite™ and the volatiles were removed under reduced pressure. The crude product was purified by chromate-graphy (Interchim® cartridge 50SiHP/12 g, Cy/EtOAc) to yield the desired compound (230 mg, 86%). LC-MS (Method 2): m/z [M+H]⁺=450.3 (MW calc.=449.49); Rₜ=1.17 min.

Step 2:

The intermediate of step 1 (230 mg, 0.512 mmol) was dissolved in MeOH (4.2 mL) and K₂CO₃ (1.06 g, 7.67 mmol) was added and the mixture was stirred for 8 h at 40° C. and for 48 h at rt. The mixture was diluted with aqueous NH₄Cl and extracted with EtOAc. The combined organic layers were dried and the volatiles were removed under reduced pressure. The residue was purified by chromatography (Interchim® cartridge 50SiHP/12 g, Cy/EtOAc) to yield the title compound of example 29 (117 mg, 65%). LC-MS (Method 2): m/z [M+H]⁺=250.2 (MW calc.=349.38); Rₜ=0.90 min.

¹H NMR (DMSO-d₆, 400 MHz), δ (ppm)=10.82 (s, 1H), 7.48 (s, 1H), 7.41-7.36 (m, 2H), 7.27-7.21 (m, 1H), 7.14 (t, J=9.1 Hz, 2H), 6.33 (s, 1H), 3.97 (s, 2H), 3.21 (t, J=5.3 Hz, 2H), 2.80 (t, J=5.3 Hz, 2H), 2.36 (s, 3H).

### Synthesis Example 30

### 4-[2-(2,6-Difluoro-phenyl)-4,5,6,7-tetrahydro-1H-pyrrolo[3,2-c]pyridin-5-yl]-5-methyl-2-pyrimidin-5-yl-thiazole

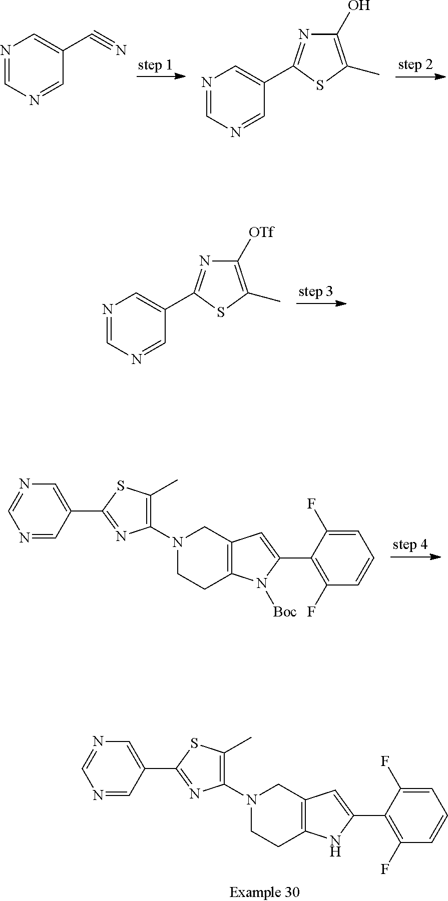 [SMILES: N#Cc1cncnc1 ; Cc1sc(-c2cncnc2)nc1O ; Cc1sc(-c2cncnc2)nc1OS(=O)(=O)C(F)(F)F ; Cc1sc(-c2cncnc2)nc1N1CCc2c(cc(-c3c(F)cccc3F)n2C(=O)OC(C)(C)C)C1 ; Cc1sc(-c2cncnc2)nc1N1CCc2[nH]c(-c3c(F)cccc3F)cc2C1]

Step 1:

To a mixture pyrimidine-5-carbonitrile (2.0 g, 19.0 mmol) and 2-mercapto-propionic acid (2.1 g, 19.0 mmol) was added pyridine (0.5 mL, 6.28 mmol) and the mixture was heated to 100° C. for 2 h. The mixture was cooled to it and EtOH (100 mL) was added to the RM which was stirred at it for 30 min. The formed precipitate was isolated though filtration and was washed with hexane and was dried under reduced pressure to yield the desired compound (2.0 g, 54%)

LC-MS (Method 3): m/z [M+H]⁺=194.2 (MW calc.=193.23); Rₜ=1.99 min.

Step 2:

To a solution of the intermediate of step 1 (2.0 g, 10.38 mmol) in DMF (20 mL) was added NaH (60% in paraffin oil, 621 mg, 15.6 mmol) at 0° C. and the mixture was stirred at it for 20 min, followed by addition of (CF₃SO₂)₂NPh (4.4 g, 12.4 mmol) at 0° C. and the mixture was stirred at it for 16 h. The RM was quenched with aqueous NH₄Cl and was extracted with EtOAc. The combined organic layers were washed with water and brine, dried and the volatiles were removed under reduce pressure. The residue was purified by CC (silica gel, EtOAc/Hex) to yield the desired compound (2.2 g, 65%).

LC-MS (method 3): m/z [M+H]⁺=325.8 (MW calc.=325.29); Rₜ=3.61 min.

Step 3:

A mixture of BB-2 (400 mg, 1.19 mmol) and the intermediate of step 2 (505 mg, 1.55 mmol) in toluene (10 mL) was degassed through purging with N₂ for 15 min followed by addition of Cs₂CO₃ (1.1 g, 3.57 mmol), X-phos (57 mg, 0.119 mmol) and Pd₂(dba)₃ (108 mg, 0.119 mmol). The resulting RM was heated in sealed tube to 110° C. for 16 h. The RM was filtered over a pad of Celite™ and the volatiles were removed under reduced pressure. The residue was purified by CC (neutral alumina, EtOAc/Hex) to afford the desired compound (80 mg).

LC-MS (Method 1): m/z [M+H]⁺=510.2 (MW calc.=509.57); Rₜ=4.49 min.

Step 4:

To a solution of the intermediate of step 3 (70 mg, 0.137 mmol) in MeOH (5 mL) was added K₂CO₃ (57 mg, 0.412 mmol) and the RM was heated to reflux for 16 h. The volatiles were removed under reduced pressure and the residue was diluted with CH₂Cl₂ and was washed with water and brine. The organic layer was dried the volatiles were removed under reduced pressure. The residue was purified by CC (silica gel, EtOAc/Hex) to yield the title compound of example 30 (38 mg).

LC-MS (method 3): m/z [M+H]⁺=410.2 (MW calc.=409.46); Rₜ=3.76 min.

¹H NMR (DMSO-d₆, 400 MHz), δ (ppm)=10.80 (s, 1H), 9.21 (s, 1H), 9.20 (s, 2H), 7.26-7.21 (m, 1H), 7.14 (t, J=8.48 Hz, 2H), 6.32 (s, 1H), 4.20 (s, 2H), 3.44 (t, J=5.40 Hz, 2H), 2.85 (t, J=5.36 Hz, 2H), 2.32 (s, 3H).

### Synthesis Example 31

### 4-[2-(2,6-Difluoro-phenyl)-4,5,6,7-tetrahydro-1H-pyrrolo[3,2-c]pyridin-5-yl]-5-ethyl-2-pyridin-3-yl-thiazole

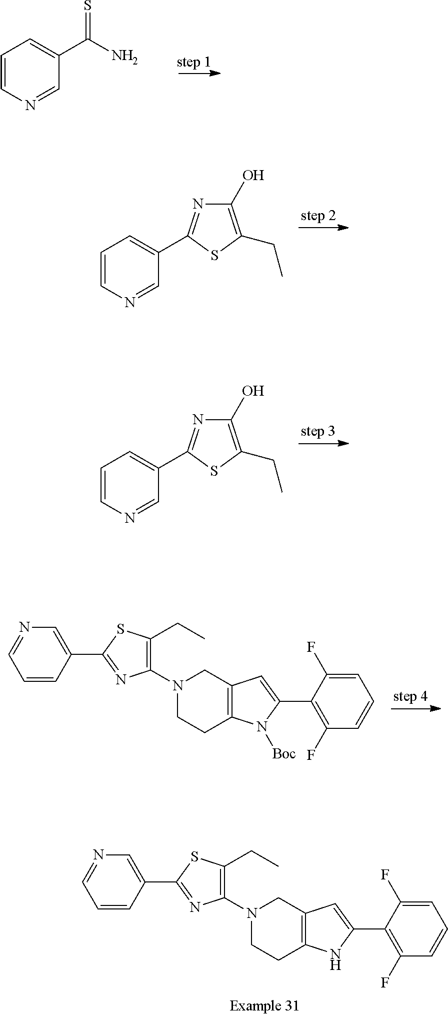 [SMILES: NC(=S)c1cccnc1 ; CCc1sc(-c2cccnc2)nc1O ; CCc1sc(-c2cccnc2)nc1O ; CCc1sc(-c2cccnc2)nc1N1CCc2c(cc(-c3c(F)cccc3F)n2C(=O)OC(C)(C)C)C1 ; CCc1sc(-c2cccnc2)nc1N1CCc2[nH]c(-c3c(F)cccc3F)cc2C1]

Step 1:

To a solution thionicotinamide (5.0 g, 36.2 mmol) in EtOH (50 mL) were added pyridine (4.9 mL, 61.6 mmol) and 2-bromo-butyric acid methyl ester (5.2 mL, 43.5 mmol) and the RM was heated to reflux for 16 h. The volatiles were removed under reduced pressure and the residue was diluted with CH₂Cl₂, was washed with water and brine, dried and the volatiles were removed under reduced pressure. The residue was triturated with EtOAc to yield the desired compound (3.3 g, 39%).

Step 2:

To a solution of the intermediate of step 1 (1.5 g, 7.81 mmol) in CH₂Cl₂ (20 mL) were added pyridine (1.76 mL, 21.8 mmol) and (CF₃SO₂)₂O (1.79 mL, 10.19 mmol) at 0° C. and the mixture was stirred at it for 16 h. The RM was diluted with CH₂Cl₂, was washed with water and brine, dried and the volatiles were removed under reduced pressure. The residue was purified by CC (silica gel, EtOAc/Hex) to yield the desired compound (1.0 g, 40%).

LC-MS (method 3): m/z [M+H]⁺=339.2 (MW calc.=338.33); Rₜ=3.65 min.

Step 3:

A mixture of BB-2 (494 mg, 1.47 mmol) and the intermediate of step 2 (500 mg, 1.47 mmol) in toluene (10 mL) was degassed through purging with N₂ for 15 min followed by the addition of Cs₂CO₃ (1.44 g, 4.43 mmol), X-phos (70 mg, 0.147 mmol) and Pd₂(dba)₃ (134 mg, 0.14 mmol) and the resulting mixture was heated to reflux for 14 h. The RM was diluted with EtOAc, washed with water and brine, dried and the volatiles were removed under reduced pressure. The residue was purified by CC (silica gel, Acetone/Hex) to yield the desired compound (120 mg, 15%)

Step 4:

To a solution of the intermediate of step 3 (120 mg, 0.229 mmol) in MeOH (5 mL) was added K₂CO₃ (158 mg, 1.15 mmol) and the RM was heated to reflux for 16 h. The volatiles were removed under reduced pressure and the residue was diluted with EtOAc, washed with water and brine, dried and the volatiles were removed under reduced pressure. The residue was purified by CC (silica gel, Acetone/Hex) to yield the title compound of example 31 (30 mg, 30%).

LC-MS (method 3): m/z [M+H]⁺=423.2 (MW calc.=422.5); Rₜ=4.20 min.

¹H NMR (DMSO-d₆, 400 MHz), δ (ppm)=10.79 (s, 1H), 9.04 (d, J=1.84 Hz, 1H), 8.60 (dd, J=4.76 Hz, 1.2 Hz, 1H), 8.21-8.19 (m, 1H), 7.52-7.48 (m, 1H), 7.28-7.22 (m, 1H), 7.14 (t, J=8.56 Hz, 2H), 6.32 (s, 1H), 4.14 (s, 2H), 3.39 (t, J=5.56 Hz, 2H), 2.86-2.81 (m, 4H), 1.27 (t, J=7.48 Hz, 3H).

### Synthesis Example 32

### 5-(6-Chloro-5-methyl-pyridazin-3-yl)-2-(2,6-difluoro-phenyl)-4,5,6,7-tetrahydro-1H-pyrrolo[3,2-c]pyridine

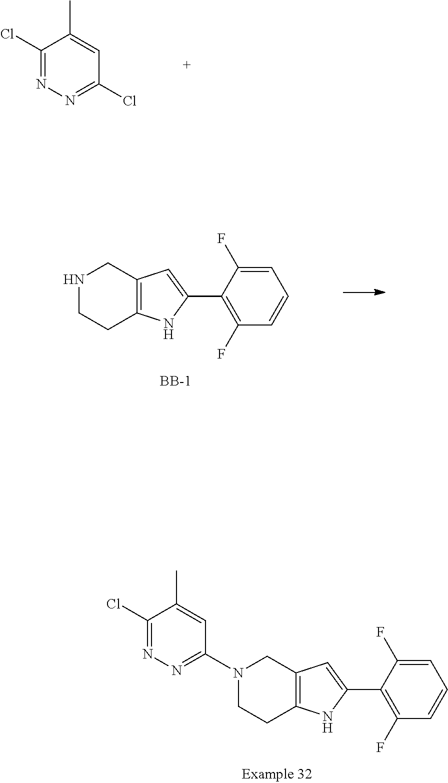

A mixture BB-1 (400 mg, 1.70 mmol), 3,6-dichloro-4-methyl-pyridazine (554 mg, 3.4 mmol) and Cs₂CO₃ (1.38 g, 4.25 mmol) in dioxane (10 mL) was heated in seal tube to 110° C. for 16 h. The RM was filtered and the solvents were removed under reduced pressure. The residue was purified by preparative

HPLC to yield the title compound of example 32 (60 mg, 10%)

LC-MS (Method 3): m/z [M+H]⁺=361.2 (MW calc.=360.79); Rₜ=3.51 min.

¹H NMR (DMSO-d₆, 400 MHz), δ (ppm)=10.84 (s, 1H), 7.43 (s, 1H), 7.27 (m, 1H), 7.14 (t, J=8.56 Hz, 2H), 6.35 (s, 1H), 4.59 (s, 2H), 3.97 (t, J=5.60 Hz, 2H), 2.78 (t, J=5.52 Hz, 2H), 2.27 (s, 3H).

### Synthesis Example 33

### 5-[2-(2,6-Difluoro-phenyl)-4,5,6,7-tetrahydro-1H-pyrrolo[3,2-c]pyridin-5-yl]-6-methoxy-2,3-dihydro-benzo[b]thiophene 1,1-dioxide

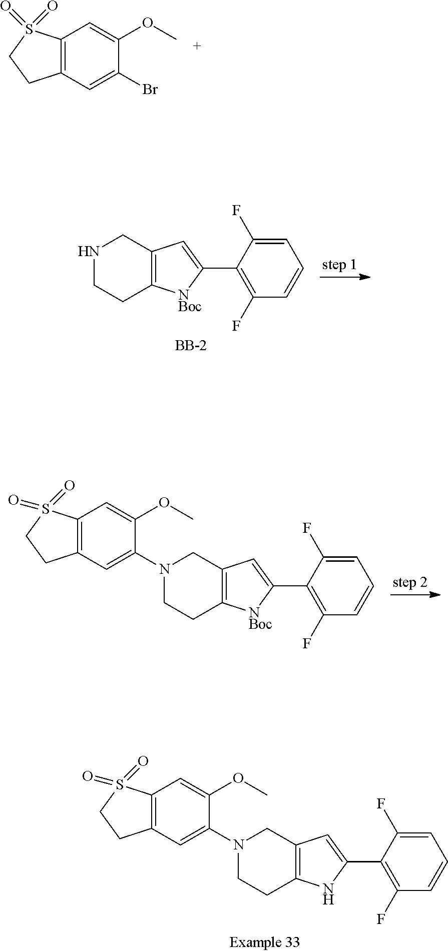 [SMILES: COc1cc2c(cc1Br)CCS2(=O)=O ; CC(C)(C)OC(=O)n1c(-c2c(F)cccc2F)cc2c1CCNC2 ; "BB-2" ; COc1cc2c(cc1N1CCc3c(cc(-c4c(F)cccc4F)n3C(=O)OC(C)(C)C)C1)CCS2(=O)=O ; COc1cc2c(cc1N1CCc3[nH]c(-c4c(F)cccc4F)cc3C1)CCS2(=O)=O]

Step 1:

A mixture of BB-2 (200 mg, 0.598 mmol) and 5-Brom-6-methoxy-2,3-dihydro-benzo[b]thiophen 1,1-dioxid (331 mg, 1.20 mmol) in toluene (7.0 mL) was degassed through purging with N₂ for 15 min followed by addition of Cs₂CO₃ (578 mg, 1.79 mmol), rac-BINAP (21 mg, 0.021 mmol) and Pd₂(dba)₃ (18 mg, 0.018 mmol) and the resulting mixture was heated in sealed tube to 110° C. for 14 h. The RM was filtered over a pad of Celite™ and the volatiles were removed under reduced pressure. The crude product was purified by chromatography (Interchim® cartridge 50SiHP/12 g, Cy/EtOAc) to yield the desired compound (320 mg, quant.).

LC-MS (Method 2): m/z [M+H]⁺=531.4 (MW calc.=530.58); Rₜ=1.07 min.

Step 2:

The intermediate of step 1 (320 mg, 0.603 mmol) was dissolved in MeOH (4.9 mL) and K₂CO₃ (1.25 g, 9.05 mmol) was added and the mixture was stirred for 8 h at 50° C. and for 24 h at rt. The mixture was diluted with aqueous NH₄Cl and extracted with EtOAc. The combined organic layers were dried and the volatiles were removed under reduced pressure. The residue was purified by chromatography (Interchim® cartridge 50SiHP/12 g, Cy/EtOAc) to yield the title compound of example 33 (50 mg, 19%). LC-MS (Method 2): m/z [M+H]⁺=431.1 (MW calc.=430.47); Rₜ=0.77 min.

¹H NMR (DMSO-d₆, 400 MHz), δ (ppm)=10.80 (s, 1H), 7.26-7.21 (m, 1H), 7.17-7.11 (m, 3H), 6.99 (s, 1H), 6.33 (s, 1H), 4.12 (s, 2H), 3.89 (s, 3H), 3.51 (t, J=6.8 Hz, 2H), 3.44 (t, J=5.6 Hz, 2H), 3.21 (t, J=6.8 Hz, 2H), 2.78 (t, J=5.6 Hz, 2H).

### Synthesis Example 34

### 5-(4-Methyl-6-methylsulfonyl-pyridin-3-yl)-2-phenyl-4,5,6,7-tetrahydro-1H-pyrrolo[3,2-c]pyridine

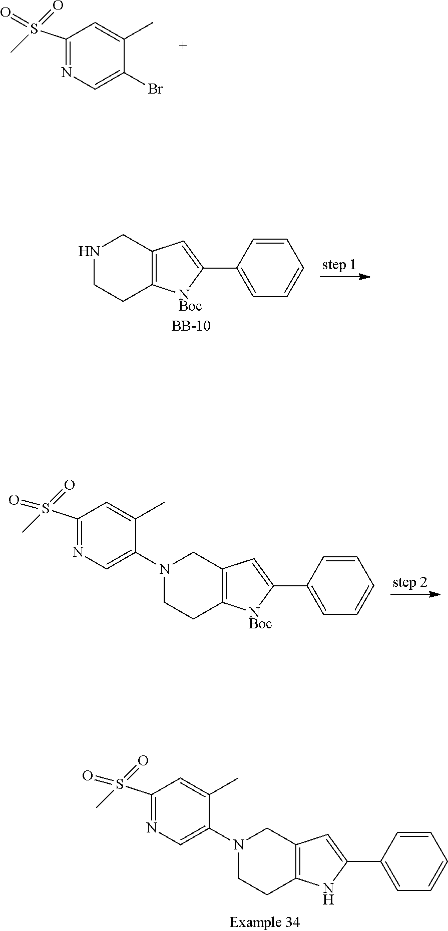

Step 1:

A mixture of BB-10 (120 mg, 0.402 mmol) and 5-bromo-2-methanesulfonyl-4-methyl-pyridine (201 mg, 0.804 mmol) in toluene (4.0 mL) was degassed through purging with N₂ for 15 min followed by addition of Cs₂CO₃ (389 mg, 1.21 mmol), rac-BINAP (14 mg, 0.014 mmol) and Pd₂(dba)₃ (12 mg, 0.012 mmol) and the resulting mixture was heated in sealed tube to 110° C. for 14 h. The RM was filtered over a pad of Celite™ and the volatiles were removed under reduced pressure. The crude product was purified by chromatography (Interchim® cartridge 50SiHP/12 g, Cy/EtOAc) to yield the desired compound (150 mg, 80%).

LC-MS (Method 2): m/z [M+H]⁺=468.4 (MW calc.=467.58); Rₜ=1.07 min.

Step 2:

The intermediate of step 1 (150 mg, 0.321 mmol) was dissolved in MeOH (2.6 mL) and K₂CO₃ (662 mg, 4.82 mmol) was added and the mixture was stirred for 2 h at 50° C. The mixture was diluted with aqueous NH₄Cl and extracted with EtOAc. The combined organic layers were dried and the volatiles were removed under reduced pressure. The residue was purified by chromatography (Interchim® cartridge 50SiHP/12 g, Cy/EtOAc) to yield the title compound of example 34 (70 mg, 59%).

LC-MS (Method 2): m/z [M+H]⁺=368.1 (MW calc.=367.46); Rₜ=0.72 min.

¹H NMR (DMSO-d₆, 400 MHz), δ (ppm)=11.03 (s, 1H), 8.40 (s, 1H), 7.83 (s, 1H), 7.57 (d, J=8.1 Hz, 2H), 7.32 (t, J=7.8 Hz, 2H), 7.11 (t, J=7.8 Hz, 1H), 6.34 (s, 1H), 4.17 (s, 2H), 3.38 (t, J=5.3 Hz, 2H), 3.20 (s, 3H), 2.80 (t, J=5.3 Hz, 2H), 2.42 (s, 3H).

### Synthesis Example 35

### 5-(4-Methyl-6-methylsulfonyl-pyridin-3-yl)-2-o-tolyl-4,5,6,7-tetrahydro-1H-pyrrolo[3,2-c]pyridine

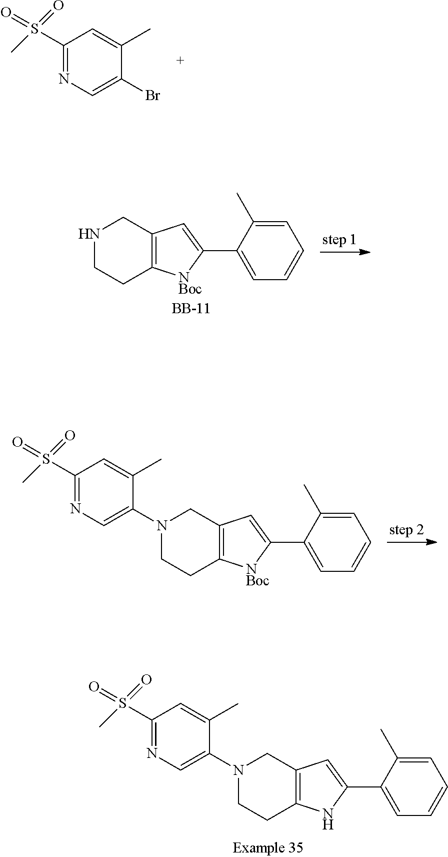

Step 1:

A mixture of BB-11 (150 mg, 0.480 mmol) and 5-bromo-2-methanesulfonyl-4-methyl-pyridine (180 mg, 0.720 mmol) in toluene (5.6 mL) was degassed through purging with N₂ for 15 min followed by addition of Cs₂CO₃ (465 mg, 1.44 mmol), rac-BINAP (17 mg, 0.017 mmol) and Pd₂(dba)₃ (14 mg, 0.014 mmol) and the resulting mixture was heated in sealed tube to 110° C. for 14 h. The RM was filtered over a pad of Celite™ and the volatiles were removed under reduced pressure. The crude product was purified by chromatography (Interchim® cartridge 50SiHP/12 g, Cy/EtOAc) to yield the desired compound (120 mg, 52%).

LC-MS (Method 2): m/z [M+H]⁺=482.4 (MW calc.=481.61); Rₜ=1.17 min.

Step 2:

The intermediate of step 1 (115 mg, 0.239 mmol) was dissolved in MeOH (1.9 mL) and K₂CO₃ (492 mg, 3.58 mmol) was added and the mixture was stirred for 3 h at 50° C. The mixture was diluted with aqueous NH₄Cl and extracted with EtOAc. The combined organic layers were dried and the volatiles were removed under reduced pressure. The residue was purified by chromatography (Interchim® cartridge 50SiHP/12 g, Cy/EtOAc) to yield the title compound of example 35 (60 mg, 66%).

LC-MS (Method 2): m/z [M+H]⁺=382.2 (MW calc.=381.49); Rₜ=0.77 min.

¹H NMR (DMSO-d₆, 400 MHz), δ (ppm)=10.77 (s, 1H), 8.41 (s, 1H), 7.83 (s, 1H), 7.42-7.36 (m, 1H), 7.26-7.17 (m, 2H), 7.15-7.08 (m, 1H), 6.08 (s, 1H), 4.20 (s, 2H), 3.45-3.35 (m, 2H), 3.20 (s, 3H), 2.84-2.77 (m, 2H), 2.41 (s, 3H).

### Synthesis Example 36

### 2-(2-Chloro-6-fluoro-phenyl)-5-(4-methyl-6-methylsulfonyl-pyridin-3-yl)-4,5,6,7-tetrahydro-1H-pyrrolo[3,2-c]pyridine

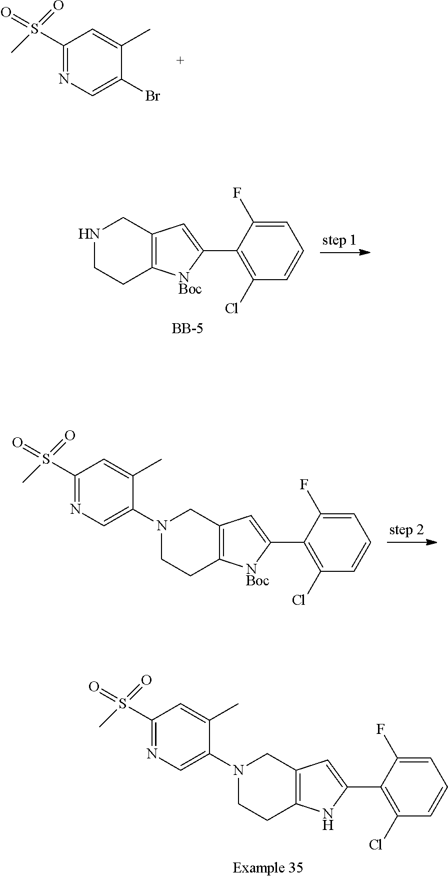 [SMILES: Cc1cc(S(C)(=O)=O)ncc1Br ; CC(C)(C)OC(=O)n1c(-c2c(F)cccc2Cl)cc2c1CCNC2 ; "BB-5" ; Cc1cc(S(C)(=O)=O)ncc1N1CCc2c(cc(-c3c(F)cccc3Cl)n2C(=O)OC(C)(C)C)C1 ; Cc1cc(S(C)(=O)=O)ncc1N1CCc2[nH]c(-c3c(F)cccc3Cl)cc2C1]

Step 1:

A mixture of BB-5 (150 mg, 0.428 mmol) and 5-bromo-2-methanesulfonyl-4-methyl-pyridine (214 mg, 0.855 mmol) in toluene (5.0 mL) was degassed through purging with N₂ for 15 min followed by addition of Cs₂CO₃ (414 mg, 1.28 mmol), rac-BINAP (16 mg, 0.026 mmol) and Pd₂(dba)₃ (13 mg, 0.013 mmol) and the resulting mixture was heated in sealed tube to 110° C. for 14 h. The RM was filtered over a pad of Celite™ and the volatiles were removed under reduced pressure. The crude product was purified by chromatography (Interchim® cartridge 50SiHP/12 g, Cy/EtOAc) to yield the desired compound (120 mg, 54%).

LC-MS (Method 2): m/z [M+H]⁺=520.4 (MW calc.=520.02); Rₜ=1.08 min.

Step 2:

The intermediate of step 1 (120 mg, 0.231 mmol) was dissolved in MeOH (1.9 mL) and K₂CO₃ (477 mg, 3.46 mmol) was added and the mixture was stirred for 2 h at 50° C. The mixture was diluted with aqueous NH₄Cl and extracted with EtOAc. The combined organic layers were dried and the volatiles were removed under reduced pressure. The residue was purified by chromatography (Interchim® cartridge 50SiHP/12 g, Cy/EtOAc) to yield the title compound of example 36 (75 mg, 77%).

LC-MS (Method 2): m/z [M+H]⁺=420.1 (MW calc.=419.90); Rₜ=0.77 min.

¹H NMR (DMSO-d₆, 400 MHz), δ (ppm)=10.89 (s, 1H), 8.41 (s, 1H), 7.84 (s, 1H), 7.41-7.24 (m, 3H), 6.18 (s, 1H), 4.21 (s, 2H), 3.41-3.36 (m, 2H), 3.20 (s, 3H), 2.83-2.79 (m, 2H), 2.43 (s, 3H).

### Synthesis Example 37

### 2-(4-Fluorophenyl)-5-(4-methyl-6-methylsulfonyl-pyridin-3-yl)-4,5,6,7-tetrahydro-1H-pyrrolo[3,2-c]pyridine

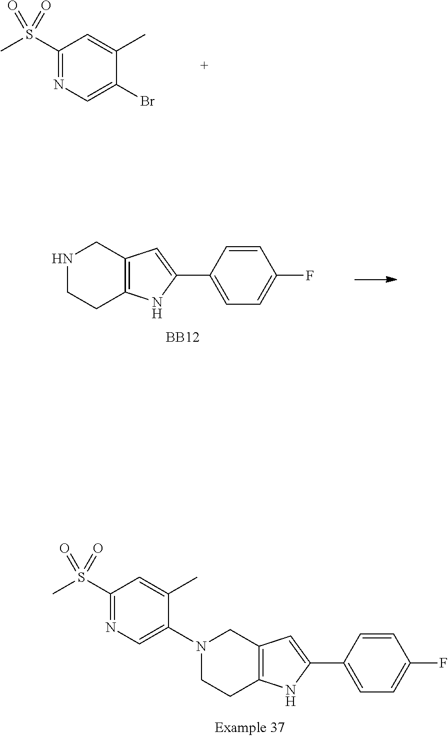

A degassed mixture BB-12 (100 mg, 0.462 mmol), 5-bromo-2-methanesulfonyl-4-methyl-pyridine (139 mg, 0.555 mmol) and Cs₂CO₃ (301 mg, 0.925 mmol) in dioxane (3.2 mL) was heated in seal tube to 110° C. for 16 h. The RM was filtered and the solvents were removed under reduced pressure. The residue was purified by chromatography (Interchim® cartridge 50SiHP/12 g, Cy/EtOAc) to yield the title compound of example 37 (20 mg, 11%).

LC-MS (Method 2): m/z [M+H]⁺=386.1 (MW calc.=385.46); Rₜ=0.73 min.

¹H NMR (DMSO-d₆, 400 MHz), δ (ppm)=11.02 (s, 1H), 8.40 (s, 1H), 7.83 (s, 1H), 7.63-7.56 (m, 2H), 7.16 (t, J=8.5 Hz, 2H), 6.30 (s, 1H), 4.16 (s, 2H), 3.41-3.36 (m, 2H), 3.19 (s, 3H), 2.83-2.77 (m, 2H), 2.42 (s, 3H).

### General Procedure for the Synthesis of Example Compounds in a Library Setup:

### Method A:

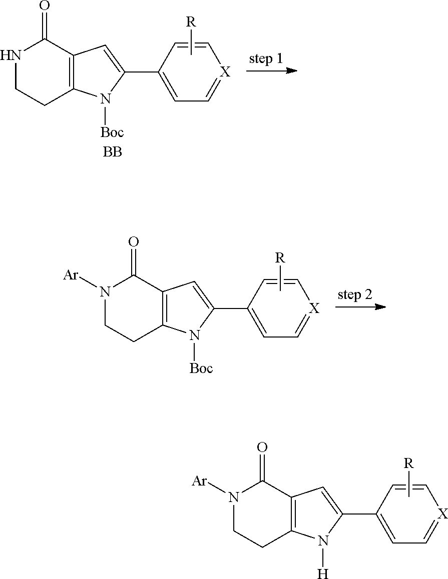

Step 1:

To a solution of the required building block (1 eq.) in toluene (10 mL) in a sealed tube, K₂CO₃ (3 eq.), the corresponding bromo compound (1.2 eq) and N,N′-dimethylethylene diamine (0.5 eq) were added. The RM was degassed by purging Ar for 30 min. Then, CuI (0.5 eq.) was added and Argon was purged for further 30 min. The tube was sealed with a Teflon screw cap and the RM was stirred at 100° C. for 2 to 6 d. The RM was cooled to rt and diluted with toluene (20 mL) and filtered over a plug of Celite™. The combined filtrate was concentrated under reduced pressure. The residue was purified by CC (silica gel) to afford the respective products.

Step 2:

To a solution of the intermediate of step 1 (1 eq) in THF (5 mL) a solution of NaOMe (5 eq.) in MeOH (5 mL) was added at rt and the RM was stirred for 4 h. The RM was concentrated under reduced pressure, the residue was diluted with water (10 mL) and the obtained precipitate was filtered and purified by CC (silica gel) to afford desired products (Table 1, Table 2)

### Method B:

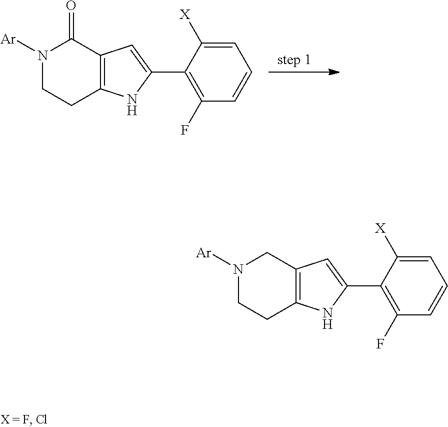

Step 1:

To the corresponding amide (synthesized according to method A) (1 eq.) in THF (5 mL), BH₃.DMS (5 eq.) was added at it and the RM was stirred at 75° C. for 4 h. The solvent was evaporated, MeOH (5 mL) was added and the RM was refluxed for 3 h. The volatiles were removed under reduced pressure to give a residue that was diluted with water (5 mL) and extracted with EtOAc (5 mL). The layers were separated and the combined organic phases were dried and concentrated under reduced pressure. The residue was purified by chromatography (silica gel) to yield the desired products.

TABLE 1

| | | | Yield | | |
|---|---|---|---|---|---|
| Example | | | [mg/ | | LC-MS |
| No | Structure | Name | (% yield)] | Method | (m/z, R₁) |
| 38 | 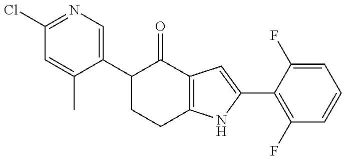 [SMILES: Cc1cc(Cl)ncc1C1CCc2[nH]c(-c3c(F)cccc3F)cc2C1=O] | 5-(6-Chloro-4-methyl-pyridin- 3-yl)-2-(2,6-difluoro-phenyl)- 1,5,6,7-tetrahydro-pyrrolo- [3,2-c]pyridin-4-one | 20 (15)¹ 9 (27)² | A | Method 2 [M + H]⁺ = 374.1 MW_{calc} = 373.08 Rₜ = 0.66 min |
| 39 | 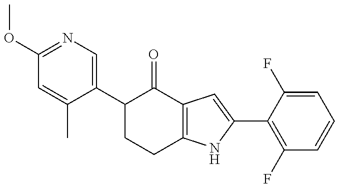 [SMILES: COc1cc(C)c(C2CCc3[nH]c(-c4c(F)cccc4F)cc3C2=O)cn1] | 2-(2,6-Difluoro-phenyl)-5-(6- methoxy-4-methyl-pyridin-3- yl)-1,5,6,7-tetrahydro- pyrrolo[3,2-c]pyridin-4-one | 30 (28) 30 (28) | A | method 2 [M + H]⁺ = 370.2 MW_{calc} = 369.13 Rₜ = 0.64 min |
| 40 | 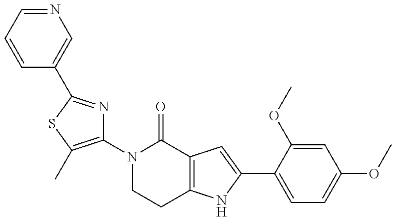 [SMILES: COc1ccc(-c2cc3c([nH]2)CCN(c2nc(-c4cccnc4)sc2C)C3=O)c(OC)c1] | 2-(2,4-Dimethoxy-phenyl)-5- (5-methyl-2-pyridin-3-yl-thia- zol-4-yl)-1,5,6,7-tetrahydro- pyrrolo[3,2-c]pyridin-4-one | 80 (82) 42 (63) | A | method 2 [M + H]⁺ = 447.2 MW_{calc} = 446.14 Rₜ = 0.64 min |
| 41 | 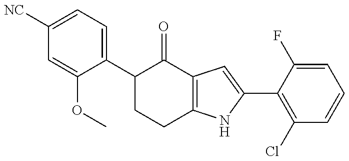 [SMILES: COc1cc(C#N)ccc1C1CCc2[nH]c(-c3c(F)cccc3Cl)cc2C1=O] | 4-[2-(2-Chloro-6-fluoro- phenyl)-4-oxo-1,5,6,7-tetra- hydro-pyrrolo[3,2-c]pyridin-5- yl]-3-methoxy-benzonitrile | 30 (28) 10 (42) | A | method 2 [M + H]⁺ = 396.1 MW_{calc} = 395.08 Rₜ = 0.66 min |
| 42 | 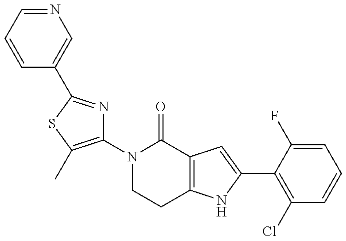 [SMILES: Cc1sc(-c2cccnc2)nc1N1CCc2[nH]c(-c3c(F)cccc3Cl)cc2C1=O] | 2-(2-Chloro-6-fluoro-phenyl)- 5-(5-methyl-2-pyridin-3-yl- thiazol-4-yl)-1,5,6,7-tetra- hydro-pyrrolo[3,2-c]pyridin-4- one | 80 (68) 40 (61) | A | method 2 [M ' H]⁺ = 439.1 MW_{calc} = 438.07 Rₜ = 0.63 min |
| 43 | 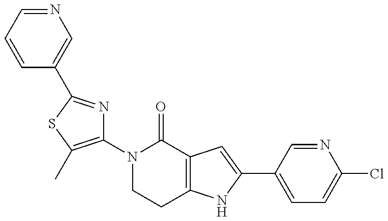 [SMILES: Cc1sc(-c2cccnc2)nc1N1CCc2[nH]c(-c3ccc(Cl)nc3)cc2C1=O] | 2-(6-Chloro-pyridin-3-yl)-5- (5-methyl-2-pyridin-3-yl-thia- zol-4-yl)-1,5,6,7-tetrahydro- pyrrolo[3,2-c]pyridin-4-one | | A | method 2 [M + H]⁺ = 422.1 MW_{calc} = 421.09 Rₜ = 0.57 min |
| 44 | 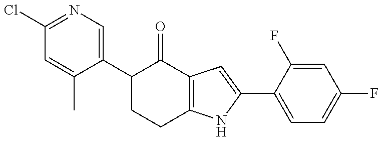 [SMILES: Cc1cc(Cl)ncc1C1CCc2[nH]c(-c3ccc(F)cc3F)cc2C1=O] | 5-(6-Chloro-4-methyl-pyridin- 3-yl)-2-(2,4-difluoro-phenyl)- 1,5,6,7-tetrahydro-pyrrolo- [3,2-c]pyridin-4-one | 18 (13) 6 (42) | A | method 2 [M + H]⁺ = 374.1 MW_{calc} = 373.08 Rₜ = 0.67 min |
| 45 | 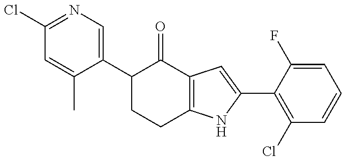 [SMILES: Cc1cc(Cl)ncc1C1CCc2[nH]c(-c3c(F)cccc3Cl)cc2C1=O] | 2-(2-Chloro-6-fluoro-phenyl)- 5-(6-chloro-4-methyl-pyridin- 3-yl)-1,5,6,7-tetrahydro- pyrrolo[3,2-c]pyridin-4-one | 20 (19) 5 (31) | A | method 2 [M + H]⁺ = 390.1 MW_{calc} = 389.05 Rₜ = 0.67 min |
| 46 | 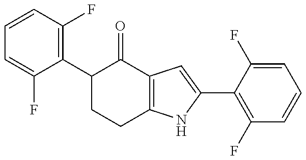 [SMILES: O=C1c2cc(-c3c(F)cccc3F)[nH]c2CCC1c1c(F)cccc1F] | 2,5-Bis(2,6-difluoro-phenyl)- 1,5,6,7-tetrahydro- pyrrolo[3,2-c]pyridin-4-one | 25 (19) 9 (55) | A | method 2 [M + H]⁺ = 361.1 MW_{calc} = 360.09 Rₜ = 0.67 min |
| 47 | 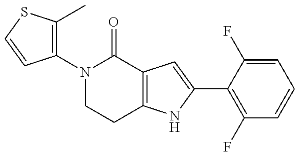 [SMILES: Cc1sccc1N1CCc2[nH]c(-c3c(F)cccc3F)cc2C1=O] | 2-(2,6-Difluoro-phenyl)-5-(2- methyl-thiophen-3-yl)- 1,5,6,7-tetrahydro-pyrrolo- [3,2-c]pyridin-4-one | 70 (55) 35 (75) | A | method 2 [M + H]⁺ = 345.1 MW_{calc} = 344.08 Rₜ = 0.67 min |
| 48 | 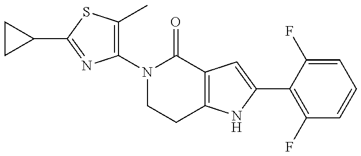 [SMILES: Cc1sc(C2CC2)nc1N1CCc2[nH]c(-c3c(F)cccc3F)cc2C1=O] | 5-(2-Cyclopropyl-5-methyl- thiazol-4-yl)-2-(2,6-difluoro- phenyl)-1,5,6,7-tetrahydro- pyrrolo[3,2-c]pyridin-4-one | 70 (50) 45 (81) | A | method 2 [M + H]⁺ = 386.1 MW_{calc} = 385.11 Rₜ = 0.68 min |
| 49 | 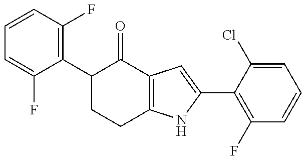 [SMILES: O=C1c2cc(-c3c(F)cccc3Cl)[nH]c2CCC1c1c(F)cccc1F] | 2-(2-Chloro-6-fluoro-phenyl)- 5-(2,6-difluoro-phenyl)- 1,5,6,7-tetrahydro-pyrrolo- [3,2-c]pyridin-4-one | 25 (18) 12 (61) | A | method 2 [M + H]⁺ = 377.1 MW_{calc} = 376.06 Rₜ = 0.68 min |
| 50 | 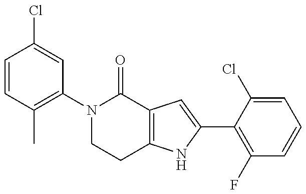 [SMILES: Cc1ccc(Cl)cc1N1CCc2[nH]c(-c3c(F)cccc3Cl)cc2C1=O] | 2-(2-Chloro-6-fluoro-phenyl)- 5-(5-chloro-2-methyl- phenyl)-1,5,6,7-tetrahydro- pyrrolo[3,2-c]pyridin-4-one | 70 (51) 24 (60) | A | method 2 [M + H]⁺ = 389.1 MW_{calc} = 388.05 Rₜ = 0.73 |
| 51 | 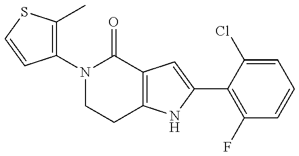 [SMILES: Cc1sccc1N1CCc2[nH]c(-c3c(F)cccc3Cl)cc2C1=O] | 2-(2-Chloro-6-fluoro-phenyl)- 5-(2-methyl-thiophen-3-yl)- 1,5,6,7-tetrahydro-pyrrolo- [3,2-c]pyridin-4-one | 90 (47) 40 (85) | A | method 2 [M + H]⁺ = 361.0 MW_{calc} = 360.05 Rᵣ = 0.67 min |
| 52 | 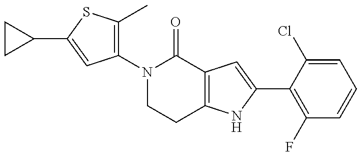 [SMILES: Cc1sc(C2CC2)cc1N1CCc2[nH]c(-c3c(F)cccc3Cl)cc2C1=O] | 2-(2-Chloro-6-fluoro-phenyl)- 5-(2-cyclopropyl-5-methyl- thiazol-4-yl)-1,5,6,7-tetra- hydro-pyrrolo[3,2-c]pyridin-4- one | 80 (58) 42 (65) | A | method 2 [M + H]⁺ = 402.1 MW_{calc} = 401.08 Rₜ = 0.69 min |
| 53 | 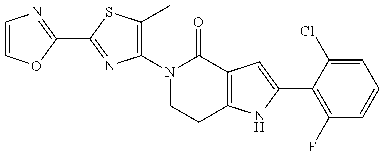 [SMILES: Cc1sc(-c2ncco2)nc1N1CCc2[nH]c(-c3c(F)cccc3Cl)cc2C1=O] | 2-(2-Chloro-6-fluoro-phenyl)- 5-(5-methyl-2-oxazol-2-yl- thiazol-4-yl)-1,5,6,7-tetra- hydro-pyrrolo[3,2-c]pyridin-4- one | 175 (80) 6 (50) | A | method 2 [M + H]⁺ = 429.0 MW_{calc} = 428.05 Rₜ = 0.67 min |
| 54 | 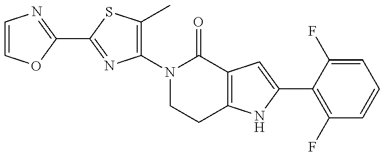 [SMILES: Cc1sc(-c2ncco2)nc1N1CCc2[nH]c(-c3c(F)cccc3F)cc2C1=O] | 2-(2,6-Difluoro-phenyl)-5-(5- methyl-2-oxazol-2-yl-thiazol- 4-yl)-1,5,6,7-tetrahydro- pyrrol[3,2-c]pyridin-4-one | 100 (45) 120 (75) | A | method 2 [M + H]⁺ = 413.0 MW_{calc} = 412.08 Rₜ = 0.67 min |
| 55 | 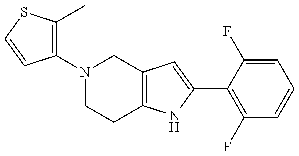 [SMILES: Cc1sccc1N1CCc2[nH]c(-c3c(F)cccc3F)cc2C1] | 2-(2,6-Difluoro-phenyl)-5-(2- methyl-thiophen-3-yl)- 4,5,6,7-tetrahydro-1H- pyrrolo[3,2-c]pyridine | 70 (73) | B | method 2 [M + H]⁺ = 331.0 MW_{calc} = 330.10 Rₜ = 0.81 min |
| 56 | 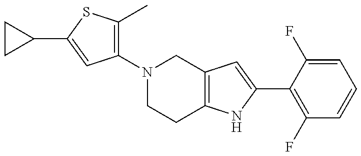 [SMILES: Cc1sc(C2CC2)cc1N1CCc2[nH]c(-c3c(F)cccc3F)cc2C1] | 2-Cyclopropyl-4-[2-(2,6-di- fluoro-phenyl)-4,5,6,7-tetra- hydro-1H-pyrrolo[3,2-c]- pyridin-5-yl]-5-methyl- thiazole | 52 (54) | B | method 2 [M + H]⁺ = 372.0 MW_{calc} = 371.13 Rₜ = 0.92 min |
| 57 | 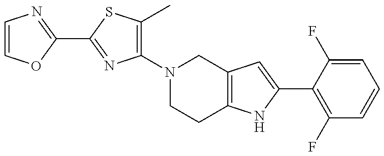 [SMILES: Cc1sc(-c2ncco2)nc1N1CCc2[nH]c(-c3c(F)cccc3F)cc2C1] | 2-[4-[2-(2,6-Difluoro-phenyl)- 4,5,6,7-tetrahydro-1H- pyrrolo[3,2-c]pyridin-5-yl]-5- methyl-thiazol-2-yl]-oxazole | 14 (14) | B | method 2 [M + H]⁺ = 399.0 MW_{calc} = 398.10 Rₜ = 0.87 min |
| 58 | 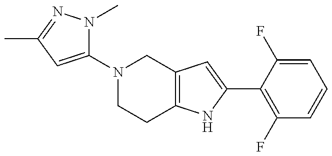 [SMILES: Cc1cc(N2CCc3[nH]c(-c4c(F)cccc4F)cc3C2)n(C)n1] | 2-(2,6-Difluoro-phenyl)-5- (2,5-dimethyl-2H-pyrazol-3- yl)-4,5,6,7-tetrahydro-1H- pyrrolo[3,2-c]pyridine | 15 (21) | B | method 2 [M + H]⁺ = 329.1 MW_{calc} = 328.15 Rₜ = 0.73 min |
| 59 | 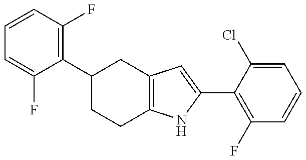 [SMILES: Fc1cccc(Cl)c1-c1cc2c([nH]1)CCC(c1c(F)cccc1F)C2] | 2-(2-Chloro-6-fluoro-phenyl)- 5-(2,6-difluoro-phenyl)- 4,5,6,7-tetrahydro-1H- pyrrolo[3,2-c]pyridine | 16 (83) | B | method 2 [M + H]⁺ = 363.0 MW_{calc} = 362.08 Rₜ = 0.94 min |
| 60 | 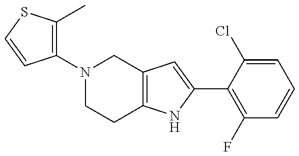 [SMILES: Cc1sccc1N1CCc2[nH]c(-c3c(F)cccc3Cl)cc2C1] | 2-(2-Chloro-6-fluoro-phenyl)- 5-(2-methyl-thiophen-3-yl)- 4,5,6,7-tetrahydro-1H- pyrrolo[3,2-c]pyridine | 48 (72) | B | method 2 [M + H]⁺ = 347.0 MW_{calc} = 346.07 Rₜ = 0.82 min |
| 61 | 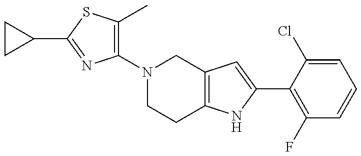 [SMILES: Cc1sc(C2CC2)nc1N1CCc2[nH]c(-c3c(F)cccc3Cl)cc2C1] | 4-[2-(2-Chloro-6-fluoro- phenyl)-4,5,6,7-tetrahydro- 1H-pyrrolo[3,2-c]pyridin-5- yl]-2-cyclopropyl-5-methyl- thiazole | 21 (31) | B | method 2 [M + H]⁺ = 388.0 MW_{calc} = 387.10 Rₜ = 0.92 min |
| 62 | 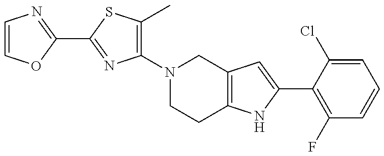 [SMILES: Cc1sc(-c2ncco2)nc1N1CCc2[nH]c(-c3c(F)cccc3Cl)cc2C1] | 2-[4-[2-(2-Chloro-6-fluoro- phenyl)-4,5,6,7-tetrahydro- 1H-pyrrolo[3,2-c]pyridin-5- yl]-5-methyl-thiazol-2-yl]- oxazole | 16 (16) | B | method 2 [M + H]⁺ = 415.0 MW_{calc} = 414.07 Rₜ = 0.88 min |
| 63 | 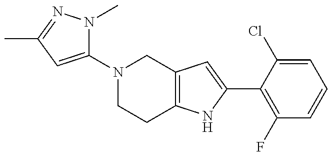 [SMILES: Cc1cc(N2CCc3[nH]c(-c4c(F)cccc4Cl)cc3C2)n(C)n1] | 2-(2-Chloro-6-fluoro-phenyl)- 5-(2,5-dimethyl-2H-pyrazol- 3-yl)-4,5,6,7-tetrahydro-1H- pyrrolo[3,2-c]pyridine | 16 (22) | B | method 2 [M + H]⁺ = 345.0 MW_{calc} = 344.12 Rₜ = 0.74 min |
| 64 | 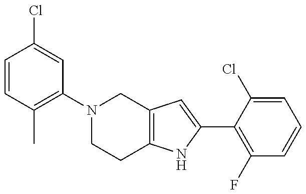 [SMILES: Cc1ccc(Cl)cc1N1CCc2[nH]c(-c3c(F)cccc3Cl)cc2C1] | 2-(2-Chloro-6-fluoro-phenyl)- 5-(5-chloro-2-methyl- phenyl)-4,5,6,7-tetrahydro- 1H-pyrrolo[3,2-c]pyridine | 29 (53) | B | method 2 [M + H]⁺ = 375.0 MW_{calc} = 374.08 Rₜ = 1.05 min |
| 65 | 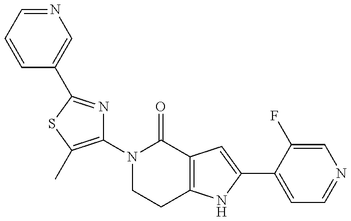 [SMILES: Cc1sc(-c2cccnc2)nc1N1CCc2[nH]c(-c3ccncc3F)cc2C1=O] | 2-(3-Fluoro-pyridin-4-yl)-5- (5-methyl-2-pyridin-3-yl- thiazol-4-yl)-1,5,6,7- tetrahydro-pyrrolo[3,2- c]pyridin-4-one | 220 (88) | A | method 2 [M + H]⁺ = 406.2 MW_{calc} = 405.11 Rₜ = 0.47 min |

¹yield step 1;
²yield step 2.

### Synthesis Example 66

### 4-[2-(2,6-Difluoro-phenyl)-3-iodo-4,5,6,7-tetrahydro-1H-pyrrolo[3,2-c]pyridin-5-yl]-5-methyl-2-pyridin-3-yl-thiazole

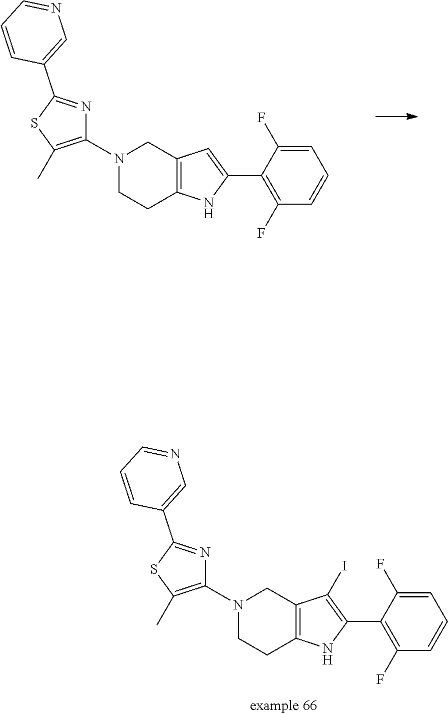 [SMILES: Cc1sc(-c2cccnc2)nc1N1CCc2[nH]c(-c3c(F)cccc3F)cc2C1 ; Cc1sc(-c2cccnc2)nc1N1CCc2[nH]c(-c3c(F)cccc3F)c(I)c2C1]

To a solution of the title compound of example 2 (150 mg, 370 μmol) in dry DMF (5 mL), N-iodosuccinimide (160 mg, 0.75 mmol) was added at it and the RM stirred for 16 h. The RM was diluted with EtOAc and was washed with water. The organic layer was dried, the volatiles were removed under reduced pressure and the residue was purified by CC (SiO₂) to yield the desired compound (34 mg, 17%).

LC-MS (method 2): m/z: [(M+H]⁺=535.1 (MW calc.=534.36); Rₜ=0.90 min.

¹H NMR (400 MHz, CDCl₃): 11.49 (s, 1H), 9.04-9.03 (m, 1H), 8.62 (dd, J=5.2, 1.2 Hz, 1H), 8.22-8.19 (m, 1H), 7.53-7.48 (m, 2H), 7.23-7.19 (m, 2H), 3.98 (s, 2H), 3.44-3.41 (m, 2H), 2.83-2.80 (m, 2H), 2.41 (s, 3H).

### Synthesis Example 67

### 4-[2-(2,6-Difluoro-phenyl)-4,5,6,7-tetrahydro-1H-pyrrolo[3,2-c]pyridin-5-yl]-2-fluoro-5-methyl-benzonitrile

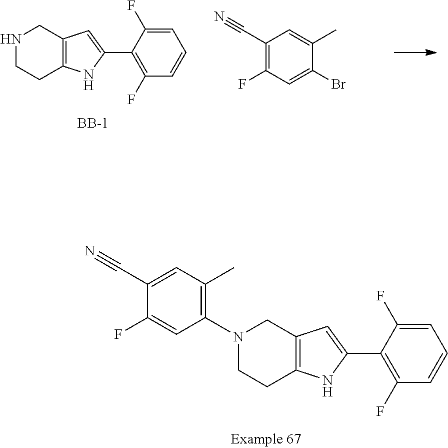 [SMILES: Fc1cccc(F)c1-c1cc2c([nH]1)CCNC2 ; Cc1cc(C#N)c(F)cc1Br ; "BB-1" ; Cc1cc(C#N)c(F)cc1N1CCc2[nH]c(-c3c(F)cccc3F)cc2C1]

Example 67 was synthesized in analogy to the previous examples through the reaction of BB-1 (150 mg, 641 μmol) with 4-Bromo-2-fluoro-5-methylbenzonitrile (164 mg, 769 μmol) (160 mg, 68%).

LC-MS (method 2): m/z: [(M+H]⁺=368.2 (MW calc.=367.37); Rₜ=0.93 min.

¹H NMR (600 MHz, DMSO-d₆) δ (ppm)=10.86 (s, 1H), 7.62 (dd, J=7.8, 1.6 Hz, 1H), 7.26 (tt, J=8.3, 6.3 Hz, 1H), 7.19-7.11 (m, 2H), 7.06 (dd, J=12.2, 1.3 Hz, 1H), 6.34 (q, J=2.1 Hz, 1H), 4.09 (s, 2H), 3.35 (t, J=5.7 Hz, 2H), 2.82 (t, J=5.7 Hz, 2H), 2.28 (s, 3H), 2.11-2.05 (m, 1H).

### Synthesis Example 68

### 6-[2-(2,6-Difluoro-phenyl)-4,5,6,7-tetrahydro-1H-pyrrolo[3,2-c]pyridin-5-yl]-5-methyl-nicotinonitrile

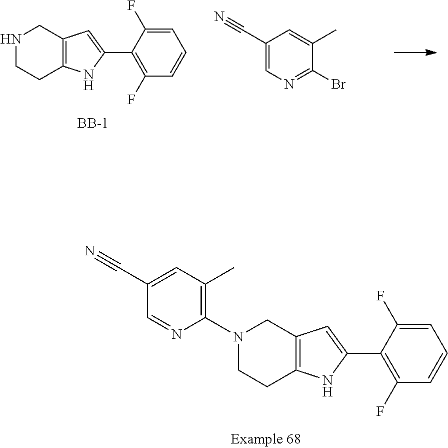

Example 68 was synthesized in analogy to the previous examples through the reaction of BB-1 (130 mg, 641 μmol) with 6-bromo-5-methylnicotinonitrile (131 mg, 666 μmol) (136 mg, 70%).

LC-MS (method 2): m/z: [(M+H]⁺=351.3 (MW calc.=350.37); Rₜ=0.88 min.

¹H NMR (600 MHz, DMSO-d₆) δ (ppm)=10.83 (s, 1H), 8.46 (d, J=2.6 Hz, 1H), 7.83 (d, J=2.7 Hz, 1H), 7.25 (ddd, J=14.4, 8.4, 6.3 Hz, 1H), 7.14 (t, J=8.4 Hz, 2H), 6.34 (q, J=2.1 Hz, 1H), 4.39 (s, 2H), 3.64 (t, J=5.6 Hz, 2H), 2.88 (t, J=5.7 Hz, 2H), 2.33 (s, 3H).

### Synthesis Example 69

### 2-(2,6-Difluoro-phenyl)-5-(6-ethoxy-4-methyl-pyridin-3-yl)-4,5,6,7-tetrahydro-1H-pyrrolo[3,2-c]pyridine

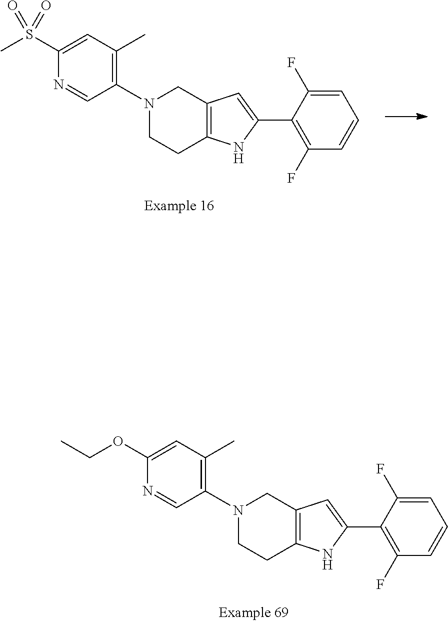 [SMILES: Cc1cc(S(C)(=O)=O)ncc1N1CCc2[nH]c(-c3c(F)cccc3F)cc2C1 ; CCOc1cc(C)c(N2CCc3[nH]c(-c4c(F)cccc4F)cc3C2)cn1]

To a solution of example 16 (310 mg, 0.77 mmol) was added freshly prepared 1 m solution of NaOEt in EtOH (2.3 mL, 2.30 mmol) and the RM was heated at reflux for 48 h. Subsequently the RM was concentrated under reduced pressure and the residue was dissolved in EtOAc and dried over sodium sulfate. The solvent was evaporated under reduced pressure and the crude product was purified by CC (6% EtOAc/Hexane) to yield the title compound (80 mg, 28%).

LC-MS (Method 3): m/z [M+H]⁺=370.0 (MW calc. 369.41); Rₜ=3.95 min.

¹H NMR (DMSO-d₆, 400 MHz), δ (ppm)=10.79 (s, 1H), 7.88 (s, 1H), 7.26 (m, 1H), 7.14 (t, J=8.6 Hz, 2H), 6.63 (s, 1H), 6.30 (s, 1H), 4.22 (q, J=7.1 Hz, 2H), 3.91 (s, 2H), 3.15 (t, J=5.2 Hz, 2H), 2.77 (t, J=5.0 Hz, 2H), 2.25 (s, 3H), 1.28 (t, J=7.0 Hz, 3H).

### Synthesis Example 70

### 2-(2,6-Difluoro-phenyl)-5-[4-methyl-6-(methylsulfinyl)-pyridin-3-yl]-4,5,6,7-tetrahydro-1H-pyrrolo[3,2-c]pyridine

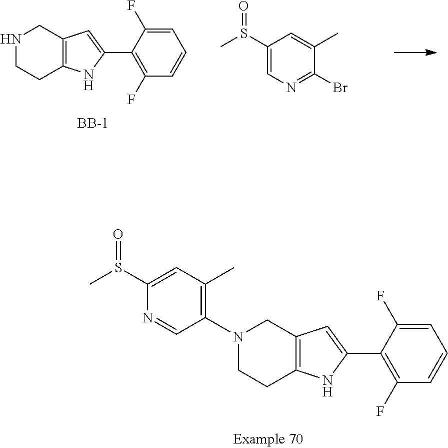

Example 70 was synthesized in analogy to the previous examples through the reaction of BB-1 (250 mg, 1.07 mmol) with 5-bromo-4-methyl-2-(methylsulfinyl)pyridine (299 mg, 1.28 mmol) (47 mg, 12%). LC-MS (method 2): m/z: [(M+H]⁺=388.1 (MW calc.=387.45); Rₜ=0.86 min.

¹H NMR (600 MHz, DMSO-d₆) δ (ppm)=10.85, (s, 1H), 8.36 (s, 1H), 7.69 (s, 1H), 7.25 (tt, J=8.7, 6.4 Hz, 1H), 7.14 (t, J=8.5 Hz, 2H), 6.35 (d, J=2.2 Hz, 1H), 4.12 (s, 2H), 3.32 (t, J=6.0 Hz, 2H), 2.81 (t, J=5.8 Hz, 2H), 2.75 (s, 3H), 2.42 (s, 3H).

### Synthesis Example 71

### 5-(4-Chloro-5-fluoro-2-methyl-phenyl)-2-(2,6-difluoro-phenyl)-4,5,6,7-tetrahydro-1H-pyrrolo[3,2-c]pyridine

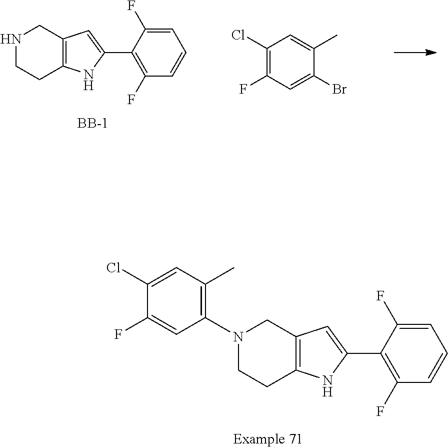

Example 71 was synthesized in analogy to the previous examples through the reaction of BB-1 (150 mg, 641 μmol) with 1-bromo-4-chloro-5-fluoro-2-methylbenzene (171 mg, 769 μmol) (38 mg, 16%). LC-MS (method 2): m/z: [(M+H]⁺=377.2 (MW calc.=376.81); Rₜ=1.02 min.

¹H NMR (600 MHz, DMSO-d₆) δ (ppm)=10.81 (s, 1H), 7.36 (d, J=8.7 Hz, 1H), 7.29-7.20 (m, 1H), 7.14 (t, J=8.4 Hz, 2H), 7.09 (d, J=11.6 Hz, 1H), 6.33 (d, J=2.1 Hz, 1H), 3.92 (s, 2H), 3.18 (t, J=5.7 Hz, 2H), 2.78 (t, J=5.7 Hz, 2H), 2.25 (s, 3H), 2.06 (s, 1H).

### Synthesis Example 72

### 2-Cyclohexyl-5-(4-methyl-6-methylsulfonyl-pyridin-3-yl)-4,5,6,7-tetrahydro-1H-pyrrolo[3,2-c]pyridine

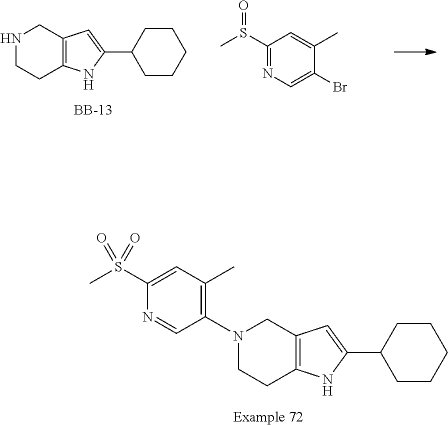

Example 72 was synthesized in analogy to the previous examples through the reaction of BB-13 (180 mg, 748 μmol) with 5-bromo-4-methyl-2-(methylsulfonyl)pyridine (225 mg, 897 μmol) (80 mg, 29%). LC-MS (method 2): m/z: [(M+H]⁺=374.2 (MW calc.=373.52); Rₜ=0.99 min.

¹H NMR (600 MHz, DMSO-d₆) δ (ppm)=10.19 (d, J=2.5 Hz, 1H), 8.34 (s, 1H), 7.80 (s, 1H), 5.52 (d, J=2.3 Hz, 1H), 4.07 (s, 2H), 3.32 (t, J=5.6 Hz, 2H), 3.18 (s, 3H), 2.67 (t, J=5.7 Hz, 2H), 2.50-2.40 (m, 1H), 2.39 (s, 3H), 1.89 (d, J=8.0 Hz, 2H), 1.74 (dd, J=9.5, 4.9 Hz, 2H), 1.69-1.62 (m, 1H), 1.37-1.26 (m, 1H), 1.30 (s, 3H), 1.31-1.12 (m, 2H).

### Synthesis Example 73

### 2-(2,6-Difluoro-phenyl)-5-[4-methyl-6-(trifluoromethyl)-pyridin-3-yl]-4,5,6,7-tetrahydro-1H-pyrrolo[3,2-c]pyridine

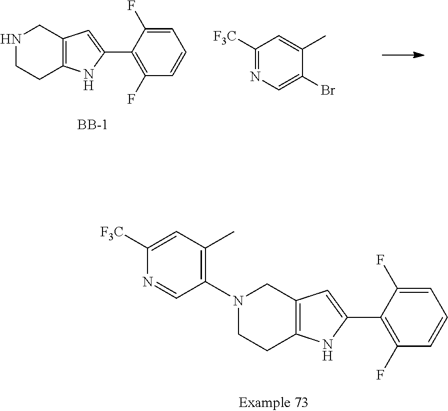 [SMILES: Fc1cccc(F)c1-c1cc2c([nH]1)CCNC2 ; Cc1cc(C(F)(F)F)ncc1Br ; "BB-1" ; Cc1cc(C(F)(F)F)ncc1N1CCc2[nH]c(-c3c(F)cccc3F)cc2C1]

Example 73 was synthesized in analogy to the previous examples through the reaction of BB-1 (180 mg, 748 μmol) with 5-bromo-4-methyl-2-(trifluoromethyl)pyridine (221 mg, 922 μmol) (45 mg, 15%).

LC-MS (method 2): m/z: [(M+H]⁺=394.3 (MW calc.=393.36); Rₜ=0.91 min.

¹H NMR (600 MHz, DMSO-d₆) δ (ppm)=10.89 (s, 1H), 8.41 (s, 1H), 7.68 (s, 1H), 7.29-7.21 (m, 1H), 7.19-7.10 (m, 2H), 6.36 (t, J=2.1 Hz, 1H), 4.16 (s, 2H), 3.35 (t, J=5.7 Hz, 2H), 2.82 (t, J=5.7 Hz, 2H), 2.40 (s, 3H).

### Synthesis Example 74

### 2-Butyl-5-(4-methyl-6-methylsulfonyl-pyridin-3-yl)-4,5,6,7-tetrahydro-1H-pyrrolo[3,2-c]pyridine

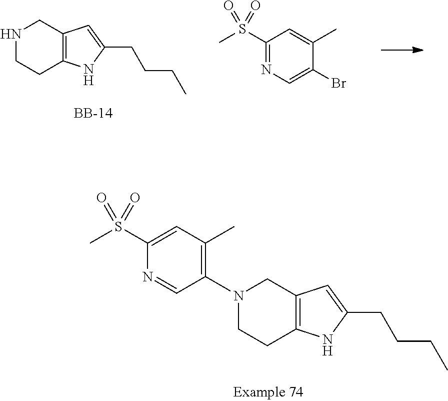 [SMILES: CCCCc1cc2c([nH]1)CCNC2 ; Cc1cc(S(C)(=O)=O)ncc1Br ; "BB-14" ; CCCCc1cc2c([nH]1)CCN(c1cnc(S(C)(=O)=O)cc1C)C2]

Example 74 was synthesized in analogy to the previous examples through the reaction of BB-14 (200 mg, 931 μmol) with 5-bromo-4-methyl-2-(methylsulfonyl)pyridine (280 mg, 1.12 mmol) (40 mg, 13%). LC-MS (method 2): m/z: [(M+H]⁺=348.2 (MW calc.=347.48); Rₜ=0.76 min.

¹H NMR (600 MHz, DMSO-d₆) δ (ppm)=10.26-10.17 (m, 1H), 8.35 (s, 1H), 7.81 (s, 1H), 5.54 (d, J=2.4 Hz, 1H), 4.08 (s, 2H), 3.32 (t, J=5.6 Hz, 1H), 3.19 (s, 3H), 2.67 (t, J=5.7 Hz, 2H), 2.47 (t, J=7.6 Hz, 3H), 2.40 (s, 3H), 1.52 (p, J=7.5 Hz, 2H), 1.32 (h, J=7.4 Hz, 2H), 0.89 (t, J=7.4 Hz, 3H).

### Synthesis Example 75

### 5-(6-Cyclopropyl-4-methyl-pyridin-3-yl)-2-(2,6-difluoro-phenyl)-4,5,6,7-tetrahydro-1H-pyrrolo[3,2-c]pyridine

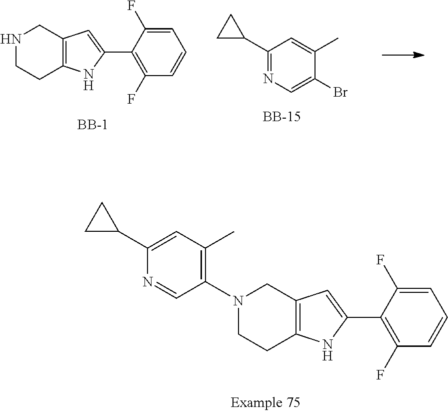

Example 75 was synthesized in analogy to the previous examples through the reaction of BB-1 (200 mg, 854 μmol) with BB-15 (217 mg, 1.02 mmol) (22 mg, 7%).

LC-MS (method 2): m/z: [(M+H]⁺=366.2 (MW calc.=365.43); Rₜ=0.60 min.

¹H NMR (600 MHz, DMSO-d₆) δ (ppm)=10.81 (s, 1H), 8.27 (s, 1H), 8.14 (s, 1H), 7.40-7.14 (m, 3H), 7.08 (s, 1H), 6.32 (s, 1H), 3.96 (s, 2H), 3.19 (s, 2H), 2.77 (s, 2H), 2.26 (s, 3H), 2.07 (s, 1H), 1.97 (s, 1H), 0.86 (s, 2H), 0.82 (s, 2H).

### Synthesis Example 76

### 5-(4-Methyl-6-methylsulfonyl-pyridin-3-yl)-2-tetrahydro-pyran-4-yl-4,5,6,7-tetrahydro-1H-pyrrolo[3,2-c]pyridine

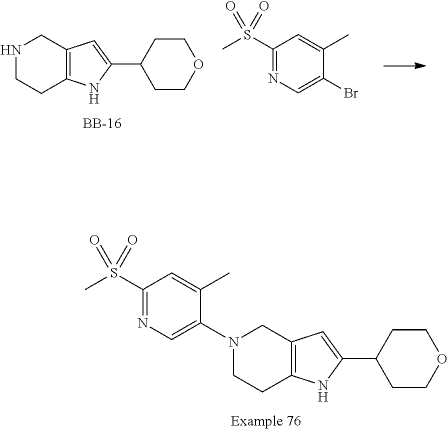

Example 76 was synthesized in analogy to the previous examples through the reaction of BB-16 (150 mg, 618 μmol) with 5-bromo-4-methyl-2-(methylsulfonyl)pyridine (186 mg, 741 mmol) (90 mg, 39%).

LC-MS (method 2): m/z: [(M+H]⁺=376.2 (MW calc.=375.49); Rₜ=0.62 min.

¹H NMR (400 MHz, DMSO-d₆) δ (ppm)=10.30 (s, 1H), 8.35 (s, 1H), 7.81 (s, 1H), 5.57 (s, 1H), 4.08 (s, 2H), 3.91-3.86 (m, 2H), 3.38 (t, J=11.3 Hz, 2H), 3.32 (t, J=5.6 Hz, 2H), 3.18 (s, 3H), 2.74-2.66 (m, 3H), 2.39 (s, 3H), 1.78 (dd, J=12.8 Hz, 3.8 Hz, 2H), 1.57 (ddd, J=12.8 Hz, 4.5

### Synthesis Example 77

### 5-[2-(2,6-Difluoro-phenyl)-4,5,6,7-tetrahydro-1H-pyrrolo[3,2-c]pyridin-5-yl]-6-methyl-pyridine-2-carbonitrile

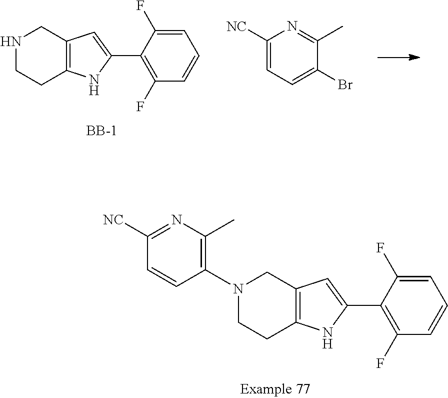

Example 77 was synthesized in analogy to the previous examples through the reaction of BB-1 (150 mg, 641 μmol) with 5-bromo-6-methylpicolinonitrile (151 mg, 769 mmol) (90 mg, 40%).

LC-MS (method 2): m/z: [(M+H]⁺=351.1 (MW calc.=350.37); Rₜ=0.81 min.

¹H NMR (400 MHz, DMSO-d₆) δ (ppm)=10.87 (s, 1H), 7.77 (d, J=8.3 Hz, 1H), 7.50 (d, J=9.1

### Synthesis Example 78

### 2-(2,4-Difluoro-phenyl)-5-(4-methyl-6-methylsulfonyl-pyridin-3-yl)-4,5,6,7-tetrahydro-1H-pyrrolo[3,2-c]pyridine

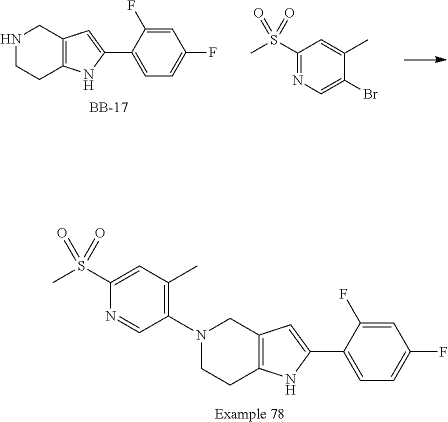

Example 78 was synthesized in analogy to the previous examples through the reaction of BB-17 (170 mg, 628 μmol) with 5-bromo-4-methyl-2-(methylsulfonyl)pyridine (189 mg, 754 mmol) (60 mg, 24%).

LC-MS (method 2): m/z: [(M+H]⁺=404.2 (MW calc.=403.45); Rₜ=0.75 min.

¹H NMR (400 MHz, DMSO-d₆) δ (ppm)=11.04 (s, 1H), 8.40 (s, 1H), 7.83 (s, 1H), 7.73-7.67 (m, 1H), 7.28-7.22 (m, 1H), 7.15-7.08 (m, 1H), 6.34 (s, 1H), 4.18 (s, 2H), 3.38 (t, J=5.6 Hz, 2H), 3.30 (s, 3H), 2.81 (t, J=5.6 Hz, 2H), 2.42 (s, 3H).

### Synthesis Example 79

### 2-(2-Fluorophenyl)-5-(4-methyl-6-methylsulfonyl-pyridin-3-yl)-4,5,6,7-tetrahydro-1H-pyrrolo[3,2-c]pyridine

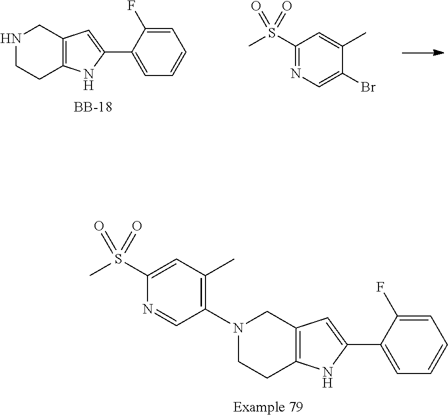

Example 79 was synthesized in analogy to the previous examples through the reaction of BB-18 (150 mg, 594 μmol) with 5-bromo-4-methyl-2-(methylsulfonyl)pyridine (179 mg, 712 mmol) (120 mg, 52%).

LC-MS (method 2): m/z: [(M+H]⁺=386.2 (MW calc.=385.46); Rₜ=0.77 min.

¹H NMR (400 MHz, DMSO-d₆) δ (ppm)=11.04 (s, 1H), 8.40 (s, 1H), 7.84 (s, 1H), 7.69 (t, J=6.8 Hz, 1H), 7.24-7.12 (m, 2H), 6.40 (s, 1H), 4.19 (s, 2H), 3.39 (t, J=5.3 Hz, 2H), 3.20 (s, 3H), 2.82 (t, J=5.3 Hz, 2H), 2.43 (s, 3H).

### Synthesis Example 80

### 4-[2-(2,6-Difluoro-phenyl)-4,5,6,7-tetrahydro-1H-pyrrolo[3,2-c]pyridin-5-yl]-N,3-dimethyl-benzamide

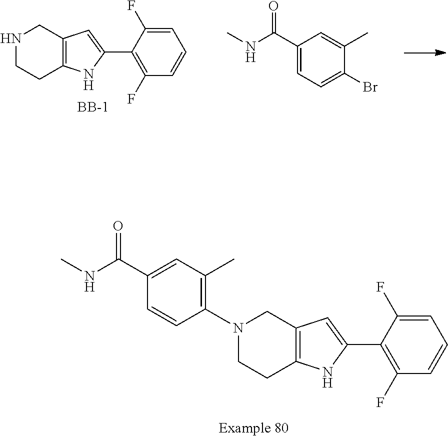

Example 80 was synthesized in analogy to the previous examples through the reaction of BB-1 (200 mg, 854 μmol) with 4-bromo-N,3-dimethylbenzamide (233 mg, 1.03 mmol) (86 mg, 26%).

LC-MS (method 2): m/z: [(M+H]⁺=382.3 (MW calc.=381.43); Rₜ=0.77 min.

¹H NMR (400 MHz, DMSO-d₆) δ (ppm)=10.81 (s, 1H), 8.23 (q, J=4.5 Hz, 1H), 7.67 (d, J=2.2 Hz, 1H), 7.62 (dd, J=8.3, 2.2 Hz, 1H), 7.29-7.18 (m, 1H), 7.19-7.09 (m, 3H), 6.34 (d, J=2.3 Hz, 1H), 3.98 (s, 2H), 3.23 (t, J=5.6 Hz, 2H), 2.82-2.74 (m, 5H), 2.32 (s, 3H).

### Synthesis Example 81

### 4-[2-(2,6-Difluoro-phenyl)-4,5,6,7-tetrahydro-1H-pyrrolo[3,2-c]pyridin-5-yl]-N,N,3-trimethyl-benzamide

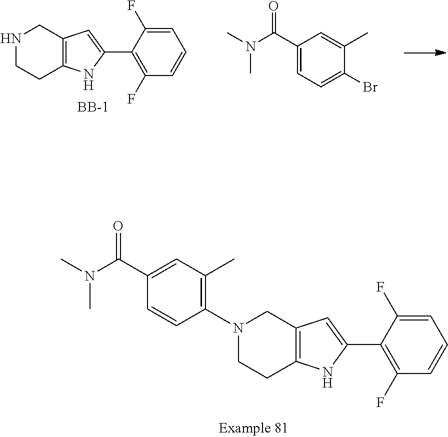

Example 81 was synthesized in analogy to the previous examples through the reaction of BB-1 (200 mg, 854 μmol) with 4-bromo-N,N,3-trimethylbenzamide (248 mg, 1.03 mmol) (170 mg, 50%).

LC-MS (method 2): m/z: [(M+H]⁺=396.3 (MW calc.=395.45); Rₜ=0.82 min.

¹H NMR (400 MHz, DMSO-d₆) δ (ppm)=10.80 (s, 1H), 7.27-7.10 (m, 6H), 6.33 (s, 1H), 3.97 (s, 2H), 3.21 (t, J=5.6 Hz, 2H), 2.95 (s, 6H), 2.79 (t, J=5.6 Hz, 2H), 2.31 (s, 3H).

### Synthesis Example 82

### 1-[4-[2-(2,6-Difluoro-phenyl)-4,5,6,7-tetrahydro-1H-pyrrolo[3,2-c]pyridin-5-yl]-3-methyl-phenyl]-ethanone

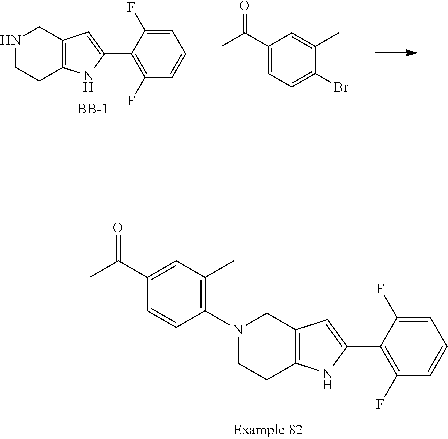

Example 82 was synthesized in analogy to the previous examples through the reaction of BB-1 (150 mg, 641 μmol) with 1-(4-bromo-3-methylphenyl)ethanone (163 mg, 769 mmol) (66 mg, 28%).

LC-MS (method 2): m/z: [(M+H]⁺=367.3 (MW calc.=366.41); Rₜ=0.90 min.

¹H NMR (400 MHz, DMSO-d₆) δ (ppm)=10.82 (s, 1H), 7.79-7.74 (m, 2H), 7.8-7.73 (m, 1H), 7.30-7.20 (m, 1H), 7.14 (t, J=8.1 Hz, 3H), 6.35 (s, 1H), 4.05 (s, 2H), 3.29 (t, J=5.6 Hz, 2H), 2.81 (t, J=5.7 Hz, 3H), 2.50 (s, 3H), 2.35 (s, 3H).

### Synthesis Example 83

### 5-(4-Methyl-6-methylsulfonyl-pyridin-3-yl)-2-(2-methyl-pyridin-3-yl)-4,5,6,7-tetrahydro-1H-pyrrolo[3,2-c]pyridine

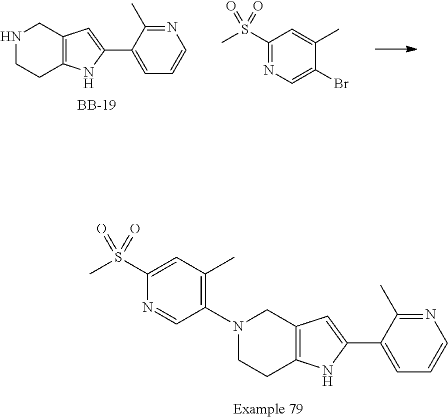

Example 83 was synthesized in analogy to the previous examples through the reaction of BB-19 (200 mg, 801 μmol) with 5-bromo-4-methyl-2-(methylsulfonyl)pyridine (240 mg, 961 mmol) (110 mg, 36%).

LC-MS (method 2): m/z: [(M+H]⁺=383.2 (MW calc.=382.49); Rₜ=0.46 min.

¹H NMR (400 MHz, DMSO-d₆) δ (ppm)=10.95 (s, 1H), 8.41 (s, 1H), 8.29 (dd, J=4.5, 1.5 Hz, 1H), 7.84 (s, 1H), 7.75 (dd, J=7.5, 1.5 Hz, 1H), 7.26-7.21 (m, 1H), 6.19 (d, J=3.0 Hz, 1H), 4.21 (s, 2H), 3.39 (t, J=5.6 Hz, 2H), 3.20 (s, 3H), 2.82 (t, J=5.6 Hz, 2H), 2.62 (s, 3H), 2.43 (s, 3H).

### Synthesis Example 84

### 2-(2,6-Difluoro-phenyl)-5-[5-methoxy-2-(trifluoromethyl)-pyrimidin-4-yl]-4,5,6,7-tetrahydro-1H-pyrrolo[3,2-c]pyridine

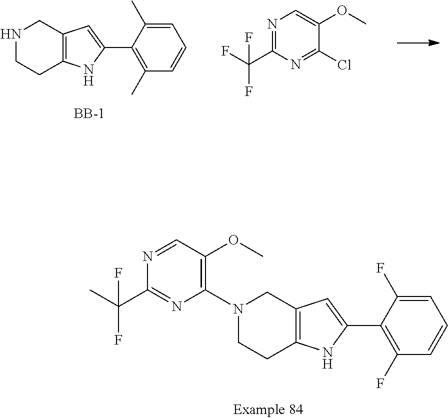

Example 84 was synthesized in analogy to the previous examples through the reaction of BB-1 (200 mg, 801 μmol) with 4-chloro-5-methoxy-2-(trifluoromethyl)pyrimidine (217 mg, 1.01 mmol) (117 mg, 33%).

LC-MS (method 2): m/z: [(M+H]⁺=411.3 (MW calc.=410.35); Rₜ=0.90 min.

¹H NMR (400 MHz, DMSO-d₆) δ (ppm)=10.85 (s, 1H), 8.13 (s, 1H), 7.28-7.21 (m, 1H), 7.17-7.11 (m, 2H), 6.35 (s, 1H), 4.76 (s, 2H), 4.04 (t, J=5.6 Hz, 2H), 3.96 (s, 3H), 2.85 (t, J=5.6 Hz, 2H).

### Synthesis Example 85

### [2-(2,6-Difluoro-phenyl)-5-(5-methyl-2-pyridin-3-yl-thiazol-4-yl)-4,5,6,7-tetrahydro-1H-pyrrolo[3,2-c]pyridin-3-yl]-methanol

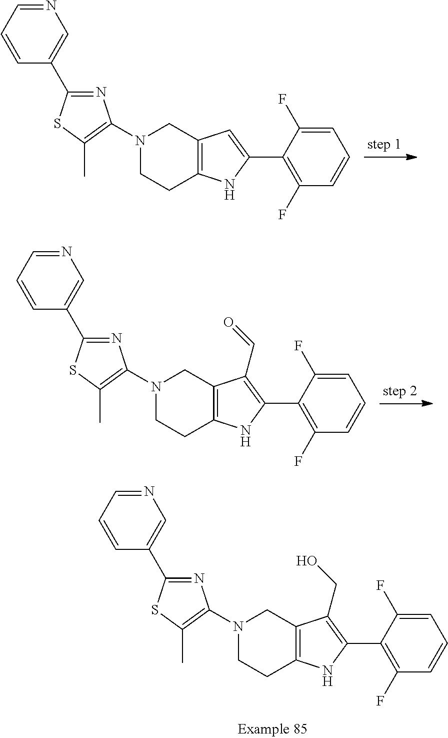 [SMILES: Cc1sc(-c2cccnc2)nc1N1CCc2[nH]c(-c3c(F)cccc3F)cc2C1 ; Cc1sc(-c2cccnc2)nc1N1CCc2[nH]c(-c3c(F)cccc3F)c(C=O)c2C1 ; Cc1sc(-c2cccnc2)nc1N1CCc2[nH]c(-c3c(F)cccc3F)c(CO)c2C1]

Step 1:

DMF (0.05 mL, 612 μmol) was added to the POCl₃ (0.05 mL, 612 μmol) at 0° C. and the RM was stirred for 10 min. A solution of the tilte compound of example 2 (50 mg, 122 μmol) in dry DMF (2 mL) was added and the RM was stirred at it for 4 h. The RM was cooled to 0° C. and cold water and saturated aqueous solution of NaHCO3 were consecutively added. The mixture was extracted with EtOAc, the organic layer was washed with water, was dried and the volatiles were removed under reduced pressure. The residue was purified by CC (SiO₂) to yield the desired compound (15 mg, 28%).

¹H NMR (400 MHz, CDCl₃): 9.71 (t, J=2.4 Hz, 1H), 9.09 (s, 1H), 9.0 (s, 1H), 8.59 (d, J=4.8 Hz, 1H), 8.44 (d, J=8 Hz, 1H), 7.65-7.62 (m, 1H), 7.42-7.35 (m, 1H), 7.01-7.02 (m, 2H), 4.56 (s, 2H), 3.64-3.61 (m, 2H), 3.01 (br s, 2H), 2.52 (s, 3H).

Step 2:

To a solution of step 1 intermediate (100 mg, 0.23 mmol) in dry MeOH (20 mL), NaBH₄ (17.3 mg, 0.46 mmol) was added at 0° C. and the RM was warmed to it and stirred for 1 h. The RM was cooled to 0° C., water was added and mixture was extracted with EtOAc The combined organic layers were washed with brine, were dried and the volatiles were removed under reduced pressure. The residue was purified by CC (SiO₂, CH₂Cl₂/MeOH) to yield the desired compound (70 mg, 70%).

LC-MS (method 2): m/z: [(M+H]⁺=439.3 (MW calc.=438.49); Rₜ=0.70 min.

¹H NMR (400 MHz, CDCl₃): 10.70 (s, 1H), 9.03 (s, 1H), 8.61 (d, J=3.6 Hz, 1H), 8.21-8.18 (m, 1H), 7.50 (dd, J=8, 4.8 Hz, 1H), 7.45-7.37 (m, 1H), 7.18-7.14 (m, 2H), 4.41 (t, J=5.4 Hz, 1H), 4.24-4.21 (m, 4H), 3.42 (t, J=5.6 Hz, 2H), 2.82-2.80 (m, 1H), 2.41 (s, 3H).

### Synthesis Example 86

### 2-(2,6-Difluoro-phenyl)-5-[2-methyl-4-(trifluoromethylsulfonyl)-phenyl]-4,5,6,7-tetrahydro-1H-pyrrolo[3,2-c]pyridine

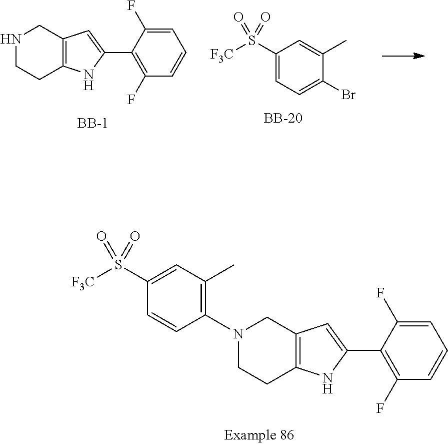 [SMILES: Fc1cccc(F)c1-c1cc2c([nH]1)CCNC2 ; Cc1cc(S(=O)(=O)C(F)(F)F)ccc1Br ; "BB-20" ; "BB-1" ; Cc1cc(S(=O)(=O)C(F)(F)F)ccc1N1CCc2[nH]c(-c3c(F)cccc3F)cc2C1]

Example 86 was synthesized in analogy to the previous examples through the reaction of BB-1 (150 mg, 641 μmol) with BB-20 (233 mg, 769 mmol) (197 mg, 67%).

LC-MS (method 2): m/z: [(M+H]⁺=457.3 (MW calc.=456.43); Rₜ=0.97 min.

¹H NMR (400 MHz, DMSO-d₆) δ (ppm)=10.88 (s, 1H), 7.85-7.79 (m, 2H), 7.35 (d, J=9 Hz, 1H), 7.28-7.2 (m, 2H), 7.18-7.12 (m, 2H), 6.36 (s, 1H), 4.22 (s, 2H), 3.49-3.43 (m, 2H), 2.87.2.84 (m, 2H), 2.43 (s, 3H).

### Synthesis Example 87

### 2-(2,6-Difluoro-phenyl)-5-[2-methyl-4-(methylsulfinyl)-phenyl]-4,5,6,7-tetrahydro-1H-pyrrolo[3,2-c]pyridine

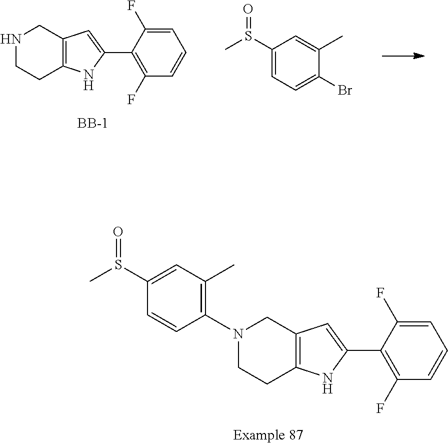

Example 87 was synthesized in analogy to the previous examples through the reaction of BB-1 (210 mg, 897 μmol) with 1-bromo-2-methyl-4-(methylsulfinyl)benzene (251 mg, 1.08 mol) (150 mg, 43%).

LC-MS (method 2): m/z: [(M+H]⁺=387.2 (MW calc.=386.46); Rₜ=0.79 min.

¹H NMR (400 MHz, DMSO-d₆) δ (ppm)=10.82 (s, 1H), 7.50 (dd, J=2.3, 0.8 Hz, 1H), 7.46 (dd, J=8.3, 2.3 Hz, 1H), 7.30-7.20 (m, 2H), 7.20-7.10 (m, 2H), 6.35 (d, J=2.1 Hz, 1H), 4.00 (s, 2H), 3.24 (t, J=5.7 Hz, 2H), 2.81 (t, J=5.7 Hz, 2H), 2.71 (s, 3H), 2.36 (s, 3H).

### Synthesis Example 88

### 2-(2,6-Difluoro-phenyl)-5-(2-methyl-4-methylsulfonyl-phenyl)-4,5,6,7-tetrahydro-1H-pyrrolo[3,2-c]pyridine

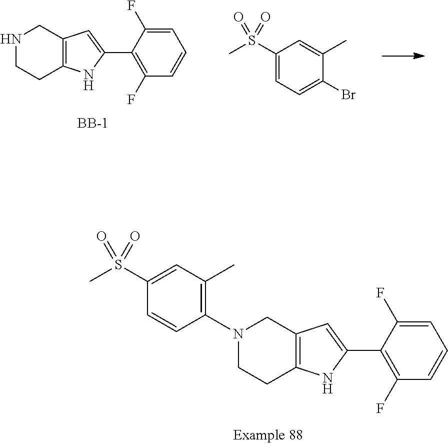 [SMILES: Fc1cccc(F)c1-c1cc2c([nH]1)CCNC2 ; Cc1cc(S(C)(=O)=O)ccc1Br ; "BB-1" ; Cc1cc(S(C)(=O)=O)ccc1N1CCc2[nH]c(-c3c(F)cccc3F)cc2C1]

Example 88 was synthesized in analogy to the previous examples through the reaction of BB-1 (150 mg, 641 μmol) with 1-bromo-2-methyl-4-(methylsulfonyl)benzene (190 mg, 769 mmol) (140 mg, 54%).

LC-MS (method 2): m/z: [(M+H]⁺=403.2 (MW calc.=402.46); Rₜ=0.83 min.

¹H NMR (400 MHz, DMSO-d₆) δ (ppm)=10.85 (s, 1H), 7.74-7.65 (m, 2H), 7.30-7.18 (m, 2H), 7.15 (t, J=8.4 Hz, 2H), 6.36 (d, J=2.2 Hz, 1H), 4.07 (s, 2H), 3.30 (t, J=5.6 Hz, 1H), 3.14 (s, 3H), 2.83 (t, J=5.6 Hz, 2H), 2.38 (s, 3H).

### Synthesis Example 89

### 4-Methyl-5-[5-(4-methyl-6-methylsulfonyl-pyridin-3-yl)-4,5,6,7-tetrahydro-1H-pyrrolo[3,2-c]pyridin-2-yl]-[1,2,3]thiadiazole

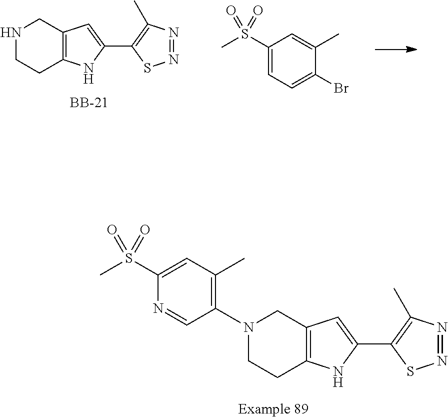

Example 89 was synthesized in analogy to the previous examples through the reaction of BB-21 (200 mg, 779 μmol) with 5-bromo-4-methyl-2-(methylsulfonyl)pyridine (233 mg, 935 mmol) (20 mg, 7%).

LC-MS (method 2): m/z: [(M+H]⁺=390.2 (MW calc.=389.50); Rₜ=0.66 min.

¹H NMR (600 MHz, DMSO-d₆) δ (ppm)=11.46 (s, 1H), 8.41 (s, 1H), 7.84 (s, 1H), 6.46 (s, 1H), 4.21 (s, 2H), 3.39 (t, J=5.3 Hz, 2H), 3.20 (s, 3H), 2.62 (t, J=5.3 Hz, 2H), 2.42 (s, 3H).

### Synthesis Example 90

### 2-(3-Fluoro-pyridin-4-yl)-5-(4-methyl-6-methylsulfonyl-pyridin-3-yl)-4,5,6,7-tetrahydro-1H-pyrrolo[3,2-c]pyridine

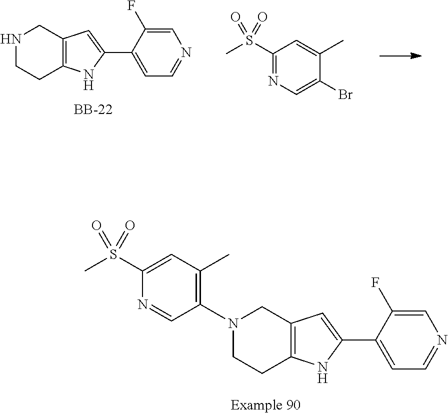

Example 90 was synthesized in analogy to the previous examples through the reaction of BB-22 (320 mg, 1.26 mmol) with 5-bromo-4-methyl-2-(methylsulfonyl)pyridine (380 mg, 1.51 mmol) (120 mg, 25%). LC-MS (method 2): m/z: [(M+H]⁺=387.1 (MW calc.=386.45); Rₜ=0.50 min.

¹H NMR (600 MHz, DMSO-d₆) δ (ppm)=11.46 (s, 1H), 8.48 (d, J=3.4 Hz, 1H), 8.41 (s, 1H), 8.33 (dd, J=5.2, 1.1 Hz, 1H), 7.85 (s, 1H), 7.70 (dd, J=7.2, 5.1 Hz, 1H), 6.68 (s, 1H), 4.21 (s, 2H), 3.21 (s, 3H), 2.87 (t, J=5.7 Hz, 2H), 2.43 (s, 3H).

### Synthesis Example 91

### 3-Bromo-2-(2,6-difluoro-phenyl)-5-(4-methyl-6-methylsulfonyl-pyridin-3-yl)-4,5,6,7-tetrahydro-1H-pyrrolo[3,2-c]pyridine

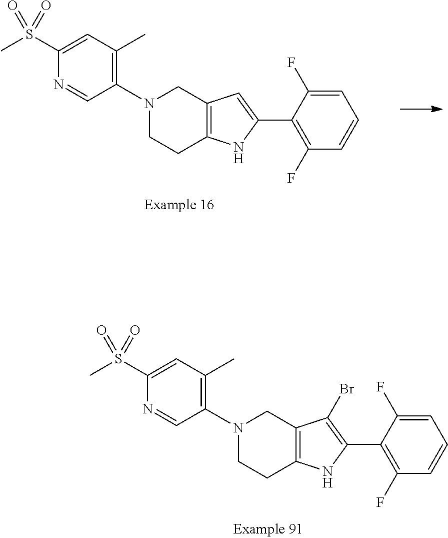 [SMILES: Cc1cc(S(C)(=O)=O)ncc1N1CCc2[nH]c(-c3c(F)cccc3F)cc2C1 ; Cc1cc(S(C)(=O)=O)ncc1N1CCc2[nH]c(-c3c(F)cccc3F)c(Br)c2C1]

To a solution of example 16 (120 mg, 298 μmol) in dry THF (0.1 mL) was added NBS (51 mg, 298 μmol) at 0° C. and the mixture was stirred for 1 h. Water was added and the aqueous phase was extracted with a mixture of cyclohexane and EtOAc. The combined organic layers were dried and the volatiles were removed under reduced pressure and the residue was purified by chromatography (Interchim® cartridge 50SiHP/12 g, Cy/EtOAc) to yield the desired compound (65 mg, 45%).

LC-MS (method 2): m/z: [(M+H]⁺=482.1 (MW calc.=482.35); Rₜ=0.74 min.

¹H NMR (400 MHz, DMSO-d₆) δ (ppm)=11.45 (s, 1H), 8.45 (s, 1H), 7.86 (s, 1H), 7.54-6.46 (m, 1H), 7.25-7.18 (m, 2H), 4.09 (s, 2H), 3.41-3.37 (m, 2H), 3.21 (s, 3H), 2.82-2.76 (m, 2H), 2.43 (s, 3H).

### Synthesis Example 92

### 2-(4,6-Dimethyl-pyridin-3-yl)-5-(4-methyl-6-methylsulfonyl-pyridin-3-yl)-4,5,6,7-tetrahydro-1H-pyrrolo[3,2-c]pyridine

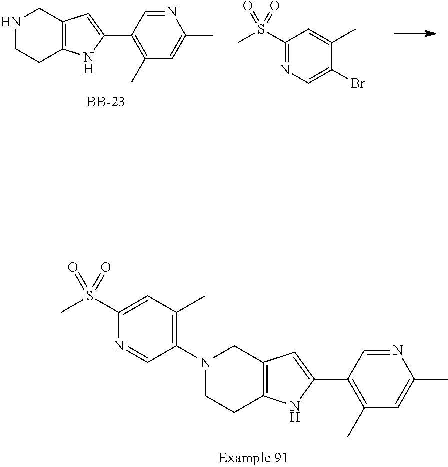

Example 92 was synthesized in analogy to the previous examples through the reaction of BB-23 (200 mg, 594 μmol) with 5-bromo-4-methyl-2-(methylsulfonyl)pyridine (178 mg, 713 μmol) (90 mg, 38%).

LC-MS (method 2): m/z: [(M+H]⁺=397.2 (MW calc.=396.51); Rₜ=0.47 min.

¹H NMR (400 MHz, DMSO-d₆) δ (ppm)=10.91 (s, 1H), 8.43 (s, 1H), 8.41 (s, 1H), 7.84 (s, 1H), 7.11 (s, 1H), 6.12 (d, J=2.3 Hz, 1H), 4.20 (s, 2H), 3.41-3.37 (m, 2H), 3.20 (s, 3H), 2.83-2.79 (m, 2H), 2.43 (s, 3H), 2.41 (s, 3H), 2.38 (s, 3H).

### Synthesis Example 93

### 5-[4-Methyl-6-(trifluoromethyl)-pyridin-3-yl]-2-tetrahydro-pyran-4-yl-4,5,6,7-tetrahydro-1H-pyrrolo[3,2-c]pyridine

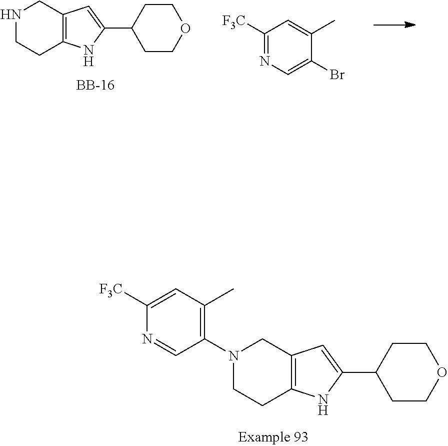

Example 93 was synthesized in analogy to the previous examples through the reaction of BB-16 (150 mg, 618 μmol) with 5-bromo-4-methyl-2-(trifluoromethyl)pyridine (178 mg, 742 μmol) (35 mg, 16%). LC-MS (method 2): m/z: [(M+H]⁺=366.2 (MW calc.=365.40); Rₜ=0.77 min.

¹H NMR (600 MHz, DMSO-d₆) δ (ppm)=10.28 (s, 1H), 8.36 (s, 1H), 7.66 (s, 1H), 5.56 (d, J=1.5 Hz, 1H), 4.04 (s, 2H), 3.89 (dd, J=11.3, 3 Hz, 2H), 3.41-3.36 (m, 2H), 3.30-3.37 (m, 2H), 2.74-2.66 (m, 3H), 2.37 (s, 3H), 1.80-1.75 (m, 2H), 1.61-1.53 (m, 2H).

### Synthesis Example 94

### 2-(2,6-Difluoro-phenyl)-5-(5-fluoro-4-methyl-pyridin-3-yl)-4,5,6,7-tetrahydro-1H-pyrrolo[3,2-c]pyridine

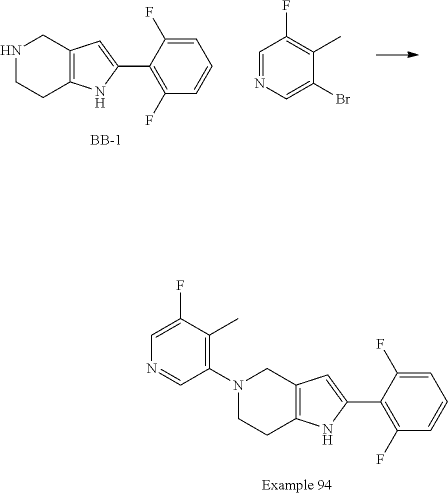

Example 94 was synthesized in analogy to the previous examples through the reaction of BB-1 (200 mg, 854 μmol) with 3-bromo-5-fluoro-4-methylpyridine (194 mg, 1.03 mmol) (80 mg, 27%).

LC-MS (method 2): m/z: [(M+H]⁺=344.2 (MW calc.=343.35); Rₜ=0.77 min.

¹H NMR (400 MHz, DMSO-d₆) δ (ppm)=10.83 (s, 1H), 8.21 (s, 1H), 8.18 (s, 1H), 7.29-7.20 (m, 1H), 7.17-7.11 (m, 2H), 6.34 (s, 1H), 4.08 (s, 2H), 3.30-3.26 (m, 2H), 2.80 (t, J=5.6 Hz, 2H), 2.23 (s, 3H).

### Synthesis Example 95

### [5-[2-(2,6-Difluoro-phenyl)-4,5,6,7-tetrahydro-1H-pyrrolo[3,2-c]pyridin-5-yl]-4-methyl-pyridin-2-yl]-amine

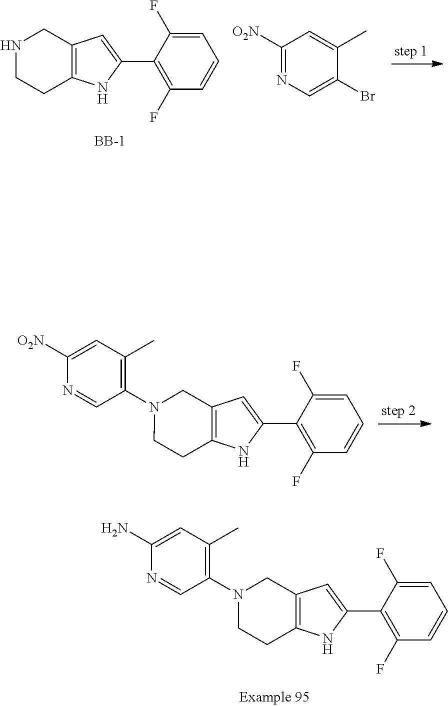

Step 1:

Step was performed in analogy to the previous examples through the reaction of BB-1 (200 mg, 854 μmol) with 5-bromo-4-methyl-2-nitropyridine (222 mg, 1.03 mmol) (50 mg, 16%).

LC-MS (method 2): m/z: [(M+H]⁺=371.3 (MW calc.=370.35); Rₜ=0.99 min.

Step 2:

A solution of step 1 intermediate (120 mg, 324 μmol, combined from 2 batches) in EtOH (6.8 mL) and EtOAc (6.8 mL) was hydrogenated utilizing a H-Cube® continuous-flow hydrogenation reactor (10% Pd/C, CatCart 30, 20 bar, 0.5 mL/min). The volatiles were removed under reduced pressure and the residue was purified by chromatography (Interchim® cartridge 50SiHP/12 g, Cy/EtOAc) to yield the desired compound (49 mg, 44%).

LC-MS (method 2): m/z: [(M+H]⁺=341.2 (MW calc.=340.38); Rₜ=0.56 min.

¹H NMR (600 MHz, DMSO-d₆) δ (ppm)=10.75 (s, 1H), 7.71 (s, 1H), 7.23 (tt, J=8.5, 6.3 Hz, 1H), 7.13 (t, J=8.4 Hz, 2H), 6.33 (s, 1H), 6.29 (q, J=2.1 Hz, 1H), 3.83 (s, 2H), 3.50-3.48 (m, 2H), 3.10 (t, J=5.6 Hz, 2H), 2.74 (t, J=5.6 Hz, 2H), 2.15 (s, 3H).

### Synthesis Example 96

### 2-(5-Fluoro-4-methyl-pyridin-3-yl)-5-(4-methyl-6-methylsulfonyl-pyridin-3-yl)-4,5,6,7-tetrahydro-1H-pyrrolo[3,2-c]pyridine

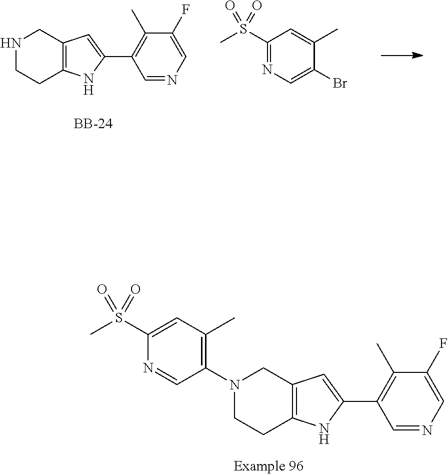

Example 96 was synthesized in analogy to the previous examples through the reaction of BB-23 (170 mg, 635 μmol) with 5-bromo-4-methyl-2-(methylsulfonyl)pyridine (191 mg, 762 μmol) (30 mg, 12%). LC-MS (method 2): m/z: [(M+H]⁺=401.2 (MW calc.=400.48); Rₜ=0.85 min.

¹H NMR (400 MHz, DMSO-d₆) δ (ppm)=11.11 (s, 1H), 8.46 (s, 1H), 8.42 (s, 1H), 8.31 (s, 1H), 7.84 (s, 1H), 6.26 (d, J=2.3 Hz, 1H), 4.21 (s, 2H), 3.42-3.38 (m, 2H), 3.20 (s, 3H), 2.86-2.83 (m, 2H), 2.43 (s, 3H), 2.37 (d, J=1.5 Hz, 3H).

### Synthesis Example 97

### 2-Cyclohexyl-5-[4-methyl-6-(trifluoromethyl)-pyridin-3-yl]-4,5,6,7-tetrahydro-1H-pyrrolo[3,2-c]pyridine

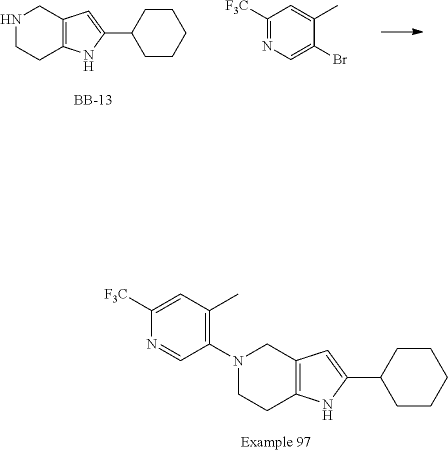 [SMILES: c1c(C2CCCCC2)[nH]c2c1CNCC2 ; Cc1cc(C(F)(F)F)ncc1Br ; "BB-13" ; Cc1cc(C(F)(F)F)ncc1N1CCc2[nH]c(C3CCCCC3)cc2C1]

Example 97 was synthesized in analogy to the previous examples through the reaction of BB-13 (150 mg, 734 μmol) with 5-bromo-4-methyl-2-(trifluoromethyl)pyridine (211 mg, 881 μmol) (25 mg, 9%).

LC-MS (method 2): m/z: [(M+H]⁺=364.2 (MW calc.=363.42); Rₜ=0.91 min.

¹H NMR (600 MHz, DMSO-d₆) δ (ppm)=10.18 (s, 1H), 8.35 (s, 1H), 7.65 (s, 1H), 5.52 (d, J=2.5 Hz, 1H), 4.03 (s, 2H), 3.29 (t, J=5.6 Hz, 2H), 2.74-2.64 (m, 2H), 2.44 (ddt, J=11.0, 7.0, 3.4 Hz, 1H), 2.37 (s, 3H), 1.95-1.87 (m, 1H), 1.89 (s, 1H), 1.79-1.62 (m, 3H), 1.37-1.13 (m, 4H).

### Synthesis Example 98

### 2-(4-Methoxy-pyridin-3-yl)-5-(4-methyl-6-methylsulfonyl-pyridin-3-yl)-4,5,6,7-tetrahydro-1H-pyrrolo[3,2-c]pyridine

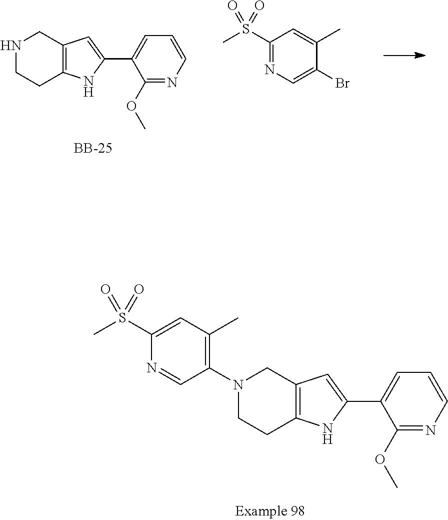

Example 98 was synthesized in analogy to the previous examples through the reaction of BB-25 (250 mg, 941 μmol) with 5-bromo-4-methyl-2-(methylsulfonyl)pyridine (282 mg, 1.13 μmol) (100 mg, 28%). LC-MS (method 2): m/z: [(M+H]⁺=399.2 (MW calc.=398.48); Rₜ=0.70 min.

¹H NMR (600 MHz, DMSO-d₆) δ (ppm)=10.86 (s, 1H) 8.39 (s, 1H), 7.97-7.89 (m, 2H), 7.83 (s, 1H), 7.00 (dd, J=7.5, 4.9 Hz, 1H), 6.55 (d, J=2.5 Hz, 1H), 4.18 (s, 2H), 3.97 (s, 3H), 3.38 (t, J=5.7 Hz, 2H), 3.19 (s, 3H), 2.82 (t, J=5.7 Hz, 2H), 2.42 (s, 3H) ppm.

### Synthesis Example 99

### 4-[2-(2,6-Difluoro-phenyl)-4,5,6,7-tetrahydro-1H-pyrrolo[3,2-c]pyridin-5-yl]-5-methyl-2-(1-methyl-1H-pyrazol-3-yl)-thiazole

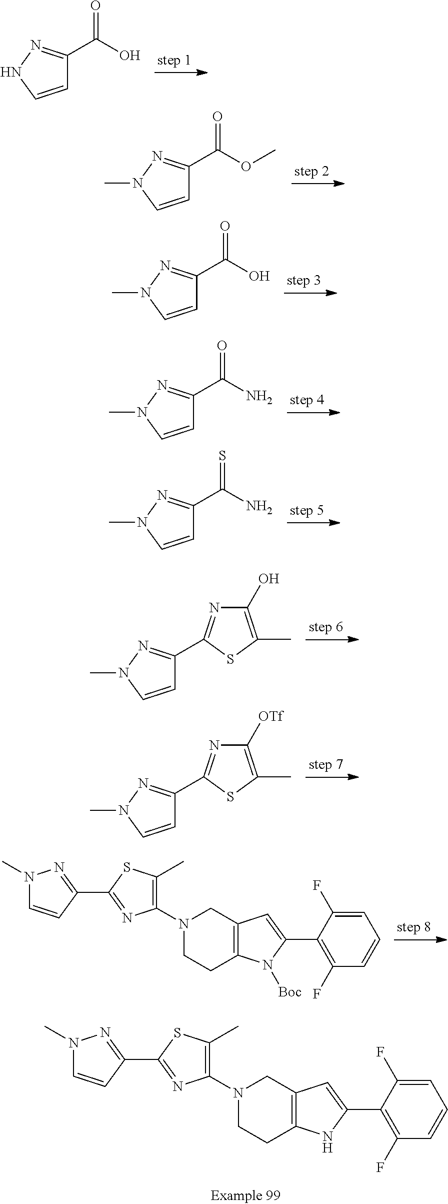

Step 1:

To a mixture of 1H-pyrazole-3-carboxylic acid (15.0 g, 134 mmol) and Cs₂CO₃ (109 g, 335 mmol) in CH₃CN (250 mL) was added CH₃I (29.2 ml, 469 mmol) and the RM was stirred at it for 14 h. The RM was filtered and filtrate was concentrated under reduced pressure, diluted with EtOAc and washed with water and brine before being dried. The solvent was evaporated under reduced pressure to give the crude product which was purified by CC (SiO₂; EtOAc/Hex) to yield the desired product (10.0 g, 53%).

LC-MS (Method 3): m/z [M+H]⁺=141.3 (MW calc. 140.14); Rₜ=1.55 min.

Step 2:

To a solution of step 1 intermediate (7.0 g, 50 mmol) in a mixture of THF (20 mL) and MeOH (20 mL) was added a solution of LiOH.H₂O (4.2 g, 100 mmol) in water (20 mL) at 0° C. The RM was stirred at it for 3 h. The RM was concentrated and subsequently diluted with water. The aqueous layer was acidified with sat. NaHSO₄ solution up to pH˜4-5 and extracted with EtOAc. The combined organic layers were dried and concentrated under reduced pressure to yield the desired product (6.2 g, 98%).

LC-MS (Method 3): m/z [M+H]⁺=127.2 (MW calc. 126.11); Rₜ=0.44 min.

Step 3:

To solution of step 2 intermediate (6.0 g, 47.6 mmol) in CH₂Cl₂ (50 mL) were added oxalyl chloride (6.12 mL, 71.4 mmol) and catalytic amount of DMF (0.5 mL) at 0° C. The RM was stirred at it for 2 h. The RM was concentrated under reduced pressure and residue was diluted with CH₂Cl₂. NH₃ gas was passed through RM at 0° C. for 30 min. After that the RM was concentrated under reduced pressure and residue was suspended in THF (50 ml) and filtered. The filtrate was concentrated under reduced pressure to yield the desired compound (3.5 g, 59%).

LC-MS (Method 3): m/z [M+H]⁺=126 (MW calc.=125.13); Rₜ=0.76 min.

Step 4:

To a solution of step 3 intermediate (3.5 g, 28.0 mmol) in THF (80 mL) was added Lawesson's reagent (13.6 g, 33.6 mmol) and the RM was stirred at it for 3 h. The RM was diluted with water and extracted with EtOAc. The combined organic layers were washed with brine and dried. The organic layer was concentrated under reduced pressure to give the crude product which was purified by CC (SiO₂, EtOAc/Hex) to yield the desired product (1.3 g, 33%).

Step 5:

A mixture of step 4 intermediate (1.8 g, 12.7 mmol), 2-bromo-propionic acid methyl ester (1.7 mL, 15.3 mmol) and pyridine (3 mL, 38.28 mmol) in dioxane (20 mL) was heated at reflux for 14 h. The RM was concentrated under reduced pressure and the residue was diluted with EtOAc, washed with water and brine and subsequently dried. The solvent was evaporated under reduced pressure and the residue was triturated with EtOH to yield the desired product (1.2 g, 48%).

LC-MS (Method 3): m/z [M+H]⁺=196.1 (MW calc.=195.24; Rₜ=2.18 min.

Step 6:

To a solution of the intermediate from step 5 (1.2 g, 6.15 mmol) in THF (20 mL) was added NaH (368 mg, 9.2 mmol) at 0° C. and the mixture was stirred at it for 30 min. N-phenyl-bis(trifluromethane-sulfonamide) (2.63 g, 7.38 mmol) was added and the RM stirred at it for 6 h. The RM was diluted with EtOAc and washed with water and brine. The organic layer was dried and concentrated under reduce pressure to give the crude compound which was purified by CC to yield the desired product (700 mg, 35%).

LC-MS (Method 3): m/z [M+H]⁺=328.10 (MW calc.=327.31; Rₜ=3.54 min.

Step 7:

A mixture of BB-2 (254 mg, 0.76 mmol), the intermediate from step 6 (250 mg, 0.76 mmol) and Cs₂CO₃ (0.74 g, 2.28 mmol) in toluene (6 mL) was degassed with Ar for 15 min followed by the addition of X-Phos (52 mg, 0.11 mmol) and Pd₂(dba)₃ (104 mg, 0.114 mmol). The resulting RM was heated at 110° C. in a sealed tube for 14 h. The RM was filtered through celite and the filtrate was concentrated under reduced pressure. The residue was purified by CC (SiO₂, EtOAc/Hex) to yield the desired product (149 mg, 18%).

LC-MS (Method 3): m/z [M+H]⁺=512.20 (MW calc.=511.59); Rₜ=4.12 min.

Step 8:

To a solution of the intermediate from step 7 (140 mg, 0.27 mmol) in MeOH (6 mL) was added K₂CO₃ (113 mg, 0.82 mmol) and the RM was heated at reflux for 16 h. The solvent was evaporated under reduced pressure and residue was diluted with EtOAc, washed with water and dried. The solvent was evaporated under reduced pressure to give the crude product which was purified by CC (SiO₂, EtOAc/Hex) to afford the title compound (70 mg, 63%).

LC-MS (Method 3): m/z [M+H]⁺=412.1 (MW calc.=411.47); Rₜ=3.66 min.

¹H NMR (DMSO-d₆, 400 MHz), δ (ppm)=10.77 (s, 1H), 7.78 (d, J=1.7 Hz, 1H), 7.25-7.20 (m, 1H), 7.14 (t, J=8.4 Hz, 2H), 6.62 (d, J=1.8 Hz, 1H), 6.31 (s, 1H), 4.09 (s, 2H), 3.88 (s, 3H), 3.34 (t, J=5.4 Hz, 2H), 2.82 (t, J=5.3 Hz, 2H), 2.33 (s, 3H).

### Synthesis Example 100

### 2-(2,6-Difluoro-phenyl)-5-[6-ethoxy-4-(trifluoromethyl)-pyridin-3-yl]-4,5,6,7-tetrahydro-1H-pyrrolo[3,2-c]pyridine

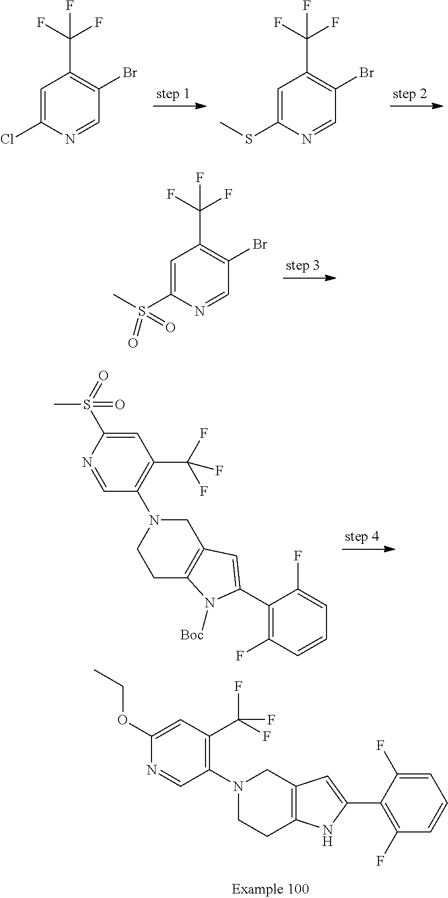

Step 1:

A mixture of 5-bromo-2-chloro-4-trifluoromethyl-pyridine (1.0 g, 3.8 mmol) and sodium thiomethoxide (400 mg, 5.79 mmol) in dioxane (15 mL) was heated at reflux for 14 h. The RM was concentrated under reduced pressure and diluted with EtOAc and subsequently washed with water and brine (60 mL). The organic layer was dried and concentrated under reduced pressure to give the crude product which was purified by CC (SiO₂, EtOAc/Hex) to yield the desired compound (800 mg, 77%).

Step 2:

To a cooled (0° C.) solution of 5-bromo-2-methylsulfanyl-4-trifluoromethyl-pyridine (800 mg, 2.94 mmol) in CH₂Cl₂ (20 mL) was added 70% m-CPBA (1.44 g, 5.88 mmol) and the RM was stirred at it for 4 h. The RM was diluted with CH₂Cl₂, washed with water, NaHCO₃ solution and brine. The organic layer was dried and concentrated under reduced pressure to give the crude product which was purified by CC (SiO₂, EtOAc/Hex) to yield 5-bromo-2-methanesulfonyl-4-trifluoromethyl-pyridine (600 mg, 67%).

Step 3:

The synthesis was performed in analogy to example 99 employing BB-2 and the intermediate from step 2 (180 mg, 27%).

Step 4:

To a solution of the intermediate from step 3 (180 mg, 320 μmol) in ethanol was added a freshly prepared 1 m NaOEt solution in ethanol (1.0 mL, 0.97 mmol) and heated at reflux for 3 h. The RM was concentrated under reduced pressure and diluted with EtOAc and washed with water and brine. The organic layer was dried and concentrated under reduced pressure to give the crude product which was purified by CC (SiO₂; 10% EtOAc/Hex) to yield the title compound (60 mg, 43%).

LC-MS (Method 3): m/z [M+H]⁺=424.2 (MW calc.=423.38); Rₜ=4.22 min.

¹H NMR (DMSO-d₆, 400 MHz), δ (ppm)=10.81 (s, 1H), 8.52 (s, 1H), 7.26-7.21 (m, 1H), 7.14 (t, J=8.5 Hz, 2H), 7.06 (s, 1H), 6.27 (s, 1H), 4.34 (q, J=7.0 Hz, 2H), 3.95 (s, 2H), 3.19 (t, J=5.2 Hz, 2H), 2.74 (t, J=5.12 Hz, 2H), 1.33 (t, J=7.0 Hz, 3H).

### Synthesis Example 101

### 2-(2,6-Difluoro-phenyl)-5-(5-fluoro-2-methyl-4-methylsulfonyl-phenyl)-4,5,6,7-tetrahydro-1H-pyrrolo[3,2-c]pyridine

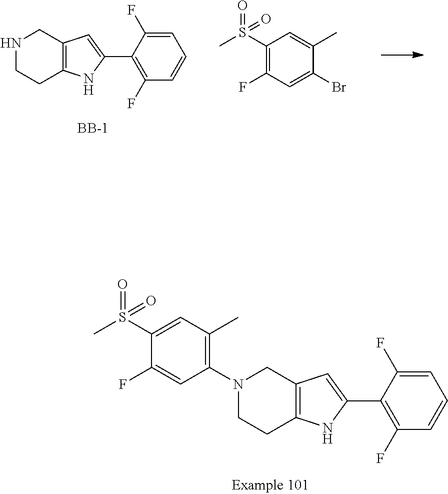

Example 101 was synthesized in analogy to the previous examples through the reaction of BB-13 (220 mg, 914 μmol) with 1-bromo-5-fluoro-2-methyl-4-(methylsulfonyl)benzene (303 mg, 1.10 mmol) (35 mg, 10%).

LC-MS (method 2): m/z: [(M+H]⁺=421.1 (MW calc.=420.45); Rₜ=0.80 min.

### Synthesis Example 102

### 6-(2-Cyclohexyl-4,5,6,7-tetrahydro-1H-pyrrolo[3,2-c]pyridin-5-yl)-5-methyl-nicotinonitrile

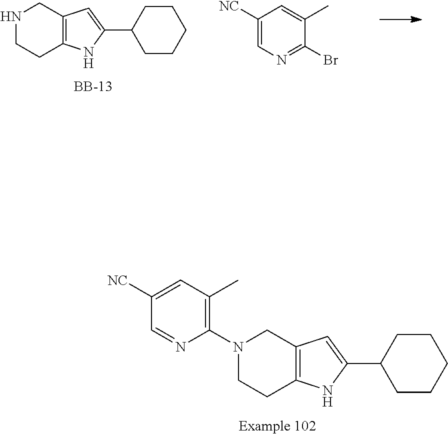

Example 102 was synthesized in analogy to the previous examples through the reaction of BB-1 (150 mg, 623 μmol) with 6-bromo-5-methylnicotinonitrile (147 mg, 747 mmol) (40 mg, 20%).

LC-MS (method 2): m/z: [(M+H]⁺=321.2 (MW calc.=320.43); Rₜ=0.87 min.

¹H NMR (400 MHz, DMSO-d₆) δ (ppm)=10.15 (d, J=2.5 Hz, 1H), 8.41 (t, J=1.6 Hz, 1H), 7.78 (dd, J=2.4, 1.2 Hz, 1H), 5.50 (d, J=2.4 Hz, 1H), 4.26 (s, 2H), 3.57 (t, J=5.6 Hz, 2H), 2.74 (t, J=5.7 Hz, 2H), 2.42 (tt, J=7.0, 3.5 Hz, 1H), 2.29 (s, 3H), 1.87 (dd, J=8.2, 4.4 Hz, 2H), 1.77-1.60 (m, 3H), 1.33-1.24 (m, 4H), 1.20-1.13 (m, 1H) ppm.

### Synthesis Example 103

### 5-(2-Cyclohexyl-4,5,6,7-tetrahydro-1H-pyrrolo[3,2-c]pyridin-5-yl)-6-methoxy-2,3-dihydro-benzo[b]thiophene 1,1-dioxide

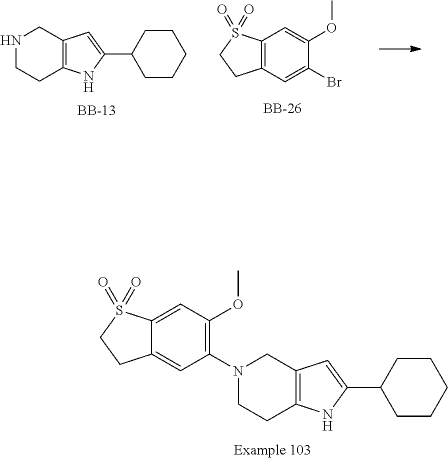

Example 103 was synthesized in analogy to the previous examples through the reaction of BB-13 (220 mg, 914 μmol) with BB-26 (303 mg, 1.10 mmol) (35 mg, 10%).

LC-MS (method 2): m/z: [(M+H]⁺=401.2 (MW calc.=400.53); Rₜ=1.00 min.

### Synthesis Example 104

### 2-(2,6-Difluoro-phenyl)-5-[4-methyl-6-(methylsulfonyl-methyl)-pyridin-3-yl]-4,5,6,7-tetrahydro-1H-pyrrolo[3,2-c]pyridine

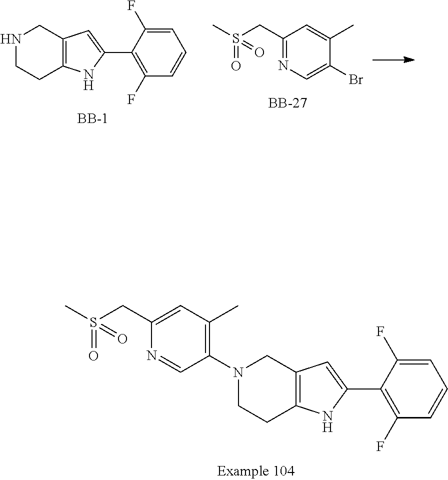

Example 104 was synthesized in analogy to the previous examples through the reaction of BB-1 (200 mg, 854 μmol) with BB-27 (270 mg, 1.03 mmol) (82 mg, 23%).

LC-MS (method 2): m/z: [(M+H]⁺=418.2 (MW calc.=417.47); Rₜ=0.68 min.

¹H NMR (400 MHz, DMSO-d₆) δ (ppm)=10.83 (d, J=2.5 Hz, 1H), 8.30 (s, 1H), 7.30 (s, 1H), 7.30-7.20 (m, 1H), 7.20-7.10 (m, 2H), 6.35 (q, J=2.1 Hz, 1H), 4.50 (s, 2H), 4.07 (s, 2H), 3.28 (t, J=5.7 Hz, 2H), 2.99 (s, 3H), 2.80 (t, J=5.7 Hz, 2H), 2.33 (s, 3H).

### Synthesis Example 105

### 2-(2,6-Difluoro-phenyl)-5-[4-(methoxymethyl)-2-methyl-phenyl]-4,5,6,7-tetrahydro-1H-pyrrolo[3,2-c]pyridine

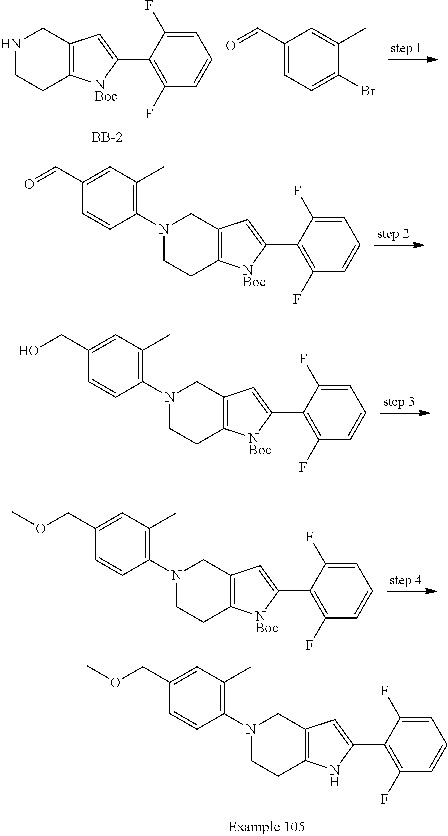

Step 1

was performed in analogy to previous examples through reaction of BB-2 (500 mg, 1.50 mmol) with 4-bromo-3-methylbenzaldehyde (590 mg, 2.99 mmol) (440 mg, 65).

Step 2:

To a solution of step 1 intermediate (200 mg, 442 mmol) in EtOH (1.8 mL) and THF (0.4 mL) was added NaBH₄ (147 mg, 3.89 mmol) in portions and the mixture was stirred overnight at rt. Water was added and the mixture was extracted with EtOAc. The organic layer was dried and the volatiles were removed under reduced pressure to yield the desired compound (184 mg, 92%).

Step 3:

To a solution of step 2 intermediate (150 mg, 330 μmol) in THF (1.4 mL) was added NaH (60% in mineral oil, 15 mg, 363 μmol) and the RM was stirred for 10 min. CH₃I (25 μL, 396 μmol) was added and the mixture was stirred for 1 h at rt. Water was added and the mixture was extracted with EtOAc. The combined organic layers were dried and the volatiles were removed under reduced pressure to yield the desired compound (110 mg, 71%).

Step 4:

A mixture of step 3 intermediate (109 mg, 234 μmol) and K₂CO₃ (645 mg, 4.68 mmol) in MeOH (1.9 mL) was stirred at 45° C. for 3 h and at it for 72 h. Saturated NH₄Cl was added and the mixture was extracted with EtOAc. The combined organic layers were dried and the volatiles were removed under reduced pressure. The residue was purified through chromatography (Interchim® cartridge 50SiHP/12 g, Cy/EtOAc) to yield the desired compound (56 mg, 65%).

LC-MS (method 2): m/z: [(M+H]⁺=369.2 (MW calc.=368.42); Rₜ=0.84 min.

¹H NMR (600 MHz, DMSO-d₆) b (ppm)=10.78 (s, 1H), 7.29-7.18 (m, 1H), 7.19-7.09 (m, 3H), 7.09 (d, J=1.2 Hz, 2H), 6.33 (q, J=2.1 Hz, 1H), 4.32 (s, 2H), 3.90 (s, 2H), 3.26 (s, 3H), 3.16 (t, J=5.6 Hz, 2H), 2.79 (t, J=5.7 Hz, 2H), 2.28 (s, 3H) ppm.

### Synthesis Example 106

### 5-(5-Cyclopropyl-2-methyl-2H-pyrazol-3-yl)-2-(2,6-difluoro-phenyl)-4,5,6,7-tetrahydro-1H-pyrrolo[3,2-c]pyridine

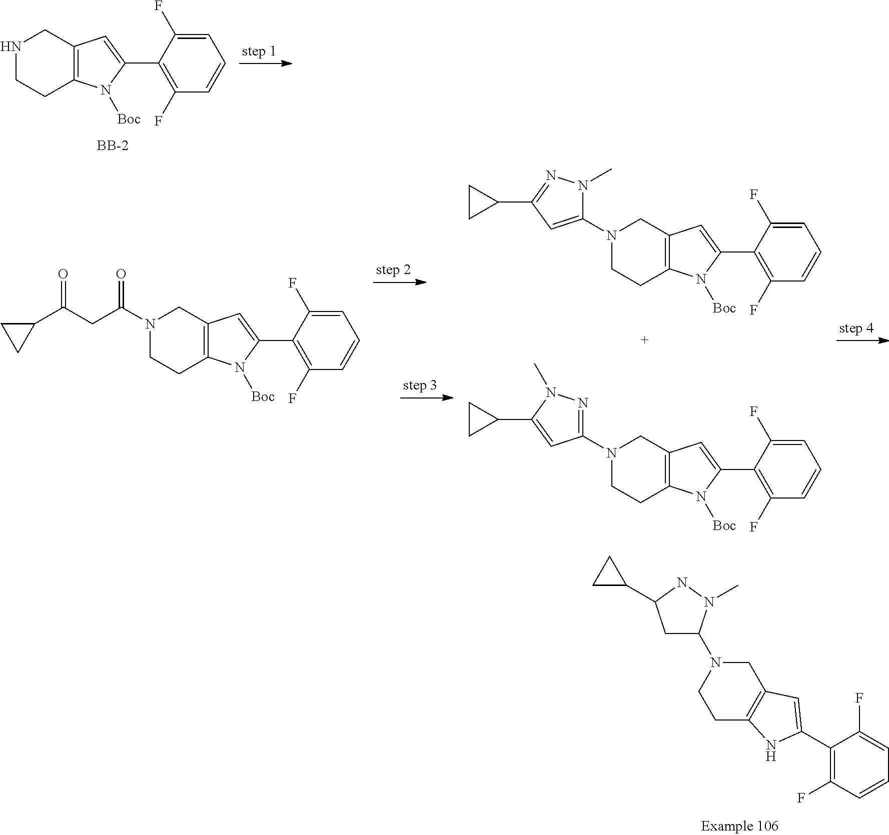

Step 1:

To a solution of BB-2 (300 mg, 0.898 mmol) and 3-cyclopropyl-3-oxo-propionic acid methyl ester (255 mg, 1.769 mmol) in toluene (6 mL) was added DMAP (22 mg, 0.179 mmol) and the suspension was irradiated in microwave at 150° C. for 30 min. The RM was concentrated under reduced pressure and crude mixture was purified by CC (SiO₂, EtOAc/Hex) to afford the desired compound.

LC-MS (Method 3): m/z [M+H]⁺=445.2 (MW calc.=444.47); Rₜ=3.49 min.

Step 2:

To a solution of the intermediate from step 1 (900 mg, 2.02 mmol) in toluene (20 mL) was added Lawesson's reagent (818 mg, 2.02 mmol) and pyridine (0.24 mL, 3.03 mmol) and the RM was heated at reflux for 5 h. The RM was cooled to it and diluted with EtOAc and subsequently washed with water and brine. The organic layer was dried and concentrated under reduced pressure to give the crude product which was purified by CC (SiO₂, EtOAc/Hex) to afford a mixture of 5-(3-cyclopropyl-3-oxo-thiopropionyl)-2-(2,6-difluoro-phenyl)-4,5,6,7-tetrahydro-pyrrolo[3,2-c]pyridine-1-carboxylic acid tert-butyl ester (major) and 5-(3-cyclopropyl-3-thioxo-thiopropionyl)-2-(2,6-difluoro-phenyl)-4,5,6,7-tetrahydro-pyrrolo[3,2-c]-pyridine-1-carboxylic acid tert-butyl ester (minor) which was used in the next step.

LC-MS (Method 3): m/z [M+H]⁺=461.2 and 477.3 (MW calc.=460.54 and 476.60); Rₜ=3.75 and 3.98 min.

Step 3:

To a mixture of 5-(3-cyclopropyl-3-oxo-thiopropionyl)-2-(2,6-difluoro-phenyl)-4,5,6,7-tetrahydro-pyrrolo[3,2-c]pyridine-1-carboxylic acid tert-butyl ester and 5-(3-cyclopropyl-3-thioxo-thiopropionyl)-2-(2,6-difluoro-phenyl)-4,5,6,7-tetrahydro-pyrrolo[3,2]pyridine-1-carboxylic acid tert-butyl ester (530 mg, 1.15 mmol) in EtOH (15 mL) was added methylhydrazine (0.3 mL, 5.76 mmol) and the RM was heated at reflux for 48 h. The RM was concentrated and the residue was purified by CC (SiO₂, EtOAc/Hex) to give a mixture of regioisomers. The regioisomers were separated by prep HPLC to yield 5-(5-cyclopropyl-2-methyl-2H-pyrazol-3-yl)-2-(2,6-difluoro-phenyl)-4,5,6,7-tetrahydro-pyrrolo[3,2-c]pyridine-1-carboxylic acid tert-butyl ester (110 mg, 21%) and 5-(5-cyclopropyl-1-methyl-1H-pyrazol-3-yl)-2-(2,6-difluoro-phenyl)-4,5,6,7-tetrahydro-pyrrolo[3,2-c]pyridine-1-carboxylic acid tert-butyl ester (90 mg, 17%).

LC-MS (Method 3): m/z [M+H]⁺=455.2 (MW calc.=454.51); Rₜ=3.88 min (5-(5-cyclopropyl-2-methyl-2H-pyrazol-3-yl)-2-(2,6-difluoro-phenyl)-4,5,6,7-tetrahydro-pyrrolo[3,2-c]pyridine-1-carboxylic acid tert-butyl ester).

LC-MS (Method 3): m/z [M+H]⁺=455.2 (MW calc.=454.51); Rₜ=3.88 min (5-(5-cyclopropyl-1-methyl-1H-pyrazol-3-yl)-2-(2,6-difluoro-phenyl)-4,5,6,7-tetrahydro-pyrrolo[3,2-c]pyridine-1-carboxylic acid tert-butyl ester).

Step 4:

5-(5-cyclopropyl-2-methyl-2H-pyrazol-3-yl)-2-(2,6-difluoro-phenyl)-4,5,6,7-tetrahydro-pyrrolo[3,2-c]pyridine-1-carboxylic acid tert-butyl ester (100 mg, 0.22 mmol) was transformed into the title compound in analogy to example 99 (70 mg, 89%).

LC-MS (Method 3): m/z [M+H]⁺=355.0 (MW calc. 354.40); Rₜ=3.55 min.

¹H NMR (DMSO-d₆, 400 MHz), δ (ppm)=10.82 (s, 1H), 7.28-7.20 (m, 1H), 7.14 (t, J=8.5 Hz, 2H), 6.28 (s, 1H), 5.59 (s, 1H), 3.86 (s, 2H), 3.55 (s, 3H), 3.12 (t, J=5.5 Hz, 2H), 2.78 (t, J=5.40 Hz, 2H), 1.78-1.68 (m, 1H), 0.78-0.75 (m, 2H), 0.59-0.56 (m, 2H).

### Synthesis Example 107

### 5-(5-Cyclopropyl-1-methyl-1H-pyrazol-3-yl)-2-(2,6-difluoro-phenyl)-4,5,6,7-tetrahydro-1H-pyrrolo[3,2-c]pyridine

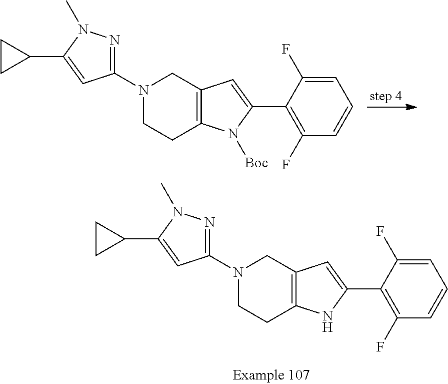

5-(5-cyclopropyl-1-methyl-1H-pyrazol-3-yl)-2-(2,6-difluoro-phenyl)-4,5,6,7-tetrahydro-pyrrolo[3,2-c]-pyridine-1-carboxylic acid tert-butyl ester was transformed into the title compound in analogy to example 99 (65 mg, 83%).

LC-MS (Method 3): m/z [M+H]⁺=354.9.0 (MW calc. 354.40); Rₜ=3.46 min.

¹H NMR (DMSO-d₆, 400 MHz), δ (ppm)=10.70 (s, 1H), 7.26-7.19 (m, 1H), 7.12 (t, J=8.5 Hz, 2H), 6.28 (s, 1H), 5.41 (s, 1H), 4.05 (s, 2H), 3.63 (s, 3H), 3.40 (t, J=5.5 Hz, 2H), 2.68 (t, J=5.4 Hz, 2H), 1.80-1.73 (m, 1H), 0.91-0.86 (m, 2H), 0.61-0.56 (m, 2H).

### Synthesis Example 108

### 2-(3-Chloro-pyridin-4-yl)-5-(4-methyl-6-methylsulfonyl-pyridin-3-yl)-4,5,6,7-tetrahydro-1H-pyrrolo[3,2-c]pyridine

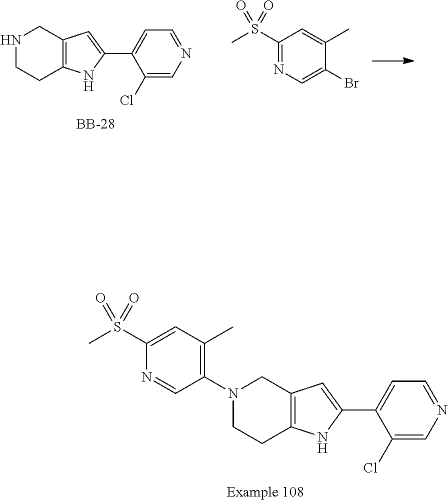

Example 108 was synthesized in analogy to the previous examples through the reaction of BB-28 (150 mg, 555 μmol) with 5-bromo-4-methyl-2-(methylsulfonyl)pyridine (167 mg, 666 μmol) (60 mg, 27%).

LC-MS (method 2): m/z: [(M+H]⁺=403.1 (MW calc.=402.90); Rₜ=0.57 min.

¹H NMR (600 MHz, DMSO-d₆) δ (ppm)=10.43 (s, 1H), 8.53 (s, 1H), 8.41-8.39 (m, 2H), 7.84 (s, 1H), 7.61 (d, J=5.2 Hz, 1H), 6.92 (d, J=2.4 Hz, 1H), 4.21 (s, 2H), 3.40 (t, J=5.7 Hz, 2H), 3.20 (s, 3H), 2.86 (t, J=5.7 Hz, 2H), 2.42 (s, 3H).

### Synthesis Example 109

### 5-[4-(Azetidin-1-ylsulfonyl)-2-methyl-phenyl]-2-(2,6-difluoro-phenyl)-4,5,6,7-tetrahydro-1H-pyrrolo[3,2-c]pyridine

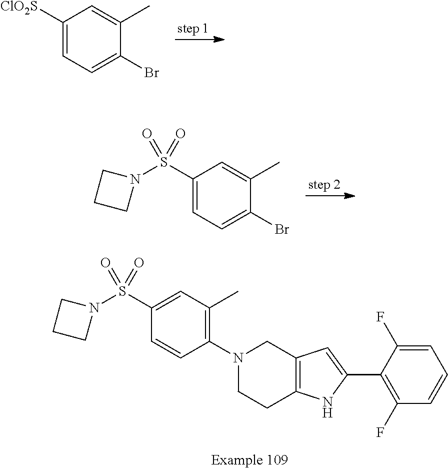 [SMILES: Cc1cc(S(=O)(=O)Cl)ccc1Br ; Cc1cc(S(=O)(=O)N2CCC2)ccc1Br ; Cc1cc(S(=O)(=O)N2CCC2)ccc1N1CCc2[nH]c(-c3c(F)cccc3F)cc2C1]

Step 1:

To a solution of 4-bromo-3-methylbenzene-1-sulfonyl chloride (200 mg, 741 μmol) and NEt₃ (112 μL, 816 μmol) in CH₂Cl₂ (1.7 mL) was added azetidine (55 μL, 816 μmol) and the solution was stirred at it overnight. The RM was diluted with CH₂Cl₂ and was washed with water and brine. The organic layer was dried and the volatiles were removed to give the desired compound (153 mg, 71%).

Step 2

was performed in analogy to the previous examples through the reaction of BB-1 (100 mg, 427 μmol) with step 1 intermediate (148 mg, 512 μmol) (121 mg, 64%).

LC-MS (method 2): m/z: [(M+H]⁺=444.2 (MW calc.=443.51); Rₜ=0.83 min.

¹H NMR (600 MHz, DMSO-d₆) δ (ppm)=10.85 (s, 1H), 7.59-7.54 (m, 2H), 7.30 (d, J=8.1 Hz, 1H), 7.28-7.22 (m, 1H), 7.15 (t, J=8.4 Hz, 2H), 6.36 (d, J=2.2 Hz, 1H), 4.09 (s, 2H), 3.64 (t, J=7.6 Hz, 4H), 3.33 (t, J=5.6 Hz, 2H), 2.83 (t, J=5.7 Hz, 2H), 2.40 (s, 3H), 2.02-1.95 (m, 2H).

### Synthesis Example 110

### 2-Cyclohexyl-5-(5-fluoro-2-methyl-4-methylsulfonyl-phenyl)-4,5,6,7-tetrahydro-1H-pyrrolo[3,2-c]pyridine

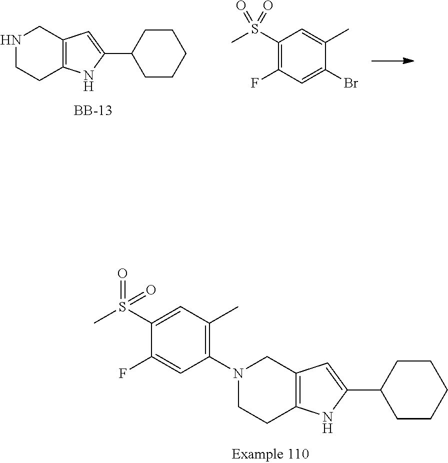

Example 108 was synthesized in analogy to the previous examples through the reaction of BB-13 (200 mg, 831 μmol) with 1-bromo-5-fluoro-2-methyl-4-(methylsulfonyl)benzene (266 mg, 997 μmol) (23 mg, 7%).

LC-MS (method 2): m/z: [(M+H]⁺=391.2 (MW calc.=390.51); Rₜ=0.85 min.

¹H NMR (400 MHz, DMSO-d₆) ti (ppm)=10.19 (s, 1H), 7.55 (d, J=8.5 Hz, 1H), 7.01 (d, J=13.1 Hz, 1H), 5.51 (d, J=2.1 Hz, 1H), 3.96 (s, 2H), 3.27 (t, J=5.7 Hz, 2H), 3.22 (s, 3H), 2.68 (t, J=5.7 Hz, 2H), 2.48-2.40 (m, 1H), 1.95-1.84 (m, 2H), 1.78-1.61 (m, 3H), 1.37-1.22 (m, 5H).

### Synthesis Example 111

### 2-(2,6-Difluoro-phenyl)-5-[4-methyl-6-(trifluoromethyloxy)-pyridin-3-yl]-4,5,6,7-tetrahydro-1H-pyrrolo[3,2-c]pyridine

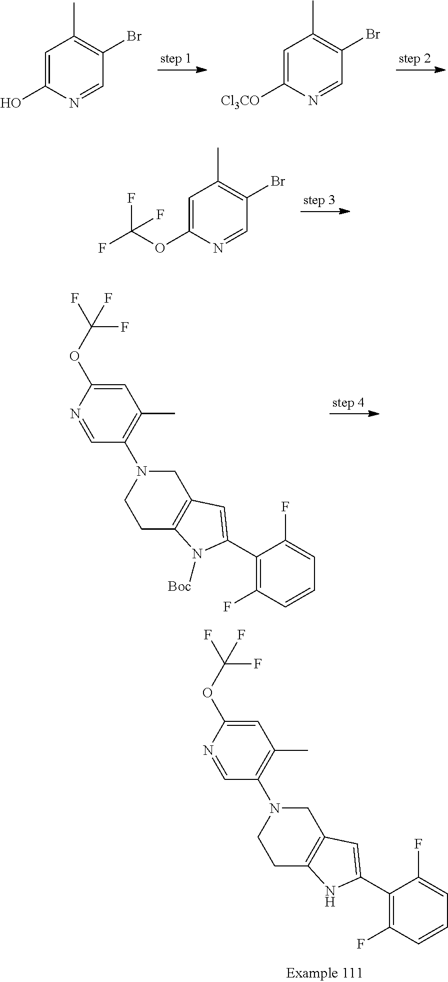

Step 1:

To a suspension of 5-bromo-4-methyl-pyridin-2-ol (5.0 g, 26.6 mmol) in 5% aq. NaOH (26 mL) was added drop-wise a solution of thiophosgene (2.4 ml, 31.9 mmol) in CHCl₃ (20 mL) at 0° C. and the RM was stirred at 0° C. for 2 h. The RM was diluted with CHCl₃ and the layers were separated. The aqueous layer was extracted with CHCl₃. The combined organic layers were washed with 1N HCl and water and dried and filtered. Cl₂ gas was passed through the filtrate until the RM was warmed and stirred for 2 h. Cl₂ gas again was passed through the RM until the yellow solution was formed and the RM was stirred at it for 24 h. The excess Cl₂ gas was removed by passing Ar through the RM. The RM was concentrated under reduced pressure to give crude 5-bromo-4-methyl-2-trichloromethoxy-pyridine (7.5 g) which was used in the next step without purification.

Step 2:

5-bromo-4-methyl-2-trichloromethoxy-pyridine (7.5 g, 24.67 mmol) was added to pre-heated mixture of SbF₃ (8.78 g, 49.3 mmol) and SbCl₅ (468 μL, 3.70 mmol) and the RM was heated at 150° C. for 14 h. The RM was diluted with CH₂Cl₂, basified with sat.NaHCO₃ solution to pH-8-9 and washed with 20% KF solution and dried. The solvent was evaporated under reduced pressure at 0-5° C. (compound is volatile) to give the crude product which was purified by CC (SiO₂, Et₂O/Hex) to yield 5-bromo-4-methyl-2-trifluoromethoxy-pyridine (150 mg, 2.3%).

Step 3:

The synthesis was performed in analogy to step 7 of example 99 employing the intermediate of step 2 to yield the desired product (90 mg, 20%).

LC-MS (Method 3): m/z [M+H]⁺=510.2 (MW calc.=509.47); Rₜ=2.57 min.

Step 4:

The intermediate from step 3 was transformed into the title compound in analogy to example 99 (35 mg, 50%).

LC-MS (Method 3): m/z [M+H]⁺=410.2 (MW calc.=409.35); Rₜ=3.92 min.

¹H NMR (DMSO-d₆, 400 MHz), δ (ppm)=10.85 (s, 1H), 8.04 (s, 1H), 7.27-7.21 (m, 1H), 7.17-7.13 (m, 3H), 6.32 (s, 1H), 4.03 (s, 2H), 3.24 (t, J=5.5 Hz, 2H), 2.78 (t, J=5.3 Hz, 2H), 2.36 (s, 3H).

### Synthesis Example 112

### 2-(2,6-Difluoro-phenyl)-5-[4-methoxy-6-(trifluoromethyl)-pyridin-3-yl]-4,5,6,7-tetrahydro-1H-pyrrolo[3,2-c]pyridine

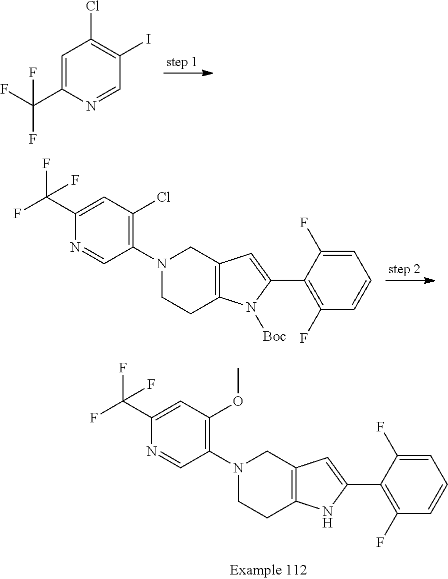

Step 1:

A mixture of BB-2 (1.0 g, 2.99 mmol), 4-chloro-5-iodo-2-trifluoromethyl-pyridine (919 mg, 2.99 mmol) and Cs₂CO₃ (2.9 g, 8.98 mmol) in toluene (30 mL) was degassed with Ar for 15 min. Pd(OAc)₂ (67 mg, 0.30 mmol) and BINAP (185 mg, 0.30 mmol) were added to the RM and heated at 110° C. for 16 h. The RM was filtered through celite, washed with CH₂Cl₂ and the filtrate was concentrated under reduced pressure to give the crude product which was purified by CC (SiO₂, EtOAc/Hex) to yield the desired product (610 mg, 40%).

LC-MS (Method 3): m/z [M+H]⁺=514.2 (MW calc.=513.89); Rₜ=4.30 min.

Step 2:

To a solution of the intermediate from step 1 (150 mg, 290 μmol) in MeOH (3 mL) was added a 25% NaOMe solution in MeOH (0.2 mL, 877 μmol) and the RM was heated at reflux for 2 h. The RM was concentrated under reduced pressure, diluted with EtOAc, and washed with water and brine. The organic layer was dried and concentrated under reduced pressure to give the crude product which was purified by CC (SiO₂, EtOAc/Hex) to yield the title compound (55 mg, 46%).

LC-MS (Method 3): m/z [M+H]⁺=410.3 (MW calc.=409.35); Rₜ=3.72 min.

¹H NMR (DMSO-d₆, 400 MHz), δ (ppm)=10.83 (s, 1H), 8.20 (s, 1H), 7.39 (s, 1H), 7.26-7.21 (m, 1H), 7.14 (t, J=8.3 Hz, 2H), 6.33 (s, 1H), 4.20 (s, 2H), 3.99 (s, 3H), 3.49 (t, J=5.44 Hz, 2H), 2.76 (t, J=5.2 Hz, 2H).

### Synthesis Example 113

### 5-[4-Cyclopropyl-6-(trifluoromethyl)-pyridin-3-yl]-2-(2,6-difluoro-phenyl)-4,5,6,7-tetrahydro-1H-pyrrolo[3,2-c]pyridine

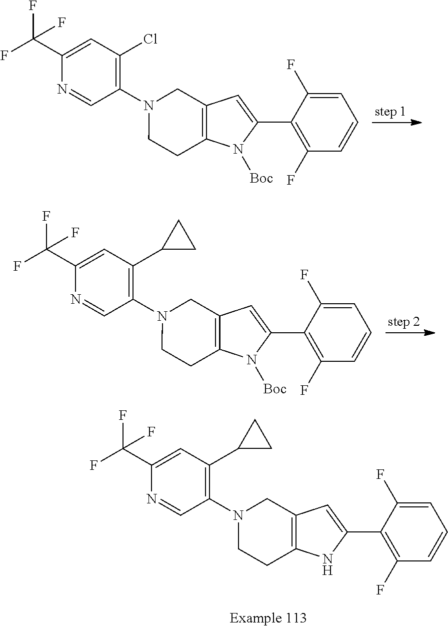

Step 1:

A mixture of the intermediate from step 1 of the synthesis of example 112 (300 mg, 583 μmol), potassium cyclopropyltrifluoroborate (129 mg, 875 μmol) and Cs₂CO₃ (947 mg, 2.92 mmol) in toluene (16 mL) and water (4 mL) was degassed through purging with Ar for 30 min. Pd(OAc)₂ (13 mg, 58 μmol) and di-(1-adamantyl)-n-butylphosphine (41 mg, 116 μmol) were added to the RM and heated at 100° C. for 16 h. The RM was diluted with EtOAc and washed with water and brine. The organic layer was dried and concentrated under reduced pressure to give the crude product which was purified by CC (SiO₂, EtOAc/Hex) to yield the desired product (270 mg, 89%).

LC-MS (Method 3): m/z [M+H]⁺=520.2 (MW calc.=519.51); Rₜ=4.46 min.

Step 2:

The intermediate from step 1 was transformed into the title compound in analogy to previous examples (90 mg, 62%).

LC-MS (Method 3): m/z [M+H]⁺=419.9 (MW calc.=419.39); Rₜ=3.95 min.

¹H NMR (DMSO-d₆, 400 MHz), δ (ppm)=10.88 (s, 1H), 8.40 (s, 1H), 7.27-7.22 (m, 1H), 7.17-7.13 (m, 3H), 6.35 (s, 1H), 4.20 (s, 2H), 3.47 (t, J=5.5 Hz, 2H), 2.84 (t, J=5.44 Hz, 2H), 2.23-2.19 (m, 1H), 1.19-1.14 (m, 2H), 0.99-0.95 (m, 2H).

### Synthesis Example 114

### 4-[2-(2,6-Difluoro-phenyl)-4,5,6,7-tetrahydro-1H-pyrrolo[3,2-c]pyridin-5-yl]-N,3-dimethyl-benzenesulfonic acid amide

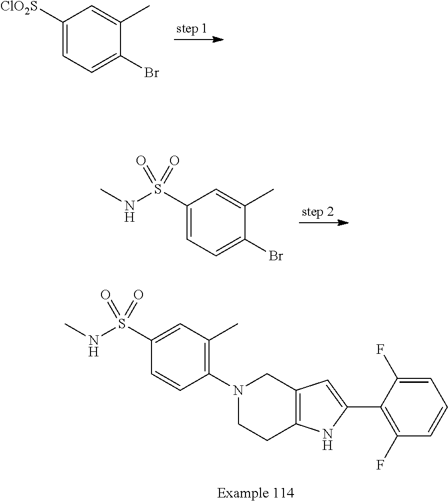

Example 114 was synthesized in analogy to example 109 in two steps (35% over two steps). LC-MS (Method 2): m/z [M+H]⁺=418.1 (MW calc.=417.47); Rₜ=0.78 min.

¹H NMR (DMSO-d₆, 400 MHz), δ (ppm)=10.82 (s, 1H), 7.58-7.48 (m, 2H), 7.30-7.08 (m, 4H), 6.34 (s, 1H), 4.03 (s, 2H), 3.57 (s, 1H), 3.27 (t, J=5.7 Hz, 2H), 2.81 (t, J=5.7 Hz, 2H), 2.39 (d, J=5.3 Hz, 3H), 2.36 (s, 3H).

### Synthesis Example 115

### 5-(4-Methyl-6-methylsulfonyl-pyridin-3-yl)-2-tetrahydro-pyran-3-yl-4,5,6,7-tetrahydro-1H-pyrrolo[3,2-c]pyridine

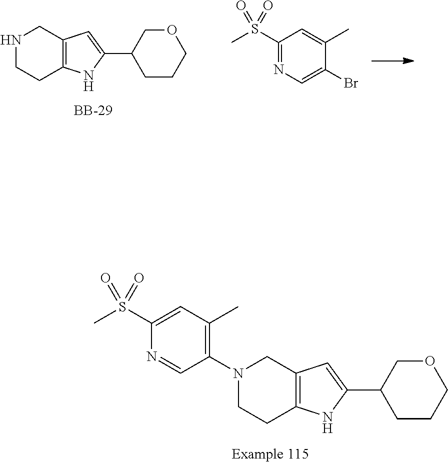

Example 115 was synthesized in analogy to the previous examples through the reaction of BB-29 (150 mg, 618 μmol) with 5-bromo-4-methyl-2-(methylsulfonyl)pyridine (185 mg, 741 μmol) (50 mg, 22%). LC-MS (method 2): m/z: [(M+H]⁺=376.2 (MW calc.=375.49); Rₜ=0.67 min.

¹H NMR (400 MHz, DMSO-d₆) δ (ppm)=10.33 (s, 1H), 8.35 (s, 1H), 7.81 (s, 1H), 5.59 (d, J=2.3 Hz, 1H), 4.07 (s, 2H), 3.91-3.80 (m, 2H), 3.32 (t, 2H), 3.23.3.17 (m, 4H), 2.74-2.64 (m, 3H), 2.39 (s, 3H), 2.01-1.93 (m, 1H), 1.67-1.54 (m, 3H).

### Synthesis Example 116

### 2-(2,6-Difluoro-phenyl)-5-[4-ethoxy-6-(trifluoromethyl)-pyridin-3-yl]-4,5,6,7-tetrahydro-1H-pyrrolo[3,2-c]pyridine

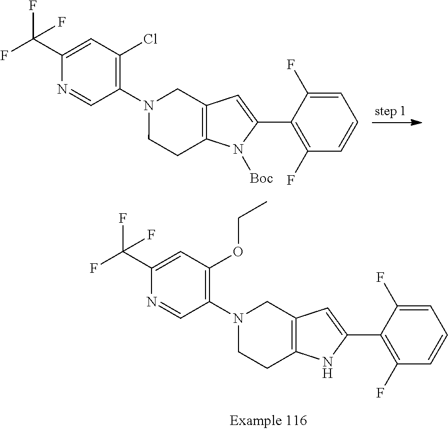

Step 1:

To a solution of the intermediate from step 1 of the synthesis of example 112 (250 mg, 0.48 mmol) in EtOH (3 mL) was added a 21% solution of NaOEt in EtOH (0.6 ml, 1.94 mmol) and the RM was heated at reflux for 48 h. The RM was concentrated under reduced pressure, diluted with EtOAc and subsequently washed with water and brine. The organic layer was dried and concentrated under reduced pressure to give the crude product which was purified by CC (SiO₂; 20% EtOAc/Hex) to yield the title compound (90 mg, 44%).

LC-MS (Method 3): m/z [M+H]⁺=424.0 (MW calc.=423.38); Rₜ=3.80 min.

¹H NMR (DMSO-d₆, 400 MHz), δ (ppm)=10.81 (s, 1H), 8.19 (s, 1H), 7.35 (s, 1H), 7.28-7.21 (m, 1H), 7.14 (t, 2H, J=8.52 Hz), 6.34 (s, 1H), 4.28-4.21 (m, 4H), 3.50 (t, 2H, J=5.32 Hz), 2.76 (t, 2H, J=4.96 Hz), 1.42 (t, 3H, J=6.88 Hz).

### Synthesis Example 117

### 4-[[4-[2-(2,6-Difluoro-phenyl)-4,5,6,7-tetrahydro-1H-pyrrolo[3,2-c]pyridin-5-yl]-3-methyl-phenyl]sulfonyl]-morpholine

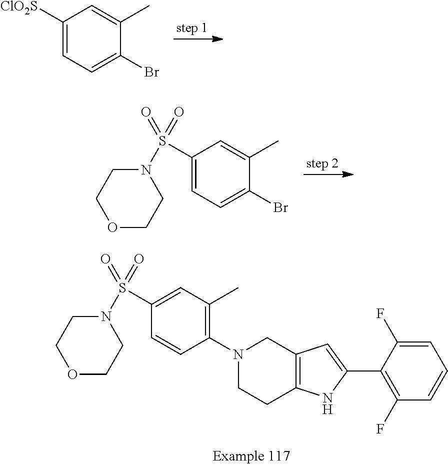

Example 117 was synthesized in analogy to example 109 in two steps (44% over two steps). LC-MS (Method 2): m/z [M+H]⁺=474.2 (MW calc.=473.54); Rₜ=0.86 min.

¹H NMR (DMSO-d₆, 400 MHz), δ (ppm)=10.84 (s, 1H), 7.54-7.47 (m, 2H) 7.28-7.22 (m, 2H), 7.18-7.12 (m, 2H), 6.35 (s, 1H), 4.08 (s, 2H), 3.65-3.60 (m, 4H), 3.31 (t, 2H), 2.87-2.70 (m, 6H), 2.39 (s, 3H).

### Synthesis Example 118

### 2-Cyclopentyl-5-(4-methyl-6-methylsulfonyl-pyridin-3-yl)-4,5,6,7-tetrahydro-1H-pyrrolo[3,2-c]pyridine

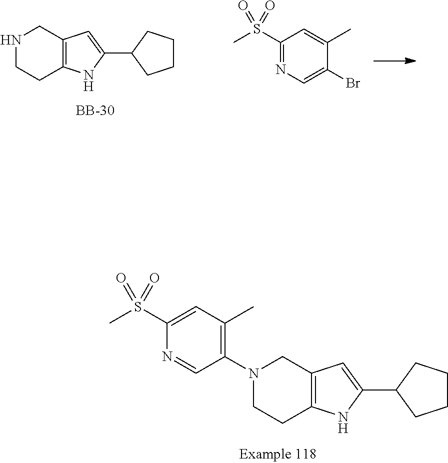

Example 118 was synthesized in analogy to the previous examples through the reaction of BB-30 (200 mg, 882 μmol) with 5-bromo-4-methyl-2-(methylsulfonyl)pyridine (264 mg, 1.06 μmol) (40 mg, 13%).

LC-MS (method 2): m/z: [(M+H]⁺=360.2 (MW calc.=359.49); Rₜ=0.79 min.

¹H NMR (600 MHz, DMSO-d₆) δ (ppm)=10.21 (s, 1H), 8.35 (s, 1H), 7.81 (s, 1H), 5.55 (d, J=2.3 Hz, 1H), 4.07 (s, 2H), 3.34-3.32 (m, 2H), 3.18 (s, 3H), 2.90 (quint, J=8.1 Hz, 1H), 2.68 (t, J=5.5 Hz, 2H), 2.39 (s, 3H), 1.95-1.88 (m, 2H), 1.73-1.64 (m, 2H), 1.63-1.48 (m, 4H).

### Synthesis Example 119

### 2-(2,6-Difluoro-phenyl)-5-[2-methyl-4-(piperidin-1-ylsulfonyl)-phenyl]-4,5,6,7-tetrahydro-1H-pyrrolo[3,2-c]pyridine

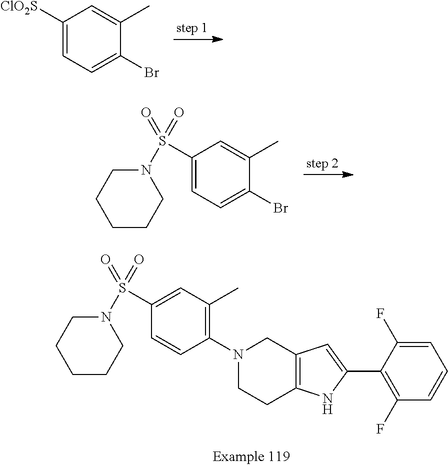

Example 119 was synthesized in analogy to example 109 in two steps (32% over two steps). LC-MS (Method 2): m/z [M+H]⁺=472.3 (MW calc.=471.56); Rₜ=0.94 min.

¹H NMR (DMSO-d₆, 400 MHz), δ (ppm)=10.83 (s, 1H), 7.51-7.45 (m, 2H), 7.28-7.22 (m, 2H), 7.18-7.12 (m, 2H), 6.35 (s, 1H), 4.06 (s, 2H), 3.32-3.26 (m, 2H), 2.89-2.84 (m, 4H), 2.81 (t, J=5.3 Hz, 2H), 2.37 (s, 3H), 1.57-1.51 (m, 4H), 1.38-1.33 (m, 2H).

### Synthesis Example 120

### N-Cyclopropyl-4-[2-(2,6-difluoro-phenyl)-4,5,6,7-tetrahydro-1H-pyrrolo[3,2-c]pyridin-5-yl]-N,3-dimethyl-benzenesulfonic acid amide

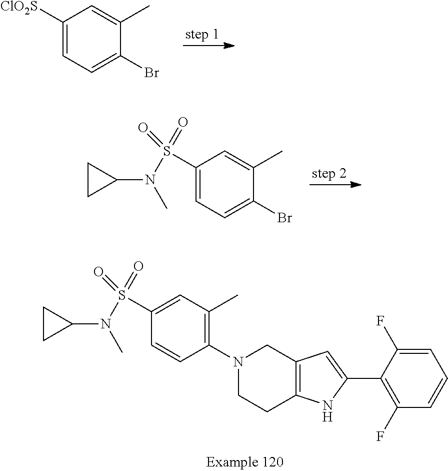 [SMILES: Cc1cc(S(=O)(=O)Cl)ccc1Br ; Cc1cc(S(=O)(=O)N(C)C2CC2)ccc1Br ; Cc1cc(S(=O)(=O)N(C)C2CC2)ccc1N1CCc2[nH]c(-c3c(F)cccc3F)cc2C1]

Example 120 was synthesized in analogy to example 109 in two steps (18% over two steps). LC-MS (Method 2): m/z [M+H]⁺=458.2 (MW calc.=457.54); Rₜ=0.92 min.

¹H NMR (DMSO-d₆, 400 MHz), δ (ppm)=10.84 (s, 1H), 7.61-7.53 (m, 2H), 7.28-7.21 (m, 2H), 7.18-7.11 (m, 2H), 6.35 (s, 1H), 4.07 (s, 2H), 3.36-3.27 (m, 2H), 2.82 (t, J=4.9 Hz, 2H), 2.64-2.58 (m, 3H), 2.40-2.35 (m, 3H), 1.84-1.77 (m, 1H), 0.75-0.64 (m, 4H).

### Synthesis Example 121

### 2-(2,6-Difluoro-phenyl)-5-[2-methyl-4-(pyrrolidin-1-ylsulfonyl)-phenyl]-4,5,6,7-tetrahydro-1H-pyrrolo[3,2-c]pyridine

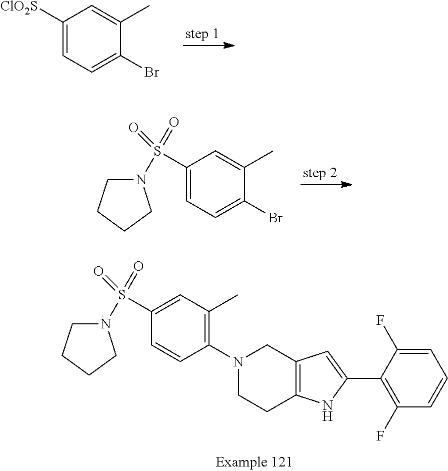

Example 121 was synthesized in analogy to example 109 in two steps (36% over two steps). LC-MS (Method 2): m/z [M+H]⁺=458.2 (MW calc.=457.54); Rₜ=0.89 min.

¹H NMR (DMSO-d₆, 400 MHz), δ (ppm)=10.83 (s, 1H), 7.60-7.54 (m, 2H), 7.29-7.21 (m, 2H), 7.18-7.11 (2H), 6.34 (s, 1H), 4.05 (s, 2H), 3.33-3.27 (m, 2H), 3.12 (t, J=6.8 t, J=5.3

### Synthesis Example 122

### 2-(4,4-Difluoro-cyclohexyl)-5-(4-methyl-6-methylsulfonyl-pyridin-3-yl)-4,5,6,7-tetrahydro-1H-pyrrolo[3,2-c]pyridine

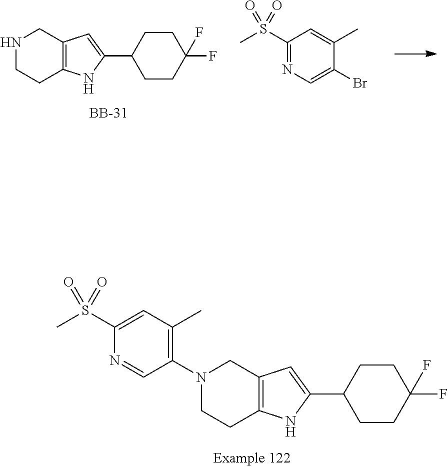

Example 122 was synthesized in analogy to the previous examples through the reaction of BB-31 (200 mg, 723 μmol) with 5-bromo-4-methyl-2-(methylsulfonyl)pyridine (216 mg, 1867 μmol) (70 mg, 24%). LC-MS (method 2): m/z: [(M+H]⁺=410.2 (MW calc.=409.49); Rₜ=0.76 min.

¹H NMR (400 MHz, DMSO-d₆) δ (ppm)=10.34 (s, 1H), 8.35 (s, 1H), 7.81 (s, 1H), 5.58 (s, 1H), 4.08 (s, 2H), 3.33-3.30 (m, 2H), 3.18 (s, 3H), 2.70-2.60 (m, 3H), 2.39 (s, 3H), 2.09-1.83 (m, 6H), 1.65-1.55 (m, 2H).

### Synthesis Example 123

### 2-(2,6-Difluoro-phenyl)-5-[2-methyl-5-[1-(trifluoromethyl)-cyclopropyl]-2H-pyrazol-3-yl]-4,5,6,7-tetrahydro-1H-pyrrolo[3,2-c]pyridine

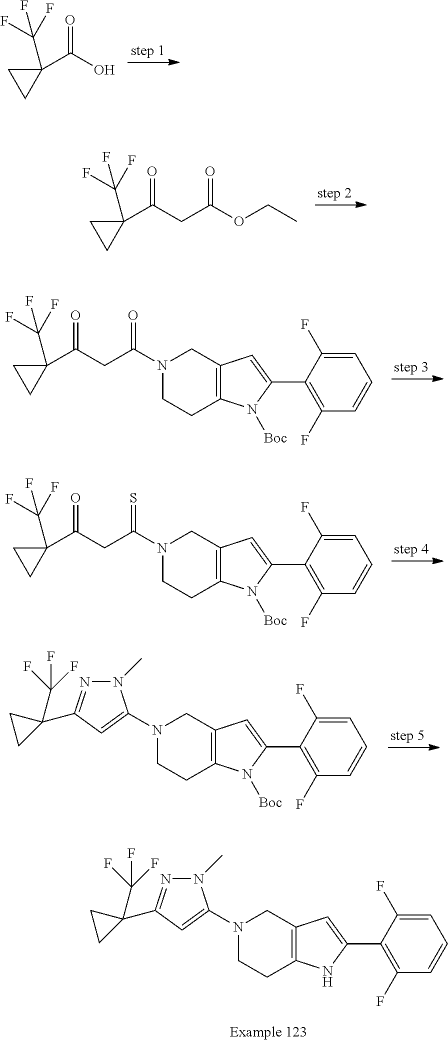 [SMILES: O=C(O)C1(C(F)(F)F)CC1 ; CCOC(=O)CC(=O)C1(C(F)(F)F)CC1 ; CC(C)(C)OC(=O)n1c(-c2c(F)cccc2F)cc2c1CCN(C(=O)CC(=O)C1(C(F)(F)F)CC1)C2 ; CC(C)(C)OC(=O)n1c(-c2c(F)cccc2F)cc2c1CCN(C(=S)CC(=O)C1(C(F)(F)F)CC1)C2 ; Cn1nc(C2(C(F)(F)F)CC2)cc1N1CCc2c(cc(-c3c(F)cccc3F)n2C(=O)OC(C)(C)C)C1 ; Cn1nc(C2(C(F)(F)F)CC2)cc1N1CCc2[nH]c(-c3c(F)cccc3F)cc2C1]

Step 1:

NEt₃ (5.78 mL, 41.6 mmol) and MgCl₂ (3.08 g, 32.5 mmol) were added to a suspension of potassium ethylmalonate (4.63 g, 27.3 mmol) in CH₃CN (25 mL) and stirred at it for 2 h. A pre-stirred mixture of CD (2.52 g, 15.6 mmol) and 1-trifluoromethyl-cyclopropanecarboxylic acid (2.0 g, 13.0 mmol) in CH₃CN (25 mL) was added at 0° C. and the resulting RM stirred at it for 14 h. The RM was diluted with EtOAc and washed with water and brine before being dried. Solvent was evaporated under reduced pressure to give the crude compound which was purified by CC (SiO₂, EtOAc/Hex) to yield the desired product (1.4 g, 48%).

LC-MS (Method 3): m/z [M+H]⁺=225.0 (MW calc.=224.18); Rₜ=3.23 min (higher mass present).

Step 2:

To a mixture of BB-2 (1.0 g, 2.99 mmol) and 3-oxo-3-(1-trifluoromethyl-cyclopropyl)-propionic acid ethyl ester (1.34 g, 5.98 mmol) in toluene (15 mL) was added DMAP (73 mg, 0.60 mmol) and the RM was heated at reflux for 14 h. The RM was diluted with EtOAc and washed with water and brine before being dried. The solvent was evaporated under reduced pressure to give the crude product which was purified by CC (SiO₂; 20% EtOAc/Hex) to yield the desired product (1.05 g, ˜64% pure (LCMS)) that was used in the next step.

LC-MS (Method 3): m/z [M+H]⁺=513.2 (MW calc.=512.47); Rₜ=3.75 min.

Step 3:

To a solution of the intermediate from step 2 (500 mg, 0.97 mmol) in toluene (10 mL) were added Lawesson's reagent (433 mg, 1.07 mmol) and pyridine (0.117 mL, 1.46 mmol) and the resulting RM was heated at reflux for 30 min. The RM was diluted with EtOAc washed with sat. NaHCO₃ solution, water and brine and subsequently dried. The solvent was evaporated under reduced pressure to give the desired compound along with impurities (500 mg) which was used in the next step without purification.

LC-MS (Method 3): m/z [M+H]⁺=529.0 (MW calc.=528.54); Rₜ=3.83 min.

Step 4:

To a solution of the intermediate from step 3 (500 mg, 0.95 mmol) in EtOH (10 mL) was added methylhydrazine (0.25 ml, 4.73 mmol) and the RM was heated at reflux for 14 h. The RM was concentrated under reduced pressure and residue was dissolved in EtOAc and washed with water and brine. The organic layer was dried and concentrated under reduced pressure to give the crude product which was purified by CC (SiO₂, EtOAc/Hex) to yield the desired product (120 mg, 24%, two steps).

LC-MS (Method 3): m/z [M+H]⁺=523.3 (MW calc.=522.51); Rₜ=4.15 min.

Step 5:

The intermediate from step 4 was transformed into the title compound in analogy to previous examples (55 mg, 57%).

LC-MS (Method 3): m/z [M+H]⁺=423.4 (MW calc.=422.39); Rₜ=3.83 min.

¹H NMR (DMSO-d₆, 400 MHz), δ (ppm)=10.83 (s, 1H), 7.28-7.22 (m, 1H), 7.14 (t, 2H, J=8.44 Hz), 6.30 (s, 1H), 5.94 (s, 1H), 3.91 (s, 2H), 3.62 (s, 3H), 3.17 (t, 2H, J=5.56 Hz), 2.80 (t, 2H, J=5.28 Hz), 1.26-1.17 (m, 4H).

### Synthesis Example 124

### 2-(2,6-Difluoro-phenyl)-5-[2-ethyl-5-[1-(trifluoromethyl)-cyclopropyl]-2H-pyrazol-3-yl]-4,5,6,7-tetrahydro-1H-pyrrolo[3,2-c]pyridine

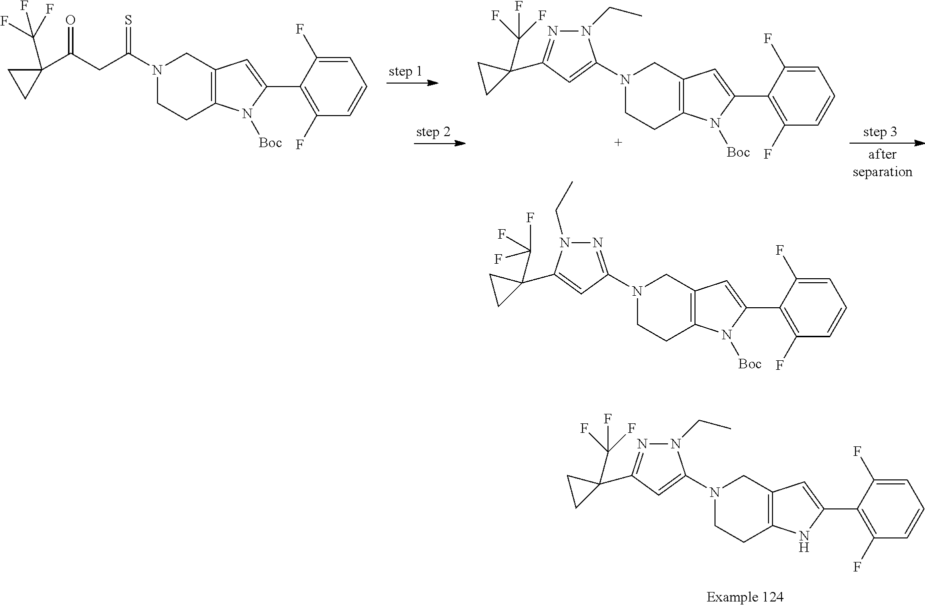 [SMILES: CC(C)(C)OC(=O)n1c(-c2c(F)cccc2F)cc2c1CCN(C(=S)CC(=O)C1(C(F)(F)F)CC1)C2 ; CCn1nc(C2(C(F)(F)F)CC2)cc1N1CCc2c(cc(-c3c(F)cccc3F)n2C(=O)OC(C)(C)C)C1 ; CCn1nc(N2CCc3c(cc(-c4c(F)cccc4F)n3C(=O)OC(C)(C)C)C2)cc1C1(C(F)(F)F)CC1 ; CCn1nc(C2(C(F)(F)F)CC2)cc1N1CCc2[nH]c(-c3c(F)cccc3F)cc2C1]

Step 1:

To a solution of the intermediate of step 3 from example 123 (1.65 g, 3.12 mmol) in EtOH (60 mL) was added ethylhydrazine oxalate (2.34 g, 15.6 mmol) and the RM was heated at reflux for 16 h. The RM was concentrated under reduced pressure and the residue was dissolved in EtOAc and washed with water and brine. The organic layer was dried and concentrated under reduced pressure to give the crude material (1.59 g) which was used in next the step without purification.

Step 2:

To a solution of the material from step 1 (1.59 g, 3.12 mmol) in DMF (20 mL) was added NaH (125 mg, 3.12 mmol, 60% in mineral oil) at 0° C. and the RM was stirred for 15 min. Ethyl iodide (0.128 mL, 0.155 mmol) was added and the RM stirred at it for 1 h. Ice cold water was added to the RM and extracted with EtOAc. The combined organic layers were washed with water and brine and subsequently dried. The solvent was evaporated under reduced pressure to give the crude compound which was purified by CC (SiO₂, EtOAc/Hex) to yield 2-(2,6-difluoro-phenyl)-5-[2-ethyl-5-(1-trifluoromethyl-cyclo-propyl)-2H-pyrazol-3-yl]-4,5,6,7-tetrahydro-pyrrolo[3,2-c]pyridine-1-carboxylic acid tert-butyl ester (90 mg, 5% after 3 steps) and 2-(2,6-difluoro-phenyl)-5-[1-ethyl-5-(1-trifluoromethyl-cyclopropyl)-1H-pyrazol-3-yl]-4,5,6,7-tetrahydro-pyrrolo[3,2-c]pyridine-1-carboxylic acid tert-butyl ester (150 mg, 9% after 3 steps).

LC-MS (Method 3): m/z [M+H]⁺=537.0 (MW calc.=536.54); Rₜ=2.51 min (2-(2,6-difluoro-phenyl)-5-[2-ethyl-5-(1-trifluoromethyl-cyclopropyl)-2H-pyrazol-3-yl]-4,5,6,7-tetrahydro-pyrrolo[3,2-c]pyridine-1-carboxylic acid tert-butyl ester).

LC-MS (Method 3): m/z [M+H]⁺=537.3 (MW calc.=536.54); Rₜ=4.33 min (2-(2,6-difluoro-phenyl)-5-[1-ethyl-5-(1-trifluoromethyl-cyclopropyl)-1H-pyrazol-3-yl]-4,5,6,7-tetrahydro-pyrrolo[3,2-c]pyridine-1-carboxylic acid tert-butyl ester).

Step 3:

2-(2,6-difluoro-phenyl)-5-[2-ethyl-5-(1-trifluoromethyl-cyclopropyl)-2H-pyrazol-3-yl]-4,5,6,7-tetrahydro-pyrrolo[3,2-c]pyridine-1-carboxylic acid tert-butyl ester was transformed into the title compound in analogy to previous examples (55 mg, 75%).

LC-MS (Method 3): m/z [M+H]⁺=437.3 (MW calc.=436.42); Rₜ=3.97 min.

¹H NMR (DMSO-d₆, 400 MHz), δ (ppm)=10.83 (s, 1H), 7.28-7.22 (m, 1H), 7.14 (t, 2H, J=8.38 Hz), 6.29 (s, 1H), 5.98 (s, 1H), 3.95 (q, 2H, J=7.24 Hz), 3.89 (s, 2H), 3.14 (t, 2H, J=5.62 Hz), 2.80 (t, 2H, J=5.26 Hz), 1.31 (t, 3H, J=7.20 Hz), 1.26-1.19 (m, 4H).

### Synthesis Example 125

### 2-(2,6-Difluoro-phenyl)-5-[1-ethyl-5-[1-(trifluoromethyl)-cyclopropyl]-1H-pyrazol-3-yl]-4,5,6,7-tetrahydro-1H-pyrrolo[3,2-c]pyridine

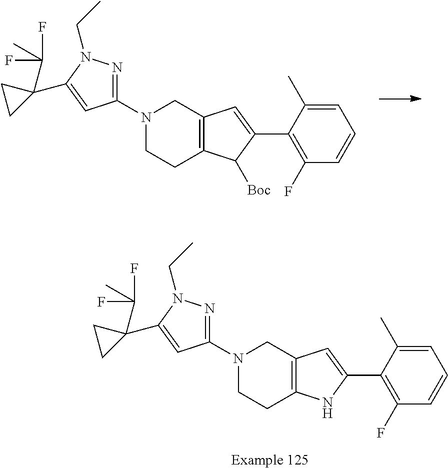 [SMILES: CCn1nc(N2CCC3=C(C=C(c4c(C)cccc4F)C3C(=O)OC(C)(C)C)C2)cc1C1(C(C)(F)F)CC1 ; CCn1nc(N2CCc3[nH]c(-c4c(C)cccc4F)cc3C2)cc1C1(C(C)(F)F)CC1]

2-(2,6-difluoro-phenyl)-5-[1-ethyl-5-(1-trifluoromethyl-cyclopropyl)-1H-pyrazol-3-yl]-4,5,6,7-tetrahydro-pyrrolo[3,2-c]pyridine-1-carboxylic acid tert-butyl was transformed into the title compound in analogy to previous examples (65 mg, 53%).

LC-MS (Method 2): m/z [M+H]⁺=437.2 (MW calc.=436.42); Rₜ=0.98 min.

¹H NMR (DMSO-d₆, 400 MHz), δ (ppm)=10.73 (s, 1H), 7.26-7.21 (m, 1H), 7.13 (t, J=8.4 Hz, 2H), 6.30 (s, 1H), 5.88 (s, 1H), 4.12 (s, 2H), 3.95 (q, J=7.2 Hz, 2H), 3.47 (t, J=5.5 Hz, 2H), 2.71 (t, J=4.78 Hz, 2H), 1.47-1.41 (m, 2H), 1.33 (t, J=7.16 Hz, 3H), 1.17 (s, 2H).

### Synthesis Example 126

### 5-(5-Cyclopropyl-2-ethyl-2H-pyrazol-3-yl)-2-(2,6-difluoro-phenyl)-4,5,6,7-tetrahydro-1H-pyrrolo[3,2-c]pyridine

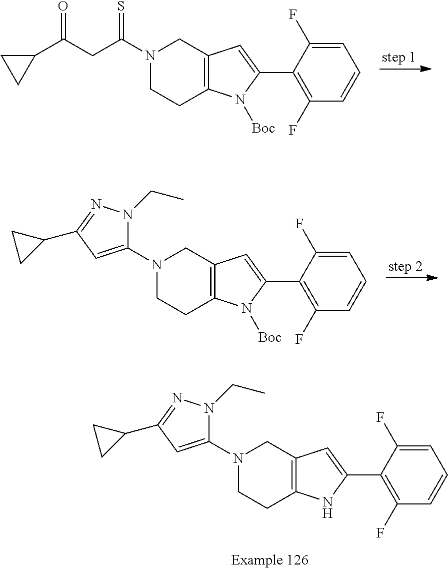 [SMILES: CC(C)(C)OC(=O)n1c(-c2c(F)cccc2F)cc2c1CCN(C(=S)CC(=O)C1CC1)C2 ; CCn1nc(C2CC2)cc1N1CCc2c(cc(-c3c(F)cccc3F)n2C(=O)OC(C)(C)C)C1 ; CCn1nc(C2CC2)cc1N1CCc2[nH]c(-c3c(F)cccc3F)cc2C1]

Step 1:

To a suspension of 5-(3-cyclopropyl-3-oxo-thiopropionyl)-2-(2,6-difluoro-phenyl)-4,5,6,7-tetrahydro-pyrrolo[3,2-c]pyridine-1-carboxylic acid tert-butyl ester (300 mg, 0.65 mmol, synthesized under conditions similar to example 106) in EtOH (5 mL) was added ethylhydrazine oxalate (489 mg, 3.26 mmol) and NEt₃ (0.8 mL, 6.52 mmol) and the RM was heated at reflux for 16 h. The RM was concentrated under reduced pressure to give the crude product which was purified by CC (SiO₂, EtOAc/Hex) to yield the desired product (85 mg, 28%).

LC-MS (Method 3): m/z [M+H]⁺=469.1 (MW calc.=468.54); Rₜ=4.03.

Step 2:

The intermediate from step 1 was transformed into the title compound in analogy to previous examples (42 mg, 63%).

LC-MS (Method 3): m/z [M+H]⁺=369.0 (MW calc.=368.42); Rₜ=3.66 min.

¹H NMR (DMSO-d₆, 400 MHz), δ (ppm)=10.82 (s, 1H), 7.28-7.22 (m, 1H), 7.14 (t, 2H, J=8.52 Hz), 6.28 (s, 1H), 5.60 (s, 1H), 3.91-3.84 (m, 4H), 3.09 (t, 2H, J=5.52 Hz), 2.78 (t, 2H, J=5.48 Hz), 1.78-1.74 (m, 1H), 0.80-0.76 (m, 2H), 0.60-0.57 (m, 2H).

### Synthesis Example 127

### 4-[2-(4,6-Dimethyl-pyridin-3-yl)-4,5,6,7-tetrahydro-1H-pyrrolo[3,2-c]pyridin-5-yl]-N,3-dimethyl-benzenesulfonic acid amide

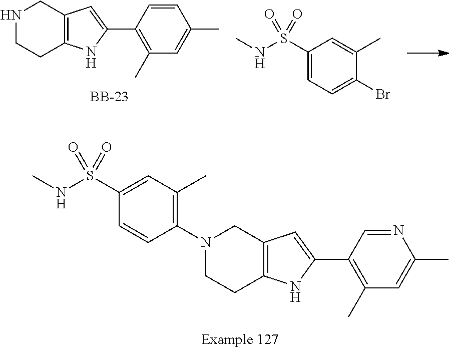

Example 127 was synthesized in analogy to the previous examples through the reaction of BB-23 (150 mg, 569 μmol) with step 1 intermediate of example 92 (150 mg, 568 μmol) (45 mg, 19%).

LC-MS (method 2): m/z: [(M+H]⁺=411.3 (MW calc.=410.53); Rₜ=0.53 min.

¹H NMR (400 MHz, DMSO-d₆) δ (ppm)=10.87 (d, J=1.8 Hz, 1H), 8.44 (s, 1H), 7.58-7.51 (m, 2H), 7.25-7.16 (m, 2H), 7.10 (s, 1H), 6.10 (d, J=2.3

### Synthesis Example 128

### 4-[2-(4,4-Difluoro-cyclohexyl)-4,5,6,7-tetrahydro-1H-pyrrolo[3,2-c]pyridin-5-yl]-N,3-dimethyl-benzenesulfonic acid amide

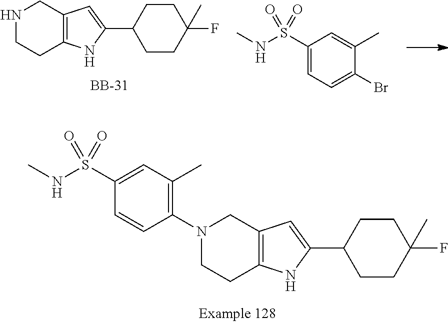

Example 128 was synthesized in analogy to the previous examples through the reaction of BB-31 (150 mg, 542 μmol) with step 1 intermediate of example 92 (171 mg, 650 μmol) (40 mg, 17%).

LC-MS (method 2): m/z: [(M+H]⁺=424.3 (MW calc.=423.52); Rₜ=0.78 min.

¹H NMR (400 MHz, DMSO-d₆) δ (ppm)=10.30 (s, 1H), 7.58-7.49 (m, 2H), 7.23-7.12 (m, 2H), 5.57 (d, J=2.3 Hz, 1H), 3.91 (s, 2H), 3.20 (t, J=5.5 Hz, 2H), 2.74-2.58 (m, 3H), 2.38 (d, J=5.8 Hz, 3H), 2.33 (s, 3H), 2.14-1.91 (m, 7H), 1.68-1.50 (m, 2H).

### Synthesis Example 129

### 4-(2-Cyclohexyl-4,5,6,7-tetrahydro-1H-pyrrolo[3,2-c]pyridin-5-yl)-N-methyl-benzenesulfonic acid amide

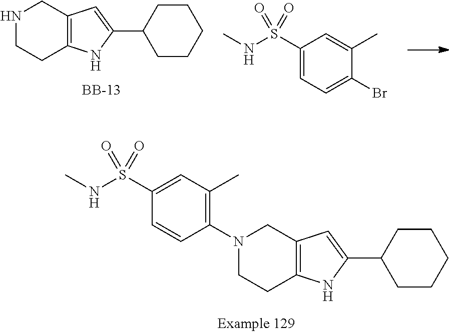

Example 129 was synthesized in analogy to the previous examples through the reaction of BB-13 (200 mg, 831 μmol) with step 1 intermediate of example 92 (262 mg, 997 μmol) (65 mg, 20%).

LC-MS (method 2): m/z: [(M+H]⁺=388.3 (MW calc.=387.54); Rₜ=0.86 min.

¹H NMR (400 MHz, DMSO-d₆) δ (ppm)=10.15 (s, 1H), 7.57-7.47 (m, 2H), 7.23-7.12 (m, 2H), 5.50 (d, J=2.1 Hz, 1H), 3.90 (s, 2H), 3.20 (t, J=5.5 Hz, 2H), 2.67 (t, J=5.5 Hz, 2H), 2.38 (d, J=5.2 Hz, 3H), 2.32 (s, 3H), 1.93-1.86 (m, 2H), 1.79-1.58 (m, 4H), 1.36-1.15 (m, 5H).

### Synthesis Example 130

### 2-(2,6-Difluoro-phenyl)-5-[2-ethyl-5-(trifluoromethyl)-2H-pyrazol-3-yl]-4,5,6,7-tetrahydro-1H-pyrrolo[3,2-c]pyridine

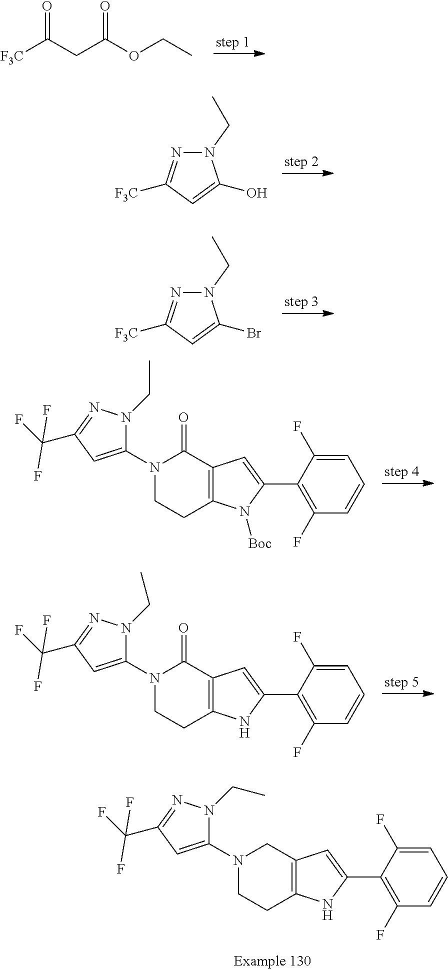

Step 1:

A mixture of 4,4,4-trifluoro-3-oxo-butyric acid ethyl ester (10.0 g, 54.3 mmol) and ethylhydrazine oxalate (9.78 g, 65.2 mmol) in EtOH (150 mL) was heated at reflux for 14 h. The RM was concentrated under reduced pressure, diluted with EtOAc and subsequently washed with water and brine and dried. The solvent was evaporated under reduced pressure and the residue was purified by CC (SiO₂, EtOAc/Hex) to afford the desired product (6.0 g, 61%).

LC-MS (Method 3): m/z [M+H]⁺=181.0 (MW calc.=180.13); Rₜ=1.36 min.

Step 2:

A mixture of the intermediate from step 1 (2.0 g, 11.1 mmol) and POBr₃ (7.9 g, 27.7 mmol) was heated at 120° C. for 16 h. The RM was basified with NaHCO₃ solution (50 mL) at 0° C. and extracted with CH₂Cl₂. The combined organic layers were washed with water and brine, dried and concentrated under reduced pressure. The residue was purified by CC (SiO₂, EtOAc/Hex) to afford 5-bromo-1-ethyl-3-trifluoromethyl-1H-pyrazole (800 mg, 29%).

Step 3:

A mixture of BB-3 (250 mg, 0.72 mmol), 5-bromo-1-ethyl-3-trifluoromethyl-1H-pyrazole (226 mg, 0.93 mmol) and K₂CO₃ (297 mg, 2.15 mmol) in toluene (5 mL) was degassed with Ar for 15 min. CuI (68 mg, 358 μmol) and N,N′-dimethylethylenediamine (39 μl, 0.36 mmol) were added to the RM and the resulting RM was heated in a sealed tube at 100° C. for 16 h. The RM was diluted with EtOAc, washed with water and brine and subsequently dried. The solvent was evaporated under reduced pressure and the residue was purified by CC (SiO₂, EtOAc/Hex) to afford the desired product (110 mg, 30%).

LC-MS (Method 3): m/z [M+H]⁺=511.1 (MW calc.=510.46); Rₜ=3.76 min.

Step 4:

To the intermediate from step 3 (110 mg, 215 μmol) in MeOH (5 mL) was added K₂CO₃ (89 mg, 647 μmol) at 0° C. and the RM was stirred at it for 3 h. The RM was concentrated and diluted with EtOAc, washed with water and brine and subsequently dried. The solvent was evaporated under reduced pressure to yield 2-(2,6-difluoro-phenyl)-5-(2-ethyl-5-trifluoromethyl-2H-pyrazol-3-yl)-1,5,6,7-tetrahydro-pyrrolo[3,2-c]pyridin-4-one (85 mg, 96%).

LC-MS (Method 3): m/z [M+H]⁺=410.9 (MW calc.=410.34); Rₜ=3.33 min.

Step 5:

To a solution of 2-(2,6-difluoro-phenyl)-5-(2-ethyl-5-trifluoromethyl-2H-pyrazol-3-yl)-1,5,6,7-tetrahydro-pyrrolo[3,2-c]pyridin-4-one (85 mg, 207 μmol) in THF (5 mL) was added BH₃.DMS (196 μl, 2.07 mmol) at 0° C. and the RM was stirred at it for 36 h. The RM was quenched with MeOH at 0° C. and RM was heated at reflux for 1 h. The RM was concentrated under reduced pressure and diluted with CH₂Cl₂ and washed with water and brine. The organic layer was dried and concentrated under reduced pressure. The residue was mixed with another 65 mg scale reaction and purified by CC (SiO₂; 15% EtOAc/Hex) to afford the title compound (68 mg, 47%).

LC-MS (Method 3): m/z [M+H]⁺=397.2 (MW calc.=396.36); Rₜ=3.90 min.

¹H NMR (DMSO-d₆, 400 MHz), δ (ppm)=10.87 (s, 1H), 7.28-7.20 (m, 1H), 7.15 (t, 2H, J=8.42 Hz), 6.41 (s, 1H), 6.30 (s, 1H), 4.09 (q, 2H, J=7.28 Hz), 3.96 (s, 2H), 3.20 (t, 2H, J=5.60 Hz), 2.82 (t, 2H, J=5.40 Hz), 1.37 (t, 3H, J=7.24 Hz).

### Synthesis Example 131

### 4-[2-(2,6-Difluoro-phenyl)-4,5,6,7-tetrahydro-1H-pyrrolo[3,2-c]pyridin-5-yl]-N,3-dimethyl-benzenesulfonic acid amide

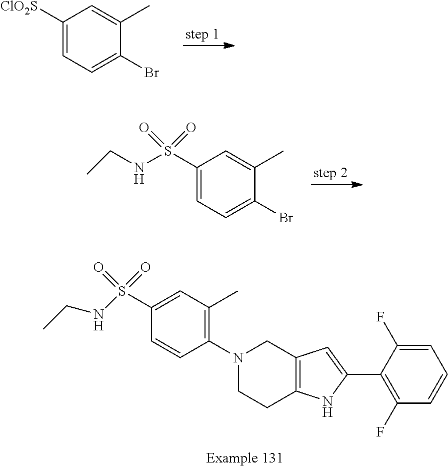 [SMILES: Cc1cc(S(=O)(=O)Cl)ccc1Br ; CCNS(=O)(=O)c1ccc(Br)c(C)c1 ; CCNS(=O)(=O)c1ccc(N2CCc3[nH]c(-c4c(F)cccc4F)cc3C2)c(C)c1]

Example 131 was synthesized in analogy to example 109 in two steps (28% yield over two steps). LC-MS (Method 2): m/z [M+H]⁺=432.2 (MW calc.=431.50); Rₜ=0.84 min.

¹H NMR (DMSO-d₆, 400 MHz), δ (ppm)=10.82 (s, 1H), 7.59-7.53 (m, 2H), 7.30 (t, J=6.0 Hz, 1H), 7.28-7.20 (m, 2H), 7.18-7.12 (m, 2H), 6.34 (s, 1H), 4.03 (s, 2H), 3.27 (t, J=5.6 t, J=5.3

### Synthesis Example 132

### N-[4-[2-(2,6-Difluoro-phenyl)-4,5,6,7-tetrahydro-1H-pyrrolo[3,2-c]pyridin-5-yl]-3-methyl-phenyl]-N-methyl-methanesulfonic acid amide

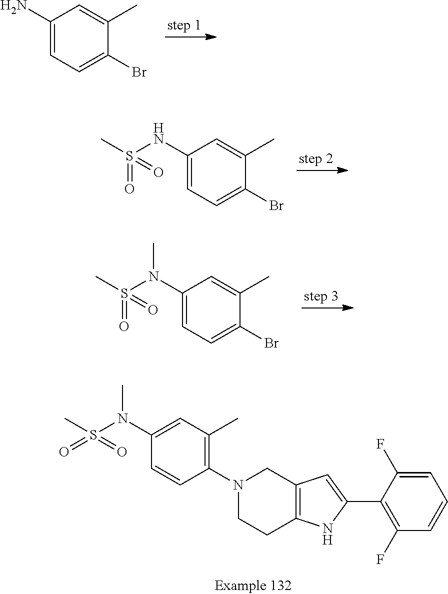

Step 1:

To 4-Bromo-3-methylaniline (600 mg, 3.22 mmol) in a mixture of dioxane (1.2 mL) and aqueous NaOH (1 m, 1.2 mL) was added methansulfonyl chloride (503 μL, 6.45 mmol) at 0° C. and the RM was stirred overnight at rt. Water was added and the mixture was extracted with EtOAc. The combined organic layers were dried and the volatiles were removed under reduced pressure to yield the desired compound (550 mg, 65%).

Step 2:

To a mixture of step 1 intermediate (170 mg, 644 μmol) and K₂CO₃ (175 mg, 1.29 mmol) in THF (1.5 mL) was added CH₃I (113 mg, 805 μmol) and the RM was stirred at it overnight. Water was added and the mixture was extracted with EtOAc. The combined organic layers were dried and the volatiles were removed under reduced pressure to yield the desired compound (190 mg, >99%).

Step 3:

Example 131 was synthesized in analogy to the previous examples through the reaction of BB-1 (115 mg, 425 μmol) with step 2 intermediate (143 mg, 510 μmol) (68 mg, 37%).

LC-MS (method 2): m/z: [(M+H]⁺=432.2 (MW calc.=431.50); Rₜ=0.83 min.

¹H NMR (400 MHz, DMSO-d₆) δ (ppm)=10.79 (s, 1H), 7.28-7.11 (m, 6H), 6.33 (s, 1H), 3.92 (s, 2H), 3.19 (s, 3H), 3.17 (t, J=5.6 Hz, 2H), 2.92 (s, 3H), 2.79 (t, J=5.6 Hz, 2H), 2.29 (s, 3H).

### Synthesis Example 133

### 4-[2-(2,6-Difluoro-phenyl)-4,5,6,7-tetrahydro-1H-pyrrolo[3,2-c]pyridin-5-yl]-5-methyl-2-methylsulfonyl-thiazole

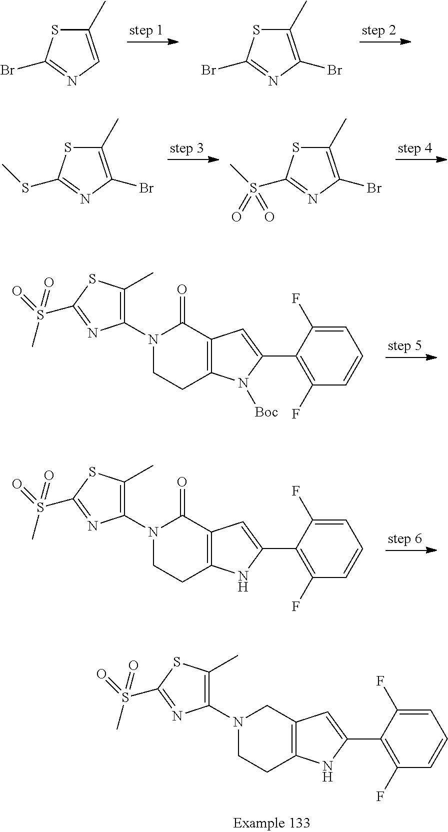

Step 1:

To a solution of 2-bromo-5-methyl-thiazole (3.0 g, 16.9 mmol) in a mixture of CHCl₃ (30 mL) and CH₃CN (30 mL) was added bromine (2.6 mL, 50.6 mmol) and the RM was heated at reflux for 16 h. The RM was quenched with sodium thiosulfate solution and extracted with CH₂Cl₂. The combined organic layers were washed with water and brine and dried. The solvent was evaporated under reduced pressure and the residue was purified by CC (SiO₂, EtOAc/Hex) to yield 2,4-dibromo-5-methyl-thiazole (1.6 g, 36%).

Step 2:

To a solution of 2,4-dibromo-5-methyl-thiazole (1.6 g, 6.2 mmol) in EtOH (20 mL) was added sodium thiomethoxide (1.3 g, 18.7 mmol) at 0° C. and the RM was stirred at it for 3 h. The RM was concentrated and diluted with EtOAc, washed with water and brine and dried. The solvent was evaporated under reduced pressure to give the crude product which was purified by CC (SiO₂, EtOAc/Hex) to afford 4-bromo-5-methyl-2-methylsulfanyl-thiazole (1.0 g, 71%).

LC-MS (Method 3): m/z [M+H]⁺=225.8 (MW calc.=224.14); Rₜ=3.52 min.

Step 3:

To a solution of 4-bromo-5-methyl-2-methylsulfanyl-thiazole (1.0 g, 4.46 mmol) in CH₂Cl₂ (20 mL) was added m-CPBA (2.7 g, 11.2 mmol, 70% in water) at 0° C. and the RM was stirred at it for 16 h. Sodium thiosulfate solution was added to the RM and extracted with CH₂Cl₂. The organic layer was washed with sat.NaHCO₃ solution, water and dried. The Solvent was evaporated under reduced pressure and the residue was purified by CC (SiO₂, EtOAc/Hex) to yield 4-bromo-2-methanesulfonyl-5-methyl-thiazole (900 mg, 78%).

LC-MS (Method 3): m/z [M+H]⁺=256.0 and 258.0 (MW calc.=256.14); Rₜ=2.85 min.

Step 4:

BB-3 (300 mg, 0.862 mmol) and 4-bromo-2-methanesulfonyl-5-methyl-thiazole (265 mg, 1.034 mmol) were transformed in analogy to step 3 of example 129 to afford 2-(2,6-difluoro-phenyl)-5-(2-methanesulfonyl-5-methyl-thiazol-4-yl)-4-oxo-4,5,6,7-tetrahydro-pyrrolo[3,2-c]pyridine-1-carboxylic acid tert-butyl ester (200 mg, 44%).

LC-MS (Method 3): m/z [M+H]⁺=524.3 (MW calc.=523.57; Rₜ=3.51 min.

Step 5:

2-(2,6-difluoro-phenyl)-5-(2-methanesulfonyl-5-methyl-thiazol-4-yl)-4-oxo-4,5,6,7-tetrahydro-pyrrolo[3,2-c]pyridine-1-carboxylic acid tert-butyl ester was transformed in analogy to step 4 of example 129 to yield 2-(2,6-difluoro-phenyl)-5-(2-methanesulfonyl-5-methyl-thiazol-4-yl)-1,5,6,7-tetrahydro-pyrrolo[3,2-c]pyridin-4-one (160 mg, 99%).

LC-MS (Method 3): m/z [M+H]⁺=424.2 (MW calc.=423.46); Rₜ=2.98 min.

Step 6:

The intermediate of step 5 was transformed in analogy to step 5 of example 130 to yield the title compound (56 mg, 36%).

LC-MS (Method 3): m/z [M+H]⁺=410.1 (MW calc.=409.48); Rₜ=3.54 min.

¹H NMR (DMSO-d₆, 400 MHz), δ (ppm)=10.83 (s, 1H), 7.26-7.21 (m, 1H), 7.14 (t, 2H, J=8.4 Hz), 6.31 (s, 1H), 4.15 (s, 2H), 3.41-3.38 (m, 5H), 2.83 (t, 2H), 2.45 (s, 3H).

### Synthesis Example 134

### 5-[4-Methyl-6-(trifluoromethyl)-pyridin-3-yl]-2-tetrahydro-pyran-3-yl-4,5,6,7-tetrahydro-1H-pyrrolo[3,2-c]pyridine

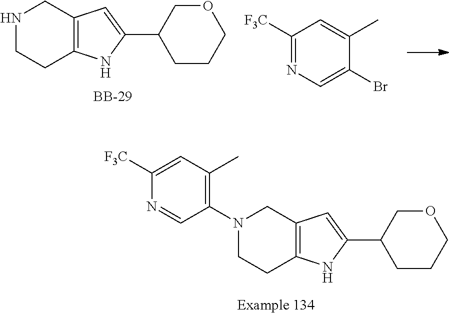

Example 134 was synthesized in analogy to the previous examples through the reaction of BB-29 (200 mg, 824 μmol) with -bromo-4-methyl-2-(trifluoromethyl)pyridine (236 mg, 989 μmol) (60 mg, 20%).

LC-MS (method 2): m/z: [(M+H]⁺=366.2 (MW calc.=365.39); Rₜ=0.79 min.

¹H NMR (400 MHz, DMSO-d₆) δ (ppm)=10.32 (s, 1H), 8.35 (s, 1H), 7.66 (s, 1H), 5.59 (s, 1H), 4.03 (s, 2H), 3.93-3.80 (m, 2H), 3.30-3.26 (m, 2H), 2.75-2.64 (m, 3H), 2.37 (s, 3H), 2.01-1.95 (m, 1H), 1.65-1.55 (m, 3H).

### Synthesis Example 135

### 2-(4,6-Dimethyl-pyridin-3-yl)-5-[4-methyl-6-(trifluoromethyl)-pyridin-3-yl]-4,5,6,7-tetrahydro-1H-pyrrolo[3,2-c]pyridine

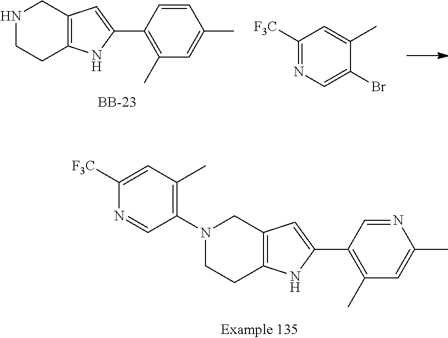

Example 135 was synthesized in analogy to the previous examples through the reaction of BB-23 (120 mg, 569 μmol) with 5-bromo-4-methyl-2-(trifluoromethyl)pyridine (163 mg, 682 μmol) (40 mg, 18%).

LC-MS (method 2): m/z: [(M+H]⁺=387.1 (MW calc.=386.41); Rₜ=0.58 min.

¹H NMR (400 MHz, DMSO-d₆) δ (ppm)=10.90 (s, 1H), 8.44 (s, 1H), 8.41 (s, 1H), 7.68 (s, 1H), 7.11 (s, 1H), 6.11 (d, J=2.5 Hz, 1H), 4.16 (s, 2H), 3.36 (t, J=5.8 Hz, 2H), 2.81 (t, J=5.8 Hz, 2H), 2.41 (s, 3H), 2.40 (s, 3H), 2.38 (s, 3H).

### Synthesis Example 136

### 4-[2-(2,6-Difluoro-phenyl)-4,5,6,7-tetrahydro-1H-pyrrolo[3,2-c]pyridin-5-yl]-3-methyl-N-(2,2,2-trifluoro-ethyl)-benzenesulfonic acid amide

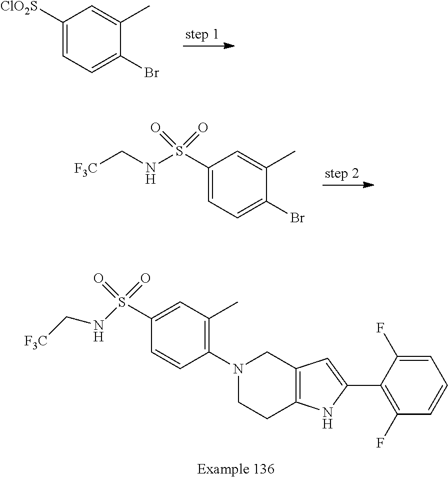 [SMILES: Cc1cc(S(=O)(=O)Cl)ccc1Br ; Cc1cc(S(=O)(=O)NCC(F)(F)F)ccc1Br ; Cc1cc(S(=O)(=O)NCC(F)(F)F)ccc1N1CCc2[nH]c(-c3c(F)cccc3F)cc2C1]

Example 136 was synthesized in analogy to example 109 in two steps (14% over two steps). LC-MS (Method 2): m/z [M+H]⁺=486.1 (MW calc.=485.47); Rₜ=0.85 min.

¹H NMR (DMSO-d₆, 400 MHz), δ (ppm)=10.83 (s, 1H), 8.35 8 s, 1H), 7.66-7.52 (m, 2H), 7.28-7.20 (m, 2H), 7.18-7.10 (m, 2H), 6.34 8 s, 1H), 4.04 (s, 2H), 3.63 (q, J=9.4 Hz, 2H), 3.34-3.25 (m, 2H), 2.81 (t, J=4.9 Hz, 2H), 2.36 (s, 3H).

### Synthesis Example 137

### 2-(2,6-Difluoro-phenyl)-5-[5-(1-methoxy-cyclopropyl)-2-methyl-2H-pyrazol-3-yl]-4,5,6,7-tetrahydro-1H-pyrrolo[3,2-c]pyridine

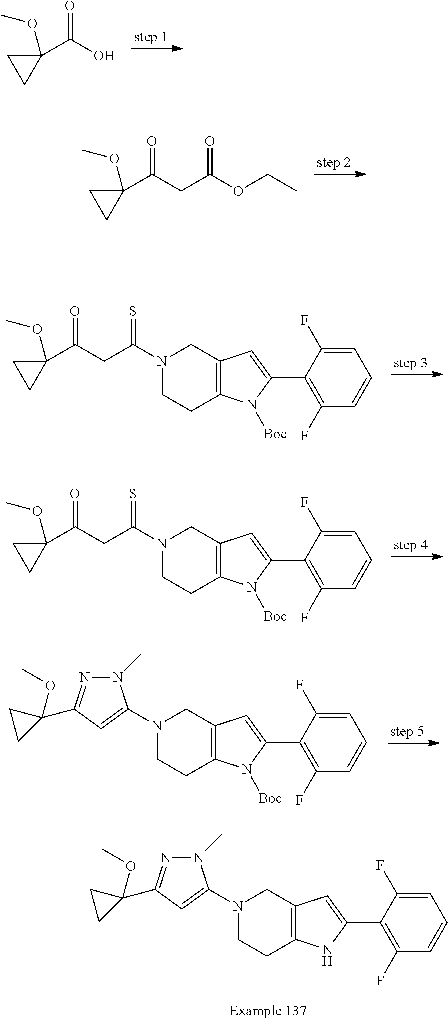

Step 1:

1-methoxy-cyclopropanecarboxylic acid was transformed in analogy to step 1 of example 123 to yield 3-(1-methoxy-cyclopropyl)-3-oxo-propionic acid methyl ester (2.5 g, 31%).

LC-MS: (Method 3): m/z [M+H]⁺=187.2 (MW calc.=186.21); Rₜ=2.73.

Step 2:

3-(1-methoxy-cyclopropyl)-3-oxo-propionic acid methyl ester and BB-2 were transformed in analogy to step 2 of example 123 to yield 2-(2,6-difluoro-phenyl)-5-[3-(1-methoxy-cyclopropyl)-3-oxo-propionyl]-4,5,6,7-tetrahydro-pyrrolo[3,2-c]pyridine-1-carboxylic acid tert-butyl ester (2.5 g, 58%). LC-MS (Method 3): m/z [M+H]⁺=475.3 (MW calc.=474.5); Rₜ=3.52.

Step 3:

The intermediate from step 2 was reacted in analogy to step 3 of example 123 to give 2-(2,6-difluoro-phenyl)-5-[3-(1-methoxy-cyclopropyl)-3-oxo-thiopropionyl]-4,5,6,7-tetrahydro-pyrrolo[3,2-c]-pyridine-1-carboxylic acid tert-butyl ester along with other impurities that was used in the next step without purification.

LC-MS (Method 3): m/z [M+H]⁺=491.2 (MW calc.=490.56); Rₜ=3.72.

Step 4:

The synthesis was performed in analogy to step 4 of example 123 to afford 2-(2,6-difluoro-phenyl)-5-[5-(1-methoxy-cyclopropyl)-2-methyl-2H-pyrazol-3-yl]-4,5,6,7-tetrahydro-pyrrolo[3,2-c]pyridine-1-carboxylic acid tert-butyl ester (120 mg, 23%).

LC-MS (Method 3): m/z [M+H]⁺=484.9 (MW calc.=484.54); Rₜ=3.91.

Step 5:

The intermediate from step 4 was transformed in analogy to step 5 of example 123 to yield the title compound (45 mg, 81%).

LC-MS: LC-MS (Method 3): m/z [M+H]⁺=385.2 (MW calc.=384.42); Rₜ=3.48.

¹H NMR (DMSO-d₆, 400 MHz), δ (ppm)=10.83 (s, 1H), 7.29-7.23 (m, 1H), 7.14 (t, 2H, J=8.4 Hz), 6.30 (s, 1H), 5.86 (s, 1H), 3.91 (s, 2H), 3.60 (s, 3H), 3.20 (s, 3H), 3.17 (t, 2H, J=5.60 Hz), 2.80 (t, 2H, J=5.20 Hz), 0.99-0.96 (m, 2H), 0.89-0.86 (m, 2H).

### Synthesis Example 138

### 2-(2,6-Difluoro-phenyl)-4-methyl-5-(4-methyl-6-methylsulfonyl-pyridin-3-yl)-4,5,6,7-tetrahydro-1H-pyrrolo[3,2-c]pyridine

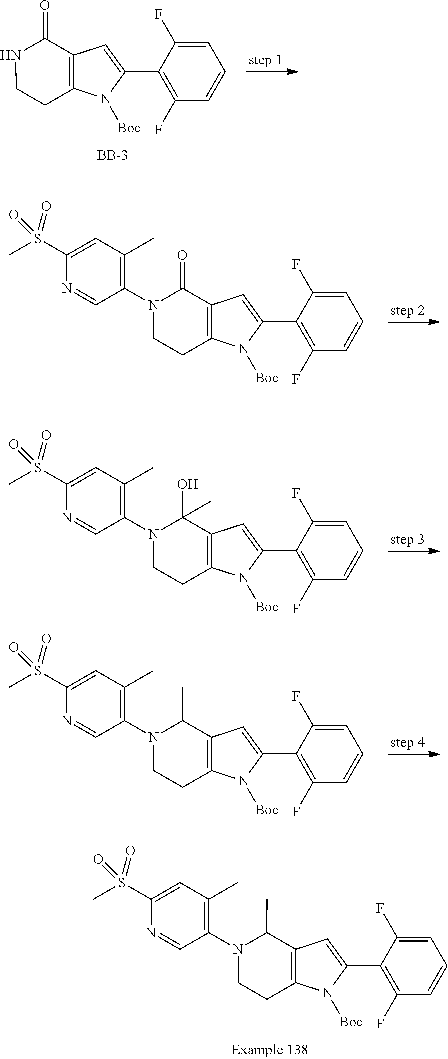

Step 1:

BB-3 and 5-bromo-4-methyl-2-(methylsulfonyl)pyridine were reacted in analogy to step 3 of example 130 to afford the desired product (275 mg, 26%).

LC-MS (Method 3): m/z [M+H]⁺=518.2 (MW calc.=517.55); Rₜ=3.38 min.

Step 2:

To a solution of the intermediate from step 1 (900 mg, 1.74 mmol) in THF (50 mL) was added a 1.6M solution of MeLi in ether (2.17 ml, 3.48 mmol) at −78° C. and the RM was stirred at same temperature for 30 min. The RM was quenched with sat. NH₄Cl solution and extracted with EtOAc. The combined organic layers were washed with water and brine and subsequently dried. The solvent was evaporated under reduced pressure to give the crude product which was purified by CC (SiO₂; 24% Acetone/Hex) to yield the desired product (310 mg, 33%).

LC-MS (Method 3): m/z [M+H]⁺=534.3 (MW calc.=533.59); Rₜ=3.56 min.

Step 3:

A solution of the intermediate from step 2 (210 mg, 0.39 mmol) in MeOH (10 mL) was degassed through purging with Ar for 15 min followed by addition of Pd—C(210 mg). The RM was stirred at it under H₂ for 16 h. The RM was filtered through celite and washed with MeOH. The filtrate was concentrated under reduced pressure to give the crude product which was purified by CC (SiO₂, EtOAc/Hex) to yield 2-(2,6-difluoro-phenyl)-5-(6-methanesulfonyl-4-methyl-pyridin-3-yl)-4-methyl-4,5,6,7-tetrahydro-pyrrolo[3,2-c]pyridine-1-carboxylic acid tert-butyl ester (110 mg, 36%).

LC-MS (Method 3): m/z [M+H]⁺=518.3 (MW calc.=517.59); Rₜ=3.81 min.

Step 4:

To a solution of 2-(2,6-difluoro-phenyl)-5-(6-methanesulfonyl-4-methyl-pyridin-3-yl)-4-methyl-4,5,6,7-tetrahydro-pyrrolo[3,2-c]pyridine-1-carboxylic acid tert-butyl ester (110 mg, 212 mmol) in MeOH (5 mL) was added K₂CO₃ (88 mg, 638 mmol) at 0° C. and the RM was heated at 60° C. for 16 h. The RM was concentrated and diluted with EtOAc, washed with water and brine and dried. The solvent was evaporated under reduced pressure to give the crude product which was purified by CC (SiO₂, EtOAc/Hex) to yield the title compound (53 mg, 60%).

LC-MS (Method 3): m/z [M+H]⁺=418.2 (MW calc.=417.47); Rₜ=3.51 min.

¹H NMR (DMSO-d₆, 400 MHz), δ (ppm)=8.38 (s, 1H), 7.82 (s, 1H), 7.26-7.19 (m, 1H), 7.11 (t, J=8.2 Hz, 2H), 6.35 (s, 1H), 4.58-4.53 (m, 1H), 3.48-3.41 (m, 1H), 3.32-3.25 (m, 1H), 3.16 (s, 3H), 2.76-2.67 (m, 2H), 2.37 (s, 3H), 1.19 (d, J=6.4 Hz, 3H).

### Synthesis Example 139

### 2-(2,6-Difluoro-phenyl)-5-[2-ethyl-5-(1-methoxy-cyclopropyl)-2H-pyrazol-3-yl]-4,5,6,7-tetrahydro-1H-pyrrolo[3,2-c]pyridine

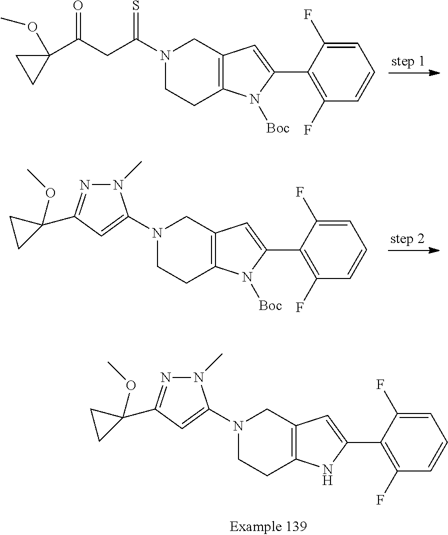 [SMILES: COC1(C(=O)CC(=S)N2CCc3c(cc(-c4c(F)cccc4F)n3C(=O)OC(C)(C)C)C2)CC1 ; COC1(c2cc(N3CCc4c(cc(-c5c(F)cccc5F)n4C(=O)OC(C)(C)C)C3)n(C)n2)CC1 ; COC1(c2cc(N3CCc4[nH]c(-c5c(F)cccc5F)cc4C3)n(C)n2)CC1]

Step 1:

To a solution of the intermediate from step 3 of example 137 (2.32 mmol) in i-PrOH (20 mL) were added ethylhydrazine-HCl (1.5 g, 16.2 mmol) and DIPEA (4.0 mL, 23.2 mmol). The RM was heated at reflux for 48 h, cooled to it and concentrated. The residue was diluted with CH₂Cl₂, washed with sat. NH₄Cl solution and brine, and dried. The solvent was evaporated under reduced pressure to give the crude product which was purified by CC (SiO₂, EtOAc/Hex) followed by prep. HPLC to afford 2-(2,6-difluoro-phenyl)-5-[2-ethyl-5-(1-methoxy-cyclopropyl)-2H-pyrazol-3-yl]-4,5,6,7-tetrahydro-pyrrolo[3,2-c]-pyridine-1-carboxylic acid tert-butyl ester (100 mg, 9%).

LC-MS (Method 3): m/z [M+H]⁺=499.3 (MW calc.=498.56); Rₜ=4.11 min.

Step 2:

To a solution of 2-(2,6-difluoro-phenyl)-5-[2-ethyl-5-(1-methoxy-cyclopropyl)-2H-pyrazol-3-yl]-4,5,6,7-tetrahydro-pyrrolo[3,2-c]pyridine-1-carboxylic acid tert-butyl ester (100 mg, 0.20 mmol) in methanol (4 mL) was added K₂CO₃ (83 mg, 0.6 mmol) and the RM was heated at reflux for 16 h. The solvent was evaporated under reduced pressure and the residue was diluted with CH₂Cl₂, washed with water and brine, and dried. The organic layer was concentrated under reduced pressure to give the crude product which was purified by CC (SiO₂, EtOAc/Hex) to afford the title compound (51 mg, 64%).

LC-MS (Method 3): m/z [M+H]⁺=398.9 (MW calc.=398.45); Rₜ=3.60 min.

¹H NMR (DMSO-d₆, 400 MHz), δ (ppm)=10.84 (s, 1H), 7.29-7.21 (m, 1H), 7.14 (t, J=8.52 Hz, 2H), 6.30 (s, 1H), 5.89 (s, 1H), 3.95 (m, 4H), 3.21 (s, 3H), 3.14 (t, J=5.4 Hz, 2H), 2.80 (t, J=5.3 Hz, 2H), 1.30 (t, J=7.2 Hz, 3H), 1.0-0.97 (m, 2H), 0.90-0.87 (m, 2H).

### Synthesis Example 140

### 5-[6-(Difluoro-methoxy)-4-methoxy-pyridin-3-yl]-2-(2,6-difluoro-phenyl)-4,5,6,7-tetrahydro-1H-pyrrolo[3,2-c]pyridine

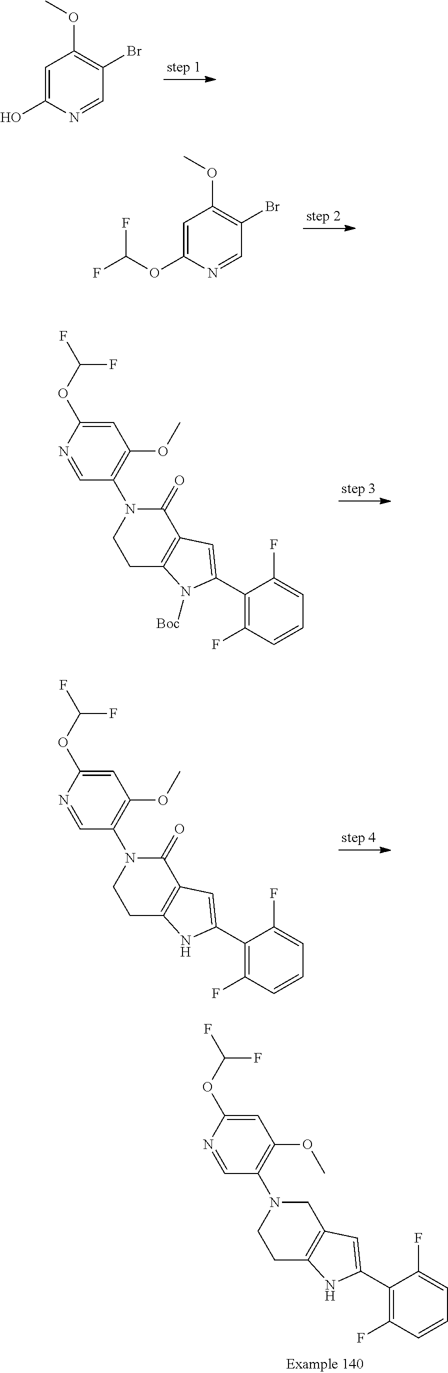

Step 1:

A mixture of 5-bromo-4-methoxy-pyridin-2-ol (1.0 g, 4.90 mmol), sodium difluorochloroacetate (893 mg, 5.88 mmol) and Cs₂CO₃ (2.38 g, 7.35 mmol) in DMF (15 mL) was heated at 100° C. for 2 h. The RM was diluted with ice cold water and extracted with EtOAc. The combined organic layers were washed with water and brine, dried and concentrated under reduced pressure to give the crude product which was purified by CC (SiO₂, EtOAc/Hex) to yield 5-bromo-2-difluoromethoxy-4-methoxy-pyridine (570 mg, 46%).

Step 2:

BB-3 and 5-bromo-2-difluoromethoxy-4-methoxy-pyridine were reacted in analogy to step 3 of example 130 to afford the desired product (250 mg, 33%).

LC-MS (Method 3): m/z [M+H]⁺=522.1 (MW calc. 521.46); Rₜ=3.63 min.

Step 3:

The intermediate from step 2 was transformed into the desired product in analogy to previous examples (155 mg, 96%).

LC-MS (Method 3): m/z [M+H]⁺=422.1 (MW calc. 421.35); Rₜ=3.15 min.

Step 4: 5-(6-difluoromethoxy-4-methoxy-pyridin-3-yl)-2-(2,6-difluoro-phenyl)-1,5,6,7-tetrahydro-pyrrolo-[3,2-c]pyridin-4-one was transformed into the title compound in analogy to step 5 of example 130 (95 mg, 65%).

LC-MS (Method 3): m/z [M+H]⁺=408.2 (MW calc. 407.36); Rₜ=3.72 min.

¹H NMR (CDCl₃, 400 MHz), δ (ppm)=8.75 (s, 1H), 7.72 (s, 1H), 7.55-7.19 (t, J=73.6 Hz, 1H), 7.08-7.01 (m, 1H), 6.93 (t, J=8.4 Hz, 2H), 6.64 (s, 1H), 6.37 (s, 1H), 4.18 (s, 2H), 3.92 (s, 3H), 3.44 (t, J=5.6 Hz, 2H), 2.79 (t, J=5.6 Hz, 2H).

### Synthesis Example 141

### 2-(5-Fluoro-4-methyl-pyridin-3-yl)-5-[4-methyl-6-(trifluoromethyl)-pyridin-3-yl]-4,5,6,7-tetrahydro-1H-pyrrolo[3,2-c]pyridine

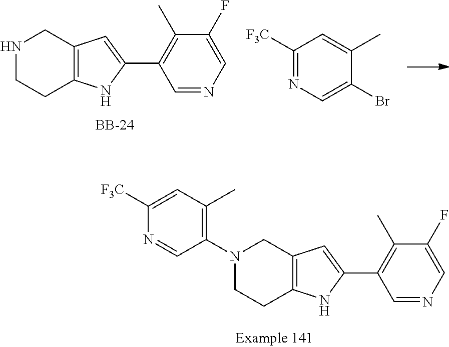

Example 141 was synthesized in analogy to the previous examples through the reaction of BB-23 (149 mg, 560 μmol) with 5-bromo-4-methyl-2-(trifluoromethyl)pyridine (161 mg, 672 μmol) (50 mg, 23%). LC-MS (method 2): m/z: [(M+H]⁺=391.1 (MW calc.=390.38); Rₜ=0.82 min.

¹H NMR (400 MHz, DMSO-d₆) δ (ppm)=11.10 (s, 1H), 8.47 (s, 1H), 8.42 (s, 1H), 8.30 (s, 1H), 7.69 (s, 1H), 6.25 (d, J=1.5 Hz, 1H), 4.17 (s, 2H), 3.37 (t, J=5.6 Hz, 1H), 2.83 (t, J=5.6 Hz, 1H), 2.41 (s, 3H), 2.37 (s, 3H).

### Synthesis Example 142

### 2-(2,6-Difluoro-phenyl)-3-fluoro-5-(4-methyl-6-methylsulfonyl-pyridin-3-yl)-4,5,6,7-tetrahydro-1H-pyrrolo[3,2-c]pyridine

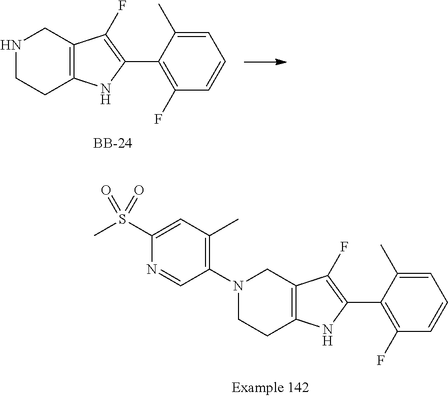 [SMILES: Cc1cccc(F)c1-c1[nH]c2c(c1F)CNCC2 ; "BB-24" ; Cc1cc(S(C)(=O)=O)ncc1N1CCc2[nH]c(-c3c(C)cccc3F)c(F)c2C1]

A mixture of BB-24 (120 mg, 0.476 mmol) and 5-bromo-2-methanesulfonyl-4-methyl-pyridine (142 mg, 0.57 mmol) in dioxane (3 mL) was degassed with N₂ for 15 min followed by addition of Cs₂CO₃ (310 mg, 0.95 mmol), BINAP (30 mg, 0.04 mmol) and Pd(OAc)₂ (11.0 mg, 0.04 mmol). The resulting RM was heated in a sealed tube at 110° C. for 16 h. The RM was filtered through celite and the filtrate was concentrated under reduced pressure to give the crude product which was purified by CC (SiO₂, EtOAc/Hex) to afford the title compound (70 mg, 35%).

LC-MS (Method 3): m/z [M+H]⁺=422.1 (MW calc.=421.44); Rt=3.31 min.

¹H NMR (DMSO-d₆, 400 MHz), δ (ppm)=10.93 (s, 1H), 8.45 (s, 1H), 7.84 (s, 1H), 7.45-7.37 (m, 1H), 7.19 (t, J=8.0 Hz, 2H), 4.19 (s, 2H), 3.35 (t, J=5.6 Hz, 2H), 3.21 (s, 3H), 2.78 (t, J=5.5 Hz, 2H), 2.42 (s, 3H).

### Synthesis Example 144

### 2-(2,6-Difluoro-phenyl)-3-fluoro-5-(4-ethoxy-6-trifluormethyl-pyridin-3-yl)-4,5,6,7-tetrahydro-1H-pyrrolo[3,2-c]pyridine

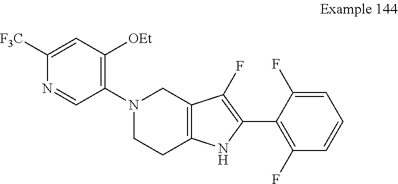 [SMILES: CCOc1cc(C(F)(F)F)ncc1N1CCc2[nH]c(-c3c(F)cccc3F)c(F)c2C1]

The title compound was prepared employing analogous procedures as for example 142.

### Synthesis Example 143

### 2-(2,6-Difluoro-phenyl)-5-(6-ethoxy-4-methoxy-pyridin-3-yl)-4,5,6,7-tetrahydro-1H-pyrrolo[3,2-c]pyridine

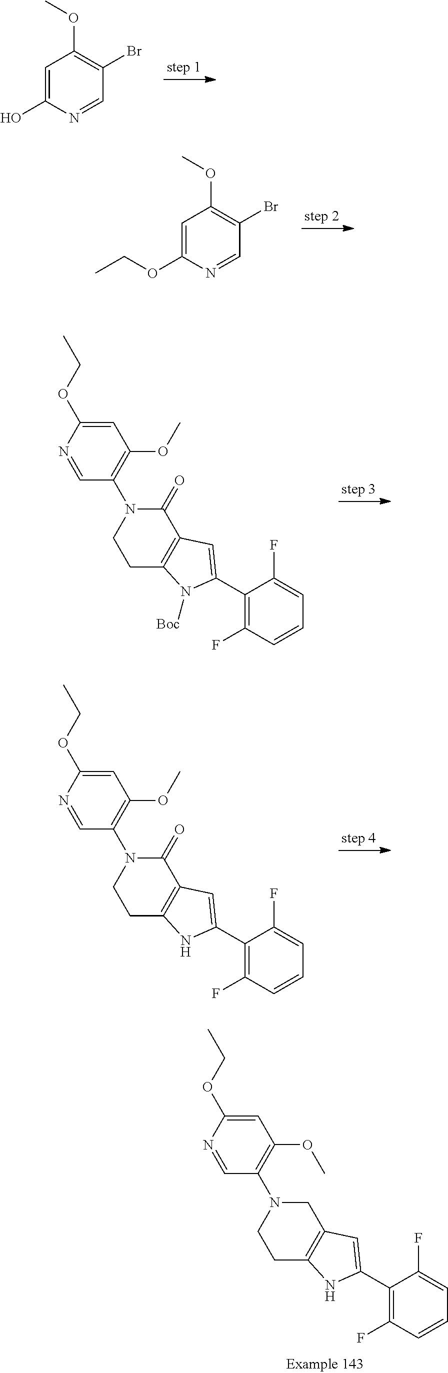

Step 1:

A mixture of 5-bromo-4-methoxy-pyridin-2-ol (750 mg, 3.67 mmol), ethyl iodide (1.47 mL, 18.4 mmol) and Ag₂CO₃ (1.3 g, 4.78 mmol) in CHCl₃ (25 mL) was stirred at it for 14 h. The RM was filtered and filtrate was concentrated under reduced pressure to give the crude product which was purified by CC (SiO₂, EtOAc/Hex) to yield 5-bromo-2-ethoxy-4-methoxy-pyridine (550 mg, 64%).

Step 2:

BB-3 and product 5-bromo-2-ethoxy-4-methoxy-pyridine were reacted in analogy to step 3 of example 130 to afford the desired compound (33%).

LC-MS (Method 3): m/z [M+H]⁺=500.3 (MW calc. 499.51); Rₜ=3.67 min.

Step 3:

The intermediate from step 2 was transformed into the desired product in analogy to previous examples (148 mg, 98%).

LC-MS (Method 3): m/z [M+H]⁺=400.2 (MW calc. 499.39); Rₜ=3.09 min.

Step 4:

2-(2,6-difluoro-phenyl)-5-(6-ethoxy-4-methoxy-pyridin-3-yl)-1,5,6,7-tetrahydro-pyrrolo[3,2-c]-pyridin-4-one was transformed into the title compound in analogy to step 5 of example 130 (48 mg, 35%).

LC-MS (Method 3): m/z [M+H]^{E}=386.2 (MW calc. 385.41); Rₜ=3.73 min.

¹H NMR (DMSO-d₆, 400 MHz), δ (ppm)=10.75 (s, 1H), 7.63 (s, 1H), 7.27-7.20 (m, 1H), 7.13 (t, J=8.5 Hz, 2H), 6.35 (s, 1H), 6.29 (s, 1H), 4.22 (q, J=7.0 Hz, 2H), 3.94 (s, 2H), 3.83 (s, 3H), 3.25 (t, J=5.2 Hz, 2H), 2.71 (s, 2H), 1.28 (t, J=7.0 Hz, 3H).

### Pharmacological Methods

Compounds of the invention have been tested for their effects on CRAC channels according to the following or similar procedures.

### HEK Calcium Influx Assay

The effect of compounds of the invention on intracellular [Ca²⁺] was tested in the HEK293 cell line (ECACC).

HEK293 cells were cultured in DMEM/F12/Glutamax (Gibco) containing 10% FCS (Gibco), and maintained at 37° C., 5% CO₂. Cell were split twice a week [3*10⁶ (Mon-Thu) and 1*10⁶ (Thu-Mon) cells/50 ml medium in 1-175 ZK culture flasks, respectively]. Twenty four hours pre-experiment, cells were seeded on 96 well plates (Poly-D-Lysine 96 well Black/Clear Plate, BD Biocoat REF 356640) at a density of 40,000 cells/well in DMEM/F12 (Gibco) containing 10% FCS (Gibco), and maintained at 37° C., 5% CO₂.

Prior to store-depletion, cell culture medium was removed and cells were loaded with the Calcium-sensitive fluorescent dye comprised within the Calcium-4-assay kit (Molecular Devices) in nominally Ca²⁺-free HBS buffer (140 mM NaCl, 4 mM KCl, 0.2 mM MgCl₂, 11 mM D-glucose, and 10 mM HEPES, pH 7.4) according to manufacturer's instruction for 60 min at 37° C., 5% CO₂.

Passive depletion of intracellular Ca²⁺-stores was then triggered by employing the SERCA inhibitor thapsigargin (2 μM final) for 10 min in the dark (RT). To prevent immediate Ca²⁺-entry via the activated store-operated channels (SOCs), cells were maintained in Ca²⁺-free HBS buffer comprising 100 μM EGTA during store-depletion.

Intracellular changes in [Ca²⁺] were subsequently monitored with the FLIPR device (Molecular Devices). In brief, baseline imaging post-store depletion was allowed for 1 min before adjusting the extracellular buffer to 3 mM CaCl₂. Increases in intracellular [Ca²⁺] due to pre-activated SOC channels were monitored for 15 min until intracellular Ca²⁺ levels had declined into a steady-state. Finally, compounds were administered, and Ca²⁺ signals were recorded for additional 10 min.

Inhibition of endogenous SOC in HEK293 cells was quantified employing the average Ca²⁺ signal measured from 9.5-10 min post-administration. Zero percent inhibition (MAX) was defined as the Ca²⁺ signal recorded from wells to which DMSO-only had been added instead of compound. Hundred percent inhibition (MIN) was defined as the signal obtained from wells in which cells haven't been treated with TG prior to Ca²⁺ addition and to which DMSO-only had been added instead of compound. For routine IC₅₀ determinations of compounds, 8 concentrations of a serial dilution (1:3.16) were tested, starting off from 10 μM. Reliable IC50's could consequently be determined only, if they were at least sub 2.5-3 μM.

### Jurkat IL-2 Production Assay

The effect of compounds of the invention on Interleukin-2 (IL-2) production/release was tested in the Jurkat T cell line (ECACC) clone E6-1.

Jurkat T cells were cultured in DMEM/F12/Glutamax (Gibco) containing 10% FCS (Gibco), and maintained at 37° C., 5% CO₂. Cells were split twice a week [5*10⁶ (Mon-Thu) and 1*10⁷ (Thu-Mon) cells/50 ml medium in 1-175 ZK culture flasks, respectively].

Prior to experiment, cells were seeded on 96 well plates (Cellstar 96 Well; Cat No. 655180, Greiner bio-one) at a density of 5*10⁵ cells/well in DMEM/F12/Glutamax (Gibco) containing 10% FCS (Gibco), and incubated for 60 min at 37° C., 5% CO₂. Subsequently, compounds were added and cells were allowed to incubate for 30 min at 37° C., 5% CO₂. Cells were then stimulated with 15 μg/ml Phyto-hemagglutinin (PHA; Sigma) for 22 hours at 37° C., 5% CO₂.

Before sampling of the supernatants, cells were spun down (200*g/5 min/RT). The amount of IL-2 released into the supernatant was quantified with the human IL-2 AlphaLisa kit (Perkin Elmer) according to manufacturer's instructions. Luminescence proximity measurements were carried out in the Synergy H4 reader (BioTek) employing the fluorescence setting of the reader (ex: 680/30 nm; em: 620/40 nm).

Inhibition of IL-2 production/release in/from Jurkat T cells was quantified as follows: Zero percent inhibition (MAX) was defined as the [IL-2] determined in supernatants derived from cells to which PHA & DMSO-only had been added instead of compound. Hundred percent inhibition (MIN) was defined as the [IL-2] determined in supernatants derived from cells that had been pre-treated with 1 μM CyclosporineA (Sigma) before the addition of 15 μg/ml PHA.

For routine IC50 determinations of compounds, 8 concentrations of a serial dilution (1:3.16) were tested, starting off from 10 μM.

TABLE 2
Selected compounds of the invention exhibit inhibition
of the CRAC channel (Calcium influx assay/FLPR)
and inhibition of the IL-2 prodction in these assays
within the following ranges:
% inhibition @ 10 μM: >70% (A); 50%-70% (B);
<50% (C);
IC₅₀ values: <0.5 μM (A); 0.5-1.0 μM (B);
>1.0-5.0 μM (C); not determined (nd)

| | % inhib. | IC₅₀ |
|---|---|---|
| Ex. | [@ 10 μM] | [μM] |
| No. | FLIPR | IL-2 |
| 1 | B | C |
| 2 | A | A |
| 4 | A | A |
| 5 | B | nd |
| 6 | B | A |
| 7 | C | C |
| 8 | A | A |
| 9 | C | C |
| 10 | A | C |
| 11 | C | B |
| 12 | B | A |
| 13 | C | B |
| 14 | C | A |
| 15 | A | C |
| 16 | A | A |
| 17 | A | nd |
| 18 | A | A |
| 19 | A | A |
| 20 | C | C |
| 21 | A | C |
| 22 | A | C |
| 23 | C | C |
| 24 | A | A |
| 25 | C | C |
| 26 | A | A |
| 28 | A | B |
| 29 | B | nd |
| 30 | A | B |
| 31 | C | A |
| 38 | A | nd |
| 39 | B | nd |
| 41 | B | nd |
| 42 | A | nd |
| 43 | B | nd |
| 44 | B | nd |
| 45 | A | nd |
| 46 | B | nd |
| 47 | A | nd |
| 48 | A | nd |
| 49 | A | nd |
| 50 | B | nd |
| 51 | A | nd |
| 52 | A | nd |
| 53 | A | nd |
| 54 | A | nd |
| 55 | A | nd |
| 56 | A | nd |
| 57 | A | nd |
| 58 | A | nd |
| 60 | A | nd |
| 61 | A | nd |
| 62 | A | nd |
| 63 | A | nd |
| 64 | C | C |
| 66 | C | C |
| 67 | C | B |
| 68 | A | A |
| 69 | A | B |
| 70 | A | C |
| 71 | C | C |
| 72 | A | A |
| 73 | A | A |
| 74 | A | C |
| 75 | A | C |
| 77 | A | C |
| 78 | A | B |
| 79 | A | B |
| 80 | A | nd |
| 81 | A | nd |
| 82 | A | B |
| 83 | A | C |
| 84 | B | nd |
| 85 | A | nd |
| 86 | C | C |
| 87 | A | C |
| 88 | B | A |
| 89 | B | nd |
| 90 | A | C |
| 91 | B | C |
| 92 | A | C |
| 93 | A | C |
| 94 | A | nd |
| 95 | A | nd |
| 96 | A | B |
| 97 | B | C |
| 98 | A | nd |
| 99 | A | A |
| 100 | C | C |
| 101 | A | A |
| 102 | A | B |
| 103 | C | A |
| 104 | A | C |
| 105 | A | B |
| 106 | A | B |
| 107 | A | C |
| 108 | C | B |
| 109 | C | A |
| 110 | C | C |
| 111 | C | A |
| 112 | C | A |
| 113 | C | A |
| 114 | C | A |
| 115 | C | C |
| 116 | A | A |
| 117 | A | B |
| 118 | A | C |
| 119 | C | C |
| 120 | C | C |
| 121 | A | C |
| 122 | B | C |
| 123 | A | B |
| 124 | C | A |
| 125 | A | nd |
| 126 | A | B |
| 127 | A | C |
| 128 | C | C |
| 129 | C | C |
| 130 | A | A |
| 131 | C | C |
| 132 | C | C |
| 133 | A | B |
| 134 | A | C |
| 135 | C | B |
| 136 | C | C |
| 137 | A | C |
| 138 | C | C |
| 139 | A | A |
| 140 | A | A |
| 141 | A | A |
| 142 | A | A |
| 143 | C | C |

1. A compound of formula (I), 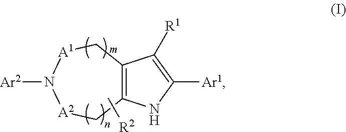
wherein A¹ and A² represent direct bond or C(═O), with the proviso that 0 or 1 of A¹ and A² represents C(═O);
m and n independently denote 0, 1, 2 or 3, with the proviso that the sum [n+m] is 1, 2, 3 or 4;
R¹ denotes H, F, Cl, Br, I, CN, CF₃, CF₂H, CFH₂, CO₂H, CO₂R¹³, R¹³, OH, O—R¹³, NH₂, N(H)R¹³, or N(R¹³)₂;
R² represents 0 to 4 substituents, each independently selected from the group consisting of F, Cl, Br, CN, CF₃, CF₂H, CFH₂, R¹³, OH, O—R¹³, NH₂, N(H)R¹³ and N(R¹³)₂;
Ar¹ represents phenyl or 5- or 6-membered heteroaryl, in each case unsubstituted or substituted with one, two, three or four substituents, independently selected from the group consisting of F, Cl, Br, CN, CF₃, CF₂H, CFH₂, R¹³ and O—R¹³;
or
C₃₋₆-cycloalkyl or 3 to 7 membered heterocycloalkyl, in each case unsubstituted or mono- or polysubstituted;
Ar² represents phenyl or 5- or 6-membered heteroaryl, wherein said phenyl or said heteroaryl is unsubstituted or mono- or polysubstituted and may be condensed with a 4-, 5-, 6- or 7-membered ring, being carbocyclic or heterocyclic, wherein said condensed ring may be saturated, partially unsaturated or aromatic and is unsubstituted or mono- or polysubstituted;
and
each R¹³ independently of each other denotes
C₁₋₈-alkyl, unsubstituted or mono- or polysubstituted; or
C₃₋₆-cycloalkyl or 3 to 7 membered heterocycloalkyl, in each case unsubstituted or mono- or polysubstituted; or
C₃₋₆-cycloalkyl or 3 to 7 membered heterocycloalkyl, in each case unsubstituted or mono- or polysubstituted, and in each case connected via a C₁₋₄-aliphatic group, unsubstituted or mono- or polysubstituted;
in the form of the free compound or a physiologically acceptable salt thereof or a physiologically acceptable solvate thereof.

2. A compound according to claim 1, wherein A¹ and A² each represent direct bond.

3. A compound according to claim 1, wherein the compound is selected from the group of consisting of compounds according to formula (I-a) or (I-b),

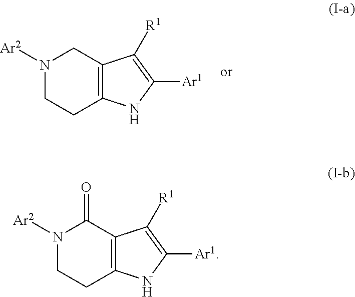

4. A compound according to claim 1, wherein R¹ represents H or F.

5. A compound according to claim 1, wherein Ar¹ represents substructure (II), 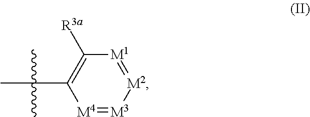
wherein
R^{3a} denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃,
and
M¹, M², M³ m and M⁴ independently represent N, CH or CR^{3b},
wherein R^{3b} denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃,
with the proviso, that 0 or 1 of the substituents M¹, M², M³ and M⁴ represent N.

6. A compound according to claim 5, wherein M¹, M² and M³ independently represent N or CH, and M⁴ represents N, CH or CR^{3b}.

7. A compound according to claim 1, wherein Ar² represents substructure (III), 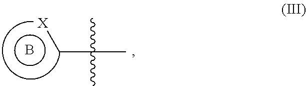
wherein
X represents CR⁴ or NR⁵,
wherein
R⁴ denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃,
and
R⁵ denotes CF₃, CF₂H, CFH₂, CH₃, CH₂CH₃ or cyclopropyl,
and B is phenyl or 5- or 6-membered heteroaryl, including the structural element “C—X”,
wherein B is unsubstituted or mono- or polysubstituted and wherein B may be condensed with a 4-, 5-, 6- or 7-membered ring, being carbocyclic or heterocyclic, wherein said condensed ring may be saturated, partially unsaturated or aromatic and is unsubstituted or mono- or polysubstituted,
wherein said substituents are independently selected from the group consisting of F; Cl; Br; CN; CF₃; CF₂H; CFH₂; CF₂C1; CFCl₂; R¹³; R¹⁴; C(═O)OH; C(═O)—R¹³; C(═O)R¹⁴; C(═O)—OR¹³; C(═O)—OR¹⁴; C(═O)NH₂; C(═O)—N(H)R¹³; C(═O)—N(R¹³)₂; C(═O)—N(H)R¹⁴; C(═O)—N(R¹⁴)₂; C(═O)—N(R¹³)(R¹⁴); C(═O)—N(R^{a})(R^{b}); OH; OR¹³; OCF₃; OCF₂H; OCFH₂; OCF₂Cl; OCFCl₂; OR¹⁴; O—C(═O)R¹³; O—C(═O)R¹⁴; O—C(═O)—N(H)R¹³; O—C(═O)—N(H)R¹⁴; O—C(═O)—N(R¹³)₂; O—C(═O)—N(R¹⁴)₂; O—C(═O)—N(R¹³)(R¹⁴); O—C(═O)—N(R^{a})(R^{b}); NH₂; N(H)R¹³; N(R¹³)₂; N(H)R¹⁴; N(R¹⁴)₂; N(R¹³)(R¹⁴); N(R^{a})(R^{b}); NH—C(═O)—R¹⁴; NH—C(═O)—R¹³; N(R¹³)C(═O)R¹³; N(R¹³)—C(═O)R¹⁴; NH—S(═O)₂R¹³; N(R¹³)S(═O)₂R¹³; NHS(═O)₂R¹⁴; N(R¹³)S(═O)₂R¹⁴; N(H)—C(═O)—OR¹³; N(H)—C(═O)—OR¹⁴; N(R¹³)—C(═O)—OR¹³; N(R¹³)—C(═O)—OR¹⁴; N(H)—C(═O)—NH₂; N(H)—C(═O)—N(H)R¹³; N(H)—C(═O)—N(H)R¹⁴; N(H)—C(═O)—N(R¹³)₂; N(H)—C(═O)—N(R¹⁴)₂; N(H)—C(═O)—N(R¹³)(R¹⁴); N(H)—C(═O)—N(R^{a})(R^{b}); N(R¹³)—C(═O)—NH₂; N(R¹³)—C(═O)—N(H)R¹³; N(R¹³)—C(═O)—N(H)R¹⁴; N(R¹³)—C(═O)—N(R¹³)₂; N(R¹³)—C(═O)—N(R¹⁴)₂; N(R¹³)—C(═O)—N(R¹³)(R¹⁴); N(R¹³)—C(═O)—N(R^{a})(R^{b}); SH; S—R¹³; SCF₃; S—R¹⁴; S(═O)₂OH; S(═O)₂—R¹³; S(═O)₂—R¹⁴; S(═O)—R¹³; S(═O)—R¹⁴; S(═O)₂—OR¹³; S(═O)₂—OR¹⁴; S(═O)₂—N(H)(R¹³); S(═O)₂—N(R¹³)₂; S(═O)₂—N(H)(R¹⁴); S(═O)₂—N(R¹³)(R¹⁴); and S(═O)₂—N(R^{a})(R^{b});
wherein
each R¹³ independently of each other denotes
C₁₋₈-alkyl, unsubstituted or mono- or polysubstituted; or
C₃₋₆-cycloalkyl or 3 to 7 membered heterocycloalkyl, in each case unsubstituted or mono- or polysubstituted; or
C₃₋₆-cycloalkyl or 3 to 7 membered heterocycloalkyl, in each case unsubstituted or mono- or polysubstituted, and in each case connected via a C₁₋₄-aliphatic group, unsubstituted or mono- or polysubstituted;
each R¹⁴ independently of each other denotes
aryl and heteroaryl, in each case independently of one another unsubstituted or mono- or polysubstituted, or
aryl and heteroaryl, in each case independently of one another unsubstituted or mono- or polysubstituted and in each case connected via a C₁₋₄-aliphatic group, unsubstituted or mono- or polysubstituted; and
R^{a} and R^{b} together with the N-atom connecting them form a 3 to 7 membered heterocycloalkyl, unsubstituted or mono- or polysubstituted.

8. A compound according to claim 1, wherein Ar² is selected from the group consisting of 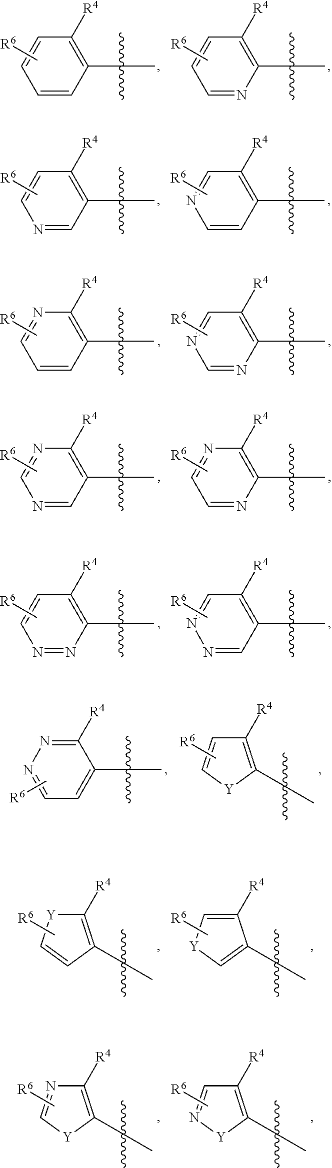 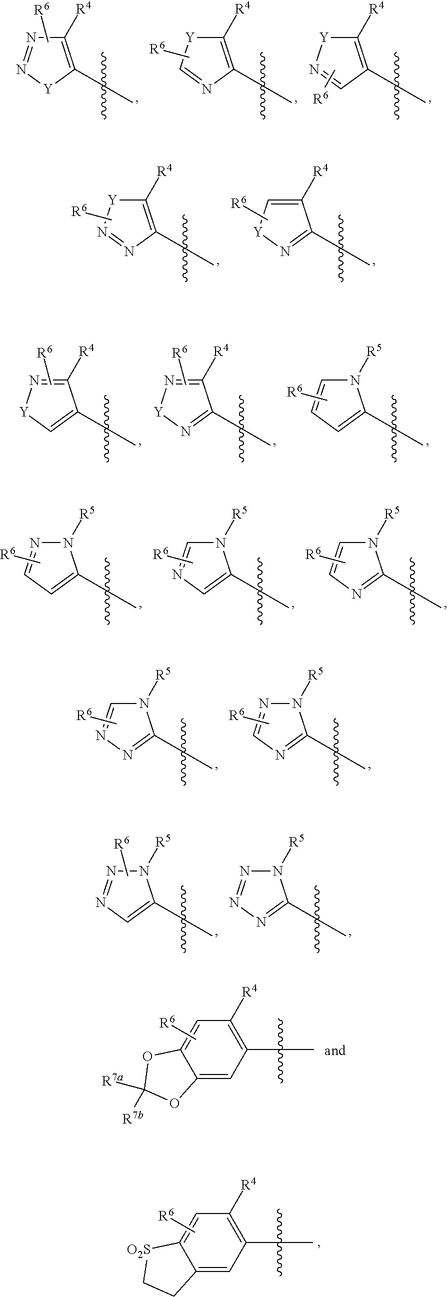
wherein
Y represents O, S or NR⁸;
R⁴ denotes F, Cl, CN, CF₃, CF₂H, CFH₂, CH₃, CH₂CH₃, cyclopropyl, OCH₃ or OCH₂CH₃;
R⁵ denotes CF₃, CF₂H, CFH₂, cyclopropyl, CH₃ or CH₂CH₃;
R^{7a} and R^{7b} each independently represent H, F or C₁₋₄-alkyl;
R⁸ denotes H, C₁₋₄-alkyl or C(═O)C₁₋₄-alkyl, wherein C₁₋₄-alkyl may be unsubstituted or substituted by one or more substituents selected from the group consisting of F, Cl, OH, NH₂, N(H)C₁₋₄-alkyl, N(C₁₋₄-alkyl)₂, N(H)C(═O)C₁₋₄-alkyl, N(C₁₋₄-alkyl)C(═O)C₁₋₄-alkyl, OH, OCH₃ and OCH₂CH₃;
and
R⁶ denotes 0, 1, 2 or 4 substituents, independently selected from the group consisting of F; Cl; Br; CN; CF₃; CF₂H; CFH₂; CF₂Cl; CFCl₂; R¹³; R¹⁴; C(═O)OH; C(═O)—R¹³; C(═O)R¹⁴; C(═O)—OR¹³; C(═O)—OR¹⁴; C(═O)NH₂; C(═O)—N(H)R¹³; C(═O)—N(R¹³)₂; C(═O)—N(H)R¹⁴; C(═O)—N(R¹⁴)₂; C(═O)—N(R¹³)(R¹⁴); C(═O)—N(R^{a})(R^{b}); OH; OR¹³; OCF₃; OCF₂H; OCFH₂; OCF₂Cl; OCFCl₂; OR¹⁴; O—C(═O)R¹³; O—C(═O)R¹⁴; O—C(═O)—N(H)R¹³; O—C(═O)—N(H)R¹⁴; O—C(═O)—N(R¹³)₂; O—C(═O)—N(R¹⁴)₂; O—C(═O)—N(R¹³)(R¹⁴); O—C(═O)—N(R^{a})(R^{b}); NH₂; N(H)R¹³; N(R¹³)₂; N(H)R¹⁴; N(R¹⁴)₂; N(R¹³)(R¹⁴); N(R^{a})(R^{b}); NH—C(═O)—R¹⁴; NH—C(═O)—R¹³; N(R¹³)—C(═O)—R¹³; N(R¹³)—C(═O)—R¹⁴; NH—S(═O)₂—R¹³; N(R¹³)—S(═O)₂—R¹³; NH—S(═O)₂—R¹⁴; N(R¹³)—S(═O)₂—R¹⁴; N(H)—C(═O)—OR¹³; N(H)—C(═O)—OR¹⁴; N(R¹³)—C(═O)—OR¹³; N(R¹³)—C(═O)—OR¹⁴; N(H)—C(═O)—NH₂; N(H)—C(═O)—N(H)R¹³; N(H)—C(═O)—N(H)R¹⁴; N(H)—C(═O)—N(R¹³)₂; N(H)—C(═O)—N(R¹⁴)₂; N(H)—C(═O)—N(R¹³)(R¹⁴); N(H)—C(═O)—N(R^{a})(R^{b}); N(R¹³)—C(═O)—NH₂; N(R¹³)—C(═O)—N(H)R¹³; N(R¹³)—C(═O)—N(H)R¹⁴; N(R¹³)—C(═O)—N(R¹³)₂; N(R¹³)—C(═O)—N(R¹⁴)₂; N(R¹³)—C(═O)—N(R¹³)(R¹⁴); N(R¹³)—C(═O)—N(R^{a})(R^{b}); SH; S—R¹³; SCF₃; S—R¹⁴; S(═O)₂OH; S(═O)₂—R¹³; S(═O)₂—R¹⁴; S(═O)—R¹³; S(═O)—R¹⁴; S(═O)₂—OR¹³; S(═O)₂—OR¹⁴; S(═O)₂—N(H)(R¹³); S(═O)₂—N(R¹³)₂; S(═O)₂—N(H)(R¹⁴); S(═O)₂—N(R¹³)(R¹⁴) and S(═O)₂—N(R^{a})(R^{b});
wherein
each R¹³ independently of each other denotes
C₁₋₈-alkyl, unsubstituted or mono- or polysubstituted; or
C₃₋₆-cycloalkyl or 3 to 7 membered heterocycloalkyl, in each case unsubstituted or mono- or polysubstituted; or
C₃₋₆-cycloalkyl or 3 to 7 membered heterocycloalkyl, in each case unsubstituted or mono- or polysubstituted, and in each case connected via a C₄-aliphatic group, unsubstituted or mono- or polysubstituted,
each R¹⁴ independently of each other denotes
aryl and heteroaryl, in each case independently of one another unsubstituted or mono- or polysubstituted, or
aryl and heteroaryl, in each case independently of one another unsubstituted or mono- or polysubstituted and in each case connected via a C₁₋₄-aliphatic group, unsubstituted or mono- or polysubstituted; and
R^{a} and R^{b} together with the N-atom connecting them form a 3 to 7 membered heterocycloalkyl, unsubstituted or mono- or polysubstituted.

9. A compound according to claim 8, wherein
R⁶ is selected from the group consisting of
F, Cl, Br, CN, C(═O)O—C₁₋₄-alkyl, CF₃, CF₂H, CFH₂, C(═O)NH₂, C(═O)—N(H)C₁₋₄-alkyl, C(═O)—N(C₁₋₄-alkyl)₂, C(═O)—N(H)(C₁₋₄-alkylene-OH), OCF₃, OCF₂H, OCFH₂, C₁₋₄-alkyl, OH, O—C₁₋₄-alkyl, S(═O)C₁₋₄-alkyl, SO₂—C₁₋₄-alkyl, SO₂—CF₃, SO₂—N(H)C₁₋₄-alkyl, SO₂—N(C₁₋₄-alkyl)₂, CH₂S(O)C₁₋₄-alkyl, CH₂SO₂—C₁₋₄-alkyl, CH₂N(H)SO₂—C₁₋₄-alkyl, CH₂SO₂—N(H)C₁₋₄-alkyl, CH₂SO₂—N(C₁₋₄-alkyl)₂,
C₃₋₆-cycloalkyl,
wherein the C₃₋₆-cycloalkyl is unsubstituted or mono- or di-substituted with at least one substituent selected from the group consisting of F, Cl, CN, OH, OCH₃, CF₃, CH₃ and CH₂CH₃,
3 to 7 membered heterocycloalkyl,
wherein the 3 to 7 membered heterocycloalkyl is unsubstituted or mono- or di-substituted with at least one substituent selected from the group consisting of F, Cl, CN, OH, OCH₃, ═O, CF₃, CH₃ and CH₂CH₃,
phenyl and
heteroaryl,
wherein said phenyl or said heteroaryl is unsubstituted or mono- or di-substituted with at least one substituent selected from the group consisting of F, Cl, Br, CN, CF₃, OCF₃, OH, NH₂, CH₃, OCH₃, CH₂CH₃ and OCH₂CH₃.

10. A compound according to one claim 1, wherein the compound is selected from compounds according to formula (Ia), 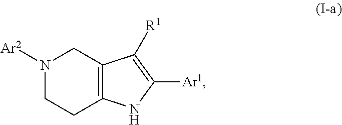
wherein R¹ represents H or F;
Ar¹ represents substructure (II), 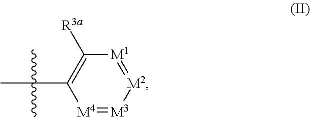
wherein
R^{1a} denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃;
M¹, M² and M³ independently represent N or CH,
and M⁴ represents N, CH or CR^{3b},
wherein R^{3b} denotes F, Cl, CN, CF₃, CF₂H, CFH₂, CH₃, CH₂CH₃, OCH₃ or OCH₂CH₃,
with the proviso, that 0 or 1 of the substituents M¹, M², M³ and M⁴ represent N, and
Ar² is selected from the group consisting of 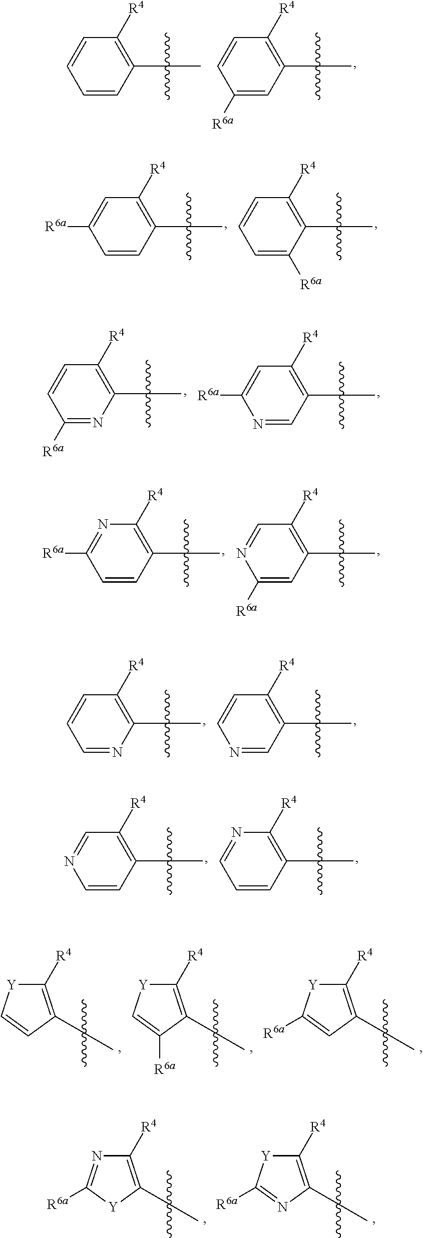 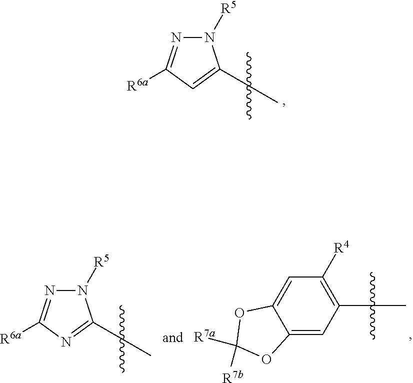
wherein Y represents S;
R⁴ denotes F, Cl, CN, CF₃, CF₂H, CFH₂, CH₃, CH₂CH₃, cyclopropyl, OCH₃, OCH₂CH₃, OCF₃, OCF₂H, OCFH₂ or OCH₂CF₃;
R⁵ denotes CF₃, CF₂H, CFH₂, cyclopropyl, CH₃ or CH₂CH₃;
R^{7a} and R^{7b} each independently represent H, F or C₁₋₄-alkyl;
and R^{6a} is selected from the group consisting of
F, Cl, Br, CN, C(═O)O—C₁₋₄-alkyl, C(═O)—N(H)C₁₋₄-alkyl, C(═O)—N(C₁₋₄-alkyl)₂, C(═O)—N(H)(C₁₋₄-alkylene-OH), CF₃, CF₂H, CFH₂, OCF₃, C₁₋₄-alkyl, OH, O—C₁₋₄-alkyl, S(═O)C₁₋₄-alkyl, SO₂—C₁₋₄-alkyl, SO₂—CF₃, SO₂—N(H)C₁₋₄-alkyl, SO₂—N(C₁₋₄-alkyl)₂, CH₂S(═O)C₁₋₄-alkyl, CH₂SO₂—C₁₋₄-alkyl, CH₂N(H)SO₂—C₁₋₄-alkyl, CH₂SO₂—N(H)C₁₋₄-alkyl, CH₂SO₂—N(C₁₋₄-alkyl)₂,
cyclopropyl, cyclobuytl, cyclopentyl, cyclohexyl,
oxetanyl, pyrrolidinyl, piperidinyl, morpholinyl,
phenyl,
oxazolyl, isoxazolyl, thiazolyl, thienyl, pyrrolyl, pyrazolyl, oxadiazolyl, tetrazolyl, pyridinyl, pyrazinyl, and pyrimidinyl,
wherein said cyclopropyl, cyclobuytl, cyclopentyl, cyclohexyl, pyrrolidinyl, piperidinyl, morpholinyl, phenyl, oxazolyl, isoxazolyl, thiazolyl, thienyl, pyrrolyl, pyrazolyl, oxadiazolyl, tetrazolyl, pyridinyl, pyrazinyl and pyrimidinyl may be unsubstituted or substituted by one or two substituents, independently selected from F, Cl, CN, CF₃, CH₃, CH₂CH₃, OH, OCH₃ or OCF₃,
in the form of a single stereoisomer or a mixture of stereoisomers, in the form of the free compound or a physiologically acceptable salt or a physiologically acceptable solvate thereof.

11. A compound according to claim 1, wherein Ar¹ is selected from the group consisting of 2,6-difluorophenyl, 2,6-difluoro-4-methoxyphenyl, 2-chlorophenyl, 2-chloro-4-fluorophenyl, 2-chloro-6-fluorophenyl, 5-fluoro-4-methyl-pyridin-3-yl, 2,4-difluorophenyl, 2,4-dimethoxyphenyl, 3-fluoro-pyridin-4-yl and 2-fluoro-pyridin-3-yl.

12. A compound according to claim 1, selected from the group consisting of
1 2-(2,6-Difluoro-phenyl)-5-(5-methyl-2-pyridin-3-yl-thiazol-4-yl)-1,5,6,7-tetrahydro-pyrrolo[3,2-c]pyridin-4-one
2 4-[2-(2,6-Difluoro-phenyl)-4,5,6,7-tetrahydro-1H-pyrrolo[3,2-c]pyridin-5-yl]-5-methyl-2-pyridin-3-yl-thiazole
3 5-(6-Chloro-4-methyl-pyridin-3-yl)-2-(2,6-difluoro-phenyl)-1,5,6,7-tetrahydro-pyrrolo[3,2-c]pyridin-4-one
4 5-(6-Chloro-4-methyl-pyridin-3-yl)-2-(2,6-difluoro-phenyl)-4,5,6,7-tetrahydro-1H-pyrrolo[3,2-c]pyridine
5 2-(2,6-Difluoro-phenyl)-5-(5-methyl-2-pyridin-2-yl-thiazol-4-yl)-1,5,6,7-tetrahydro-pyrrolo[3,2-c]pyridin-4-one
6 4-[2-(2,6-Difluoro-phenyl)-4,5,6,7-tetrahydro-1H-pyrrolo[3,2-c]pyridin-5-yl]-5-methyl-2-pyridin-2-yl-thiazole
7 2-(2,6-Difluoro-phenyl)-5-[3-(trifluoromethyl)phenyl]-4,5,6,7-tetrahydro-1H-pyrrolo[3,2-c]pyridine
8 2-[3-[2-(2,6-Difluoro-phenyl)-4,5,6,7-tetrahydro-1H-pyrrolo[3,2-c]pyridin-5-yl]-4-methyl-phenyl]-thiazole
9 5-(6-Chloro-2,2-difluoro-1,3-benzodioxol-5-yl)-2-(2,6-difluoro-phenyl)-4,5,6,7-tetrahydro-1H-pyrrolo[3,2-c]pyridine
10 2-(2,6-Difluoro-phenyl)-5-(4-methyl-pyridin-3-yl)-4,5,6,7-tetrahydro-1H-pyrrolo[3,2-c]pyridine
11 5-(4-Chloro-2-methyl-phenyl)-2-(2,6-difluoro-phenyl)-4,5,6,7-tetrahydro-1H-pyrrolo[3,2-c]pyridine
12 4-[2-(2,6-Difluoro-phenyl)-4,5,6,7-tetrahydro-1H-pyrrolo[3,2-c]pyridin-5-yl]-3-methyl-benzonitrile
13 4-[2-(2,6-Difluoro-phenyl)-4,5,6,7-tetrahydro-1H-pyrrolo[3,2-c]pyridin-5-yl]-3-methyl-benzoic acid methyl ester
14 4-[2-(2,6-Difluoro-phenyl)-4,5,6,7-tetrahydro-1H-pyrrolo[3,2-c]pyridin-5-yl]-N,N,3-trimethyl-benzenesulfonic acid amide
15 5-(6-Chloro-2-methyl-pyridin-3-yl)-2-(2,6-difluoro-phenyl)-4,5,6,7-tetrahydro-1H-pyrrolo[3,2-c]pyridine
16 2-(2,6-Difluoro-phenyl)-5-(4-methyl-6-methylsulfonyl-pyridin-3-yl)-4,5,6,7-tetrahydro-1H-pyrrolo[3,2-c]pyridine
17 4-[2-(2-Chloro-6-fluoro-phenyl)-4,5,6,7-tetrahydro-1H-pyrrolo[3,2-c]pyridin-5-yl]-5-methyl-2-pyridin-3-yl-thiazole
18 5-[2-(2,6-Difluoro-phenyl)-4,5,6,7-tetrahydro-1H-pyrrolo[3,2-c]pyridin-5-yl]-4-methyl-2-pyridin-3-yl-thiazole
19 2-(2,6-Difluoro-phenyl)-5-[2-methyl-5-(trifluoromethyl)-2H-pyrazol-3-yl]-4,5,6,7-tetrahydro-1H-pyrrolo[3,2-c]pyridine
20 5-(2,2-Difluoro-6-methyl-1,3-benzodioxol-5-yl)-2-(2,6-difluoro-phenyl)-4,5,6,7-tetrahydro-1H-pyrrolo[3,2-c]pyridine
21 2-(2,6-Difluoro-phenyl)-5-(2,5-dimethoxyphenyl)-4,5,6,7-tetrahydro-1H-pyrrolo[3,2-c]pyridine
22 2-(2,6-Difluoro-phenyl)-5-o-tolyl-4,5,6,7-tetrahydro-1H-pyrrolo[3,2-c]pyridine
23 4-[2-(2,6-Difluoro-phenyl)-4,5,6,7-tetrahydro-1H-pyrrolo[3,2-c]pyridin-5-yl]-5-methyl-2-phenyl-thiazole
24 4-[2-(2,6-Difluoro-phenyl)-4,5,6,7-tetrahydro-1H-pyrrolo[3,2-c]pyridin-5-yl]-5-methyl-2-pyridin-4-yl-thiazole
25 5-(5-Bromo-6-methyl-pyridin-2-yl)-2-(2,6-difluoro-phenyl)-4,5,6,7-tetrahydro-1H-pyrrolo[3,2-c]pyridine
26 4-[2-(2,6-Difluoro-phenyl)-4,5,6,7-tetrahydro-1H-pyrrolo[3,2-c]pyridin-5-yl]-5-methyl-2-pyrazin-2-yl-thiazole
27 4-[2-(2,6-Difluoro-phenyl)-4,5,6,7-tetrahydro-1H-pyrrolo[2,3-c]pyridin-6-yl]-5-methyl-2-pyridin-3-yl-thiazole
28 2-(2,6-Difluoro-phenyl)-5-(2-methoxy-4-methylsulfonyl-phenyl)-4,5,6,7-tetrahydro-1H-pyrrolo[3,2-c]pyridine
29 3-[2-(2,6-Difluoro-phenyl)-4,5,6,7-tetrahydro-1H-pyrrolo[3,2-c]pyridin-5-yl]-4-methyl-benzonitrile
30 4-[2-(2,6-Difluoro-phenyl)-4,5,6,7-tetrahydro-1H-pyrrolo[3,2-c]pyridin-5-yl]-5-methyl-2-pyrimidin-5-yl-thiazole
31 4-[2-(2,6-Difluoro-phenyl)-4,5,6,7-tetrahydro-1H-pyrrolo[3,2-c]pyridin-5-yl]-5-ethyl-2-pyridin-3-yl-thiazole
32 5-(6-Chloro-5-methyl-pyridazin-3-yl)-2-(2,6-difluoro-phenyl)-4,5,6,7-tetrahydro-1H-pyrrolo[3,2-c]pyridine
33 5-[2-(2,6-Difluoro-phenyl)-4,5,6,7-tetrahydro-1H-pyrrolo[3,2-c]pyridin-5-yl]-6-methoxy-2,3-dihydro-benzo[b]thiophene 1,1-dioxide
34 5-(4-Methyl-6-methylsulfonyl-pyridin-3-yl)-2-phenyl-4,5,6,7-tetrahydro-1H-pyrrolo[3,2-c]pyridine
35 5-(4-Methyl-6-methylsulfonyl-pyridin-3-yl)-2-o-tolyl-4,5,6,7-tetrahydro-1H-pyrrolo[3,2-c]pyridine
36 2-(2-Chloro-6-fluoro-phenyl)-5-(4-methyl-6-methylsulfonyl-pyridin-3-yl)-4,5,6,7-tetrahydro-1H-pyrrolo[3,2-c]pyridine
37 2-(4-Fluorophenyl)-5-(4-methyl-6-methylsulfonyl-pyridin-3-yl)-4,5,6,7-tetrahydro-1H-pyrrolo[3,2-c]pyridine
38 5-(6-Chloro-4-methyl-pyridin-3-yl)-2-(2,6-difluoro-phenyl)-1,5,6,7-tetrahydro-pyrrolo[3,2-c]pyridin-4-one
39 2-(2,6-Difluoro-phenyl)-5-(6-methoxy-4-methyl-pyridin-3-yl)-1,5,6,7-tetrahydro-pyrrolo[3,2-c]pyridin-4-one
40 2-(2,4-Dimethoxy-phenyl)-5-(5-methyl-2-pyridin-3-yl-thiazol-4-yl)-1,5,6,7-tetrahydro-pyrrolo[3,2-c]pyridin-4-one
41 4-[2-(2-Chloro-6-fluoro-phenyl)-4-oxo-1,5,6,7-tetrahydro-pyrrolo[3,2-c]pyridin-5-yl]-3-methoxy-benzonitrile
42 2-(2-Chloro-6-fluoro-phenyl)-5-(5-methyl-2-pyridin-3-yl-thiazol-4-yl)-1,5,6,7-tetrahydro-pyrrolo[3,2-c]pyridin-4-one
43 2-(6-Chloro-pyridin-3-yl)-5-(5-methyl-2-pyridin-3-yl-thiazol-4-yl)-1,5,6,7-tetrahydro-pyrrolo[3,2-c]pyridin-4-one
44 5-(6-Chloro-4-methyl-pyridin-3-yl)-2-(2,4-difluoro-phenyl)-1,5,6,7-tetrahydro-pyrrolo[3,2-c]pyridin-4-one
45 2-(2-Chloro-6-fluoro-phenyl)-5-(6-chloro-4-methyl-pyridin-3-yl)-1,5,6,7-tetrahydro-pyrrolo[3,2-c]pyridin-4-one
46 2,5-Bis(2,6-difluoro-phenyl)-1,5,6,7-tetrahydro-pyrrolo[3,2-c]pyridin-4-one
47 2-(2,6-Difluoro-phenyl)-5-(2-methyl-thiophen-3-yl)-1,5,6,7-tetrahydro-pyrrolo[3,2-c]pyridin-4-one
48 5-(2-Cyclopropyl-5-methyl-thiazol-4-yl)-2-(2,6-difluoro-phenyl)-1,5,6,7-tetrahydro-pyrrolo[3,2-c]pyridin-4-one
49 2-(2-Chloro-6-fluoro-phenyl)-5-(2,6-difluoro-phenyl)-1,5,6,7-tetrahydro-pyrrolo[3,2-c]pyridin-4-one
50 2-(2-Chloro-6-fluoro-phenyl)-5-(5-chloro-2-methyl-phenyl)-1,5,6,7-tetrahydro-pyrrolo[3,2-c]pyridin-4-one
51 2-(2-Chloro-6-fluoro-phenyl)-5-(2-methyl-thiophen-3-yl)-1,5,6,7-tetrahydro-pyrrolo[3,2-c]pyridin-4-one
52 2-(2-Chloro-6-fluoro-phenyl)-5-(2-cyclopropyl-5-methyl-thiazol-4-yl)-1,5,6,7-tetrahydro-pyrrolo[3,2-c]pyridin-4-one
53 2-(2-Chloro-6-fluoro-phenyl)-5-(5-methyl-2-oxazol-2-yl-thiazol-4-yl)-1,5,6,7-tetrahydro-pyrrolo[3,2-c]pyridin-4-one
54 2-(2,6-Difluoro-phenyl)-5-(5-methyl-2-oxazol-2-yl-thiazol-4-yl)-1,5,6,7-tetrahydro-pyrrolo[3,2-c]pyridin-4-one
55 2-(2,6-Difluoro-phenyl)-5-(2-methyl-thiophen-3-yl)-4,5,6,7-tetrahydro-1H-pyrrolo[3,2-c]pyridine
56 2-Cyclopropyl-4-[2-(2,6-difluoro-phenyl)-4,5,6,7-tetrahydro-1H-pyrrolo[3,2-c]pyridin-5-yl]-5-methyl-thiazole
57 2-[4-[2-(2,6-Difluoro-phenyl)-4,5,6,7-tetrahydro-1H-pyrrolo[3,2-c]pyridin-5-yl]-5-methyl-thiazol-2-yl]-oxazole
58 2-(2,6-Difluoro-phenyl)-5-(2,5-dimethyl-2H-pyrazol-3-yl)-4,5,6,7-tetrahydro-1H-pyrrolo[3,2-c]pyridine
59 2-(2-Chloro-6-fluoro-phenyl)-5-(2,6-difluoro-phenyl)-4,5,6,7-tetrahydro-1H-pyrrolo[3,2-c]pyridine
60 2-(2-Chloro-6-fluoro-phenyl)-5-(2-methyl-thiophen-3-yl)-4,5,6,7-tetrahydro-1H-pyrrolo[3,2-c]pyridine
61 4-[2-(2-Chloro-6-fluoro-phenyl)-4,5,6,7-tetrahydro-1H-pyrrolo[3,2-c]pyridin-5-yl]-2-cyclopropyl-5-methyl-thiazole
62 2-[4-[2-(2-Chloro-6-fluoro-phenyl)-4,5,6,7-tetrahydro-1H-pyrrolo[3,2-c]pyridin-5-yl]-5-methyl-thiazol-2-yl]-oxazole
63 2-(2-Chloro-6-fluoro-phenyl)-5-(2,5-dimethyl-2H-pyrazol-3-yl)-4,5,6,7-tetrahydro-1H-pyrrolo[3,2-c]pyridine
64 2-(2-Chloro-6-fluoro-phenyl)-5-(5-chloro-2-methyl-phenyl)-4,5,6,7-tetrahydro-1H-pyrrolo[3,2-c]pyridine
65 2-(3-Fluoro-pyridin-4-yl)-5-(5-methyl-2-pyridin-3-yl-thiazol-4-yl)-1,5,6,7-tetrahydro-pyrrolo[3,2-c]pyridin-4-one
66 4-[2-(2,6-Difluoro-phenyl)-3-iodo-4,5,6,7-tetrahydro-1H-pyrrolo[3,2-c]pyridin-5-yl]-5-methyl-2-pyridin-3-yl-thiazole
67 4-[2-(2,6-Difluoro-phenyl)-4,5,6,7-tetrahydro-1H-pyrrolo[3,2-c]pyridin-5-yl]-2-fluoro-5-methyl-benzonitrile
68 6-[2-(2,6-Difluoro-phenyl)-4,5,6,7-tetrahydro-1H-pyrrolo[3,2-c]pyridin-5-yl]-5-methyl-nicotinonitrile
69 2-(2,6-Difluoro-phenyl)-5-(6-ethoxy-4-methyl-pyridin-3-yl)-4,5,6,7-tetrahydro-1H-pyrrolo[3,2-c]pyridine
70 2-(2,6-Difluoro-phenyl)-5-[4-methyl-6-(methylsulfinyl)-pyridin-3-yl]-4,5,6,7-tetrahydro-1H-pyrrolo[3,2-c]pyridine
71 5-(4-Chloro-5-fluoro-2-methyl-phenyl)-2-(2,6-difluoro-phenyl)-4,5,6,7-tetrahydro-1H-pyrrolo[3,2-c]pyridine
72 2-Cyclohexyl-5-(4-methyl-6-methylsulfonyl-pyridin-3-yl)-4,5,6,7-tetrahydro-1H-pyrrolo[3,2-c]pyridine
73 2-(2,6-Difluoro-phenyl)-5-[4-methyl-6-(trifluoromethyl)-pyridin-3-yl]-4,5,6,7-tetrahydro-1H-pyrrolo[3,2-c]pyridine
74 2-Butyl-5-(4-methyl-6-methylsulfonyl-pyridin-3-yl)-4,5,6,7-tetrahydro-1H-pyrrolo[3,2-c]pyridine
75 5-(6-Cyclopropyl-4-methyl-pyridin-3-yl)-2-(2,6-difluoro-phenyl)-4,5,6,7-tetrahydro-1H-pyrrolo[3,2-c]pyridine
76 5-(4-Methyl-6-methylsulfonyl-pyridin-3-yl)-2-tetrahydro-pyran-4-yl-4,5,6,7-tetrahydro-1H-pyrrolo[3,2-c]pyridine
77 5-[2-(2,6-Difluoro-phenyl)-4,5,6,7-tetrahydro-1H-pyrrolo[3,2-c]pyridin-5-yl]-6-methyl-pyridine-2-carbonitrile
78 2-(2,4-Difluoro-phenyl)-5-(4-methyl-6-methylsulfonyl-pyridin-3-yl)-4,5,6,7-tetrahydro-1H-pyrrolo[3,2-c]pyridine
79 2-(2-Fluorophenyl)-5-(4-methyl-6-methylsulfonyl-pyridin-3-yl)-4,5,6,7-tetrahydro-1H-pyrrolo[3,2-c]pyridine
80 4-[2-(2,6-Difluoro-phenyl)-4,5,6,7-tetrahydro-1H-pyrrolo[3,2-c]pyridin-5-yl]-N,3-dimethyl-benzamide
81 4-[2-(2,6-Difluoro-phenyl)-4,5,6,7-tetrahydro-1H-pyrrolo[3,2-c]pyridin-5-yl]-N,N,3-trimethyl-benzamide
82 1-[4-[2-(2,6-Difluoro-phenyl)-4,5,6,7-tetrahydro-1H-pyrrolo[3,2-c]pyridin-5-yl]-3-methyl-phenyl]-ethanone
83 5-(4-Methyl-6-methylsulfonyl-pyridin-3-yl)-2-(2-methyl-pyridin-3-yl)-4,5,6,7-tetrahydro-1H-pyrrolo[3,2-c]pyridine
84 2-(2,6-Difluoro-phenyl)-5-[5-methoxy-2-(trifluoromethyl)-pyrimidin-4-yl]-4,5,6,7-tetrahydro-1H-pyrrolo[3,2-c]pyridine
[85 2-(2,6-Difluoro-phenyl)-5-(5-methyl-2-pyridin-3-yl-thiazol-4-yl)-4,5,6,7-tetrahydro-1H-pyrrolo[3,2-c]pyridin-3-yl]-methanol
86 2-(2,6-Difluoro-phenyl)-5-[2-methyl-4-(trifluoromethylsulfonyl)-phenyl]-4,5,6,7-tetrahydro-1H-pyrrolo[3,2-c]pyridine
87 2-(2,6-Difluoro-phenyl)-5-[2-methyl-4-(methylsulfinyl)-phenyl]-4,5,6,7-tetrahydro-1H-pyrrolo[3,2-c]pyridine
88 2-(2,6-Difluoro-phenyl)-5-(2-methyl-4-methylsulfonyl-phenyl)-4,5,6,7-tetrahydro-1H-pyrrolo[3,2-c]pyridine
89 4-Methyl-5-[5-(4-methyl-6-methylsulfonyl-pyridin-3-yl)-4,5,6,7-tetrahydro-1H-pyrrolo[3,2-c]pyridin-2-yl]-[1,2,3]thiadiazole
90 2-(3-Fluoro-pyridin-4-yl)-5-(4-methyl-6-methylsulfonyl-pyridin-3-yl)-4,5,6,7-tetrahydro-1H-pyrrolo[3,2-c]pyridine
91 3-Bromo-2-(2,6-difluoro-phenyl)-5-(4-methyl-6-methylsulfonyl-pyridin-3-yl)-4,5,6,7-tetrahydro-1H-pyrrolo[3,2-c]pyridine
92 2-(4,6-Dimethyl-pyridin-3-yl)-5-(4-methyl-6-methylsulfonyl-pyridin-3-yl)-4,5,6,7-tetrahydro-1H-pyrrolo[3,2-c]pyridine
93 5-[4-Methyl-6-(trifluoromethyl)-pyridin-3-yl]-2-tetrahydro-pyran-4-yl-4,5,6,7-tetrahydro-1H-pyrrolo[3,2-c]pyridine
94 2-(2,6-Difluoro-phenyl)-5-(5-fluoro-4-methyl-pyridin-3-yl)-4,5,6,7-tetrahydro-1H-pyrrolo[3,2-c]pyridine
[95 5-[2-(2,6-Difluoro-phenyl)-4,5,6,7-tetrahydro-1H-pyrrolo[3,2-c]pyridin-5-yl]-4-methyl-pyridin-2-yl]-amine
96 2-(5-Fluoro-4-methyl-pyridin-3-yl)-5-(4-methyl-6-methylsulfonyl-pyridin-3-yl)-4,5,6,7-tetrahydro-1H-pyrrolo[3,2-c]pyridine
97 2-Cyclohexyl-5-[4-methyl-6-(trifluoromethyl)-pyridin-3-yl]-4,5,6,7-tetrahydro-1H-pyrrolo[3,2-c]pyridine
98 2-(2-Methoxy-pyridin-3-yl)-5-(4-methyl-6-methylsulfonyl-pyridin-3-yl)-4,5,6,7-tetrahydro-1H-pyrrolo[3,2-c]pyridine
99 4-[2-(2,6-Difluoro-phenyl)-4,5,6,7-tetrahydro-1H-pyrrolo[3,2-c]pyridin-5-yl]-5-methyl-2-(1-methyl-1H-pyrazol-3-yl)-thiazole
100 2-(2,6-Difluoro-phenyl)-5-[6-ethoxy-4-(trifluoromethyl)-pyridin-3-yl]-4,5,6,7-tetrahydro-1H-pyrrolo[3,2-c]pyridine
101 2-(2,6-Difluoro-phenyl)-5-(5-fluoro-2-methyl-4-methylsulfonyl-phenyl)-4,5,6,7-tetrahydro-1H-pyrrolo[3,2-c]pyridine
102 6-(2-Cyclohexyl-4,5,6,7-tetrahydro-1H-pyrrolo[3,2-c]pyridin-5-yl)-5-methyl-nicotinonitrile
103 5-(2-Cyclohexyl-4,5,6,7-tetrahydro-1H-pyrrolo[3,2-c]pyridin-5-yl)-6-methoxy-2,3-dihydro-benzo[b]thiophene 1,1-dioxide
104 2-(2,6-Difluoro-phenyl)-5-[4-methyl-6-(methylsulfonyl-methyl)-pyridin-3-yl]-4,5,6,7-tetrahydro-1H-pyrrolo[3,2-c]pyridine
105 2-(2,6-Difluoro-phenyl)-5-[4-(methoxymethyl)-2-methyl-phenyl]-4,5,6,7-tetrahydro-1H-pyrrolo[3,2-c]pyridine
106 5-(5-Cyclopropyl-2-methyl-2H-pyrazol-3-yl)-2-(2,6-difluoro-phenyl)-4,5,6,7-tetrahydro-1H-pyrrolo[3,2-c]pyridine
107 5-(5-Cyclopropyl-1-methyl-1H-pyrazol-3-yl)-2-(2,6-difluoro-phenyl)-4,5,6,7-tetrahydro-1H-pyrrolo[3,2-c]pyridine
108 2-(3-Chloro-pyridin-4-yl)-5-(4-methyl-6-methylsulfonyl-pyridin-3-yl)-4,5,6,7-tetrahydro-1H-pyrrolo[3,2-c]pyridine
109 5-[4-(Azetidin-1-ylsulfonyl)-2-methyl-phenyl]-2-(2,6-difluoro-phenyl)-4,5,6,7-tetrahydro-1H-pyrrolo[3,2-c]pyridine
110 2-Cyclohexyl-5-(5-fluoro-2-methyl-4-methylsulfonyl-phenyl)-4,5,6,7-tetrahydro-1H-pyrrolo[3,2-c]pyridine
111 2-(2,6-Difluoro-phenyl)-5-[4-methyl-6-(trifluoromethyloxy)-pyridin-3-yl]-4,5,6,7-tetrahydro-1H-pyrrolo[3,2-c]pyridine
112 2-(2,6-Difluoro-phenyl)-5-[4-methoxy-6-(trifluoromethyl)-pyridin-3-yl]-4,5,6,7-tetrahydro-1H-pyrrolo[3,2-c]pyridine
113 5-[4-Cyclopropyl-6-(trifluoromethyl)-pyridin-3-yl]-2-(2,6-difluoro-phenyl)-4,5,6,7-tetrahydro-1H-pyrrolo[3,2-c]pyridine
114 4-[2-(2,6-Difluoro-phenyl)-4,5,6,7-tetrahydro-1H-pyrrolo[3,2-c]pyridin-5-yl]-N,3-dimethyl-benzenesulfonic acid amide
115 5-(4-Methyl-6-methylsulfonyl-pyridin-3-yl)-2-tetrahydro-pyran-3-yl-4,5,6,7-tetrahydro-1H-pyrrolo[3,2-c]pyridine
116 2-(2,6-Difluoro-phenyl)-5-[4-ethoxy-6-(trifluoromethyl)-pyridin-3-yl]-4,5,6,7-tetrahydro-1H-pyrrolo[3,2-c]pyridine
117 4-[[4-[2-(2,6-Difluoro-phenyl)-4,5,6,7-tetrahydro-1H-pyrrolo[3,2-c]pyridin-5-yl]-3-methyl-phenyl]sulfonyl]-morpholine
118 2-Cyclopentyl-5-(4-methyl-6-methylsulfonyl-pyridin-3-yl)-4,5,6,7-tetrahydro-1H-pyrrolo[3,2-c]pyridine
119 2-(2,6-Difluoro-phenyl)-5-[2-methyl-4-(piperidin-1-ylsulfonyl)-phenyl]-4,5,6,7-tetrahydro-1H-pyrrolo[3,2-c]pyridine
120 N-Cyclopropyl-4-[2-(2,6-difluoro-phenyl)-4,5,6,7-tetrahydro-1H-pyrrolo[3,2-c]pyridin-5-yl]-N,3-dimethyl-benzenesulfonic acid amide
121 2-(2,6-Difluoro-phenyl)-5-[2-methyl-4-(pyrrolidin-1-ylsulfonyl)-phenyl]-4,5,6,7-tetrahydro-1H-pyrrolo[3,2-c]pyridine
122 2-(4,4-Difluoro-cyclohexyl)-5-(4-methyl-6-methylsulfonyl-pyridin-3-yl)-4,5,6,7-tetrahydro-1H-pyrrolo[3,2-c]pyridine
123 2-(2,6-Difluoro-phenyl)-5-[2-methyl-5-[1-(trifluoromethyl)-cyclopropyl]-2H-pyrazol-3-yl]-4,5,6,7-tetrahydro-1H-pyrrolo[3,2-c]pyridine
124 2-(2,6-Difluoro-phenyl)-5-[2-ethyl-5-[1-(trifluoromethyl)-cyclopropyl]-2H-pyrazol-3-yl]-4,5,6,7-tetrahydro-1H-pyrrolo[3,2-c]pyridine
125 2-(2,6-Difluoro-phenyl)-5-[1-ethyl-5-[1-(trifluoromethyl)-cyclopropyl]-1H-pyrazol-3-yl]-4,5,6,7-tetrahydro-1H-pyrrolo[3,2-c]pyridine
126 5-(5-Cyclopropyl-2-ethyl-2H-pyrazol-3-yl)-2-(2,6-difluoro-phenyl)-4,5,6,7-tetrahydro-1H-pyrrolo[3,2-c]pyridine
127 4-[2-(4,6-Dimethyl-pyridin-3-yl)-4,5,6,7-tetrahydro-1H-pyrrolo[3,2-c]pyridin-5-yl]-N,3-dimethyl-benzenesulfonic acid amide
128 4-[2-(4,4-Difluoro-cyclohexyl)-4,5,6,7-tetrahydro-1H-pyrrolo[3,2-c]pyridin-5-yl]-N,3-dimethyl-benzenesulfonic acid amide
129 4-(2-Cyclohexyl-4,5,6,7-tetrahydro-1H-pyrrolo[3,2-c]pyridin-5-yl)-N-methyl-benzenesulfonic acid amide
130 2-(2,6-Difluoro-phenyl)-5-[2-ethyl-5-(trifluoromethyl)-2H-pyrazol-3-yl]-4,5,6,7-tetrahydro-1H-pyrrolo[3,2-c]pyridine
131 4-[2-(2,6-Difluoro-phenyl)-4,5,6,7-tetrahydro-1H-pyrrolo[3,2-c]pyridin-5-yl]-N-ethyl-3-methyl-benzenesulfonic acid amide
132 N-[4-[2-(2,6-Difluoro-phenyl)-4,5,6,7-tetrahydro-1H-pyrrolo[3,2-c]pyridin-5-yl]-3-methyl-phenyl]-N-methyl-methanesulfonic acid amide
133 4-[2-(2,6-Difluoro-phenyl)-4,5,6,7-tetrahydro-1H-pyrrolo[3,2-c]pyridin-5-yl]-5-methyl-2-methylsulfonyl-thiazole
134 5-[4-Methyl-6-(trifluoromethyl)-pyridin-3-yl]-2-tetrahydro-pyran-3-yl-4,5,6,7-tetrahydro-1H-pyrrolo[3,2-c]pyridine
135 2-(4,6-Dimethyl-pyridin-3-yl)-5-[4-methyl-6-(trifluoromethyl)-pyridin-3-yl]-4,5,6,7-tetrahydro-1H-pyrrolo[3,2-c]pyridine
136 4-[2-(2,6-Difluoro-phenyl)-4,5,6,7-tetrahydro-1H-pyrrolo[3,2-c]pyridin-5-yl]-3-methyl-N-(2,2,2-trifluoro-ethyl)-benzenesulfonic acid amide
137 2-(2,6-Difluoro-phenyl)-5-[5-(1-methoxy-cyclopropyl)-2-methyl-2H-pyrazol-3-yl]-4,5,6,7-tetrahydro-1H-pyrrolo[3,2-c]pyridine
138 2-(2,6-Difluoro-phenyl)-4-methyl-5-(4-methyl-6-methylsulfonyl-pyridin-3-yl)-4,5,6,7-tetrahydro-1H-pyrrolo[3,2-c]pyridine
139 2-(2,6-Difluoro-phenyl)-5-[2-ethyl-5-(1-methoxy-cyclopropyl)-2H-pyrazol-3-yl]-4,5,6,7-tetrahydro-1H-pyrrolo[3,2-c]pyridine
140 5-[6-(Difluoro-methoxy)-4-methoxy-pyridin-3-yl]-2-(2,6-difluoro-phenyl)-4,5,6,7-tetrahydro-1H-pyrrolo[3,2-c]pyridine
141 2-(5-Fluoro-4-methyl-pyridin-3-yl)-5-[4-methyl-6-(trifluoromethyl)-pyridin-3-yl]-4,5,6,7-tetrahydro-1H-pyrrolo[3,2-c]pyridine
142 2-(2,6-Difluoro-phenyl)-3-fluoro-5-(4-methyl-6-methylsulfonyl-pyridin-3-yl)-4,5,6,7-tetrahydro-1H-pyrrolo[3,2-c]pyridine
143 2-(2,6-Difluoro-phenyl)-5-(6-ethoxy-4-methoxy-pyridin-3-yl)-4,5,6,7-tetrahydro-1H-pyrrolo[3,2-c]pyridine
in the form of the free compound or a physiologically acceptable salt or solvate thereof.

13. A pharmaceutical composition comprising at least one compound according to claim 1.

14. A method of treating and/or providing prophylaxis of one or more disorders selected from the group consisting of inflammatory disorders and/or autoimmune diseases and/or allergic disorders, comprising administering a compound according to claim 1 to a patient afflicted therewith.

15. A method for treating and/or providing prophylaxis of psoriasis and/or psoriatic arthritis and/or rheumatoid arthritis and/or inflammatory bowel disease and/or asthma and/or allergic rhinitis, comprising administering a compound according to claim 1 to a patient afflicted therewith.

16. The compound of claim 1, in the form of a single stereoisomer or a mixture of stereoisomers.

17. the compound of claim 9, wherein:
(A) the C₃₋₆-cycloalkyl is selected from the group consisting of cyclopropyl, cyclobutyl, cyclopentyl and cyclohexyl;
(B) the 3 to 7 membered heterocycloalkyl is selected form the group consisting of oxetanyl, pyrrolidinyl, pyrrolinyl, pyrazolinyl, isoxazolinyl, oxazolinyl, isoxazolinyl, oxadiazolinyl, tetrahydropyranyl, dihydropyrazinyl, piperidinyl and morpholinyl,
(C) the heteroaryl is selected from the group consisting of thiazolyl, thiadiazolyl, oxazolyl, isoxazolyl, thienyl, pyrrolyl, pyrazolyl, oxadiazolyl, tetrazolyl, triazolyl, pyridyl, pyrazinyl and pyrimidinyl, or
(D) two or more of (A), (B) and (C).

18. The compound of claim 12, in the form of a single stereoisomer or a mixture of stereoisomers.

19. The compound of claim 12, in the form a physiologically acceptable solvate.

20. The pharmaceutical composition of claim 13, further comprising one or more of:
(A) one or more suitable, pharmaceutically compatible auxiliaries, and
(B) one or more further pharmacologically active compounds.
